(19) United States
(12) Patent Application Publication
(10) Pub. No.: US 2022/0144815 A1
(43) Pub. Date: May 12, 2022

(54) 1,3,4-OXADIAZOLES AND THEIR DERIVATIVES AS NEW ANTIFUNGAL AGENTS

(71) Applicant: Bayer Aktiengesellschaft, Leverkusen (DE)

(72) Inventors: Stephane BRUNET, Saint Andre De Corcy (FR); Pierre-Yves COQUERON, Lyon (FR); Philippe DESBORDES, Lyon (FR); Sophie DUCERF, Chasselay (FR); Jeremy DUFOUR, Lyon (FR); Andreas GOERTZ, Dormagen (DE); Emmanuelle HILT, Dizimieu (FR); Aurelie MALLINGER, Collonges-au-mont-D'or (FR); Sebastien NAUD, Collonges au Mont d'Or (FR); Anne-Sophie REBSTOCK, Champagne au Mont d'or (FR); Vincent THOMAS, Lyon (FR)

(21) Appl. No.: 17/415,496

(22) PCT Filed: Dec. 20, 2019
(86) PCT No.: PCT/EP2019/086681

§ 371 Date: Jun. 17, 2021

(30) Foreign Application Priority Data
Dec. 21, 2018 (EP) 18215674.5

(51) Int. Cl. C07D 413/04; C07D 413/14; A01N 43/82; A01P 3/00

(52) U.S. Cl. CPC C07D 413/04; A01P 3/00; A01N 43/82; C07D 413/14

(57) ABSTRACT

The present disclosure relates to the use of 1,3,4-oxadiazoles and derivatives thereof as fungicide. It also relates to new 1,3,4-oxadiazoles derivatives, their use as fungicide and compositions comprising thereof.

### CROSS-REFERENCE TO RELATED APPLICATIONS

This application is the National Stage entry of International Application No. PCT/EP2019/086681, filed 20 Dec. 2019, which claims priority to European Patent Application No. 18215674.5, filed 21 Dec. 2018.

### BACKGROUND

### Field

The present invention relates to the use of 1,3,4-oxadiazoles and derivatives thereof as fungicide. It also relates to new 1,3,4-oxadiazoles derivatives, their use as fungicide and compositions comprising thereof.

### Description of Related Art

1,2,4-oxadiazoles derivatives are well known to be useful as crop protection agents to combat or prevent microorganisms' infestations. For instance, WO-2018/118781 and WO-2018/080859 disclose 1,2,4-oxadiazol-3-ylpyrimidines and 1,2,4-oxadiazol-3-ylpyridines derivatives that may be used for the control of microbial pests, particularly fungal pests, on plants. Fungicidally active 1,2,4-oxadiazoles are also known from US 2018/317490.

On the other hand, 1,3,4-oxadiazoles derivatives are far less common and seldom used for the control of microbial pests. For instance, WO-2018/165520, WO-2017/065473 and WO-2017/023133 disclose 1,3,4-oxadiazol-2-ylpyrimidines and 1,3,4-oxadiazol-2-ylpyridines derivatives that may be used as metalloenzyme (histone deacetylase) inhibitors for the treatment of many human diseases.

US 2016/0157489 discloses tetrazolinone compounds comprising a pyrimidine or pyridine ring and their use for pest control.

Numerous fungicidal agents have been developed until now. However, the need remains for the development of new fungicidal compounds as such, so as to provide compounds being effective against a broad spectrum of fungi, having lower toxicity, higher selectivity, being used at lower dosage rate to reduce or avoid unfavorable environmental or toxicological effects whilst still allowing effective pest control. It may also be desired to have new compounds to prevent the emergence of fungicides resistances.

The present invention provides new fungicidal compounds which have advantages over known compounds and compositions in at least some of these aspects.

### SUMMARY

The present invention also relates to the use of a compound of formula (I) as defined herein for controlling phytopathogenic fungi:

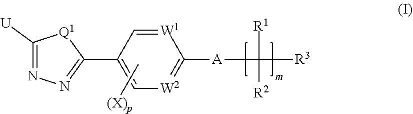

The present invention relates to compounds of the formula (I):

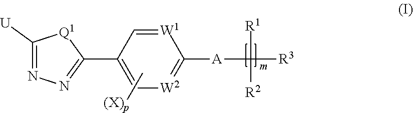

wherein R¹, R², R³, m, A, W¹, W², Q¹, X, p and U are as recited herein as well as their salts, N-oxides and solvates.

The present invention relates to a composition comprising at least one compound of formula (I) as defined herein and at least one agriculturally suitable auxiliary.

### Definitions

The term “halogen” as used herein refers to fluorine, chlorine, bromine or iodine atom.

The term “oxo” as used herein refers to an oxygen atom which is bound to a carbon atom or sulfur atom via a double bound.

The term “C₁-C₈-alkyl” as used herein refers to a saturated, branched or straight hydrocarbon chain having 1, 2, 3, 4, 5, 6, 7 or 8 carbon atoms. Examples of C₁-C₈-alkyl include but are not limited to methyl, ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Particularly, said hydrocarbon chain has 1, 2, 3 or 4 carbon atoms (“C₁-C₄-alkyl”), e.g. methyl, ethyl, propyl, iso-propyl, butyl, sec-butyl, iso-butyl or tert-butyl.

The term “C₂-C₈-alkenyl” as used herein refers to an unsaturated, branched or straight hydrocarbon chain having 2, 3, 4, 5, 6, 7 or 8 carbon atoms and comprising at least one double bond. Examples of C₂-C₈-alkenyl include but are not limited to ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl, hexa-1,5-dienyl or methylhexadienyl group.

The term “C₂-C₈-alkynyl” as used herein refers to a branched or straight hydrocarbon chain having 2, 3, 4, 5, 6, 7 or 8 carbon atoms and comprising at least one triple bond. Examples of C₂-C₈-alkynyl include but are not limited to ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₁-C₈-halogenoalkyl” or “C₁-C₈-haloalkyl” as used herein refers to a C₁-C₈-alkyl group as defined above in which one or more hydrogen atoms are replaced with one or more halogen atoms that may be the same or different. Typically, C₁-C₈-halogenoalkyl comprises up to 9 halogen atoms that can be the same or different.

The term “C₂-C₈-haloalkenyl” as used herein refers to a C₂-C₈-alkenyl group as defined above in which one or more hydrogen atoms are replaced with one or more halogen atoms that may be the same or different. Typically, C₁-C₈-halogenoalkenyl comprises up to 9 halogen atoms that can be the same or different.

The term “C₂-C₈-haloalkynyl” as used herein refers to a C₂-C₈-alkynyl group as defined above in which one or more hydrogen atoms are replaced with one or more halogen atoms that may be the same or different. Typically, C₁-C₈-halogenoalkynyl comprises up to 9 halogen atoms that can be the same or different.

The term “C₁-C₈-alkoxy” as used herein refers to a group of formula (C₁-C₈-alkyl)-O—, in which the term “C₁-C₈-alkyl” is as defined herein. Examples of C₁-C₈-alkoxy include but are not limited to methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, 1,1-dimethylpropoxy, 1,2-dimethylpropoxy, n-hexyloxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy.

The term “C₁-C₈-halogenalkoxy” or “C₁-C₈-haloalkoxy” as used herein refers to a C₁-C₈-alkoxy group as defined above in which one or more hydrogen atoms are replaced with one or more halogen atoms that may be the same or different. Examples of C₁-C₈-haloalkoxy include but are not limited to chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy.

The term “C₁-C₈-alkylsulfanyl” as used herein refers to a saturated, linear or branched group of formula (C₁-C₈-alkyl)-S—, in which the term “C₁-C₈-alkyl” is as defined herein. Examples of C₁-C₈-alkylsulfanyl include but are not limited to methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₈-halogenoalkylsulfanyl” as used herein refers to a C₁-C₈-alkylsulfanyl as defined above in which one or more hydrogen atoms are replaced with one or more halogen atoms that may be the same or different.

The term “C₁-C₈-alkylsulfinyl” s used herein refers to a saturated, linear or branched group of formula (C₁-C₈-alkyl)-S(═O)—, in which the term “C₁-C₈-alkyl” is as defined herein. Examples of C₁-C₈-alkylsulfinyl include but are not limited to saturated, straight-chain or branched alkylsulfinyl radicals having 1 to 8, preferably 1 to 6 and more preferably 1 to 4 carbon atoms, for example (but not limited to) methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

The term “C₁-C₈-halogenoalkylsulfinyl” as used herein refers to a C₁-C₈-alkylsulfinyl as defined above in which one or more hydrogen atoms are replaced with one or more halogen atoms that may be the same or different.

The term “C₁-C₈-alkylsulfonyl” s used herein refers to a saturated, linear or branched group of formula (C₁-C₈-alkyl)-S(═O)₂—, in which the term “C₁-C₈-alkyl” is as defined herein. Examples of C₁-C₈-alkylsulfonyl include but are not limited to methylsulfonyl, ethylsulfonyl, propylsulfonyl, 1-methyl-ethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 2,2-dimethylpropylsulfonyl, 1-ethylpropylsulfonyl, 1,1-dimethylpropylsulfonyl, 1,2-dimethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methyl-propylsulfonyl and 1-ethyl-2-methylpropylsulfonyl.

The term “C₁-C₈-halogenoalkylsulfonyl” as used herein refers to a C₁-C₈-alkylsulfonyl as defined above in which one or more hydrogen atoms are replaced with one or more halogen atoms that may be the same or different.

The term “C₁-C₈-alkylcarbonyl” as used herein refers to a saturated, linear or branched group of formula (C₁-C₈-alkyl)-C(═O)—, in which the term “C₁-C₈-alkyl” is as defined herein.

The term “C₁-C₈-halogenoalkylcarbonyl” as used herein refers to a C₁-C₈-alkylcarbonyl as defined above in which one or more hydrogen atoms are replaced with one or more halogen atoms that may be the same or different.

The term “C₁-C₈-alkoxycarbonyl” as used herein refers to a saturated, linear or branched group of formula (C₁-C₈-alkoxy)-C(═O)—, in which the term “C₁-C₈-alkoxy” is as defined herein.

The term “C₁-C₈-haloalkoxycarbonyl” as used herein refers to a C₁-C₈-alkoxycarbonyl as defined above in which one or more hydrogen atoms are replaced with one or more halogen atoms that may be the same or different.

The term “non-aromatic C₃-C₁₂-carbocycle” as used herein refers to a non-aromatic, saturated or unsaturated, hydrocarbon ring system in which all of the ring members, which vary from 3 to 12, are carbon atoms. The ring system may be monocyclic or polycyclic (fused, spiro or bridged). Non-aromatic C₃-C₁₂-carbocycles include but are not limited to C₃-C₁₂-cycloalkyl (mono or bicyclic), C₃-C₁₂-cycloalkenyl (mono or bicyclic), bicylic system comprising an aryl (e.g. phenyl) fused to a monocyclic C₃-C₇-cycloalkyl (e.g. tetrahydronaphthalenyl, indanyl), bicylic system comprising an aryl (e.g. phenyl) fused to a monocyclic C₃-C₈-cycloalkenyl (e.g. indenyl, dihydronaphthalenyl) and tricyclic system comprising a cyclopropyl connected through one carbon atom to a bicylic system comprising an aryl (e.g. phenyl) fused to a monocyclic C₃-C₇-cycloalkyl or to a monocyclic C₃-C₈-cycloalkenyl. The non-aromatic C₃-C₁₂-carbocycle can be attached to the parent molecular moiety through any carbon atom.

The term “C₃-C₁₂-cycloalkyl” as used herein refers to a saturated, monovalent, mono- or bicylic hydrocarbon ring which contains 3, 4, 5, 6, 7, 8, 9, 10, 11 or 12 carbon atoms. “C₃-C₇-cycloalkyl” as used herein designates monocyclic C₃-C₇-cycloalkyls which include but are not limited to cyclopropyl, cyclobutyl, cyclopentyl or cyclohexyl, cycloheptyl. Examples of bicyclic C₆-C₁₂-cycloalkyls include but are not limited to bicyclo[3.1.1]heptane, bicyclo[2.2.1]heptane, bicyclo[2.2.2]octane, bicyclo[3.2.2]nonane, bicyclo[3.3.1]nonane, bicyclo[4.2.0]octyl, octahydropentalenyl and bicyclo[4.2.1]nonane.

The term “C₃-C₁₂-cycloalkenyl” as used herein refers to an unsaturated, monovalent, mono- or bicylic hydrocarbon ring which contains 3, 4, 5, 6, 7, 8, 9, 10, 11 or 12 carbon atoms. Examples of monocyclic C₃-C₈-cycloalkenyl group include but are not limited to cyclobutenyl, cyclopentenyl, cyclohexenyl, cycloheptenyl and cyclooctenyl group. Examples of bicyclic C₆-C₁₂-cycloalkenyl group include but are not limited to bicyclo[2.2.1]hept-2-enyl or bicyclo[2.2.2]oct-2-enyl.

The term “aromatic C₆-C₁₄-carbocycle” or “aryl” as used herein refers to an aromatic hydrocarbon ring system in which all of the ring members, which vary from 6 to 14, preferably from 6 to 10, are carbon atoms. The ring system may be monocyclic or fused polycyclic (e.g. bicyclic or tricyclic). Examples of aryl include but are not limited to phenyl, azulenyl, naphthyl and fluorenyl. The aryl can be attached to the parent molecular moiety through any carbon atom. It is further understood that when said aryl group is substituted with one or more substituents, said substituent(s) may be at any positions on said aryl ring(s). Particularly, in the case of aryl being a phenyl group, said substituent(s) may occupy one or both ortho positions, one or both meta positions, or the para position, or any combination of these positions.

The term “non-aromatic 3- to 10-membered heterocycle” or “heterocyclyl” as used herein refers to a saturated or partially unsaturated non-aromatic ring system comprising 1 to 4, or 1 to 3 heteroatoms independently selected from the group consisting of oxygen, nitrogen and sulfur. If the ring system contains more than one oxygen atoms, they are not directly adjacent. Non aromatic heterocycles include but are not limited to 3- to 7-membered monocyclic non-aromatic heterocycles and 6- to 10-membered polycyclic (e.g. bicyclic or tricyclic) non-aromatic heterocycles. The non-aromatic 3- to 10-membered heterocycle can be connected to the parent molecular moiety through any carbon atom or nitrogen atom contained within the heterocycle.

The term “non-aromatic 3- to 7-membered monocyclic heterocycle” as used herein refers to a 3-, 4-, 5-, 6- or 7-membered monocyclic ring system containing 1, 2 or 3 heteroatoms independently selected from the group consisting of oxygen, nitrogen and sulfur where the ring system is saturated or unsaturated but not aromatic. For instance, the heterocycle may comprise one to three nitrogen atoms, or one or two oxygen atoms, or one or two sulfur atoms, or one to three nitrogen atoms and one oxygen atom, or one to three nitrogen atoms and a sulfur atom or one sulfur atom and one oxygen atom.

Examples of saturated non-aromatic heterocycles include but are not limited to 3-membered ring such as oxiranyl, aziridinyl, 4-membered ring such as azetidinyl, oxetanyl, thietanyl, 5-membered ring such as tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, 6-membered ring such as piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl or 7-membered ring such as oxepanyl, azepanyl, 1,4-diazepanyl and 1,4-oxazepanyl. Examples of unsaturated non-aromatic hererocyles include but are not limited to 5-membered ring such as dihydrofuranyl, 1,3-dioxolyl, dihydrothienyl, pyrrolinyl, dihydroimidazolyl, dihydropyrazolyl, isoxazolinyl, dihydrooxazolyl, dihydrothiazolyl or 6-membered ring such as pyranyl, thiopyranyl, thiazinyl and thiadiazinyl.

The term “non-aromatic 6- to 10-membered polycyclic heterocycle” as used herein refers to a 6-, 7-, 8-, 9-, 10-membered polycyclic (e.g. bicyclic or tricyclic) ring system containing 1, 2 or 3 heteroatoms independently selected from the group consisting of oxygen, nitrogen and sulfur where the ring system is saturated or unsaturated but not aromatic. Non-aromatic bicyclic heterocycles may consist of a monocyclic heteroaryl as defined herein fused to a monocyclic C₃-C₇-cycloalkyl, a monocyclic C₃-C₈-cycloalkenyl or a monocyclic non-aromatic heterocycle or may consist of a monocyclic non-aromatic heterocycle fused either to an aryl (e.g. phenyl), a monocyclic C₃-C₇-cycloalkyl, a monocyclic C₃-C₈-cycloalkenyl or a monocyclic non-aromatic heterocycle. When two monocyclic heterocycles (aromatic or non-aromatic) comprising nitrogen atoms are fused, nitrogen atom may be at the bridgehead (e.g. 4,5,6,7-tetrahydropyrazolo[1,5-a]pyridinyl, 5,6,7,8-tetrahydro-[1,2,4]triazolo[1,5-a]pyridinyl, 5,6,7,8-tetrahydroimidazo[1,2-a]pyridinyl). Non-aromatic tricyclic heterocycles may consist of a monocyclic cycloalkyl connected through one common atom to a non-aromatic bicyclic heterocycle.

The term “aromatic 5- to 14-membered heterocycle” or “heteroaryl” as used herein refers to an aromatic ring system comprising 1 to 4 heteroatoms independently selected from the group consisting of oxygen, nitrogen and sulfur. If the ring system contains more than one oxygen atom, they are not directly adjacent. Aromatic heterocycles include aromatic 5- or 6-membered monocyclic heterocycles and 6- to 14-membered polycyclic (e.g. bicyclic or tricyclic) aromatic heterocycles. The 5- to 14-membered aromatic heterocycle can be connected to the parent molecular moiety through any carbon atom or nitrogen atom contained within the heterocycle.

The term “aromatic 5- or 6-membered monocyclic heterocycle” or “monocyclic heteroaryl” as used herein refers to a 5- or 6-membered monocyclic ring system containing 1, 2, 3 or 4 heteroatoms independently selected from the group consisting of oxygen, nitrogen and sulfur. Examples of 5-membered monocyclic heteroaryl include but are not limited to furyl (furanyl), thienyl, pyrrolyl, pyrazolyl, imidazolyl, triazolyl, tetrazolyl, isoxazolyl, oxazolyl, oxadiazolyl, oxatriazolyl, isothiazolyl, thiazolyl, thiadiazolyl and thiatriazolyl. Examples of 6-membered monocyclic heteroaryl include but are not limited to pyridinyl, pyridazinyl, pyrimidinyl, pyrazinyl, triazinyl, tetrazinyl.

The term “6- to 14-membered polycyclic aromatic heterocycle” or “polycyclic heteroaryl” as used herein refers to a 6-, 7-, 8-, 9-, 10-, 11-, 12-, 13- or 14-membered polycyclic (e.g. bicyclic or tricyclic) ring system containing 1, 2 or 3 heteroatoms independently selected from the group consisting of oxygen, nitrogen and sulfur. Aromatic bicyclic heterocycles may consist of a monocyclic heteroaryl as defined herein fused to an aryl (e.g. phenyl) or to a monocyclic heteroaryl. Examples of bicyclic aromatic heterocycle include but are not limited to 9-membered ring such as indolyl, indolizinyl, isoindolyl, benzimadozolyl, imidazopyridinyl, indazolyl, benzotriazolyl, purinyl, benzofuranyl, benzothiophenyl, benzothiazolyl, benzoxazolyl and benzisoxazolyl or 10-membered ring such as quinolinyl, isoquinolinyl, cinnolinyl, quinazolinyl, quinoxalinyl, phthalazinyl, naphthyridinyl, pteridinal and benzodioxinyl. In 9- or 10-membered aromatic bicyclic heterocycles comprising two fused 5- or 6-membered monocyclic aromatic heterocycles, nitrogen atom may be at the bridgehead (e.g. imidazo[1,2-a]pyridinyl, [1,2,4]triazolo[4,3-a]pyridinyl, imidazo[1,2-a]pyridinyl, imidazo[2,1-b]oxazolyl, furo[2,3-d]isoxazolyl). Examples of tricyclic aromatic heterocyle include but are not limited to carbazolyl, acridinyl and phenazinyl.

The terms “non-aromatic C₃-C₁₂-carbocyclyloxy”, “C₃-C₇-cycloalkyloxy”, “aromatic C₆-C₁₄-carbocyclyloxy”, “aromatic 5- to 10-membered heterocyclyloxy”, “non-aromatic 5- to 10-membered heterocyclyloxy” as used herein designate a group of formula —O—R wherein R is respectively a non-aromatic C₃-C₁₂-carbocyclyl, a C₃-C₇-cycloalkyl, an aromatic C₆-C₁₄-carbocyclyl, an aromatic 5- to 14-membered heterocyclyl or a non-aromatic 5- to 14-membered heterocyclyl group as defined herein.

As used herein, when a group is said to be “substituted”, the group may be substituted with one or more substituents. The expression “one or more substituents” refers to a number of substituents that ranges from one to the maximum number of substituents possible based on the number of available bonding sites, provided that the conditions of stability and chemical feasibility are met.

The term “leaving group” as used herein is to be understood as meaning a group which is displaced from a compound in a substitution or an elimination reaction, for example a halogen atom, a trifluoromethanesulfonate (“triflate”) group, alkoxy, methanesulfonate, p-toluenesulfonate, etc.

The terms “as described herein” when referring to a variable xxxx incorporates by reference the broad definition of the variable as well as preferred, more preferred and even more preferred definitions, if any.

### DETAILED DESCRIPTION

The present invention relates to the use of compounds of formula (I) for controlling phytopathogenic fungi:

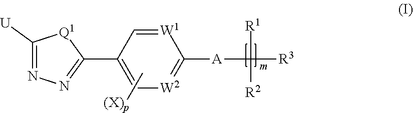

wherein
U is a C₁-C₃-haloalkyl comprising 2 to 7 halogen atoms that can be the same or different and selected from the group consisting of fluorine and chlorine;
Q¹ is O or S;
W¹ and W² are independently N, CH or CF;
A is selected from the group consisting of direct bond, O, S, S═O, S(═O)₂, NR⁴, —(C═O)—, —(C═S)—, —O—(C═O), —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)═O—;
m=0, 1 or 2, wherein, if m is 2, the two [CR¹R²] groups may be the same or different;
p=0, 1 or 2;
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with, respectively, one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with, respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of O, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted with, respectively one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that compound of formula (I) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
2-isopropyl-5,6-dimethyl-3-{4-[5-(trifluoromethyl)-1,3,4-oxadiazol-2-yl]benzyl}pyridin-4-ol [2133324-02-2];

as well as their salts, N-oxides, solvates and optically active isomers or geometric isomers.

Not encompassed herein are compounds resulting from combinations which are against natural laws and which the person skilled in the art would therefore exclude based on his/her expert knowledge. For instance, ring structures having three or more adjacent oxygen atoms are excluded.

According to the invention, the compounds of formula (I) can be used for controlling phytopathogenic fungi in plants, plant parts, seeds, fruits or the soil in which the plants grow. Preferably, the phytopathogenic fungi are selected from the group consisting of the Puccinia species, for example Puccinia recondita, Puccinia graminis or Puccinia striiformis; the Uromyces species, for example Uromyces appendiculatus; and the rust disease pathogens, in particular selected from the group consisting of the Gymnosporangium species, for example Gymnosporangium sabinae; Hemileia species, for example Hemileia vastatrix, and Phakopsora species, for example Phakopsora pachyrhizi or Phakopsora meibomiae. Especially preferred are the rust disease pathogens, in particular Phakopsora pachyrhizi and Phakopsora meibomiae.

The compounds of formula (I) can suitably be in their free form, salt form, N-oxide form or solvate form (e.g. hydrate).

Depending on the nature of the substituents, the compound of formula (I) may be present in the form of different stereoisomers. These stereoisomers are, for example, enantiomers, diastereomers, atropisomers or geometric isomers. Accordingly, the invention encompasses both pure stereoisomers and any mixture of these isomers. Where a compound can be present in two or more tautomer forms in equilibrium, reference to the compound by means of one tautomeric description is to be considered to include all tautomer forms.

Any of the compounds of the present invention can also exist in one or more geometric isomer forms depending on the number of double bonds in the compound. Geometric isomers by nature of substituents about a double bond or a ring may be present in cis (=Z-) or trans (=E-) form. The invention thus relates equally to all geometric isomers and to all possible mixtures, in all proportions.

Depending on the nature of the substituents, the compound of formula (I) may be present in the form of the free compound and/or a salt thereof, such as an agrochemically active salt.

Agrochemically active salts include acid addition salts of inorganic and organic acids well as salts of customary bases. Examples of inorganic acids are hydrohalic acids, such as hydrogen fluoride, hydrogen chloride, hydrogen bromide and hydrogen iodide, sulfuric acid, phosphoric acid and nitric acid, and acidic salts, such as sodium bisulfate and potassium bisulfate. Useful organic acids include, for example, formic acid, carbonic acid and alkanoic acids such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, saturated or mono- or diunsaturated fatty acids having 6 to 20 carbon atoms, alkylsulfuric monoesters, alkylsulfonic acids (sulfonic acids having straight-chain or branched alkyl radicals having 1 to 20 carbon atoms), arylsulfonic acids or aryldisulfonic acids (aromatic radicals, such as phenyl and naphthyl, which bear one or two sulfonic acid groups), alkylphosphonic acids (phosphonic acids having straight-chain or branched alkyl radicals having 1 to 20 carbon atoms), arylphosphonic acids or aryldiphosphonic acids (aromatic radicals, such as phenyl and naphthyl, which bear one or two phosphonic acid radicals), where the alkyl and aryl radicals may bear further substituents, for example p-toluenesulfonic acid, salicylic acid, p-aminosalicylic acid, 2-phenoxybenzoic acid, 2-acetoxybenzoic acid, etc.

Solvates of the compounds of the invention or their salts are stoichiometric compositions of the compounds with solvents.

The compounds of the invention may exist in multiple crystalline and/or amorphous forms. Crystalline forms include unsolvated crystalline forms, solvates and hydrates.

Compounds of formula (I) are herein referred to as “active ingredient(s)”.

In the above formula (I), U is preferably C₁-haloalkyl comprising 2 to 3 halogen atoms that can be the same or different and selected from the group consisting of fluorine and chlorine. More preferably, U is selected from CHF₂, CClF₂ and CF₃, still more preferably, U is CHF₂ or CF₃, even more preferably, U is CHF₂.

In some embodiments, in the above formula (I), Q¹ is preferably O.

In the above formula (I), W¹ is preferably N or CH.

In the above formula (I), W² is preferably N or CH.

In some embodiments in the above formula (I), W¹ and W² are N, or W¹ is N and W² is CH, more preferably, W¹ and W² are N.

In the above formula (I), A is preferably a direct bond, O, S, NR⁴, —N(R⁴)—(C═O), —(C═O)—O—, —(C═O)—N(R⁵)—, —(C═O)—N(R⁴)—N(R⁵)—, —N(R⁴)—N(R⁵)—, —N(R⁴)—(C═O)—O— or —N(R⁴)—(C═O)—N(R⁵)—.

In some embodiments, A is a direct bond, O, NR⁴, —N(R⁴)—(C═O)—, —(C═O)—O— or —(C═O)—N(R⁵)—, more preferably O, NH, —(C═O)—O—, —(C═O)—NH— or —(C═O)—NMe-, even more preferably O or NH.

In some embodiments, in above formula (I), A is NR⁴, preferably NH.

In some embodiments, in above formula (I), A is O.

In some embodiments, in the above formula (I), m is preferably 0 or 1, more preferably m is 1.

In the above formula (I), p is preferably 0 or 1, more preferably p is 0.

In the above formula (I), each R¹ and each R² are preferably independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or
R¹ and R² may form, together with the carbon atom to which they are linked, a C₃-C₇-cycloalkyl or oxetanyl ring, or
two consecutive R¹, when m is 2, may form, together with the carbon atoms to which they are linked, a C₃-C₇-cycloalkyl ring.

In the above formula (I), it is more preferred that R¹ is selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl, and R² is selected from the group consisting of hydrogen, fluorine, chlorine, methyl, ethyl, trifluoromethyl and difluoromethyl, or
R¹ and R² may form, together with the carbon atom to which they are linked, a cyclopropyl, cyclobutyl, cyclopentyl, cyclohexyl or oxetanyl ring, or
two consecutive R¹, when m is 2, may form, together with the carbon atoms to which they are linked, a cyclopropyl, cyclobutyl or cyclopentyl ring.

In the above formula (I), it is even more preferred that
m is 1,
R¹ is selected from the group consisting of hydrogen, methyl, ethyl, trifluoromethyl, ethynyl and cyclopropyl, and
R² is hydrogen,

or
R¹ and R² form, together with the carbon atom to which they are linked, a cyclopropyl, cyclobutyl or oxetanyl ring, more preferably a cyclopropyl or cyclobutyl ring, and most preferably a cyclopropyl ring.

In some embodiments, in above formula (I), there is at least [CR¹R²] group, in which R¹ and R² form, together with the carbon atom to which they are linked, a cyclopropyl, cyclobutyl, cyclopentyl, cyclohexyl or oxetanyl ring,

wherein, if m is 2, R¹ and R² of the other [CR¹R²] group are independently selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl, or form, together with the carbon atom to which they are linked, a cyclopropyl, cyclobutyl, cyclopentyl, cyclohexyl or oxetanyl ring.

In some embodiments, in above formula (I), m is 1, and R¹ and R² form, together with the carbon atom to which they are linked, a cyclopropyl, cyclobutyl, cyclopentyl, cyclohexyl or oxetanyl ring, more preferably a cyclopropyl, cyclobutyl or oxetanyl ring.

In some preferred embodiments, m is 1, and R¹ and R² form, together with the carbon atom to which they are linked, a cyclopropyl or cyclobutyl ring, preferably a cyclopropyl ring.

In some other preferred embodiments, m is 1, and R¹ and R² form, together with the carbon atom to which they are linked, a oxetanyl ring.

In still some other preferred embodiments,
m is 1,
R¹ is selected from the group consisting of hydrogen, methyl, ethyl, trifluoromethyl and cyclopropyl, and
R² is hydrogen.

In the above formula (I), R³ is preferably selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy, wherein said 1,3,2-dioxaborolan-2-yl may be substituted with one to four C₁-C₃-alkyl substituents, wherein said C₁-C₈-alkyl may be substituted with one C₁-C₈-alkoxy or C₁-C₈-haloalkoxy substituent, and wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl.

In the above formula (I), R³ is more preferably selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;

and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

In some preferred embodiments, R³ is selected from aryl and heteroaryl. In these embodiments, it is preferred that the aryl is selected from phenyl and naphthyl; the heteroaryl is selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; and the aryl and heteroaryl may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl

In some embodiments, R³ is selected from phenyl and pyridine, wherein the phenyl and the pyridine may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In these embodiments, R³ is more preferably phenyl, which is unsubstituted or substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl, preferably unsubstituted or substituted with one or two substituents independently selected from fluorine, chlorine, bromine, methyl and methoxy.

In the above formula (I), R⁴ and R⁵ are preferably independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms.

In the above formula (I), R⁴ and R⁵ are more preferably independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms.

In the above formula (I), R⁴ and R⁵ are most preferably independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms.

In some particularly preferred embodiments, R⁴ and R⁵ are hydrogen.

The above mentioned preferences with regard to the substituents of the compounds according to the invention can be combined in various manners. These combinations of preferred features thus provide sub-classes of compounds according to the invention. Examples of such sub-classes of preferred compounds according to the invention are:
preferred features of U with one or more preferred features of Q¹, W¹, W², A, p, m, R¹, R², R³, R⁴ and R⁵;
preferred features of Q¹ with one or more preferred features of U, W¹, W², A, p, m, R¹, R², R³, R⁴ and R⁵;
preferred features of W¹ with one or more preferred features of U, Q¹, W², A, p, m, R¹, R², R³, R⁴ and R⁵;
preferred features of W² with one or more preferred features of U, Q, W, A, p, m, R¹, R², R³, R⁴ and R⁵;
preferred features of A with one or more preferred features of U, Q¹, W¹, W², p, m, R¹, R², R³, R⁴ and R⁵;
preferred features of p with one or more preferred features of U, Q¹, W¹, W², A, m, R¹, R², R³, R⁴ and R⁵;
preferred features of m with one or more preferred features of U, Q¹, W¹, W², A, p, R, R², R³, R⁴ and R⁵;
preferred features of R¹ with one or more preferred features of U, Q¹, W¹, W², A, p, m, R², R³, R⁴ and R⁵;
preferred features of R² with one or more preferred features of U, Q¹, W¹, W², A, p, m, R¹, R³, R⁴ and R⁵;
preferred features of R³ with one or more preferred features of U, Q¹, W¹, W², A, p, m, R¹, R², R⁴ and R⁵;
preferred features of R⁴ with one or more preferred features of U, Q¹, W¹, W², A, p, m, R¹, R², R³, and R⁵;
preferred features of R⁵ with one or more preferred features of U, Q¹, W¹, W², A, p, m, R¹, R², R³ and R⁴;

In these combinations of preferred features of the substituents of the compounds according to the invention, the said preferred features can also be selected among the more preferred features of each of U, Q¹, W¹, W², A, p, m, R¹, R², R³, R⁴ and R⁵ so as to form most preferred subclasses of compounds according to the invention.

Preferred is the use of compounds of formula (I):

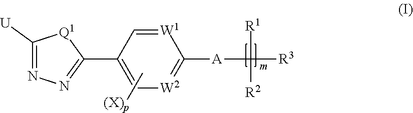

wherein

U is selected from CHF₂, CClF₂ and CF₃;

Q¹ is O or S;

W¹ and W² are independently N or CH;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0 or 1;

X is fluorine;

each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, preferably hydrogen, C₁-C₈-alkyl, C₁-C₈-halogenoalkyl C₂-C₈-alkynyl and C₃-C₇-cycloalkyl, wherein said C₁-C₈-alkyl may be substituted with one or more substituents selected from hydroxy and C₁-C₈-alkoxy, or

R¹ and R² may form, together with the carbon atom to which they are linked, a C₃-C₇-cycloalkyl or oxetanyl ring, or

two consecutive R¹, when m is 2, may form, together with the carbon atoms to which they are linked, a C₃-C₇-cycloalkyl ring;

R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;

R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms;

provided that compound of formula (I) is not
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],

as well as their salts, N-oxides, solvates and optically active isomers or geometric isomers.

Particularly preferred is the use of compounds of formula (I), wherein

U is selected from CHF₂, CClF₂ and CF₃, in particular CHF₂ or CF₃;

Q¹ is O or S, preferably O;

W¹ and W² are independently N or CH;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0;

R¹ is selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl,

or two consecutive R¹, when m is 2, may form, together with the carbon atoms to which they are linked, a cyclopropyl, cyclobutyl or cyclopentyl ring, and

R² is selected from the group consisting of hydrogen, fluorine, chlorine, methyl, ethyl, trifluoromethyl and difluoromethyl,

or

R¹ and R² may form, together with the carbon atom to which they are linked, a cyclopropyl, cyclobutyl, cyclopentyl, cyclohexyl or oxetanyl ring;

R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxy-ethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
preferably, R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and 2-naphthyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;

R⁴ and R⁵ are independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms, preferably R⁴ and R⁵ are independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms;

provided that compound of formula (I) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],

as well as their salts, N-oxides, solvates and optically active isomers or geometric isomers.

In some embodiments according to the particularly preferred use of the compounds of formula (I) as defined above, U is CHF₂.

In some embodiments according to the particularly preferred use of the compounds of formula (I) as defined above, W¹ and W² are N, or W¹ is N and W² is CH, preferably W¹ and W² are N.

In some embodiments according to the particularly preferred use of the compounds of formula (I) as defined above, A is a direct bond, O, S, NR⁴, —N(R⁴)—(C═O)—, —(C═O)—O— or —(C═O)—N(R⁵)—, more preferably O, S or NR⁴, even more preferably O or NR⁴, most preferably NH.

In some embodiments according to the particularly preferred use of the compounds of formula (I) as defined above,
R¹ is selected from the group consisting of hydrogen, methyl, ethyl, iso-propyl, trifluoromethyl, ethenyl, ethynyl, phenyl and cyclopropyl,
R² is hydrogen,

or
R¹ and R² form, together with the carbon atom to which they are linked, a cyclopropyl, cyclobutyl or oxetanyl ring, more preferably a cyclopropyl or cyclobutyl ring, and most preferably a cyclopropyl ring.

In some embodiments according to the particularly preferred use of the compounds of formula (I) as defined above, R³ is selected from the group consisting of C₃-C₇-cycloalkyl, C₃-C₈-cycloalkenyl, aryl, heterocyclyl and heteroaryl, preferably C₃-C₇-cycloalkyl, aryl, heterocyclyl and heteroaryl; more preferably aryl or heteroaryl, and most preferably aryl;

wherein C₃-C₇-cycloalkyl is selected from cyclopropyl, cyclobutyl, cyclopentyl and cyclohexyl;

wherein C₃-C₈-cycloalkenyl is selected from cyclopentenyl and cyclohexenyl;

wherein aryl is selected from phenyl and naphthyl;

wherein heterocyclyl is selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl, preferably selected from piperidin-1-yl, piperazin-1-yl, tetrahydro-2H-pyran-4-yl, tetrahydrothiopyran-4-yl, morpholin-4-yl and 1,3-dihydro-2H-isoindol-2-yl;

wherein heteroaryl is selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl, preferably selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl;

and wherein said C₃-C₇-cycloalkyl, C₃-C₈-cycloalkenyl, aryl, heterocyclyl and heteroaryl may be substituted with one to three substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In some of said embodiments, R³ is selected from phenyl and pyridinyl, wherein said phenyl and pyridinyl may be substituted with one to three substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In these embodiments, R³ is more preferably phenyl, which is unsubstituted or substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl, in particular unsubstituted phenyl or phenyl which is substituted with one or two substituents independently selected from fluorine, chlorine, bromine, methyl and methoxy.

In some embodiments according to the particularly preferred use of the compounds of formula (I) as defined above, R⁴ and R⁵ are hydrogen.

In some embodiments according to the particularly preferred use of the compounds of formula (I) as defined above,

U is CHF₂ or CF₃;

p is 0;

W¹ and W² are N; and

A is O or NR⁴.

In some embodiments of the use according to the invention, in formula (I), the substituents are defined as follows:

U is CHF₂ or CF₃;

Q¹ is O;

p is 0;

W¹ and W² are N;

A is O or NH;

m is 1;

R¹ is selected from the group consisting of hydrogen, C₁-C₈-alkyl, C₁-C₈-halogenoalkyl, C₂-C₈-alkynyl, C₂-C₈-alkenyl, C₃-C₇-cycloalkyl and phenyl,

R² is hydrogen,

or

R¹ and R² form, together with the carbon atom to which they are linked, a C₃-C₇-cycloalkyl or oxetanyl ring; and

R³ is selected from the group consisting of C₃-C₇-cycloalkyl, C₃-C₈-cycloalkenyl, aryl, heterocyclyl and heteroaryl,

wherein C₃-C₇-cycloalkyl is selected from cyclopropyl, cyclobutyl, cyclopentyl and cyclohexyl;

wherein C₃-C₈-cycloalkenyl is selected from cyclopentenyl and cyclohexenyl;

wherein aryl is selected from phenyl and naphthyl;

wherein heterocyclyl is selected from piperidin-1-yl, piperazin-1-yl, tetrahydro-2H-pyran-4-yl, tetrahydrothiopyran-4-yl, morpholin-4-yl and 1,3-dihydro-2H-isoindol-2-yl;

wherein heteroaryl is selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl;
and wherein said C₃-C₇-cycloalkyl, C₃-C₈-cycloalkenyl, aryl, heterocyclyl and heteroaryl may be substituted with one to three substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In said embodiments, it is more preferred that R¹ is selected from the group consisting of hydrogen, methyl, ethyl, isopropyl, trifluoromethyl, ethenyl, ethynyl, phenyl and cyclopropyl, and
R² is hydrogen,

or
R¹ and R² form, together with the carbon atom to which they are linked, a cyclopropyl, cyclobutyl or oxetanyl ring, more preferably a cyclopropyl or cyclobutyl ring, and most preferably a cyclopropyl ring.

In some of these embodiments, m is 1, and R¹ and R² form, together with the carbon atom to which they are linked, a cyclopropyl, cyclobutyl, cyclopentyl, cyclohexyl or oxetanyl ring, more preferably a cyclopropyl, cyclobutyl or oxetanyl ring.

In some of these embodiments, m is 1, and R¹ and R² form, together with the carbon atom to which they are linked, a cyclopropyl or cyclobutyl ring, preferably a cyclopropyl ring.

In some of these embodiments, m is 1, and R¹ and R² form, together with the carbon atom to which they are linked, a oxetanyl ring.

In some of these embodiments,
m is 1,
R¹ is selected from the group consisting of hydrogen, methyl, ethyl, isopropyl, trifluoromethyl, ethenyl, ethynyl, phenyl and cyclopropyl, and
R² is hydrogen.

In some embodiments of the use according to the invention, the substituents are defined as follows:

U is CHF₂ or CF₃;

Q¹ is O;

p is 0;

W¹ and W² are N;

A is O or NH;

m is 1;

R¹ is selected from the group consisting of hydrogen, methyl, ethyl, isopropyl, trifluoromethyl, ethenyl, ethynyl, phenyl and cyclopropyl, and

R² is hydrogen,

or

R¹ and R² form, together with the carbon atom to which they are linked, a cyclopropyl, cyclobutyl or oxetanyl ring; and

R³ is selected from phenyl and pyridine, wherein the phenyl and the pyridine may be substituted with one to three substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

Most preferred is the use of compounds of formula (I) selected from the group consisting of
I.001 4-[5-(difluoromethyl)-1,3,4-oxadiazol-2-yl]benzoic acid
I.002 2-(difluoromethyl)-5-[4-(methylsulfanyl)phenyl]-1,3,4-oxadiazole
I.003 methyl 4-[5-(difluoromethyl)-1,3,4-oxadiazol-2-yl]benzoate
I.004 4-[5-(trifluoromethyl)-1,3,4-oxadiazol-2-yl]benzoic acid
I.005 2-(difluoromethyl)-5-[4-(1H-1,2,3-triazol-1-yl)phenyl]-1,3,4-oxadiazole
I.006 methyl 4-[5-(trifluoromethyl)-1,3,4-oxadiazol-2-yl]benzoate
I.007 2-([biphenyl]-4-yl)-5-(difluoromethyl)-1,3,4-oxadiazole
I.008 4-[5-(trifluoromethyl)-1,3,4-thiadiazol-2-yl]benzoic acid
I.009 2-(4-bromophenyl)-5-(difluoromethyl)-1,3,4-oxadiazole
I.010 2-(difluoromethyl)-5-[4-(1H-imidazol-1-ylmethyl)phenyl]-1,3,4-oxadiazole
I.011 methyl 4-[5-(trifluoromethyl)-1,3,4-thiadiazol-2-yl]benzoate
I.012 N-{4-[5-(difluoromethyl)-1,3,4-oxadiazol-2-yl]phenyl}-2,2-difluoroacetamide
I.013 N-{4-[5-(difluoromethyl)-1,3,4-oxadiazol-2-yl]phenyl}pyrimidin-2-amine
I.014 2-([biphenyl]-4-yl)-5-(trifluoromethyl)-1,3,4-oxadiazole
I.015 5-{4-[5-(difluoromethyl)-1,3,4-oxadiazol-2-yl]phenyl}-3-ethyl-1,2,4-oxadiazole
I.016 4-[5-(difluoromethyl)-1,3,4-oxadiazol-2-yl]-N-(2-methoxyethyl)benzamide
I.017 N-{4-[5-(difluoromethyl)-1,3,4-oxadiazol-2-yl]phenyl}-2-fluoroaniline
I.018 3-ethyl-5-{4-[5-(trifluoromethyl)-1,3,4-oxadiazol-2-yl]phenyl}-1,2,4-oxadiazole
I.019 2,2′-(1,4-phenylene)bis[5-(difluoromethyl)-1,3,4-oxadiazole]
I.020 N-(2-methoxyethyl)-4-[5-(trifluoromethyl)-1,3,4-oxadiazol-2-yl]benzamide
I.021 2-(difluoromethyl)-5-[4-(4,4,5,5-tetramethyl-1,3,2-dioxaborolan-2-yl)phenyl]-1,3,4-oxadiazole
I.022 4-[5-(difluoromethyl)-1,3,4-oxadiazol-2-yl]-N-methyl-N-phenylbenzamide
I.023 N-(2-methoxyethyl)-4-[5-(trifluoromethyl)-1,3,4-thiadiazol-2-yl]benzamide
I.024 2-{4-[(4-chlorophenoxy)methyl]phenyl}-5-(difluoromethyl)-1,3,4-oxadiazole
I.025 4-[5-(difluoromethyl)-1,3,4-oxadiazol-2-yl]-N-[1-(2-fluorophenyl)cyclopropyl]aniline
I.026 N-methyl-N-phenyl-4-[5-(trifluoromethyl)-1,3,4-oxadiazol-2-yl]benzamide
I.027 4-[5-(difluoromethyl)-1,3,4-oxadiazol-2-yl]-N-(2,4-difluorophenyl)benzamide
I.028 N-(2,4-difluorophenyl)-4-[5-(trifluoromethyl)-1,3,4-oxadiazol-2-yl]benzamide
I.029 N-(2,4-difluorophenyl)-4-[5-(trifluoromethyl)-1,3,4-thiadiazol-2-yl]benzamide
I.030 5-[5-(difluoromethyl)-1,3,4-oxadiazol-2-yl]pyridin-2-amine
I.031 2-chloro-5-[5-(difluoromethyl)-1,3,4-oxadiazol-2-yl]pyridine
I.032 5-[5-(difluoromethyl)-1,3,4-oxadiazol-2-yl]pyridine-2-carboxylic acid
I.033 5-[5-(difluoromethyl)-1,3,4-oxadiazol-2-yl]-N-methylpyridine-2-carboxamide
I.034 N-{5-[5-(difluoromethyl)-1,3,4-oxadiazol-2-yl]pyridin-2-yl}acetamide
I.035 5-[5-(difluoromethyl)-1,3,4-oxadiazol-2-yl]-2-(2-furyl)pyridine
I.036 5-[5-(difluoromethyl)-1,3,4-oxadiazol-2-yl]-2-(1H-pyrazol-1-yl)pyridine
I.037 5-[5-(difluoromethyl)-1,3,4-oxadiazol-2-yl]-2-(trifluoromethyl)pyridine
I.038 methyl {5-[5-(difluoromethyl)-1,3,4-oxadiazol-2-yl]pyridin-2-yl}carbamate
I.039 5-[5-(difluoromethyl)-1,3,4-oxadiazol-2-yl]-N-(prop-2-yn-1-yl)pyridine-2-carboxamide
I.040 5-[5-(difluoromethyl)-1,3,4-oxadiazol-2-yl]-2-(piperidin-1-yl)pyridine
I.041 4-{5-[5-(difluoromethyl)-1,3,4-oxadiazol-2-yl]pyridin-2-yl}morpholine
I.042 5-[5-(difluoromethyl)-1,3,4-oxadiazol-2-yl]-N′,N′-dimethylpyridine-2-carbohydrazide
I.043 5-[5-(difluoromethyl)-1,3,4-oxadiazol-2-yl]-2-phenoxypyridine
I.044 5-[5-(difluoromethyl)-1,3,4-oxadiazol-2-yl]-N-(1-methylcyclopropyl)pyridine-2-carboxamide
I.045 N-{5-[5-(difluoromethyl)-1,3,4-oxadiazol-2-yl]pyridin-2-yl}-1-methylcyclopropanecarboxamide
I.046 N-(cyclopropylmethyl)-5-[5-(difluoromethyl)-1,3,4-oxadiazol-2-yl]pyridine-2-carboxamide
I.047 5-[5-(difluoromethyl)-1,3,4-oxadiazol-2-yl]-N-(2-methoxyethyl)pyridine-2-carboxamide
I.048 5-[5-(difluoromethyl)-1,3,4-oxadiazol-2-yl]-N-(4-methylphenyl)pyridine-2-carboxamide
I.049 5-[5-(difluoromethyl)-1,3,4-oxadiazol-2-yl]-N-methyl-N-phenylpyridine-2-carboxamide
I.050 N-{5-[5-(difluoromethyl)-1,3,4-oxadiazol-2-yl]pyridin-2-yl}-2-phenylacetamide
I.051 l-{5-[5-(difluoromethyl)-1,3,4-oxadiazol-2-yl]pyridin-2-yl}-3-phenylurea
I.052 5-[5-(difluoromethyl)-1,3,4-oxadiazol-2-yl]-N-[(1RS)-1-(4-fluorophenyl)ethyl]pyridin-2-amine
I.053 5-{5-[chloro(difluoro)methyl]-1,3,4-oxadiazol-2-yl}-N-(2-fluorophenyl)pyridin-2-amine
I.054 5-[5-(difluoromethyl)-1,3,4-oxadiazol-2-yl]-N-[1-(4-fluorophenyl)cyclopropyl]pyridin-2-amine
I.055 N-{5-[5-(difluoromethyl)-1,3,4-oxadiazol-2-yl]pyridin-2-yl}-2,4-difluorobenzamide
I.056 5-[5-(difluoromethyl)-1,3,4-oxadiazol-2-yl]-N-[(1-phenylcyclopropyl)methyl]pyridine-2-carboxamide
I.057 5-{5-[chloro(difluoro)methyl]-1,3,4-oxadiazol-2-yl}-N-[1-(2-fluorophenyl)cyclopropyl]pyridin-2-amine
I.058 tert-butyl 4-{5-[5-(difluoromethyl)-1,3,4-oxadiazol-2-yl]pyridin-2-yl}piperazine-1-carboxylate
I.059 5-[5-(difluoromethyl)-1,3,4-oxadiazol-2-yl]pyrimidin-2-amine
I.060 5-[5-(difluoromethyl)-1,3,4-oxadiazol-2-yl]-N-methylpyrimidin-2-amine
I.061 2-chloro-5-[5-(difluoromethyl)-1,3,4-oxadiazol-2-yl]pyrimidine
I.062 5-[5-(difluoromethyl)-1,3,4-oxadiazol-2-yl]-N,N-dimethylpyrimidin-2-amine
I.063 5-[5-(difluoromethyl)-1,3,4-oxadiazol-2-yl]-N-(prop-2-yn-1-yl)pyrimidin-2-amine
I.064 5-[5-(difluoromethyl)-1,3,4-oxadiazol-2-yl]-2-(2,2-dimethylhydrazino)pyrimidine
I.065 2-(cyclopent-1-en-1-yl)-5-[5-(difluoromethyl)-1,3,4-oxadiazol-2-yl]pyrimidine
I.066 5-[5-(difluoromethyl)-1,3,4-oxadiazol-2-yl]-N-(1-methylcyclopropyl)pyrimidin-2-amine
I.067 N-(cyclopropylmethyl)-5-[5-(difluoromethyl)-1,3,4-oxadiazol-2-yl]pyrimidin-2-amine
I.068 5-[5-(difluoromethyl)-1,3,4-oxadiazol-2-yl]-N-(2-methoxyethyl)pyrimidin-2-amine
I.069 5-[5-(difluoromethyl)-1,3,4-oxadiazol-2-yl]-2-phenylpyrimidine
I.070 5-[5-(difluoromethyl)-1,3,4-oxadiazol-2-yl]-2-(pyridin-3-yl)pyrimidine
I.071 5-[5-(difluoromethyl)-1,3,4-oxadiazol-2-yl]-2-(2-thienyl)pyrimidine
I.072 5-[5-(difluoromethyl)-1,3,4-oxadiazol-2-yl]-2-(3-thienyl)pyrimidine
I.073 N-[(1RS)-1-cyclopropylethyl]-5-[5-(difluoromethyl)-1,3,4-oxadiazol-2-yl]pyrimidin-2-amine
I.074 4-{5-[5-(difluoromethyl)-1,3,4-oxadiazol-2-yl]pyrimidin-2-yl}morpholine
I.075 2-benzyl-5-[5-(difluoromethyl)-1,3,4-oxadiazol-2-yl]pyrimidine
I.076 5-[5-(difluoromethyl)-1,3,4-oxadiazol-2-yl]-N-phenylpyrimidin-2-amine
I.077 5-[5-(difluoromethyl)-1,3,4-oxadiazol-2-yl]-2-(4-fluorophenyl)pyrimidine
I.078 5-[5-(difluoromethyl)-1,3,4-oxadiazol-2-yl]-2-(1-ethyl-1H-pyrazol-5-yl)pyrimidine
I.079 5-[5-(difluoromethyl)-1,3,4-oxadiazol-2-yl]-2-(2-fluoropyridin-4-yl)pyrimidine
I.080 5-[5-(difluoromethyl)-1,3,4-oxadiazol-2-yl]-2-(6-fluoropyridin-3-yl)pyrimidine
I.081 5-[5-(difluoromethyl)-1,3,4-oxadiazol-2-yl]-2-(3,5-dimethyl-1,2-oxazol-4-yl)pyrimidine
I.082 N-[[1,1′-bi(cyclopropyl)]-1-yl]-5-[5-(difluoromethyl)-1,3,4-oxadiazol-2-yl]pyrimidin-2-amine
I.083 2-(cyclohexylmethyl)-5-[5-(difluoromethyl)-1,3,4-oxadiazol-2-yl]pyrimidine
I.084 N-(2-cyclopropylpropan-2-yl)-5-[5-(difluoromethyl)-1,3,4-oxadiazol-2-yl]pyrimidin-2-amine
I.085 4-{5-[5-(difluoromethyl)-1,3,4-oxadiazol-2-yl]pyrimidin-2-yl}benzonitrile
I.086 5-[5-(difluoromethyl)-1,3,4-oxadiazol-2-yl]-N-(2-methylphenyl)pyrimidin-2-amine
I.087 5-[5-(difluoromethyl)-1,3,4-oxadiazol-2-yl]-N-methyl-N-phenylpyrimidin-2-amine
I.088 N-benzyl-5-[5-(difluoromethyl)-1,3,4-oxadiazol-2-yl]pyrimidin-2-amine
I.089 5-[5-(difluoromethyl)-1,3,4-oxadiazol-2-yl]-2-(4-methoxyphenyl)pyrimidine
I.090 2-(benzyloxy)-5-[5-(difluoromethyl)-1,3,4-oxadiazol-2-yl]pyrimidine
I.091 5-[5-(difluoromethyl)-1,3,4-oxadiazol-2-yl]-2-(4-fluorobenzyl)pyrimidine
I.092 5-[5-(difluoromethyl)-1,3,4-oxadiazol-2-yl]-2-(phenylsulfanyl)pyrimidine
I.093 5-[5-(difluoromethyl)-1,3,4-oxadiazol-2-yl]-N-(2-fluorophenyl)pyrimidin-2-amine
I.094 5-[5-(difluoromethyl)-1,3,4-oxadiazol-2-yl]-N-(3-fluorophenyl)pyrimidin-2-amine
I.095 5-[5-(difluoromethyl)-1,3,4-oxadiazol-2-yl]-N-(4-fluorophenyl)pyrimidin-2-amine
I.096 5-[5-(difluoromethyl)-1,3,4-oxadiazol-2-yl]-N-(1-ethyl-1H-pyrazol-5-yl)pyrimidin-2-amine
I.097 5-[5-(difluoromethyl)-1,3,4-oxadiazol-2-yl]-N-[1′-methyl[1,1′-bi(cyclopropyl)]-1-yl]pyrimidin-2-amine
I.098 5-[5-(difluoromethyl)-1,3,4-oxadiazol-2-yl]-2-(4-fluorophenoxy)pyrimidine
I.099 2-(2-chlorophenyl)-5-[5-(difluoromethyl)-1,3,4-oxadiazol-2-yl]pyrimidine
I.100 2-(3-chlorophenyl)-5-[5-(difluoromethyl)-1,3,4-oxadiazol-2-yl]pyrimidine
I.101 2-(4-chlorophenyl)-5-[5-(difluoromethyl)-1,3,4-oxadiazol-2-yl]pyrimidine
I.102 2-(2-chloropyridin-4-yl)-5-[5-(difluoromethyl)-1,3,4-oxadiazol-2-yl]pyrimidine
I.103 2-(5-chloropyridin-3-yl)-5-[5-(difluoromethyl)-1,3,4-oxadiazol-2-yl]pyrimidine
I.104 methyl 1-({5-[5-(difluoromethyl)-1,3,4-oxadiazol-2-yl]pyrimidin-2-yl}amino)cyclopropanecarboxylate
I.105 5-[5-(difluoromethyl)-1,3,4-oxadiazol-2-yl]-N-(tetrahydro-2H-pyran-4-ylmethyl)pyrimidin-2-amine
I.106 2-{5-[5-(difluoromethyl)-1,3,4-oxadiazol-2-yl]pyrimidin-2-yl}isoindoline
I.107 5-[5-(difluoromethyl)-1,3,4-oxadiazol-2-yl]-2-(2-methoxybenzyl)pyrimidine
I.108 5-[5-(difluoromethyl)-1,3,4-oxadiazol-2-yl]-2-(2-methyl-2-phenylhydrazino)pyrimidine
I.109 5-[5-(difluoromethyl)-1,3,4-oxadiazol-2-yl]-N-(2-methoxyphenyl)pyrimidin-2-amine
I.110 N-benzyl-5-[5-(difluoromethyl)-1,3,4-oxadiazol-2-yl]-N-hydroxypyrimidin-2-amine
I.111 5-[5-(difluoromethyl)-1,3,4-oxadiazol-2-yl]-N-(2-fluorobenzyl)pyrimidin-2-amine
I.112 5-[5-(difluoromethyl)-1,3,4-oxadiazol-2-yl]-N-(4-fluorobenzyl)pyrimidin-2-amine
I.113 5-[5-(difluoromethyl)-1,3,4-thiadiazol-2-yl]-N-(2-fluorophenyl)pyrimidin-2-amine
I.114 N-(2-chlorophenyl)-5-[5-(difluoromethyl)-1,3,4-oxadiazol-2-yl]pyrimidin-2-amine
I.115 2-(2,4-difluorobenzyl)-5-[5-(difluoromethyl)-1,3,4-oxadiazol-2-yl]pyrimidine
I.116 N-(2-fluorophenyl)-5-[5-(trifluoromethyl)-1,3,4-oxadiazol-2-yl]pyrimidin-2-amine
I.117 ethyl 1-({5-[5-(difluoromethyl)-1,3,4-oxadiazol-2-yl]pyrimidin-2-yl}amino)cyclopropanecarboxylate
I.118 5-[5-(difluoromethyl)-1,3,4-oxadiazol-2-yl]-N-[(1RS)-1-phenylprop-2-yn-1-yl]pyrimidin-2-amine
I.119 N-(2-cyclopropylphenyl)-5-[5-(difluoromethyl)-1,3,4-oxadiazol-2-yl]pyrimidin-2-amine
I.120 5-[5-(difluoromethyl)-1,3,4-oxadiazol-2-yl]-2-[(1-phenylcyclopropyl)oxy]pyrimidine
I.121 5-[5-(difluoromethyl)-1,3,4-oxadiazol-2-yl]-N-[1-(pyridin-2-yl)cyclopropyl]pyrimidin-2-amine
I.122 5-[5-(difluoromethyl)-1,3,4-oxadiazol-2-yl]-N-[(1RS)-1-phenylpropyl]pyrimidin-2-amine
I.123 5-[5-(difluoromethyl)-1,3,4-oxadiazol-2-yl]-2-{[(1RS)-1-phenylethyl]sulfanyl}pyrimidine
I.124 5-[5-(difluoromethyl)-1,3,4-oxadiazol-2-yl]-N-[(1RS)-1-(2-fluorophenyl)ethyl]pyrimidin-2-amine
I.125 5-[5-(difluoromethyl)-1,3,4-oxadiazol-2-yl]-N-[(1RS)-1-(4-fluorophenyl)ethyl]pyrimidin-2-amine
I.125a 5-[5-(difluoromethyl)-1,3,4-oxadiazol-2-yl]-N-[(1R)-1-(4-fluorophenyl)ethyl]pyrimidin-2-amine
I.125b 5-[5-(difluoromethyl)-1,3,4-oxadiazol-2-yl]-N-[(1S)-1-(4-fluorophenyl)ethyl]pyrimidin-2-amine
I.126 N-(1-cyclohexylcyclopropyl)-5-[5-(difluoromethyl)-1,3,4-oxadiazol-2-yl]pyrimidin-2-amine
I.127 5-[5-(difluoromethyl)-1,3,4-oxadiazol-2-yl]-2-{[(1RS)-1-(4-fluorophenyl)ethyl]oxy}pyrimidine
I.128 N-(2-chlorobenzyl)-5-[5-(difluoromethyl)-1,3,4-oxadiazol-2-yl]pyrimidin-2-amine
I.129 N-(2,6-difluorobenzyl)-5-[5-(difluoromethyl)-1,3,4-oxadiazol-2-yl]pyrimidin-2-amine
I.130 N-(2-fluorophenyl)-5-[5-(trifluoromethyl)-1,3,4-thiadiazol-2-yl]pyrimidin-2-amine
I.131 5-{5-[chloro(difluoro)methyl]-1,3,4-oxadiazol-2-yl}-N-(2-fluorophenyl)pyrimidin-2-amine
I.132 5-[5-(difluoromethyl)-1,3,4-oxadiazol-2-yl]-N-[1-(2-methylphenyl)cyclopropyl]pyrimidin-2-amine
I.133 5-[5-(difluoromethyl)-1,3,4-oxadiazol-2-yl]-N-[1-(4-methylphenyl)cyclopropyl]pyrimidin-2-amine
I.134 5-[5-(difluoromethyl)-1,3,4-oxadiazol-2-yl]-N-(1-phenylcyclobutyl)pyrimidin-2-amine
I.135 5-[5-(difluoromethyl)-1,3,4-oxadiazol-2-yl]-N-[(1-phenylcyclopropyl)methyl]pyrimidin-2-amine
I.136 N—[(RS)-cyclopropyl(phenyl)methyl]-5-[5-(difluoromethyl)-1,3,4-oxadiazol-2-yl]pyrimidin-2-amine
I.137 5-[5-(difluoromethyl)-1,3,4-oxadiazol-2-yl]-N-[1-(pyridin-2-yl)cyclobutyl]pyrimidin-2-amine
I.138 5-[5-(difluoromethyl)-1,3,4-oxadiazol-2-yl]-N-(2-phenylbutan-2-yl)pyrimidin-2-amine
I.139 5-[5-(difluoromethyl)-1,3,4-oxadiazol-2-yl]-N-[(1RS)-2-methyl-1-phenylpropyl]pyrimidin-2-amine
I.140 5-[5-(difluoromethyl)-1,3,4-oxadiazol-2-yl]-N-[(1RS)-1-phenylbutyl]pyrimidin-2-amine
I.141 (2RS)—({5-[5-(difluoromethyl)-1,3,4-oxadiazol-2-yl]pyrimidin-2-yl}amino)(4-fluorophenyl)acetonitrile
I.142 5-[5-(difluoromethyl)-1,3,4-oxadiazol-2-yl]-2-[(3-phenyloxetan-3-yl)oxy]pyrimidine
I.143 5-[5-(difluoromethyl)-1,3,4-oxadiazol-2-yl]-2-{[3-(pyridin-2-yl)oxetan-3-yl]oxy}pyrimidine
I.144 5-[5-(difluoromethyl)-1,3,4-oxadiazol-2-yl]-N-[1-(2-fluorophenyl)cyclopropyl]pyrimidin-2-amine
I.145 5-[5-(difluoromethyl)-1,3,4-oxadiazol-2-yl]-N-[1-(3-fluorophenyl)cyclopropyl]pyrimidin-2-amine
I.146 5-[5-(difluoromethyl)-1,3,4-oxadiazol-2-yl]-N-[1-(4-fluorophenyl)cyclopropyl]pyrimidin-2-amine
I.147 N-{5-[5-(difluoromethyl)-1,3,4-oxadiazol-2-yl]pyrimidin-2-yl}-O-methyl-N-[(1RS)-1-phenylethyl]hydroxylamine
I.148 N-benzyl-N-{5-[5-(difluoromethyl)-1,3,4-oxadiazol-2-yl]pyrimidin-2-yl}-O-ethylhydroxylamine
I.149 5-[5-(difluoromethyl)-1,3,4-oxadiazol-2-yl]-2-{[1-(4-fluorophenyl)cyclopropyl]oxy}pyrimidine
I.150 5-[5-(difluoromethyl)-1,3,4-oxadiazol-2-yl]-N-[2-(4-fluorophenyl)propan-2-yl]pyrimidin-2-amine
I.151 5-[5-(difluoromethyl)-1,3,4-oxadiazol-2-yl]-N-[(1RS)-1-(2,6-difluorophenyl)ethyl]pyrimidin-2-amine
I.152 4-[1-({5-[5-(difluoromethyl)-1,3,4-oxadiazol-2-yl]pyrimidin-2-yl}amino)cyclopropyl]benzonitrile
I.153 5-[5-(difluoromethyl)-1,3,4-oxadiazol-2-yl]-N-(1-phenylcyclopentyl)pyrimidin-2-amine
I.155 5-[5-(difluoromethyl)-1,3,4-oxadiazol-2-yl]-N-[1-(2-methoxyphenyl)cyclopropyl]pyrimidin-2-amine
I.156 5-[5-(difluoromethyl)-1,3,4-oxadiazol-2-yl]-N-[1-(3-methoxyphenyl)cyclopropyl]pyrimidin-2-amine
I.157 5-[5-(difluoromethyl)-1,3,4-oxadiazol-2-yl]-N-[1-(4-methoxyphenyl)cyclopropyl]pyrimidin-2-amine
I.158 5-[5-(difluoromethyl)-1,3,4-oxadiazol-2-yl]-N-[3-(4-methylphenyl)oxetan-3-yl]pyrimidin-2-amine
I.159 5-[5-(difluoromethyl)-1,3,4-thiadiazol-2-yl]-N-(1-phenylcyclobutyl)pyrimidin-2-amine
I.160 N-(1-phenylcyclobutyl)-5-[5-(trifluoromethyl)-1,3,4-oxadiazol-2-yl]pyrimidin-2-amine
I.161 N-{5-[5-(difluoromethyl)-1,3,4-oxadiazol-2-yl]pyrimidin-2-yl}-N-(2-methoxybenzyl)-O-methylhydroxylamine
I.162 5-[5-(difluoromethyl)-1,3,4-thiadiazol-2-yl]-N-[1-(2-fluorophenyl)cyclopropyl]pyrimidin-2-amine
I.163 N-[1-(2-chlorophenyl)cyclopropyl]-5-[5-(difluoromethyl)-1,3,4-oxadiazol-2-yl]pyrimidin-2-amine
I.164 5-[5-(difluoromethyl)-1,3,4-oxadiazol-2-yl]-N-[1-(2,6-difluorophenyl)cyclopropyl]pyrimidin-2-amine
I.165 N-[1-(2-fluorophenyl)cyclopropyl]-5-[5-(trifluoromethyl)-1,3,4-oxadiazol-2-yl]pyrimidin-2-amine
I.166 N-([biphenyl]-2-yl)-5-[5-(difluoromethyl)-1,3,4-oxadiazol-2-yl]pyrimidin-2-amine
I.167 5-[5-(difluoromethyl)-1,3,4-oxadiazol-2-yl]-2-{[1-(2,6-difluorophenyl)cyclopropyl]oxy}pyrimidine
I.168 5-[5-(difluoromethyl)-1,3,4-oxadiazol-2-yl]-N-[2-(2,6-difluorophenyl)propan-2-yl]pyrimidin-2-amine
I.169 N-(4-chlorobenzyl)-N-{5-[5-(difluoromethyl)-1,3,4-oxadiazol-2-yl]pyrimidin-2-yl}-O-methylhydroxylamine
I.170 5-[5-(difluoromethyl)-1,3,4-oxadiazol-2-yl]-N-[1′-phenyl[1,1′-bi(cyclopropyl)]-1-yl]pyrimidin-2-amine
I.171 5-[5-(difluoromethyl)-1,3,4-oxadiazol-2-yl]-N-[1-(2-fluorophenyl)cyclopentyl]pyrimidin-2-amine
I.172 5-[5-(difluoromethyl)-1,3,4-oxadiazol-2-yl]-N-[1-(3-fluorophenyl)cyclopentyl]pyrimidin-2-amine
I.173 N-(1-phenylcyclobutyl)-5-[5-(trifluoromethyl)-1,3,4-thiadiazol-2-yl]pyrimidin-2-amine
I.174 N-[1-(2-chlorophenyl)cyclobutyl]-5-[5-(difluoromethyl)-1,3,4-oxadiazol-2-yl]pyrimidin-2-amine
I.175 N-[1-(3-chlorophenyl)cyclobutyl]-5-[5-(difluoromethyl)-1,3,4-oxadiazol-2-yl]pyrimidin-2-amine
I.176 5-{5-[chloro(difluoro)methyl]-1,3,4-oxadiazol-2-yl}-N-(1-phenylcyclobutyl)pyrimidin-2-amine
I.177 5-[5-(difluoromethyl)-1,3,4-oxadiazol-2-yl]-N-[1-(2,6-difluorophenyl)cyclobutyl]pyrimidin-2-amine
I.178 N-[1-(2-fluorophenyl)cyclopropyl]-5-[5-(trifluoromethyl)-1,3,4-thiadiazol-2-yl]pyrimidin-2-amine
I.179 5-{5-[chloro(difluoro)methyl]-1,3,4-oxadiazol-2-yl}-N-[1-(2-fluorophenyl)cyclopropyl]pyrimidin-2-amine
I.180 methyl 4-[1-({5-[5-(difluoromethyl)-1,3,4-oxadiazol-2-yl]pyrimidin-2-yl}amino)cyclopropyl]benzoate
I.181 5-[5-(difluoromethyl)-1,3,4-oxadiazol-2-yl]-N-[(1RS)-2,2,2-trifluoro-1-(4-fluorophenyl)ethyl]pyrimidin-2-amine
I.182 N-[1-(4-chlorophenyl)cyclohexyl]-5-[5-(difluoromethyl)-1,3,4-oxadiazol-2-yl]pyrimidin-2-amine
I.183 N-[1-(2-bromophenyl)cyclopropyl]-5-[5-(difluoromethyl)-1,3,4-oxadiazol-2-yl]pyrimidin-2-amine
I.184 N-{5-[5-(difluoromethyl)-1,3,4-oxadiazol-2-yl]pyrimidin-2-yl}-O-methyl-N-{(1RS)-1-[3-(trifluoromethyl)phenyl]ethyl}hydroxylamine
I.185 N-(2,4-difluorobenzoyl)-N-{5-[5-(difluoromethyl)-1,3,4-oxadiazol-2-yl]pyrimidin-2-yl}-2,4-difluorobenzamide
I.186 5-[5-(difluoromethyl)-1,3,4-oxadiazol-2-yl]-N-[(1RS)-1-(1-methylcyclopropyl)ethyl]pyrimidin-2-amine
I.187 5-[5-(difluoromethyl)-1,3,4-oxadiazol-2-yl]-N-(1-isopropylcyclopropyl)pyrimidin-2-amine
I.188 5-[5-(difluoromethyl)-1,3,4-oxadiazol-2-yl]-N-[(1RS)-1-(pyridin-3-yl)ethyl]pyrimidin-2-amine
I.189 5-[5-(difluoromethyl)-1,3,4-oxadiazol-2-yl]-N-[(1RS)-1-(pyridin-2-yl)ethyl]pyrimidin-2-amine
I.190 5-[5-(difluoromethyl)-1,3,4-oxadiazol-2-yl]-N-[(1RS)-1-(pyridin-4-yl)ethyl]pyrimidin-2-amine
I.191 5-[5-(difluoromethyl)-1,3,4-oxadiazol-2-yl]-N-[(1RS)-1-(pyrimidin-2-yl)ethyl]pyrimidin-2-amine
I.192 5-[5-(difluoromethyl)-1,3,4-oxadiazol-2-yl]-N-[(1RS)-1-(2-thienyl)ethyl]pyrimidin-2-amine
I.193 N-[(1RS)-1-cyclohexylethyl]-5-[5-(difluoromethyl)-1,3,4-oxadiazol-2-yl]pyrimidin-2-amine
I.194 5-[5-(difluoromethyl)-1,3,4-oxadiazol-2-yl]-N-[(1RS)-1-(1,3-thiazol-4-yl)ethyl]pyrimidin-2-amine
I.195 5-[5-(difluoromethyl)-1,3,4-oxadiazol-2-yl]-N-(1-phenylcyclopropyl)pyrimidin-2-amine
I.196 5-[5-(difluoromethyl)-1,3,4-oxadiazol-2-yl]-N-[rac-(1R,2S)-2-phenylcyclopropyl]pyrimidin-2-amine
I.197 5-[5-(difluoromethyl)-1,3,4-oxadiazol-2-yl]-N-(2-phenylpropan-2-yl)pyrimidin-2-amine
I.198 5-[5-(difluoromethyl)-1,3,4-oxadiazol-2-yl]-N-[2-(pyridin-2-yl)propan-2-yl]pyrimidin-2-amine
I.199 5-[5-(difluoromethyl)-1,3,4-oxadiazol-2-yl]-N-[2-(pyridin-3-yl)propan-2-yl]pyrimidin-2-amine
I.200 5-[5-(difluoromethyl)-1,3,4-oxadiazol-2-yl]-N-[2-(4-fluorophenyl)ethyl]pyrimidin-2-amine
I.201 N-(1-benzylcyclopropyl)-5-[5-(difluoromethyl)-1,3,4-oxadiazol-2-yl]pyrimidin-2-amine
I.202 5-[5-(difluoromethyl)-1,3,4-oxadiazol-2-yl]-N-[1-(3-methylphenyl)cyclopropyl]pyrimidin-2-amine
I.203 5-[5-(difluoromethyl)-1,3,4-oxadiazol-2-yl]-N-[1-(4-methylpyridin-2-yl)cyclopropyl]pyrimidin-2-amine
I.204 5-[5-(difluoromethyl)-1,3,4-oxadiazol-2-yl]-N-[2-(4-methylphenyl)propan-2-yl]pyrimidin-2-amine
I.205 5-[5-(difluoromethyl)-1,3,4-oxadiazol-2-yl]-N-[2-(2-methylphenyl)propan-2-yl]pyrimidin-2-amine
I.206 5-[5-(difluoromethyl)-1,3,4-oxadiazol-2-yl]-N-[rac-(1R,2R)-2-(4-fluorophenyl)cyclopropyl]pyrimidin-2-amine
I.207 5-[5-(difluoromethyl)-1,3,4-oxadiazol-2-yl]-N-[(1RS)-1-(4-methoxyphenyl)ethyl]pyrimidin-2-amine
I.208 5-[5-(difluoromethyl)-1,3,4-oxadiazol-2-yl]-N-[1-(2-furyl)cyclopentyl]pyrimidin-2-amine
I.209 5-[5-(difluoromethyl)-1,3,4-oxadiazol-2-yl]-N-[(1RS)-1-(2-methoxyphenyl)ethyl]pyrimidin-2-amine
I.210 5-[5-(difluoromethyl)-1,3,4-oxadiazol-2-yl]-N-[(2RS)-1-(4-fluorophenyl)propan-2-yl]pyrimidin-2-amine
I.211 5-[5-(difluoromethyl)-1,3,4-oxadiazol-2-yl]-N-[(2RS)-2-(4-fluorophenyl)propyl]pyrimidin-2-amine
I.212 5-[5-(difluoromethyl)-1,3,4-oxadiazol-2-yl]-N-[2-(2-fluorophenyl)propan-2-yl]pyrimidin-2-amine
I.213 N-[(1RS)-1-(3-chlorophenyl)ethyl]-5-[5-(difluoromethyl)-1,3,4-oxadiazol-2-yl]pyrimidin-2-amine
I.214 N-[(1RS)-1-(6-chloropyridin-3-yl)ethyl]-5-[5-(difluoromethyl)-1,3,4-oxadiazol-2-yl]pyrimidin-2-amine
I.215 5-[5-(difluoromethyl)-1,3,4-oxadiazol-2-yl]-N-[(1RS)-1-(2,4-difluorophenyl)ethyl]pyrimidin-2-amine
I.216 5-[5-(difluoromethyl)-1,3,4-oxadiazol-2-yl]-N-[1-(2,6-dimethylphenyl)cyclopropyl]pyrimidin-2-amine
I.217 5-[5-(difluoromethyl)-1,3,4-oxadiazol-2-yl]-N-[(1RS)-2-phenylcyclopentyl]pyrimidin-2-amine
I.218 5-[5-(difluoromethyl)-1,3,4-oxadiazol-2-yl]-N-[1-(pyridin-3-yl)cyclopentyl]pyrimidin-2-amine
I.219 5-[5-(difluoromethyl)-1,3,4-oxadiazol-2-yl]-N-[1-(pyridin-2-yl)cyclopentyl]pyrimidin-2-amine
I.220 5-[5-(difluoromethyl)-1,3,4-oxadiazol-2-yl]-N-[1-(4-fluorophenyl)cyclobutyl]pyrimidin-2-amine
I.221 5-[5-(difluoromethyl)-1,3,4-oxadiazol-2-yl]-N-[1-(2-fluorophenyl)cyclopropyl]-N-methylpyrimidin-2-amine
I.222 methyl ({5-[5-(difluoromethyl)-1,3,4-oxadiazol-2-yl]pyrimidin-2-yl}amino)(pyridin-2-yl)acetate
I.223 5-[5-(difluoromethyl)-1,3,4-oxadiazol-2-yl]-N-[1-(4-fluorophenyl)-2-methylpropan-2-yl]pyrimidin-2-amine
I.224 5-[5-(difluoromethyl)-1,3,4-oxadiazol-2-yl]-N-[2-(4-fluorophenyl)-2-methylpropyl]pyrimidin-2-amine
I.225 5-[5-(difluoromethyl)-1,3,4-oxadiazol-2-yl]-N-[1-(2-thienyl)cyclopentyl]pyrimidin-2-amine
I.226 N-[1-(4-chlorophenyl)cyclopropyl]-5-[5-(difluoromethyl)-1,3,4-oxadiazol-2-yl]pyrimidin-2-amine
I.227 N-[1-(3-chlorophenyl)cyclopropyl]-5-[5-(difluoromethyl)-1,3,4-oxadiazol-2-yl]pyrimidin-2-amine
I.228 N-[1-(4-chloropyridin-2-yl)cyclopropyl]-5-[5-(difluoromethyl)-1,3,4-oxadiazol-2-yl]pyrimidin-2-amine
I.229 5-[5-(difluoromethyl)-1,3,4-oxadiazol-2-yl]-N-[1-(3,4-difluorophenyl)cyclopropyl]pyrimidin-2-amine
I.230 N-[(1RS)-1-(4-chlorophenyl)propyl]-5-[5-(difluoromethyl)-1,3,4-oxadiazol-2-yl]pyrimidin-2-amine
I.231 N-[2-(4-chlorophenyl)propan-2-yl]-5-[5-(difluoromethyl)-1,3,4-oxadiazol-2-yl]pyrimidin-2-amine
I.232 5-[5-(difluoromethyl)-1,3,4-oxadiazol-2-yl]-N-[(1RS)-1-(2-naphthyl)ethyl]pyrimidin-2-amine
I.233 5-[5-(difluoromethyl)-1,3,4-oxadiazol-2-yl]-N-[(1RS)-1-(quinolin-2-yl)ethyl]pyrimidin-2-amine
I.234 N-[2,2-difluoro-2-(4-fluorophenyl)ethyl]-5-[5-(difluoromethyl)-1,3,4-oxadiazol-2-yl]pyrimidin-2-amine
I.235 (2RS)-2-({5-[5-(difluoromethyl)-1,3,4-oxadiazol-2-yl]pyrimidin-2-yl}amino)-2-(3,4-dimethylphenyl)propan-1-ol
I.236 5-[5-(difluoromethyl)-1,3,4-oxadiazol-2-yl]-N-[1-(2-fluoro-3-methoxyphenyl)cyclopropyl]pyrimidin-2-amine
I.237 N-[1-(4-chlorophenyl)cyclobutyl]-5-[5-(difluoromethyl)-1,3,4-oxadiazol-2-yl]pyrimidin-2-amine
I.238 5-[5-(difluoromethyl)-1,3,4-oxadiazol-2-yl]-N-(diphenylmethyl)pyrimidin-2-amine
I.239 5-[5-(difluoromethyl)-1,3,4-oxadiazol-2-yl]-N-{(1RS)-1-[3-(trifluoromethyl)phenyl]ethyl}pyrimidin-2-amine
I.240 5-[5-(difluoromethyl)-1,3,4-oxadiazol-2-yl]-N-{(1RS)-1-[2-(trifluoromethyl)phenyl]ethyl}pyrimidin-2-amine
I.241 5-[5-(difluoromethyl)-1,3,4-oxadiazol-2-yl]-N-[1-(2-fluorophenyl)cyclopropyl]-N-(prop-2-yn-1-yl)pyrimidin-2-amine
I.242 N-[1-(4-tert-butylphenyl)cyclopropyl]-5-[5-(difluoromethyl)-1,3,4-oxadiazol-2-yl]pyrimidin-2-amine
I.243 5-[5-(difluoromethyl)-1,3,4-oxadiazol-2-yl]-N-[1-(3,4-dimethoxyphenyl)cyclopropyl]pyrimidin-2-amine
I.244 N—[(R)-cyclopentyl(4-fluorophenyl)methyl]-5-[5-(difluoromethyl)-1,3,4-oxadiazol-2-yl]pyrimidin-2-amine
I.245 5-[5-(difluoromethyl)-1,3,4-oxadiazol-2-yl]-N-[(1RS)-1,2-diphenylethyl]pyrimidin-2-amine
I.246 N-{1-[3-(difluoromethoxy)phenyl]cyclopropyl}-5-[5-(difluoromethyl)-1,3,4-oxadiazol-2-yl]pyrimidin-2-amine
I.247 N-{1-[4-(difluoromethoxy)phenyl]cyclopropyl}-5-[5-(difluoromethyl)-1,3,4-oxadiazol-2-yl]pyrimidin-2-amine
I.248 N-{1-[2-(difluoromethoxy)phenyl]cyclopropyl}-5-[5-(difluoromethyl)-1,3,4-oxadiazol-2-yl]pyrimidin-2-amine
I.249 5-[5-(difluoromethyl)-1,3,4-oxadiazol-2-yl]-N-{1-[3-(trifluoromethyl)phenyl]cyclopropyl}pyrimidin-2-amine
I.250 5-[5-(difluoromethyl)-1,3,4-oxadiazol-2-yl]-N-{1-[4-(trifluoromethyl)phenyl]cyclopropyl}pyrimidin-2-amine
I.251 N-[1-(2,5-dichlorophenyl)cyclopropyl]-5-[5-(difluoromethyl)-1,3,4-oxadiazol-2-yl]pyrimidin-2-amine
I.252 N-[1-(3,4-dichlorophenyl)cyclopropyl]-5-[5-(difluoromethyl)-1,3,4-oxadiazol-2-yl]pyrimidin-2-amine
I.253 N-[1-(2,3-dichlorophenyl)cyclopropyl]-5-[5-(difluoromethyl)-1,3,4-oxadiazol-2-yl]pyrimidin-2-amine
I.254 N-[1-(3,5-dichlorophenyl)cyclopropyl]-5-[5-(difluoromethyl)-1,3,4-oxadiazol-2-yl]pyrimidin-2-amine
I.255 N-[1-(2,4-dichlorophenyl)cyclopropyl]-5-[5-(difluoromethyl)-1,3,4-oxadiazol-2-yl]pyrimidin-2-amine
I.256 5-[5-(difluoromethyl)-1,3,4-oxadiazol-2-yl]-N-{2-[3-(trifluoromethyl)phenyl]propan-2-yl}pyrimidin-2-amine
I.257 5-[5-(difluoromethyl)-1,3,4-oxadiazol-2-yl]-N-{(1RS)-1-[3-(trifluoromethoxy)phenyl]ethyl}pyrimidin-2-amine
I.258 N-[1-(3-chlorophenyl)cyclohexyl]-5-[5-(difluoromethyl)-1,3,4-oxadiazol-2-yl]pyrimidin-2-amine
I.259 N-[1-(4-chloro-3-nitrophenyl)cyclopropyl]-5-[5-(difluoromethyl)-1,3,4-oxadiazol-2-yl]pyrimidin-2-amine
I.260 N-[(1RS)-1-(2-bromophenyl)propyl]-5-[5-(difluoromethyl)-1,3,4-oxadiazol-2-yl]pyrimidin-2-amine
I.261 N-(2,2-difluoroethyl)-5-[5-(difluoromethyl)-1,3,4-oxadiazol-2-yl]-N-[1-(2-fluorophenyl)cyclopropyl]pyrimidin-2-amine
I.262 5-[5-(difluoromethyl)-1,3,4-oxadiazol-2-yl]-N-{1-[3-(trifluoromethoxy)phenyl]cyclopropyl}pyrimidin-2-amine
I.263 5-[5-(difluoromethyl)-1,3,4-oxadiazol-2-yl]-N-{1-[2-fluoro-4-(trifluoromethyl)phenyl]cyclopropyl}pyrimidin-2-amine
I.264 N-[1-(4,5-dichloro-2-fluorophenyl)cyclopropyl]-5-[5-(difluoromethyl)-1,3,4-oxadiazol-2-yl]pyrimidin-2-amine
I.265 5-[5-(difluoromethyl)-1,3,4-oxadiazol-2-yl]-N-[1-(3-phenoxyphenyl)cyclopropyl]pyrimidin-2-amine
I.266 5-[5-(difluoromethyl)-1,3,4-oxadiazol-2-yl]-N-{1-[4-(trifluoromethyl)phenyl]cyclopentyl}pyrimidin-2-amine
I.267 N-{5-[5-(difluoromethyl)-1,3,4-oxadiazol-2-yl]pyrimidin-2-yl}-2,2,2-trifluoro-N-[1-(2-fluorophenyl)cyclopropyl]acetamide
I.268 N-[1-(2-bromophenyl)cyclohexyl]-5-[5-(difluoromethyl)-1,3,4-oxadiazol-2-yl]pyrimidin-2-amine as well as their salts, N-oxides, solvates and optically active isomers or geometric isomers.

In some embodiments, the present invention relates to the use of compounds of formula (I) for controlling phytopathogenic fungi:

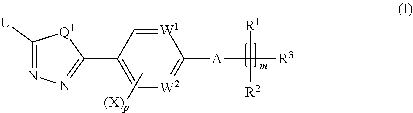

wherein
U is a C₁-C₃-haloalkyl comprising 2 to 7 halogen atoms that can be the same or different and selected from the group consisting of fluorine and chlorine;
Q¹ is O or S;
W¹ and W² are independently N, CH or CF;
A is selected from the group consisting of direct bond, O, NR⁴, —(C═O)—, —(C═S)—, —O—(C═O), —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —N(R⁴)—(C═O)—N(R⁵)—, —N(R⁴)—(C═S)—N(R⁵)—, —O—(C═O)—O—, —O—(C═S)═O—;
m=0 or 1;
p=0, 1 or 2;
X is fluorine;
R¹ and R² are independently selected from the group consisting of hydrogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with, respectively, one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with, respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of O, S and NH, wherein said C₃-C₇-cycloalkyl and 3- to 10-membered saturated or partially unsaturated heterocyclyl ring may be substituted with one or more R^{1b} substituents;
R³ is hydrogen, halogen, C₁-C₈-alkyl, C₁-C₈-halogenoalkyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl may be substituted with one or more R^{3a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, aryl, heteroaryl, aryl-C₁-C₈-alkyl, heteroaryl-C₁-C₈-alkyl and phenylsulfonyl, may be substituted with, respectively one or more R^{4b} and R^{5b} substituents;
R^{1a}, R^{2a}, R^{3a}, R^{4a} and R^{5a} are independently selected from the group consisting of nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that compound of formula (I) is not:
2-isopropyl-5,6-dimethyl-3-{4-[5-(trifluoromethyl)-1,3,4-oxadiazol-2-yl]benzyl}pyridin-4-ol [2133324-02-2];
N-{4-[5-(trifluoromethyl)-1,3,4-oxadiazol-2-yl]phenyl}cyclopropanecarboxamide [2376135-82-7],
   tert-butyl {4-[5-(trifluoromethyl)-1,3,4-oxadiazol-2-yl]phenyl}carbamate [2376135-81-6],

as well as their salts, N-oxides, solvates and optically active isomers or geometric isomers.

In the above formula (I), U is preferably C₁-haloalkyl comprising 2 to 3 halogen atoms that can be the same or different and selected from the group consisting of fluorine and chlorine, more preferably U is CHF₂ or CF₃.

In the above formula (I), Q¹ is preferably O.

In the above formula (I), W¹ is preferably N or CH.

In the above formula (I), W² is preferably N or CH.

In some embodiments in the above formula (I), W¹ and W² are N, or W¹ is N and W² is CH.

In the above formula (I), A is preferably O, NR⁴, —(C═O)—, —N(R⁴)—(C═O)—, —(C═O)—O— or —(C═O)—N(R⁵)—, more preferably A is NH, —(C═O)—O—, —(C═O)—NH— or —(C═O)—NMe-.

In some embodiments, in above formula (I), A is NR⁴, preferably NH.

In the above formula (I), p is preferably 0 or 1, more preferably p is 0.

In the above formula (I), R¹ and R² are preferably independently selected from the group consisting of hydrogen, C₁-C₈-alkyl, C₁-C₈-halogenoalkyl, C₃-C₇-cycloalkyl, aryl, heterocyclyl, heteroaryl, aryl-C₁-C₈-alkyl, heterocyclyl-C₁-C₈-alkyl and heteroaryl-C₁-C₈-alkyl, wherein said C₁-C₈-alkyl, C₁-C₈-halogenoalkyl, C₃-C₇-cycloalkyl, aryl, heterocyclyl, heteroaryl, aryl-C₁-C₈-alkyl, heterocyclyl-C₁-C₈-alkyl and heteroaryl-C₁-C₈-alkyl may be substituted as described herein, or R¹ and R² form, together with the carbon atom to which they are linked, a C₃-C₇-cycloalkyl.

In the above formula (I), R¹ is more preferably selected from the group consisting of hydrogen, C₁-C₈-alkyl.

In the above formula (I), R² is more preferably selected from the group consisting of hydrogen, C₁-C₈-alkyl.

In the above formula (I), R¹ and R² may more preferably form together with the carbon atom to which they are linked, a C₃-C₆-cycloalkyl or a 3- to 6-membered saturated or partially unsaturated heterocyclyl ring that contains 1 to 3 heteroatoms that can be the same or different and selected from the group consisting of O, S and NH, even more preferably R¹ and R² may form together with the carbon atom to which they are linked, a cyclopropyl or a cyclobutyl ring.

In the above formula (I), R³ is preferably selected from the group consisting of hydrogen, C₁-C₈-alkyl, aryl and heterocyclyl, wherein said C₁-C₈-alkyl, aryl and heterocyclyl may be substituted as described herein.

In the above formula (I), R³ is more preferably selected from the group consisting of hydrogen, C₁-C₈-alkyl that may be substituted by a C₁-C₈-alkoxy (e.g. 2-methoxyethyl), unsubstituted or substituted aryl and unsubstituted or substituted heterocyclyl.

In the above formula (I), R⁴ and R⁵ are preferably selected from the group consisting of hydrogen atom, C₁-C₈-alkyl, C₁-C₈-alkoxy, C₃-C₇-cycloalkyl, C₃-C₇-cycloalkyl-C₁-C₈-alkyl, C₁-C₈-alkylcarbonyl, C₁-C₈-halogenoalkyl-carbonyl, C₁-C₈-alkylsulfonyl, C₁-C₈-halogenoalkylsulfonyl, aryl, aryl-C₁-C₈-alkyl and phenylsulfonyl, wherein said C₁-C₈-alkyl, C₁-C₈-alkoxy, C₃-C₇-cycloalkyl, C₃-C₇-cycloalkyl-C₁-C₈-alkyl, C₁-C₈-alkylcarbonyl, C₁-C₈-halogenoalkyl-carbonyl, C₁-C₈-alkylsulfonyl, C₁-C₈-halogenoalkylsulfonyl, aryl, aryl-C₁-C₈-alkyl and phenylsulfonyl may be substituted as described herein.

In the above formula (I), R⁴ is more preferably hydrogen or C₁-C₈-alkyl, in particular hydrogen.

In the above formula (I), R⁵ is more preferably hydrogen or C₁-C₈-alkyl, in particular hydrogen.

The above mentioned preferences with regard to the substituents of the compounds according to the invention can be combined in various manners. These combinations of preferred features thus provide sub-classes of compounds according to the invention. Examples of such sub-classes of preferred compounds according to the invention are:
preferred features of U with one or more preferred features of Q¹, W¹, W², A, p, R¹, R², R³, R⁴ and R⁵;
preferred features of Q¹ with one or more preferred features of U, W¹, W², A, p, R¹, R², R³, R⁴ and R⁵;
preferred features of W¹ with one or more preferred features of U, Q¹, W², A, p, R¹, R², R³, R⁴ and R⁵;
preferred features of W² with one or more preferred features of U, Q¹, W¹, A, p, R¹, R², R³, R⁴ and R⁵;
preferred features of A with one or more preferred features of U, Q¹, W¹, W², p, R¹, R², R³, R⁴ and R⁵;
preferred features of p with one or more preferred features of U, Q¹, W¹, W², A, R¹, R², R³, R⁴ and R⁵;
preferred features of R¹ with one or more preferred features of U, Q¹, W¹, W², A, p, R², R³, R⁴ and R⁵;
preferred features of R² with one or more preferred features of U, Q¹, W¹, W², A, p, R¹, R³, R⁴ and R⁵;
preferred features of R³ with one or more preferred features of U, Q¹, W¹, W², A, p, R¹, R², R⁴ and R⁵;
preferred features of R⁴ with one or more preferred features of U, Q¹, W¹, W², A, p, R¹, R², R³, and R⁵;
preferred features of R⁵ with one or more preferred features of U, Q¹, W¹, W², A, p, R¹, R², R³ and R⁴;

In these combinations of preferred features of the substituents of the compounds according to the invention, the said preferred features can also be selected among the more preferred features of each of U, Q¹, W¹, W², A, p, R¹, R², R³, R⁴ and R⁵ so as to form most preferred subclasses of compounds according to the invention.

The present invention relates to compounds of formula (I):

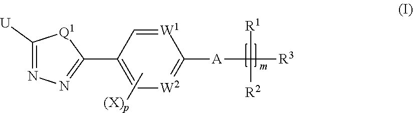

wherein
U is a C₁-C₃-haloalkyl comprising 2 to 7 halogen atoms that can be the same or different and selected from the group consisting of fluorine and chlorine;
Q¹ is O or S;
A is selected from the group consisting of direct bond, O, NR⁴, S, S═O, S(═O)₂, —(C═O)—, —(C═S)—, —O—(C═O)—, —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)═O—;
W¹ and W² are independently N, CH or CF;
m=0, 1 or 2; wherein, if m is 2, the two [CR¹R²] groups may be the same or different;
p=0, 1 or 2,
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with respectively one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of 0, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents, and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted respectively with one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that A is not NR⁴ when m is 1 or 2 and W¹ and W² are N;

provided U is not CCl₃ or CHCl₂ when W¹ and W² are CH;

provided that compound of formula (I) is not:
2,5-bis[5-(trichloromethyl)-1,3,4-oxadiazol-2-yl]pyridine [222190-08-1],
2-(bromomethyl)-5-[5-(difluoromethyl)-1,3,4-oxadiazol-2-yl]pyridine [2071232-31-8],
5-[5-(difluoromethyl)-1,3,4-oxadiazol-2-yl]-2-methylpyridine [2071232-29-4],
2-chloro-5-[5-(trifluoromethyl)-1,3,4-oxadiazol-2-yl]pyridine [2011795-38-1]
2-chloro-5-[5-(dichloromethyl)-1,3,4-oxadiazol-2-yl]pyridine [160152-11-4],
2-{5-[5-(trifluoromethyl)-1,3,4-oxadiazol-2-yl]pyridin-2-yl}-1H-benzimidazole-7-carboxamide [1103394-47-3],
5-[5-(difluoromethyl)-1,3,4-oxadiazol-2-yl]-N-{(4-fluorophenyl)[1-(methylsulfonyl)azetidin-3-yl]methyl}pyrimidin-2-amine [2243579-66-8],
5-[5-(difluoromethyl)-1,3,4-oxadiazol-2-yl]-2-(methylsulfonyl)pyrimidine [2095318-34-4],
5-[5-(difluoromethyl)-1,3,4-oxadiazol-2-yl]-2-(methylsulfanyl)pyrimidine [2095318-33-3].
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
N-[2-({5-[5-(difluoromethyl)-1,3,4-oxadiazol-2-yl]pyrimidin-2-yl}amino)-2-(tetrahydro-2H-pyran-4-yl)ethyl]methanesulfonamide [2243579-65-7],
1-[4-({5-[5-(difluoromethyl)-1,3,4-oxadiazol-2-yl]pyrimidin-2-yl}amino)-4-isopropylpiperidin-1-yl]ethanone [2243579-38-4],
N-{5-[5-(difluoromethyl)-1,3,4-oxadiazol-2-yl]pyrimidin-2-yl}-2,2-difluoro-N-[1-(4-fluorophenyl)-2-{3-[(methylsulfonyl)amino]phenyl}ethyl]acetamide [2243577-58-2],
N-[2-cyclopropyl-2-({5-[5-(difluoromethyl)-1,3,4-oxadiazol-2-yl]pyrimidin-2-yl}amino)ethyl]methane-sulfonamide [2243577-21-9],
N-[2-cyclopropyl-2-({5-[5-(difluoromethyl)-1,3,4-oxadiazol-2-yl]pyrimidin-2-yl}amino)ethyl]methane-sulfonamide [2243577-20-8],
N-[2-cyclopropyl-2-({5-[5-(difluoromethyl)-1,3,4-oxadiazol-2-yl]pyrimidin-2-yl}amino)ethyl]methane-sulfonamide [2243577-19-5],
N-[cyclopropyl(4-fluorophenyl)methyl]-N-{5-[5-(difluoromethyl)-1,3,4-oxadiazol-2-yl]pyrimidin-2-yl}-2,2-difluoroacetamide [2243576-38-5],
N-[cyclopropyl(4-fluorophenyl)methyl]-5-[5-(difluoromethyl)-1,3,4-oxadiazol-2-yl]pyrimidin-2-amine [2243576-35-2],
5-[5-(difluoromethyl)-1,3,4-oxadiazol-2-yl]-N-(1-isopropylcyclopropyl)pyrimidin-2-amine [2243576-06-7],
5-[5-(difluoromethyl)-1,3,4-oxadiazol-2-yl]-2-[(1-phenylcyclopropyl)oxy]pyrimidine [2243575-46-2] and
N-[(6-methylpyridin-2-yl)methyl]-5-[5-(trifluoromethyl)-1,3,4-oxadiazol-2-yl]pyrimidin-2-amine [2243575-26-8],
2-(difluoromethyl)-5-(4-iodophenyl)-1,3,4-oxadiazole [2244172-62-9],
2-isopropyl-5,6-dimethyl-3-{4-[5-(trifluoromethyl)-1,3,4-oxadiazol-2-yl]benzyl}pyridin-4-ol [2133324-02-2],
2-[4-(bromomethyl)-3-fluorophenyl]-5-(trifluoromethyl)-1,3,4-oxadiazole [2098919-34-5],
N-{4-[5-(difluoromethyl)-1,3,4-oxadiazol-2-yl]benzyl}butan-1-amine [2080363-69-3],
2-[4-(chloromethyl)phenyl]-5-(difluoromethyl)-1,3,4-oxadiazole [2071231-55-3],
2-[4-(bromomethyl)-3-fluorophenyl]-5-(difluoromethyl)-1,3,4-oxadiazole [2071227-85-3],
2-(difluoromethyl)-5-(3-fluoro-4-methylphenyl)-1,3,4-oxadiazole [2071227-84-2],
1-{4-[5-(trifluoromethyl)-1,3,4-oxadiazol-2-yl]phenyl}methanamine hydrochloride (1:1) [2071226-91-8],
N-{4-[5-(trifluoromethyl)-1,3,4-oxadiazol-2-yl]benzyl}methanesulfonamide [2071223-51-1],
methyl 4-[5-(trifluoromethyl)-1,3,4-oxadiazol-2-yl]benzoate [1352872-14-0],
2-(trifluoromethyl)-5-[4-(trifluoromethyl)phenyl]-1,3,4-oxadiazole [1352872-13-9],
2-(4-tert-butylphenyl)-5-(trifluoromethyl)-1,3,4-oxadiazole [1352872-12-8],
2-(4-methylphenyl)-5-(trifluoromethyl)-1,3,4-oxadiazole [1352872-11-7],
methyl {4-[5-(trifluoromethyl)-1,3,4-oxadiazol-2-yl]phenoxy}acetate [1227372-86-2],
ethyl 2-{4-[5-(trifluoromethyl)-1,3,4-oxadiazol-2-yl]phenoxy}propanoate [1227372-85-1],
2-(4-bromophenyl)-5-(trifluoromethyl)-1,3,4-oxadiazole [918476-23-0],
4-[5-(trifluoromethyl)-1,3,4-oxadiazol-2-yl]aniline [904643-35-2],
2-([biphenyl]-4-yl)-5-(trifluoromethyl)-1,3,4-oxadiazole [887267-97-2],
2-[4-(4,4,5,5-tetramethyl-1,3,2-dioxaborolan-2-yl)phenyl]-5-(trifluoromethyl)-1,3,4-oxadiazole [1056456-25-7],
2-(4-chlorophenyl)-5-(trifluoromethyl)-1,3,4-oxadiazole [627073-36-3],
2-(4-methoxyphenyl)-5-(trifluoromethyl)-1,3,4-oxadiazole [371950-64-0],
2-(dichloromethyl)-5-(4-methoxyphenyl)-1,3,4-oxadiazole [214195-06-9],
2-(4-tert-butylphenyl)-5-(dichloromethyl)-1,3,4-oxadiazole [160152-26-1]
2-(dichloromethyl)-5-(4-ethoxyphenyl)-1,3,4-oxadiazole [160152-21-6],
5-[5-(difluoromethyl)-1,3,4-oxadiazol-2-yl]-2-fluoropyridine [2137870-57-4], and
4-[5-(difluoromethyl)-1,3,4-thiadiazol-2-yl]aniline [2275439-93-3],
2-({4-[5-(difluoromethyl)-1,3,4-oxadiazol-2-yl]benzoyl}oxy)-1H-isoindole-1,3(2H)-dione [2248417-20-9],
tert-butyl 4-[5-(difluoromethyl)-1,3,4-oxadiazol-2-yl]benzoate [2241139-66-0],
methyl 4-[5-(difluoromethyl)-1,3,4-oxadiazol-2-yl]benzoate [2230804-32-5],
4-[5-(trifluoromethyl)-1,3,4-thiadiazol-2-yl]aniline [2160335-34-0],
4-[5-(difluoromethyl)-1,3,4-oxadiazol-2-yl]benzaldehyde [2138236-86-7],
2-(difluoromethyl)-5-(4-fluorophenyl)-1,3,4-oxadiazole [2137866-38-5],
2-(4-bromophenyl)-5-(difluoromethyl)-1,3,4-oxadiazole [2137697-81-3],
4-[5-(pentafluoroethyl)-1,3,4-oxadiazol-2-yl]benzoic acid [1920768-68-8],
4-[5-(1,1,2,2-tetrafluoroethyl)-1,3,4-oxadiazol-2-yl]benzoic acid [1917442-65-9],
4-[5-(difluoromethyl)-1,3,4-oxadiazol-2-yl]benzoic acid [1282022-66-5] and
4-[5-(trifluoromethyl)-1,3,4-oxadiazol-2-yl]benzoic acid [1197226-72-4].

In the above formula (I), U is preferably C₁-haloalkyl comprising 2 to 3 halogen atoms that can be the same or different and selected from the group consisting of fluorine and chlorine. More preferably, U is selected from CHF₂, CClF₂ and CF₃, still more preferably, U is CHF₂ or CF₃, even more preferably U is CHF₂.

In some embodiments, in the above formula (I), Q¹ is preferably O.

In some embodiments in the above formula (I), W¹ and W² are N, or W¹ is N and W² is CH, more preferably, W¹ and W² are N.

In the above formula (I), A is preferably a direct bond, O, S, NR⁴, —N(R⁴)—(C═O), —(C═O)—O—, —(C═O)—N(R⁵)—, —(C═O)—N(R⁴)—N(R⁵)—, —N(R⁴)—N(R⁵)—, —N(R⁴)—(C═O)—O— or —N(R⁴)—(C═O)—N(R⁵)—.

In some embodiments, A is a direct bond, O, NR⁴, —N(R⁴)—(C═O)—, —(C═O)—O— or —(C═O)—N(R⁵)—, more preferably O, NH, —(C═O)—O—, —(C═O)—NH— or —(C═O)—NMe-, even more preferably O or NH.

In some embodiments, in above formula (I), A is NR⁴, preferably NH.

In some embodiments, in above formula (I), A is O.

In some embodiments, in the above formula (I), m is preferably 0 or 1, more preferably m is 1.

In the above formula (I), p is preferably 0 or 1, more preferably p is 0.

In the above formula (I), each R¹ and each R² are preferably independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or
R¹ and R² may form, together with the carbon atom to which they are linked, a C₃-C₇-cycloalkyl or oxetanyl ring, or
two consecutive R¹, when m is 2, may form, together with the carbon atoms to which they are linked, a C₃-C₇-cycloalkyl ring.

In the above formula (I), it is more preferred that R¹ is selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl, and R² is selected from the group consisting of hydrogen, fluorine, chlorine, methyl, ethyl, trifluoromethyl and difluoromethyl,

or
R¹ and R² may form, together with the carbon atom to which they are linked, a cyclopropyl, cyclobutyl, cyclopentyl, cyclohexyl or oxetanyl ring, or
two consecutive R¹, when m is 2, may form, together with the carbon atoms to which they are linked, a cyclopropyl, cyclobutyl or cyclopentyl ring.

In the above formula (I), it is even more preferred that

m is 1,

R¹ is selected from the group consisting of hydrogen, methyl, ethyl, trifluoromethyl, ethynyl and cyclopropyl, and

R² is hydrogen,

or
R¹ and R² form, together with the carbon atom to which they are linked, a cyclopropyl, cyclobutyl or oxetanyl ring, more preferably a cyclopropyl or cyclobutyl ring, and most preferably a cyclopropyl ring.

In some embodiments, in above formula (I), there is at least [CR¹R²] group, in which R¹ and R² form, together with the carbon atom to which they are linked, a cyclopropyl, cyclobutyl, cyclopentyl, cyclohexyl or oxetanyl ring,

wherein, if m is 2, R¹ and R² of the other [CR¹R²] group are independently selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl, or form, together with the carbon atom to which they are linked, a cyclopropyl, cyclobutyl, cyclopentyl, cyclohexyl or oxetanyl ring.

In some embodiments, in above formula (I), m is 1, and R¹ and R² form, together with the carbon atom to which they are linked, a cyclopropyl, cyclobutyl, cyclopentyl, cyclohexyl or oxetanyl ring, more preferably a cyclopropyl, cyclobutyl or oxetanyl ring.

In some preferred embodiments, m is 1, and R¹ and R² form, together with the carbon atom to which they are linked, a cyclopropyl or cyclobutyl ring, preferably a cyclopropyl ring.

In some other preferred embodiments, m is 1, and R¹ and R² form, together with the carbon atom to which they are linked, a oxetanyl ring.

In still some other preferred embodiments,
m is 1,
R¹ is selected from the group consisting of hydrogen, methyl, ethyl, trifluoromethyl and cyclopropyl, and
R² is hydrogen.

In the above formula (I), R³ is preferably selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy, wherein said 1,3,2-dioxaborolan-2-yl may be substituted with one to four C₁-C₃-alkyl substituents, wherein said C₁-C₈-alkyl may be substituted with one C₁-C₈-alkoxy or C₁-C₈-haloalkoxy substituent, and wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl.

In the above formula (I), R³ is more preferably selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;

and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

In some preferred embodiments, R³ is selected from aryl and heteroaryl. In these embodiments, it is preferred that the aryl is selected from phenyl and naphthyl; the heteroaryl is selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; and the aryl and heteroaryl may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

In some embodiments, R³ in the above formula (I) is selected from phenyl and pyridine, wherein the phenyl and the pyridine may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In these embodiments, R³ is more preferably phenyl, which is unsubstituted or substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl, preferably unsubstituted or substituted with one or two substituents independently selected from fluorine, chlorine, bromine, methyl and methoxy.

In the above formula (I), R⁴ and R⁵ are preferably independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms.

In the above formula (I), R⁴ and R⁵ are more preferably independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms.

In the above formula (I), R⁴ and R⁵ are most preferably independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms.

In some particularly preferred embodiments, R⁴ and R⁵ are hydrogen.

Preferred are compounds of the formula (I):

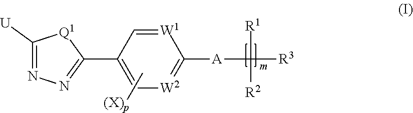

wherein

U is selected from CHF₂, CClF₂ and CF₃;

Q¹ is O or S;

W¹ and W² are independently N or CH;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0 or 1;

X is fluorine;

each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, preferably hydrogen, C₁-C₈-alkyl, C₁-C₈-halogenoalkyl C₂-C₈-alkynyl and C₃-C₇-cycloalkyl, wherein said C₁-C₈-alkyl may be substituted with one or more substituents selected from hydroxy and C₁-C₈-alkoxy, or

R¹ and R² may form, together with the carbon atom to which they are linked, a C₃-C₇-cycloalkyl or oxetanyl ring, or

two consecutive R¹, when m is 2, may form, together with the carbon atoms to which they are linked, a C₃-C₇-cycloalkyl ring;

R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;

R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms,

provided that A is not NR⁴ when m is 1 or 2 and W¹ and W² are N;

as well as their salts, N-oxides, solvates and optically active isomers or geometric isomers.

Particularly preferred are compounds of formula (I), wherein

U is selected from CHF₂, CClF₂ and CF₃, in particular CHF₂ or CF₃;

Q¹ is O or S, preferably O;

W¹ and W² are independently N or CH;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0;

R¹ is selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl,

or two consecutive R¹, when m is 2, may form, together with the carbon atoms to which they are linked, a cyclopropyl, cyclobutyl or cyclopentyl ring, and

R² is selected from the group consisting of hydrogen, fluorine, chlorine, methyl, ethyl, trifluoromethyl and difluoromethyl,

or

R¹ and R² may form, together with the carbon atom to which they are linked, a cyclopropyl, cyclobutyl, cyclopentyl, cyclohexyl or oxetanyl ring;

R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxy-ethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
preferably, R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and 2-naphthyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;

R⁴ and R⁵ are independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms, preferably R⁴ and R⁵ are independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms;

provided that A is not NR⁴ when m is 1 or 2 and W¹ and W² are N;

as well as their salts, N-oxides, solvates and optically active isomers or geometric isomers.

In some embodiments, U is CHF₂.

In some embodiments, W¹ and W² are N, or W¹ is N and W² is CH.

In some embodiments, A is a direct bond, O, S, NR⁴, —N(R⁴)—(C═O)—, —(C═O)—O— or —(C═O)—N(R⁵)—, more preferably O, S or NR⁴, even more preferably O or NR⁴, most preferably NH.

In some embodiments,

R¹ is selected from the group consisting of hydrogen, methyl, ethyl, iso-propyl, trifluoromethyl, ethenyl, ethynyl, phenyl and cyclopropyl,

R² is hydrogen,

or

R¹ and R² form, together with the carbon atom to which they are linked, a cyclopropyl, cyclobutyl or oxetanyl ring, more preferably a cyclopropyl or cyclobutyl ring, and most preferably a cyclopropyl

In some embodiments, R³ is selected from the group consisting of C₃-C₇-cycloalkyl, C₃-C₈-cycloalkenyl, aryl, heterocyclyl and heteroaryl, preferably C₃-C₇-cycloalkyl, aryl, heterocyclyl and heteroaryl; more preferably aryl or heteroaryl, and most preferably aryl;

wherein C₃-C₇-cycloalkyl is selected from cyclopropyl, cyclobutyl, cyclopentyl and cyclohexyl;

wherein C₃-C₈-cycloalkenyl is selected from cyclopentenyl and cyclohexenyl;

wherein aryl is selected from phenyl and naphthyl;

wherein heterocyclyl is selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl, preferably selected from piperidin-1-yl, piperazin-1-yl, tetrahydro-2H-pyran-4-yl, tetrahydrothiopyran-4-yl, morpholin-4-yl and 1,3-dihydro-2H-isoindol-2-yl;

wherein heteroaryl is selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl, preferably selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl;

and wherein said C₃-C₇-cycloalkyl, C₃-C₈-cycloalkenyl, aryl, heterocyclyl and heteroaryl may be substituted with one to three substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In some of said embodiments, R³ is selected from phenyl and pyridinyl, wherein said phenyl and pyridinyl may be substituted with one to three substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl. In these embodiments, R³ is more preferably phenyl, which is unsubstituted or substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl, in particular unsubstituted phenyl or phenyl which is substituted with one or two substituents independently selected from fluorine, chlorine, bromine, methyl and methoxy.

In some embodiments, R⁴ and R⁵ are hydrogen.

Most preferred are compounds of the formula (I) selected from the group consisting of
I.001 4-[5-(difluoromethyl)-1,3,4-oxadiazol-2-yl]benzoic acid
I.002 2-(difluoromethyl)-5-[4-(methylsulfanyl)phenyl]-1,3,4-oxadiazole
I.003 methyl 4-[5-(difluoromethyl)-1,3,4-oxadiazol-2-yl]benzoate
I.004 4-[5-(trifluoromethyl)-1,3,4-oxadiazol-2-yl]benzoic acid
I.005 2-(difluoromethyl)-5-[4-(1H-1,2,3-triazol-1-yl)phenyl]-1,3,4-oxadiazole
I.006 methyl 4-[5-(trifluoromethyl)-1,3,4-oxadiazol-2-yl]benzoate
I.007 2-([biphenyl]-4-yl)-5-(difluoromethyl)-1,3,4-oxadiazole
I.008 4-[5-(trifluoromethyl)-1,3,4-thiadiazol-2-yl]benzoic acid
I.009 2-(4-bromophenyl)-5-(difluoromethyl)-1,3,4-oxadiazole
I.010 2-(difluoromethyl)-5-[4-(1H-imidazol-1-ylmethyl)phenyl]-1,3,4-oxadiazole
I.011 methyl 4-[5-(trifluoromethyl)-1,3,4-thiadiazol-2-yl]benzoate
I.012 N-{4-[5-(difluoromethyl)-1,3,4-oxadiazol-2-yl]phenyl}-2,2-difluoroacetamide
I.013 N-{4-[5-(difluoromethyl)-1,3,4-oxadiazol-2-yl]phenyl}pyrimidin-2-amine
I.014 2-([biphenyl]-4-yl)-5-(trifluoromethyl)-1,3,4-oxadiazole
I.015 5-{4-[5-(difluoromethyl)-1,3,4-oxadiazol-2-yl]phenyl}-3-ethyl-1,2,4-oxadiazole
I.016 4-[5-(difluoromethyl)-1,3,4-oxadiazol-2-yl]-N-(2-methoxyethyl)benzamide
I.017 N-{4-[5-(difluoromethyl)-1,3,4-oxadiazol-2-yl]phenyl}-2-fluoroaniline
I.018 3-ethyl-5-{4-[5-(trifluoromethyl)-1,3,4-oxadiazol-2-yl]phenyl}-1,2,4-oxadiazole
I.019 2,2′-(1,4-phenylene)bis[5-(difluoromethyl)-1,3,4-oxadiazole]
I.020 N-(2-methoxyethyl)-4-[5-(trifluoromethyl)-1,3,4-oxadiazol-2-yl]benzamide
I.021 2-(difluoromethyl)-5-[4-(4,4,5,5-tetramethyl-1,3,2-dioxaborolan-2-yl)phenyl]-1,3,4-oxadiazole
I.022 4-[5-(difluoromethyl)-1,3,4-oxadiazol-2-yl]-N-methyl-N-phenylbenzamide
I.023 N-(2-methoxyethyl)-4-[5-(trifluoromethyl)-1,3,4-thiadiazol-2-yl]benzamide
I.024 2-{4-[(4-chlorophenoxy)methyl]phenyl}-5-(difluoromethyl)-1,3,4-oxadiazole
I.025 4-[5-(difluoromethyl)-1,3,4-oxadiazol-2-yl]-N-[1-(2-fluorophenyl)cyclopropyl]aniline
I.026 N-methyl-N-phenyl-4-[5-(trifluoromethyl)-1,3,4-oxadiazol-2-yl]benzamide
I.027 4-[5-(difluoromethyl)-1,3,4-oxadiazol-2-yl]-N-(2,4-difluorophenyl)benzamide
I.028 N-(2,4-difluorophenyl)-4-[5-(trifluoromethyl)-1,3,4-oxadiazol-2-yl]benzamide
I.029 N-(2,4-difluorophenyl)-4-[5-(trifluoromethyl)-1,3,4-thiadiazol-2-yl]benzamide
I.030 5-[5-(difluoromethyl)-1,3,4-oxadiazol-2-yl]pyridin-2-amine
I.031 2-chloro-5-[5-(difluoromethyl)-1,3,4-oxadiazol-2-yl]pyridine
I.032 5-[5-(difluoromethyl)-1,3,4-oxadiazol-2-yl]pyridine-2-carboxylic acid
I.033 5-[5-(difluoromethyl)-1,3,4-oxadiazol-2-yl]-N-methylpyridine-2-carboxamide
I.034 N-{5-[5-(difluoromethyl)-1,3,4-oxadiazol-2-yl]pyridin-2-yl}acetamide
I.035 5-[5-(difluoromethyl)-1,3,4-oxadiazol-2-yl]-2-(2-furyl)pyridine
I.036 5-[5-(difluoromethyl)-1,3,4-oxadiazol-2-yl]-2-(1H-pyrazol-1-yl)pyridine
I.037 5-[5-(difluoromethyl)-1,3,4-oxadiazol-2-yl]-2-(trifluoromethyl)pyridine
I.038 methyl {5-[5-(difluoromethyl)-1,3,4-oxadiazol-2-yl]pyridin-2-yl}carbamate
I.039 5-[5-(difluoromethyl)-1,3,4-oxadiazol-2-yl]-N-(prop-2-yn-1-yl)pyridine-2-carboxamide
I.040 5-[5-(difluoromethyl)-1,3,4-oxadiazol-2-yl]-2-(piperidin-1-yl)pyridine
I.041 4-{5-[5-(difluoromethyl)-1,3,4-oxadiazol-2-yl]pyridin-2-yl}morpholine
I.042 5-[5-(difluoromethyl)-1,3,4-oxadiazol-2-yl]-N′,N′-dimethylpyridine-2-carbohydrazide
I.043 5-[5-(difluoromethyl)-1,3,4-oxadiazol-2-yl]-2-phenoxypyridine
I.044 5-[5-(difluoromethyl)-1,3,4-oxadiazol-2-yl]-N-(1-methylcyclopropyl)pyridine-2-carboxamide
I.045 N-{5-[5-(difluoromethyl)-1,3,4-oxadiazol-2-yl]pyridin-2-yl}-1-methylcyclopropanecarboxamide
I.046 N-(cyclopropylmethyl)-5-[5-(difluoromethyl)-1,3,4-oxadiazol-2-yl]pyridine-2-carboxamide
I.047 5-[5-(difluoromethyl)-1,3,4-oxadiazol-2-yl]-N-(2-methoxyethyl)pyridine-2-carboxamide
I.048 5-[5-(difluoromethyl)-1,3,4-oxadiazol-2-yl]-N-(4-methylphenyl)pyridine-2-carboxamide
I.049 5-[5-(difluoromethyl)-1,3,4-oxadiazol-2-yl]-N-methyl-N-phenylpyridine-2-carboxamide
I.050 N-{5-[5-(difluoromethyl)-1,3,4-oxadiazol-2-yl]pyridin-2-yl}-2-phenylacetamide
I.051 l-{5-[5-(difluoromethyl)-1,3,4-oxadiazol-2-yl]pyridin-2-yl}-3-phenylurea
I.052 5-[5-(difluoromethyl)-1,3,4-oxadiazol-2-yl]-N-[(1RS)-1-(4-fluorophenyl)ethyl]pyridin-2-amine
I.053 5-{5-[chloro(difluoro)methyl]-1,3,4-oxadiazol-2-yl}-N-(2-fluorophenyl)pyridin-2-amine
I.054 5-[5-(difluoromethyl)-1,3,4-oxadiazol-2-yl]-N-[1-(4-fluorophenyl)cyclopropyl]pyridin-2-amine
I.055 N-{5-[5-(difluoromethyl)-1,3,4-oxadiazol-2-yl]pyridin-2-yl}-2,4-difluorobenzamide
I.056 5-[5-(difluoromethyl)-1,3,4-oxadiazol-2-yl]-N-[(1-phenylcyclopropyl)methyl]pyridine-2-carboxamide
I.057 5-{5-[chloro(difluoro)methyl]-1,3,4-oxadiazol-2-yl}-N-[1-(2-fluorophenyl)cyclopropyl]pyridin-2-amine
I.058 tert-butyl 4-{5-[5-(difluoromethyl)-1,3,4-oxadiazol-2-yl]pyridin-2-yl}piperazine-1-carboxylate
I.059 5-[5-(difluoromethyl)-1,3,4-oxadiazol-2-yl]pyrimidin-2-amine
I.060 5-[5-(difluoromethyl)-1,3,4-oxadiazol-2-yl]-N-methylpyrimidin-2-amine
I.061 2-chloro-5-[5-(difluoromethyl)-1,3,4-oxadiazol-2-yl]pyrimidine
I.062 5-[5-(difluoromethyl)-1,3,4-oxadiazol-2-yl]-N,N-dimethylpyrimidin-2-amine
I.064 5-[5-(difluoromethyl)-1,3,4-oxadiazol-2-yl]-2-(2,2-dimethylhydrazino)pyrimidine
I.065 2-(cyclopent-1-en-1-yl)-5-[5-(difluoromethyl)-1,3,4-oxadiazol-2-yl]pyrimidine
I.066 5-[5-(difluoromethyl)-1,3,4-oxadiazol-2-yl]-N-(1-methylcyclopropyl)pyrimidin-2-amine
I.068 5-[5-(difluoromethyl)-1,3,4-oxadiazol-2-yl]-N-(2-methoxyethyl)pyrimidin-2-amine
I.069 5-[5-(difluoromethyl)-1,3,4-oxadiazol-2-yl]-2-phenylpyrimidine
I.070 5-[5-(difluoromethyl)-1,3,4-oxadiazol-2-yl]-2-(pyridin-3-yl)pyrimidine
I.071 5-[5-(difluoromethyl)-1,3,4-oxadiazol-2-yl]-2-(2-thienyl)pyrimidine
I.072 5-[5-(difluoromethyl)-1,3,4-oxadiazol-2-yl]-2-(3-thienyl)pyrimidine
I.074 4-{5-[5-(difluoromethyl)-1,3,4-oxadiazol-2-yl]pyrimidin-2-yl}morpholine
I.075 2-benzyl-5-[5-(difluoromethyl)-1,3,4-oxadiazol-2-yl]pyrimidine
I.076 5-[5-(difluoromethyl)-1,3,4-oxadiazol-2-yl]-N-phenylpyrimidin-2-amine
I.077 5-[5-(difluoromethyl)-1,3,4-oxadiazol-2-yl]-2-(4-fluorophenyl)pyrimidine
I.078 5-[5-(difluoromethyl)-1,3,4-oxadiazol-2-yl]-2-(1-ethyl-1H-pyrazol-5-yl)pyrimidine
I.079 5-[5-(difluoromethyl)-1,3,4-oxadiazol-2-yl]-2-(2-fluoropyridin-4-yl)pyrimidine
I.080 5-[5-(difluoromethyl)-1,3,4-oxadiazol-2-yl]-2-(6-fluoropyridin-3-yl)pyrimidine
I.081 5-[5-(difluoromethyl)-1,3,4-oxadiazol-2-yl]-2-(3,5-dimethyl-1,2-oxazol-4-yl)pyrimidine
I.083 2-(cyclohexylmethyl)-5-[5-(difluoromethyl)-1,3,4-oxadiazol-2-yl]pyrimidine
I.085 4-{5-[5-(difluoromethyl)-1,3,4-oxadiazol-2-yl]pyrimidin-2-yl}benzonitrile
I.086 5-[5-(difluoromethyl)-1,3,4-oxadiazol-2-yl]-N-(2-methylphenyl)pyrimidin-2-amine
I.087 5-[5-(difluoromethyl)-1,3,4-oxadiazol-2-yl]-N-methyl-N-phenylpyrimidin-2-amine
I.089 5-[5-(difluoromethyl)-1,3,4-oxadiazol-2-yl]-2-(4-methoxyphenyl)pyrimidine
I.090 2-(benzyloxy)-5-[5-(difluoromethyl)-1,3,4-oxadiazol-2-yl]pyrimidine
I.091 5-[5-(difluoromethyl)-1,3,4-oxadiazol-2-yl]-2-(4-fluorobenzyl)pyrimidine
I.092 5-[5-(difluoromethyl)-1,3,4-oxadiazol-2-yl]-2-(phenylsulfanyl)pyrimidine
I.093 5-[5-(difluoromethyl)-1,3,4-oxadiazol-2-yl]-N-(2-fluorophenyl)pyrimidin-2-amine
I.094 5-[5-(difluoromethyl)-1,3,4-oxadiazol-2-yl]-N-(3-fluorophenyl)pyrimidin-2-amine
I.095 5-[5-(difluoromethyl)-1,3,4-oxadiazol-2-yl]-N-(4-fluorophenyl)pyrimidin-2-amine
I.096 5-[5-(difluoromethyl)-1,3,4-oxadiazol-2-yl]-N-(1-ethyl-1H-pyrazol-5-yl)pyrimidin-2-amine
I.098 5-[5-(difluoromethyl)-1,3,4-oxadiazol-2-yl]-2-(4-fluorophenoxy)pyrimidine
I.099 2-(2-chlorophenyl)-5-[5-(difluoromethyl)-1,3,4-oxadiazol-2-yl]pyrimidine
I.100 2-(3-chlorophenyl)-5-[5-(difluoromethyl)-1,3,4-oxadiazol-2-yl]pyrimidine
I.101 2-(4-chlorophenyl)-5-[5-(difluoromethyl)-1,3,4-oxadiazol-2-yl]pyrimidine
I.102 2-(2-chloropyridin-4-yl)-5-[5-(difluoromethyl)-1,3,4-oxadiazol-2-yl]pyrimidine
I.103 2-(5-chloropyridin-3-yl)-5-[5-(difluoromethyl)-1,3,4-oxadiazol-2-yl]pyrimidine
I.104 methyl 1-({5-[5-(difluoromethyl)-1,3,4-oxadiazol-2-yl]pyrimidin-2-yl}amino)cyclopropanecarboxylate
I.106 2-{5-[5-(difluoromethyl)-1,3,4-oxadiazol-2-yl]pyrimidin-2-yl}isoindoline
I.107 5-[5-(difluoromethyl)-1,3,4-oxadiazol-2-yl]-2-(2-methoxybenzyl)pyrimidine
I.108 5-[5-(difluoromethyl)-1,3,4-oxadiazol-2-yl]-2-(2-methyl-2-phenylhydrazino)pyrimidine
I.109 5-[5-(difluoromethyl)-1,3,4-oxadiazol-2-yl]-N-(2-methoxyphenyl)pyrimidin-2-amine
I.113 5-[5-(difluoromethyl)-1,3,4-thiadiazol-2-yl]-N-(2-fluorophenyl)pyrimidin-2-amine
I.114 N-(2-chlorophenyl)-5-[5-(difluoromethyl)-1,3,4-oxadiazol-2-yl]pyrimidin-2-amine
I.115 2-(2,4-difluorobenzyl)-5-[5-(difluoromethyl)-1,3,4-oxadiazol-2-yl]pyrimidine
I.116 N-(2-fluorophenyl)-5-[5-(trifluoromethyl)-1,3,4-oxadiazol-2-yl]pyrimidin-2-amine
I.117 ethyl 1-({5-[5-(difluoromethyl)-1,3,4-oxadiazol-2-yl]pyrimidin-2-yl}amino)cyclopropanecarboxylate
I.119 N-(2-cyclopropylphenyl)-5-[5-(difluoromethyl)-1,3,4-oxadiazol-2-yl]pyrimidin-2-amine
I.120 5-[5-(difluoromethyl)-1,3,4-oxadiazol-2-yl]-2-[(1-phenylcyclopropyl)oxy]pyrimidine
I.123 5-[5-(difluoromethyl)-1,3,4-oxadiazol-2-yl]-2-{[(1RS)-1-phenylethyl]sulfanyl}pyrimidine
I.127 5-[5-(difluoromethyl)-1,3,4-oxadiazol-2-yl]-2-{[(1RS)-1-(4-fluorophenyl)ethyl]oxy}pyrimidine
I.130 N-(2-fluorophenyl)-5-[5-(trifluoromethyl)-1,3,4-thiadiazol-2-yl]pyrimidin-2-amine
I.142 5-[5-(difluoromethyl)-1,3,4-oxadiazol-2-yl]-2-[(3-phenyloxetan-3-yl)oxy]pyrimidine
I.143 5-[5-(difluoromethyl)-1,3,4-oxadiazol-2-yl]-2-{[3-(pyridin-2-yl)oxetan-3-yl]oxy}pyrimidine
I.149 5-[5-(difluoromethyl)-1,3,4-oxadiazol-2-yl]-2-{[1-(4-fluorophenyl)cyclopropyl]oxy}pyrimidine
I.167 5-[5-(difluoromethyl)-1,3,4-oxadiazol-2-yl]-2-{[1-(2,6-difluorophenyl)cyclopropyl]oxy}pyrimidine
I.185 N-(2,4-difluorobenzoyl)-N-{5-[5-(difluoromethyl)-1,3,4-oxadiazol-2-yl]pyrimidin-2-yl}-2,4-difluorobenzamide
I.187 5-[5-(difluoromethyl)-1,3,4-oxadiazol-2-yl]-N-(1-isopropylcyclopropyl)pyrimidin-2-amine
I.222 methyl ({5-[5-(difluoromethyl)-1,3,4-oxadiazol-2-yl]pyrimidin-2-yl}amino)(pyridin-2-yl)acetate

The present invention relates to compounds of formula (I-A) (i.e. compound of formula (I) wherein W¹ is N):

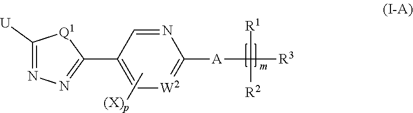

wherein
U is a C₁-C₃-haloalkyl comprising 2 to 7 halogen atoms that can be the same or different and selected from the group consisting of fluorine and chlorine;
Q¹ is O or S;
W² is CH or CF;
A is selected from the group consisting of direct bond, O, NR⁴, S, S═O, S(═O)₂, —(C═O)—, —(C═S)—, —O—(C═O)—, —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)═O—;
m=0, 1 or 2; wherein, if m is 2, the two [CR¹R²] groups may be the same or different;
p=0, 1, 2 or 3;
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with respectively one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of 0, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents, and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted respectively with one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that compound of formula (I-A) is not:
2,5-bis[5-(trichloromethyl)-1,3,4-oxadiazol-2-yl]pyridine [222190-08-1],
2-(bromomethyl)-5-[5-(difluoromethyl)-1,3,4-oxadiazol-2-yl]pyridine [2071232-31-8],
5-[5-(difluoromethyl)-1,3,4-oxadiazol-2-yl]-2-methylpyridine [2071232-29-4],
2-chloro-5-[5-(trifluoromethyl)-1,3,4-oxadiazol-2-yl]pyridine [2011795-38-1],
2-chloro-5-[5-(dichloromethyl)-1,3,4-oxadiazol-2-yl]pyridine [160152-11-4] and
2-{5-[5-(trifluoromethyl)-1,3,4-oxadiazol-2-yl]pyridin-2-yl}-1H-benzimidazole-7-carboxamide [1103394-47-3];
5-[5-(difluoromethyl)-1,3,4-oxadiazol-2-yl]-2-fluoropyridine [2137870-57-4] as well as their salts, N-oxides, solvates and optically active isomers or geometric isomers.

In the above formula (I-A), U is preferably C₁-haloalkyl comprising 2 to 3 halogen atoms that can be the same or different and selected from the group consisting of fluorine and chlorine, more preferably U is CHF₂ or CF₃, even more preferably U is CHF₂.

In the above formula (I-A), Q¹ is preferably O.

In the above formula (I-A), W² is preferably CH.

In the above formula (I-A), A is preferably O, NR⁴, —(C═O)—, —N(R⁴)—(C═O)—, —(C═O)—O— or —(C═O)—N(R⁵)—, more preferably A is O, NH, —(C═O)—O—, —(C═O)—NH— or —(C═O)—NMe-, even more preferably O or NH.

In the above formula (I-A), p is preferably 0 or 1, more preferably p is 0.

In the above formula (I-A), m is preferably 0 or 1, more preferably m is 1.

In the above formula (I-A), R¹ is preferably selected from the group consisting of hydrogen, cyano, C₁-C₈-alkyl, C₁-C₈-halogenoalkyl, C₂-C₈-alkynyl.

In the above formula (I-A), R² is preferably hydrogen or C₁-C₈-alkyl.

In the above formula (I-A), R¹ and R² may preferably form together with the carbon atom to which they are linked, a C₃-C₆-cycloalkyl or a 3- to 6-membered saturated or partially unsaturated heterocyclyl ring that contains 1 to 3 heteroatoms that can be the same or different and selected from the group consisting of O, S and NH, more preferably R¹ and R² may form together with the carbon atom to which they are linked, a cyclopropyl, a cyclobutyl ring or an oxetanyl ring.

In the above formula (I-A), R³ is preferably selected from the group consisting of hydrogen, C₁-C₈-alkyl that may be substituted by a C₁-C₈-alkoxy (e.g. 2-methoxyethyl), unsubstituted or substituted aryl, unsubstituted or substituted heterocyclyl and unsubstituted or substituted heteroaryl.

In the above formula (I-A), R⁴ is preferably hydrogen or C₁-C₈-alkyl.

In the above formula (I-A), R⁵ is preferably hydrogen or C₁-C₈-alkyl.

Preferred are compounds of the formula (I-A), wherein

U is selected from CHF₂, CClF₂ and CF₃;

Q¹ is O or S;

W² is CH; A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0 or 1;

X is fluorine;

each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, preferably selected from the group consisting of hydrogen, C₁-C₈-alkyl, C₁-C₈-halogenoalkyl, C₂-C₈-alkynyl and C₃-C₇-cycloalkyl and aryl,
wherein said C₁-C₈-alkyl may be substituted with one or more substituents selected from hydroxy and C₁-C₈-alkoxy, or

R¹ and R² may form, together with the carbon atom to which they are linked, a C₃-C₇-cycloalkyl or oxetanyl ring, or

two consecutive R¹, when m is 2, may form, together with the carbon atoms to which they are linked, a C₃-C₇-cycloalkyl ring;

R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;
R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms;

as well as their salts, N-oxides, solvates and optically active isomers or geometric isomers.

Particularly preferred are compounds of the formula (I-A), wherein

U is selected from CHF₂, CClF₂ and CF₃, in particular CHF₂ or CF₃;

Q¹ is O;

W² is CH;

A is a direct bond, O, NR⁴, —N(R⁴)—(C═O), —(C═O)—O—, —(C═O)—N(R⁵)—, —(C═O)—N(R⁴)—N(R⁵), —N(R⁴)—(C═O)—O— or —N(R⁴)—(C═O)—N(R⁵)—;

m is 0 or 1;

p is 0;

R¹ and R² are selected from the group consisting of hydrogen, methyl and ethynyl,

or

R¹ and R² may form, together with the carbon atom to which they are linked, a cyclopropyl ring;

R³ is selected from the group consisting of hydrogen, fluorine, bromine, chlorine, methyl, ethyl, trifluoromethyl, difluoromethyl, 2-methoxyethyl, 1-methoxyethyl, methoxymethyl; cyclopropyl, phenyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; and heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl;
and wherein said cyclopropyl, phenyl, heterocyclyl and heteroaryl may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;

R⁴ and R⁵ are independently selected from hydrogen and methyl;

as well as their salts, N-oxides, solvates and optically active isomers or geometric isomers.

The present invention relates to compounds of formula (I-B) (i.e. formula (I) wherein W¹ and W² are N):

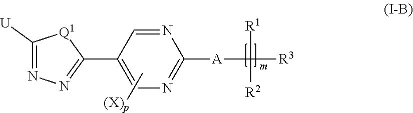

wherein
U is a C₁-C₃-haloalkyl comprising 2 to 7 halogen atoms that can be the same or different and selected from the group consisting of fluorine and chlorine;
Q¹ is O or S;
A is selected from the group consisting of direct bond, O, NR⁴, S, S═O, S(═O)₂, —(C═O)—, —(C═S)—, —O—(C═O)—, —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)═O—;
provided that A is not NR⁴ when m is 1 or 2;
m=0, 1 or 2; wherein, if m is 2, the two [CR¹R²] groups may be the same or different;
p=0, 1 or 2,
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with respectively one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of 0, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents, and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted respectively with one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that compound of formula (I-B) is not:
5-[5-(difluoromethyl)-1,3,4-oxadiazol-2-yl]-2-[(1-phenylcyclopropyl)oxy]pyrimidine [2243575-46-2],
5-[5-(difluoromethyl)-1,3,4-oxadiazol-2-yl]-2-(methylsulfonyl)pyrimidine [2095318-34-4] and
5-[5-(difluoromethyl)-1,3,4-oxadiazol-2-yl]-2-(methylsulfanyl)pyrimidine [2095318-33-3];

as well as their salts, N-oxides, hydates and optically active isomers or geometric isomers.

In the above formula (I-B), U is preferably C₁-haloalkyl comprising 2 to 3 halogen atoms that can be the same or different and selected from the group consisting of fluorine and chlorine, more preferably U is CHF₂ or CF₃, even more preferably U is CHF₂.

In the above formula (I-B), Q¹ is preferably O.

In the above formula (I-B), A is preferably O, S, NR⁴, —(C═O)—, —N(R⁴)—(C═O)—, —(C═O)—O— or —(C═O)—N(R⁵)—, more preferably A is O, S, NH, —(C═O)—O—, —(C═O)—NH— or —(C═O)—NMe-, even more preferably O or NH.

In the above formula (I-A), p is preferably 0 or 1, more preferably p is 0.

In the above formula (I-B), m is preferably 0 or 1, more preferably m is 1.

In the above formula (I-B), R¹ is preferably selected from the group consisting of hydrogen, cyano, C₁-C₈-alkyl, C₁-C₈-halogenoalkyl, C₂-C₈-alkynyl.

In the above formula (I-B), R² is preferably hydrogen or C₁-C₈-alkyl.

In the above formula (I-B), R¹ and R² may preferably form together with the carbon atom to which they are linked, a C₃-C₆-cycloalkyl or a 3- to 6-membered saturated or partially unsaturated heterocyclyl ring that contains 1 to 3 heteroatoms that can be the same or different and selected from the group consisting of O, S and NH, more preferably R¹ and R² may form together with the carbon atom to which they are linked, a cyclopropyl, a cyclobutyl ring or an oxetanyl ring.

In the above formula (I-B), R³ is preferably selected from the group consisting of hydrogen, C₁-C₈-alkyl that may be substituted by a C₁-C₈-alkoxy (e.g. 2-methoxyethyl), unsubstituted or substituted aryl, unsubstituted or substituted heterocyclyl and unsubstituted or substituted heteroaryl.

In the above formula (I-B), R⁴ is preferably hydrogen or C₁-C₈-alkyl.

In the above formula (I-B), R⁵ is preferably selected hydrogen or C₁-C₈-alkyl.

Preferred are compounds of the formula (I-B), wherein

U is selected from CHF₂, CClF₂ and CF₃;

Q¹ is O or S;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
provided that A is not NR⁴ when m is 1 or 2;

m is 0, 1 or 2; wherein, if m is 2, the two [CR¹R²] groups may be the same or different;

p is 0 or 1;

X is fluorine;

each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or

R¹ and R² may form, together with the carbon atom to which they are linked, a C₃-C₇-cycloalkyl or oxetanyl ring, or

two consecutive R¹, when m is 2, may form, together with the carbon atoms to which they are linked, a C₃-C₇-cycloalkyl ring;

R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;

R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms;

as well as their salts, N-oxides, solvates and optically active isomers or geometric isomers.

Particularly preferred are compounds of formula (I-B), wherein

U is selected from CHF₂, CClF₂ and CF₃, in particular CHF₂ or CF₃;

Q¹ is O or S, preferably O;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
provided that A is not NR⁴ when m is 1 or 2;

m is 0, 1 or 2; wherein, if m is 2, the two [CR¹R²] groups may be the same or different;

p is 0;

R¹ is selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl,

or two consecutive R¹, when m is 2, may form, together with the carbon atoms to which they are linked, a cyclopropyl, cyclobutyl or cyclopentyl ring, and

R² is selected from the group consisting of hydrogen, fluorine, chlorine, methyl, ethyl, trifluoromethyl and difluoromethyl,

or

R¹ and R² may form, together with the carbon atom to which they are linked, a cyclopropyl, cyclobutyl, cyclopentyl, cyclohexyl or oxetanyl ring;

R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxy-ethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
preferably, R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and 2-naphthyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;

R⁴ and R⁵ are independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms, preferably R⁴ and R⁵ are independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms;

as well as their salts, N-oxides, solvates and optically active isomers or geometric isomers.

The present invention relates to compounds of formula (I-C)

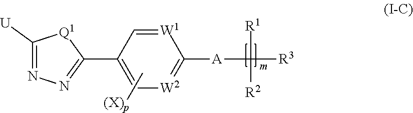

wherein
U is a C₁-C₃-haloalkyl comprising 2 to 7 halogen atoms that can be the same or different and selected from the group consisting of fluorine and chlorine; provided that U is not CCl₃ or CHCl₂;
Q¹ is O or S;
W¹ and W² are independently selected from CF and CH;
A is selected from the group consisting of direct bond, O, NR⁴, S, S═O, S(═O)₂, —(C═O)—, —(C═S)—, —O—(C═O)—, —O—(C═S)—, —N(R⁴)—(C═O)—, —N(R⁴)—(C═S)—, —(C═O)—O—, —(C═S)═O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)═O—;
m=0, 1 or 2; wherein, if m is 2, the two [CR¹R²] groups may be the same or different;
p=0, 1, 2, 3 or 4;
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with respectively one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of 0, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents, and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted respectively with one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;

provided that the compound of formula (I-C) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
2-(difluoromethyl)-5-(4-iodophenyl)-1,3,4-oxadiazole [2244172-62-9],
2-isopropyl-5,6-dimethyl-3-{4-[5-(trifluoromethyl)-1,3,4-oxadiazol-2-yl]benzyl}pyridin-4-ol [2133324-02-2],
2-[4-(bromomethyl)-3-fluorophenyl]-5-(trifluoromethyl)-1,3,4-oxadiazole [2098919-34-5],
2-[4-(chloromethyl)phenyl]-5-(difluoromethyl)-1,3,4-oxadiazole [2071231-55-3],
2-[4-(bromomethyl)-3-fluorophenyl]-5-(difluoromethyl)-1,3,4-oxadiazole [2071227-85-3],
2-(difluoromethyl)-5-(3-fluoro-4-methylphenyl)-1,3,4-oxadiazole [2071227-84-2],
methyl 4-[5-(trifluoromethyl)-1,3,4-oxadiazol-2-yl]benzoate [1352872-14-0],
2-(trifluoromethyl)-5-[4-(trifluoromethyl)phenyl]-1,3,4-oxadiazole [1352872-13-9],
2-(4-tert-butylphenyl)-5-(trifluoromethyl)-1,3,4-oxadiazole [1352872-12-8],
2-(4-methylphenyl)-5-(trifluoromethyl)-1,3,4-oxadiazole [1352872-11-7],
methyl {4-[5-(trifluoromethyl)-1,3,4-oxadiazol-2-yl]phenoxy}acetate [1227372-86-2],
ethyl 2-{4-[5-(trifluoromethyl)-1,3,4-oxadiazol-2-yl]phenoxy}propanoate [1227372-85-1],
2-[4-(4,4,5,5-tetramethyl-1,3,2-dioxaborolan-2-yl)phenyl]-5-(trifluoromethyl)-1,3,4-oxadiazole [1056456-25-7],
2-(4-bromophenyl)-5-(trifluoromethyl)-1,3,4-oxadiazole [918476-23-0],
4-[5-(trifluoromethyl)-1,3,4-oxadiazol-2-yl]aniline [904643-35-2],
2-([biphenyl]-4-yl)-5-(trifluoromethyl)-1,3,4-oxadiazole [887267-97-2],
2-(4-chlorophenyl)-5-(trifluoromethyl)-1,3,4-oxadiazole [627073-36-3],
2-(4-methoxyphenyl)-5-(trifluoromethyl)-1,3,4-oxadiazole [371950-64-0],
4-[5-(difluoromethyl)-1,3,4-thiadiazol-2-yl]aniline [2275439-93-3],
2-({4-[5-(difluoromethyl)-1,3,4-oxadiazol-2-yl]benzoyl}oxy)-1H-isoindole-1,3(2H)-dione [2248417-20-9],
tert-butyl 4-[5-(difluoromethyl)-1,3,4-oxadiazol-2-yl]benzoate [2241139-66-0],
   methyl 4-[5-(difluoromethyl)-1,3,4-oxadiazol-2-yl]benzoate [2230804-32-5],
   4-[5-(trifluoromethyl)-1,3,4-thiadiazol-2-yl]aniline [2160335-34-0],
   4-[5-(difluoromethyl)-1,3,4-oxadiazol-2-yl]benzaldehyde [2138236-86-7],
   2-(difluoromethyl)-5-(4-fluorophenyl)-1,3,4-oxadiazole [2137866-38-5],
   2-(4-bromophenyl)-5-(difluoromethyl)-1,3,4-oxadiazole [2137697-81-3],
   4-[5-(pentafluoroethyl)-1,3,4-oxadiazol-2-yl]benzoic acid [1920768-68-8],
   4-[5-(1,1,2,2-tetrafluoroethyl)-1,3,4-oxadiazol-2-yl]benzoic acid [1917442-65-9],
   4-[5-(difluoromethyl)-1,3,4-oxadiazol-2-yl]benzoic acid [1282022-66-5] and
   4-[5-(trifluoromethyl)-1,3,4-oxadiazol-2-yl]benzoic acid [1197226-72-4];

as well as their salts, N-oxides, solvates and optically active isomers or geometric isomers.

In the above formula (I-C), U is preferably C₁-haloalkyl comprising 2 to 3 halogen atoms that can be the same or different and selected from the group consisting of fluorine and chlorine provided that U is not CCl₃ or CHCl₂, more preferably U is CHF₂ or CF₃, even more preferably U is CHF₂.

In the above formula (I-C), Q¹ is preferably O.

In the above formula (I-C), A is preferably O, NR⁴, —(C═O)—, —N(R⁴)—(C═O)—, —(C═O)—O— or —(C═O)—N(R⁵)—, more preferably A is O, NH, —(C═O)—O—, —(C═O)—NH— or —(C═O)—NMe-, even more preferably O or NH.

In the above formula (I-C), p is preferably 0 or 1, more preferably p is 0.

In the above formula (I-C), m is preferably 0 or 1, more preferably m is 1.

In the above formula (I-C), R¹ is preferably selected from the group consisting of hydrogen, cyano, C₁-C₈-alkyl, C₁-C₈-halogenoalkyl, C₂-C₈-alkynyl.

In the above formula (I-C), R² is preferably hydrogen or C₁-C₈-alkyl.

In the above formula (I-C), R¹ and R² may preferably form together with the carbon atom to which they are linked, a C₃-C₆-cycloalkyl or a 3- to 6-membered saturated or partially unsaturated heterocyclyl ring that contains 1 to 3 heteroatoms that can be the same or different and selected from the group consisting of O, S and NH, more preferably R¹ and R² may form together with the carbon atom to which they are linked, a cyclopropyl, a cyclobutyl ring or an oxetanyl ring.

In the above formula (I-C), R³ is preferably selected from the group consisting of hydrogen, C₁-C₈-alkyl that may be substituted by a C₁-C₈-alkoxy (e.g. 2-methoxyethyl), unsubstituted or substituted aryl, unsubstituted or substituted heterocyclyl and unsubstituted or substituted heteroaryl.

In the above formula (I-C), R⁴ is preferably hydrogen or C₁-C₈-alkyl.

In the above formula (I-C), R⁵ is preferably hydrogen or C₁-C₈-alkyl.

Preferred are compounds of formula (I-C), wherein

U is selected from CHF₂, CClF₂ and CF₃;

Q¹ is O or S;

W¹ and W² are CH;

A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;

m is 0, 1 or 2; wherein, if m is 2, the two [CR¹R²] groups may be the same or different;

p is 0 or 1;

X is fluorine;

each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or

R¹ and R² may form, together with the carbon atom to which they are linked, a C₃-C₇-cycloalkyl or oxetanyl ring, or

two consecutive R¹, when m is 2, may form, together with the carbon atoms to which they are linked, a C₃-C₇-cycloalkyl ring;

R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;

R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms;

as well as their salts, N-oxides, solvates and optically active isomers or geometric isomers.

Particularly preferred are compounds of formula (I-C), wherein

U is CHF₂ or CF₃;

Q¹ is O or S, preferably O;

W¹ and W² are CH;

A is a direct bond, O, S, NR⁴, —N(R⁴)—(C═O), —(C═O)—O—, —(C═O)—N(R⁵)— or —(C═O)—N(R⁴)—N(R⁵);

m is 0 or 1;

p is 0;

R¹ and R² are selected from hydrogen and methyl,

or

R¹ and R² may form, together with the carbon atom to which they are linked, a cyclopropyl ring;

R³ is selected from the group consisting of hydrogen, fluorine, bromine, chlorine, methyl, ethyl, trifluoromethyl, difluoromethyl, 2-methoxyethyl, 1-methoxyethyl, methoxymethyl and 1,3,2-dioxaborolan-2-yl; phenyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl; and phenoxy;
wherein said 1,3,2-dioxaborolan-2-yl may be substituted with one to four methyl substituents, and wherein said phenyl, heterocyclyl, heteroaryl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;

R⁴ and R⁵ are independently selected from hydrogen and methyl;

as well as their salts, N-oxides, solvates and optically active isomers or geometric isomers.

The compounds of formula (I) including (I-A), (I-B) and (I-C) are useful for controlling phytopathogenic fungi (use as fungicide). Thus, the present invention relates to the use of a compound of formula (I), (I-A), (I-B) and (I-C) for controlling phytopathogenic fungi.

The present invention also relates to any compounds of formula (I) disclosed in Table 1.

Intermediates for the Preparation of the Active Ingredients

The present invention also relates to intermediates for the preparation of compounds of formula (I).

Unless indicated otherwise, the radicals and indices U, Q¹, W¹, W², A, p, m, R¹, R², R³, R⁴ and R⁵ have the meanings given above for the compounds of formula (I).

Thus, the present invention relates to compounds of formula (IIa) as well as their acceptable salts:

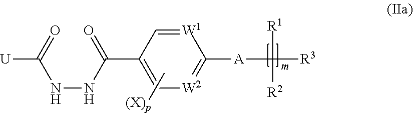

wherein
U is CHF₂ or CF₃,

provided that when m=1 and W¹ and W² are both N then A is not NH;

provided that the compound of formula (IIa) does not represent:
2-[(1-benzylcyclobutyl)amino]-N′-(difluoroacetyl)pyrimidine-5-carbohydrazide [2095319-66-5],
N′-(difluoroacetyl)-2-(methylsulfanyl)pyrimidine-5-carbohydrazide [2095318-32-2],
N′-(difluoroacetyl)-3-fluoro-4-methylbenzohydrazide [2071227-83-1],
6-chloro-N′-(trifluoroacetyl)nicotinohydrazide [2011795-44-9],
4-methoxy-N′-(trifluoroacetyl)benzohydrazide [314283-32-4] and
4-methyl-N′-(trifluoroacetyl)benzohydrazide [304668-19-7].

The following compounds of formula (IIa) are also mentioned in chemical databases and/or suppliers' databases but without any references or information which enable these to be prepared and separated:
4-(1H-tetrazol-1-yl)-N′-(trifluoroacetyl)benzohydrazide [1209426-58-3],
N-(4-{[2-(trifluoroacetyl)hydrazino]carbonyl}phenyl)benzamide [651709-76-1],
4-tert-butyl-N-(4-{[2-(trifluoroacetyl)hydrazino]carbonyl}phenyl)benzamide [642935-44-2],
4-(dimethylamino)-N′-(trifluoroacetyl)benzohydrazide [494200-26-9],
   N-(4-{[2-(trifluoroacetyl)hydrazino]carbonyl}phenyl)cyclohexanecarboxamide [443666-63-5],
N-(4-{[2-(trifluoroacetyl)hydrazino]carbonyl}phenyl)cyclopropanecarboxamide [443636-89-3],
2,2,2-trifluoro-N-(4-{[2-(trifluoroacetyl)hydrazino]carbonyl}phenyl)acetamide [357163-15-6],
2-methyl-N-(4-{[2-(trifluoroacetyl)hydrazino]carbonyl}phenyl)benzamide [356767-83-4],
N-(4-{[2-(trifluoroacetyl)hydrazino]carbonyl}phenyl)butanamide [356766-87-5],
3-methyl-N-(4-{[2-(trifluoroacetyl)hydrazino]carbonyl}phenyl)benzamide [356525-80-9],
N-(4-{[2-(trifluoroacetyl)hydrazino]carbonyl}phenyl)propanamide [349641-09-4],
4-chloro-N′-(trifluoroacetyl)benzohydrazide [349639-35-6],
N-(4-{[2-(trifluoroacetyl)hydrazino]carbonyl}phenyl)acetamide [314767-09-4] and
N′-(trifluoroacetyl)[biphenyl]-4-carbohydrazide [307339-48-6].

Preferred compounds of formula (IIa) according to the invention are:
N′-(difluoroacetyl)-4-(methylsulfanyl)benzohydrazide,
N-(4-{[2-(difluoroacetyl)hydrazino]carbonyl}phenyl)acetamide,
methyl 4-{[2-(difluoroacetyl)hydrazino]carbonyl}benzoate,
4-{[2-(trifluoroacetyl)hydrazino]carbonyl}benzoic acid,
N′-(difluoroacetyl)-4-(1H-1,2,3-triazol-1-yl)benzohydrazide,
methyl 4-{[2-(trifluoroacetyl)hydrazino]carbonyl}benzoate,
N′-(difluoroacetyl)[biphenyl]-4-carbohydrazide,
4-bromo-N′-(difluoroacetyl)benzohydrazide,
N′-(difluoroacetyl)-4-(1H-imidazol-1-ylmethyl)benzohydrazide,
N′-(difluoroacetyl)-4-(pyrimidin-2-ylamino)benzohydrazide,
N′-(difluoroacetyl)-4-(3-ethyl-1,2,4-oxadiazol-5-yl)benzohydrazide,
4-{[2-(difluoroacetyl)hydrazino]carbonyl}-N-(2-methoxyethyl)benzamide,
N′-(difluoroacetyl)-4-(2-fluoroanilino)benzohydrazide,
4-(3-ethyl-1,2,4-oxadiazol-5-yl)-N′-(trifluoroacetyl)benzohydrazide,
N-(2-methoxyethyl)-4-{[2-(trifluoroacetyl)hydrazino]carbonyl}benzamide,
4-{[2-(difluoroacetyl)hydrazino]carbonyl}-N-methyl-N-phenylbenzamide,
N′1,N′4-bis(difluoroacetyl)terephthalohydrazide,
4-[(4-chlorophenoxy)methyl]-N′-(difluoroacetyl)benzohydrazide,
N-methyl-N-phenyl-4-{[2-(trifluoroacetyl)hydrazino]carbonyl}benzamide,
4-{[2-(difluoroacetyl)hydrazino]carbonyl}-N-(2,4-difluorophenyl)benzamide,
N-(2,4-difluorophenyl)-4-{[2-(trifluoroacetyl)hydrazino]carbonyl}benzamide,
N′-(difluoroacetyl)-6-(2-furyl)nicotinohydrazide,
N′-(difluoroacetyl)-6-(trifluoromethyl)nicotinohydrazide,
N′-(difluoroacetyl)-6-(piperidin-1-yl)nicotinohydrazide,
N′-(difluoroacetyl)-6-phenoxynicotinohydrazide,
tert-butyl 4-(5-{[2-(difluoroacetyl)hydrazino]carbonyl}pyridin-2-yl)piperazine-1-carboxylate,
N′-(difluoroacetyl)-2-(morpholin-4-yl)pyrimidine-5-carbohydrazide,
N′-(difluoroacetyl)-2-(2-fluoroanilino)pyrimidine-5-carbohydrazide,
2-(2-fluoroanilino)-N′-(trifluoroacetyl)pyrimidine-5-carbohydrazide,
ethyl 1-[(5-{[2-(difluoroacetyl)hydrazino]carbonyl}pyrimidin-2-yl)amino]cyclopropanecarboxylate and
N′-(difluoroacetyl)-2-{[(1RS)-1-phenylethyl]sulfanyl}pyrimidine-5-carbohydrazide.

Processes for the Preparation of Compounds of Formula (I) and Intermediates

The present invention relates to processes for the preparation of compounds of formula (I) and their intermediates. Unless indicated otherwise, the radicals and indices U, Q¹, W¹, W², A, p, m, R¹, R², R³, R⁴ and R⁵ have the meanings given above for the compounds of formula (I). These definitions apply not only to the end products of formula (I) but also to all intermediates.

Compounds of formula (Ia), wherein Q¹=O, can be prepared, according to process P1, by reacting intermediates of formula (II) with a dehydrating agent, such as methyl N-(triethylammoniumsulfonyl)carbamate (Burgess reagent), in a suitable solvent such as tetrahydrofurane, as previously described in WO2017065473.

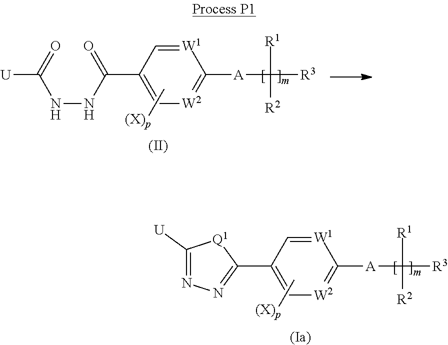

Compounds of formula (Ib), wherein Q¹=S can be prepared, according to process P2, by reacting intermediates of formula (II) with a thionation agent, like for example Lawesson's reagent (2,4-Bis(4-methoxyphenyl)-1,3,2,4-dithiadiphosphetane-2,4-dithione), in a suitable solvent such as toluene or tetrahydrofurane, as previously described in Org. Lett., 2006, 8, 1625-1628.

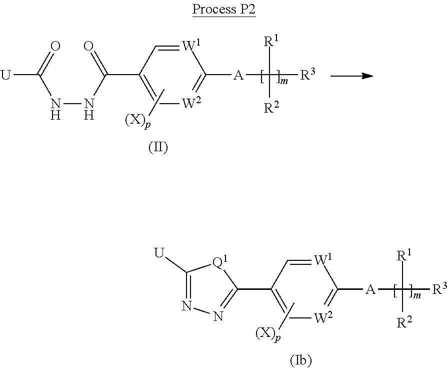

Intermediates of formula (II) can be commercially available or can be prepared according to process P3, by reacting carbohydrazides of formula (III) with C₁-C₃-haloalkylacetic anhydride or C₁-C₃-haloalkylacetyl chloride (which are either commercially available or may be prepared starting from readily available compounds according to known procedures) in a suitable solvent such as tetrahydrofurane optionally in presence of a base such as triethylamine, preferably at room temperature, as previously described in WO2017065473.

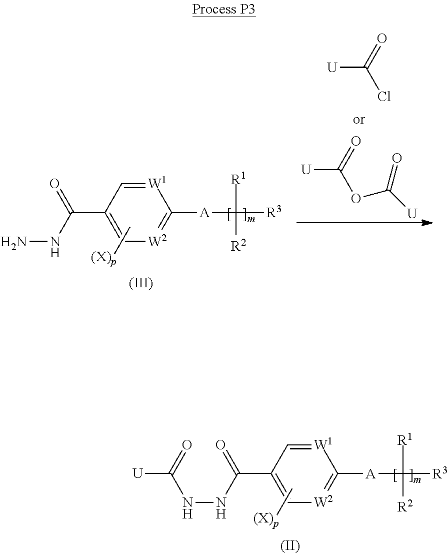

Carbohydrazides of formula (III) can be commercially available or can be prepared, according to process P4, by reacting a compound of formula (IV), wherein LG1 is a leaving group group as for example ethoxy with hydrazine hydrate in a suitable solvent such as ethanol, as previously described in WO2017065473.

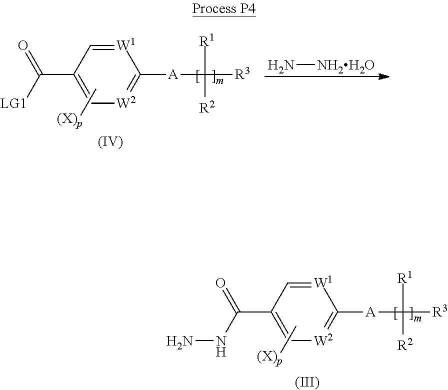

Compounds of formula (IV) can be commercially available or may be prepared starting from readily available compounds according to known procedures.

Alternatively, compounds of formula (IV′), wherein A is O or NR⁴, can be prepared, according to process P5, by reacting a compound of formula (V), wherein LG1 is an alkoxy like for example ethoxy and wherein LG2 is a leaving group like for example chlorine by nucleophilic substitution with a compound of formula (VI) (as described for example in European Journal of Medicinal Chemistry, 135, 531-543; 2017 or Bioorganic & Medicinal Chemistry, 25(17), 4553-4559; 2017) optionally in presence of a base (like for example N,N-diisopropylethylamine) or an acid (like for example p-toluenesulfonic acid) in a solvent such as for example dichloromethane or 1,4-dioxane. It may be necessary to activate the leaving group for example by oxidation with 3-chloroperbenzoic acid when LG2 is SMe.

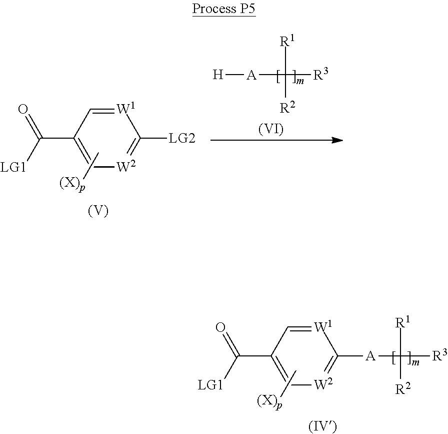

Compounds of formula (V) can be commercially available or may be prepared starting from readily available compounds according to known procedures.

Compounds of formula (VI) can be commercially available or may be prepared starting from readily available compounds according to known procedures.

Alternatively, carbohydrazides of formula (III) can be prepared, according to process P6, by reacting a compound of formula (VII) with an acid such as trifluoroacetic acid in a suitable solvent such as dichloromethane, preferably at room temperature, as previously described in Bioorganic & Medicinal Chemistry, 20(1), 487-497; 2012.

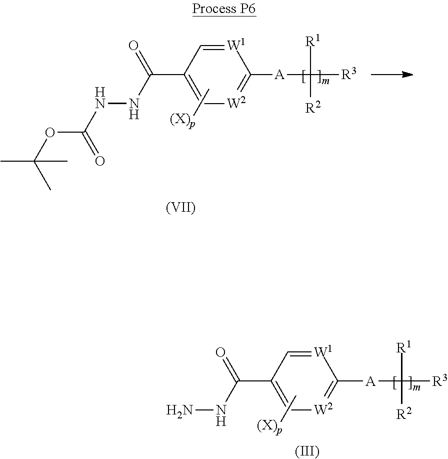

Compounds of formula (VII) can be commercially available or may be prepared, according to process P7, by reacting an acid of formula (VIII) with tert-butyl carbazate in presence of a coupling agent like for example (1-[Bis(dimethylamino)methylene]-1H-1,2,3-triazolo[4,5-b]pyridinium 3-oxid hexafluorophosphate (HATU) or 1,3-Propanediamine, N3-(ethylcarbonimidoyl)-N1,N1-dimethyl-, hydrochloride (EDCl), in a suitable solvent such as dichloromethane, optionally in presence of a base such as N,N-diisopropylethylamine, as previously described in Tetrahedron, 58(27), 5513-5523; 2002.

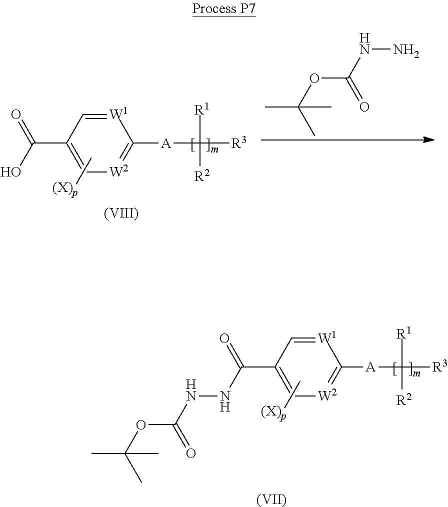

Compounds of formula (VIII) can be commercially available or may be prepared starting from readily available compounds according to known procedures.

Alternatively, compounds of formula (Ic), wherein A is O or NR⁴, can be prepared, according to process P8, from a compound of formula (IX), wherein LG3 is a leaving group by nucleophilic substitution with a compound of formula (VI) (as described for example in European Journal of Medicinal Chemistry, 135, 531-543; 2017 or WO2017065473) optionally in presence of a base (like for example triethylamine) or an acid (like for example p-toluenesulfonic acid) in a solvent such as for example dichloromethane or 1,4-dioxane. It may be necessary to activate the leaving group for example by oxidation with 3-chloroperbenzoic acid when LG2 is SMe.

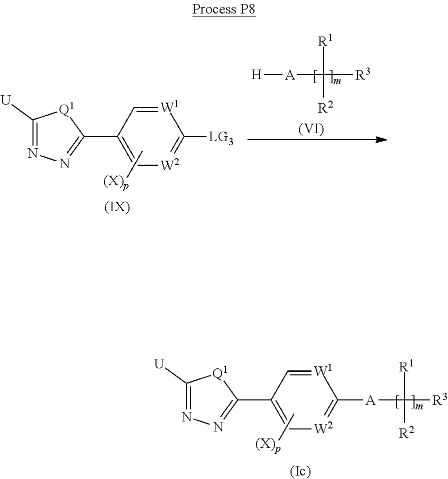

Compounds of formula (IX) can be commercially available or may be prepared starting from readily available compounds analogously to process P1 or P2 and P3 or P4 and P5.

According to the invention, processes P1 to P8 can be performed if appropriate in the presence of a solvent and if appropriate in the presence of a base.

Suitable solvents for carrying out processes P1 to P8 according to the invention are customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methyl-pyrrolidone or hexamethylphosphoric triamide; esters, such as methyl acetate or ethyl acetate, sulfoxides, such as dimethyl sulfoxide or sulfones, such as sulfolane.

Suitable bases for carrying out processes P1 to P8 according to the invention are inorganic and organic bases which are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as sodium carbonate, potassium carbonate, potassium bicarbonate, sodium bicarbonate, cesium carbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylaminopyridine, 1,4-diazabicyclo[2.2.2]octane (DABCO), 1,5-diazabicyclo[4.3.0]non-5-ene (DBN) or 1,8-diazabicyclo[5.4.0]undec-7-ene (DBU).

When carrying out processes P1 to P8, according to the invention, the reaction temperature can independently be varied within a relatively wide range. Generally, processes according to the invention are carried out at temperatures between −20° C. and 160° C. A way to control the temperature for the processes is to use microwave technology.

Processes P1 to P8 according to the invention are generally independently carried out under atmospheric pressure. However, it is also possible to operate under elevated or reduced pressure.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that can still be present.

Compounds according to the invention can be prepared according to the above described processes. It will nevertheless be understood that, on the basis of his general knowledge and of available publications, the skilled worker will be able to adapt these processes according to the specifics of each of the compounds according to the invention that is desired to be synthesized.

Aspects of the present teaching may be further understood in light of the following examples, which should not be construed as limiting the scope of the present teaching in any way.

Compositions and Formulations

The present invention further relates to a composition, in particular a composition for controlling unwanted phytopathogenic microorganisms. The compositions may be applied to the microorganisms and/or in their habitat.

The composition typically comprises at least one compound of formula (I) and at least one agriculturally suitable auxiliary, e.g. carrier(s) and/or surfactant(s).

A carrier is a solid or liquid, natural or synthetic, organic or inorganic substance that is generally inert. The carrier generally improves the application of the compounds, for instance, to plants, plants parts or seeds. Examples of suitable solid carriers include, but are not limited to, ammonium salts, natural rock flours, such as kaolins, clays, talc, chalk, quartz, attapulgite, montmorillonite and diatomaceous earth, and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as butanol or glycol), ketones (such as acetone, methyl ethyl ketone, methyl isobutyl ketone or cyclohexanone), esters (including fats and oils) and (poly)ethers, unsubstituted and substituted amines, amides (such as dimethylformamide), lactams (such as N-alkylpyrrolidones) and lactones, sulfones and sulfoxides (such as dimethyl sulfoxide). The carrier may also be a liquefied gaseous extender, i.e. liquid which is gaseous at standard temperature and under standard pressure, for example aerosol propellants such as halohydrocarbons, butane, propane, nitrogen and carbon dioxide. The amount of carrier typically ranges from 1 to 99.99%, preferably from 5 to 99.9%, more preferably from 10 to 99.5%, and most preferably from 20 to 99% by weight of the composition.

The surfactant can be an ionic (cationic or anionic) or non-ionic surfactant, such as ionic or non-ionic emulsifier(s), foam former(s), dispersant(s), wetting agent(s) and any mixtures thereof. Examples of suitable surfactants include, but are not limited to, salts of polyacrylic acid, salts of lignosulfonic acid, salts of phenolsulfonic acid or naphthalenesulfonic acid, polycondensates of ethylene and/or propylene oxide with fatty alcohols, fatty acids or fatty amines (polyoxyethylene fatty acid esters, polyoxyethylene fatty alcohol ethers, for example alkylaryl polyglycol ethers), substituted phenols (preferably alkylphenols or arylphenols), salts of sulfosuccinic esters, taurine derivatives (preferably alkyl taurates), phosphoric esters of polyethoxylated alcohols or phenols, fatty esters of polyols and derivatives of compounds containing sulfates, sulfonates, phosphates (for example, alkylsulfonates, alkyl sulfates, arylsulfonates) and protein hydrolysates, lignosulfite waste liquors and methylcellulose. A surfactant is typically used when the compound of formula (I) and/or the carrier is insoluble in water and the application is made with water. Then, the amount of surfactants typically ranges from 5 to 40% by weight of the composition.

Further examples of suitable auxiliaries include water repellents, siccatives, binders (adhesive, tackifier, fixing agent, such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone and tylose), thickeners, stabilizers (e.g. cold stabilizers, preservatives, antioxidants, light stabilizers, or other agents which improve chemical and/or physical stability), dyes or pigments (such as inorganic pigments, e.g. iron oxide, titanium oxide and Prussian Blue; organic dyes, e.g. alizarin, azo and metal phthalocyanine dyes), antifoams (e.g. silicone antifoams and magnesium stearate), preservatives (e.g. dichlorophene and benzyl alcohol hemiformal), secondary thickeners (cellulose derivatives, acrylic acid derivatives, xanthan, modified clays and finely divided silica), stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The choice of the auxiliaries is related to the intended mode of application of the compound of formula (I) and/or on the physical properties. Furthermore, the auxiliaries may be chosen to impart particular properties (technical, physical and/or biological properties) to the compositions or use forms prepared therefrom. The choice of auxiliaries may allow customizing the compositions to specific needs.

The composition may be in any customary form, such as solutions (e.g aqueous solutions), emulsions, wettable powders, water- and oil-based suspensions, powders, dusts, pastes, soluble powders, soluble granules, granules for broadcasting, suspoemulsion concentrates, natural or synthetic products impregnated with the compound of formula (I), fertilizers and also microencapsulations in polymeric substances. The compound of formula (I) may be present in a suspended, emulsified or dissolved form.

The composition may be provided to the end user as ready-for-use formulation, i.e. the compositions may be directly applied to the plants or seeds by a suitable device, such as a spraying or dusting device. Alternatively, the compositions may be provided to the end user in the form of concentrates which have to be diluted, preferably with water, prior to use.

The composition can be prepared in conventional manners, for example by mixing the compound of formula (I) with one or more suitable auxiliaries, such as disclosed herein above.

The composition contains generally from 0.01 to 99% by weight, from 0.05 to 98% by weight, preferably from 0.1 to 95% by weight, more preferably from 0.5 to 90% by weight, most preferably from 1 to 80% by weight of the compound of formula (I). It is possible that a composition comprises two or more compounds formula (I). In such case the outlined ranges refer to the total amount of compounds of the present invention.

Mixtures/Combinations

The compound of formula (I) and composition comprising thereof can be mixed with other active ingredients like fungicides, bactericides, acaricides, nematicides, insecticides, herbicides, fertilizers, growth regulators, safeners or semiochemicals. This may allow to broaden the activity spectrum or to prevent development of resistance. Examples of known fungicides, insecticides, acaricides, nematicides and bactericides are disclosed in the Pesticide Manual, 17th Edition.

Examples of especially preferred fungicides which could be mixed with the compound of formula (I) and the composition are:

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-di-chlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)—[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) Mefentrifluconazole, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Ipfentrifluconazole.

2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) Fluindapyr, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) isoflucypram, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide, (2.057) pyrapropoyne.

3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) mandestrobin, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (3.030) metyltetraprole, (3.031) florylpicoxamid.

4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.

5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.

6) Compounds capable to induce a host defense, for example (6.001) acibenzolar-S-methyl, (6.002) isotianil, (6.003) probenazole, (6.004) tiadinil.

7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

8) Inhibitors of the ATP production, for example (8.001) silthiofam.

9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Inhibitors of the lipid and membrane synthesis, for example (10.001) propamocarb, (10.002) propamocarb hydrochloride, (10.003) tolclofos-methyl.

11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.

12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).

13) Inhibitors of the signal transduction, for example (13.001) fludioxonil, (13.002) iprodione, (13.003) procymidone, (13.004) proquinazid, (13.005) quinoxyfen, (13.006) vinclozolin.

14) Compounds capable to act as an uncoupler, for example (14.001) fluazinam, (14.002) meptyldinocap.

15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) dipymetitrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) Ipflufenoquin, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) quinofumelin, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N‘-phenyl-N’-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one, (15.063) aminopyrifen.

All named mixing partners of the classes (1) to (15) as described here above can be present in the form of the free compound and/or, if their functional groups enable this, an agriculturally acceptable salt thereof.

The compound of formula (I) and the composition may also be combined with one or more biological control agents.

Examples of biological control agents which may be combined with the compound of formula (I) and composition comprising thereof are:

(A) Antibacterial Agents Selected from the Group of:

(A1) bacteria, such as (A1.1) Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); (A1.2) Bacillus amyloliquefaciens, in particular strain D747 (available as Double Nickel™ from Certis, US, having accession number FERM BP-8234 and disclosed in U.S. Pat. No. 7,094,592); (A1.3) Bacillus pumilus, in particular strain BU F-33 (having NRRL Accession No. 50185); (A1.4) Bacillus subtilis var. amyloliquefaciens strain FZB24 (available as Taegro® from Novozymes, US); (A1.5) a Paenibacillus sp. strain having Accession No. NRRL B-50972 or Accession No. NRRL B-67129 and described in International Patent Publication No. WO 2016/154297; and (A2) fungi, such as (A2.1) Aureobasidium pullulans, in particular blastospores of strain DSM14940; (A2.2) Aureobasidium pullulans blastospores of strain DSM14941; (A2.3) Aureobasidium pullulans, in particular mixtures of blastospores of strains DSM14940 and DSM14941;

(B) Fungicides Selected from the Group of:

(B1) bacteria, for example (B1.1) Bacillus subtilis, in particular strain QST713/AQ713 (available as SERENADE OPTI or SERENADE ASO from Bayer CropScience LP, US, having NRRL Accession No.

B21661 and described in U.S. Pat. No. 6,060,051); (B1.2) Bacillus pumilus, in particular strain QST2808 (available as SONATA® from Bayer CropScience LP, US, having Accession No. NRRL B-30087 and described in U.S. Pat. No. 6,245,551); (B1.3) Bacillus pumilus, in particular strain GB34 (available as Yield Shield® from Bayer AG, DE); (B1.4) Bacillus pumilus, in particular strain BU F-33 (having NRRL Accession No. 50185); (B1.5) Bacillus amyloliquefaciens, in particular strain D747 (available as Double Nickel™ from Certis, US, having accession number FERM BP-8234 and disclosed in U.S. Pat. No. 7,094,592); (B1.6) Bacillus subtilis Y1336 (available as BIOBAC® WP from Bion-Tech, Taiwan, registered as a biological fungicide in Taiwan under Registration Nos. 4764, 5454, 5096 and 5277); (B1.7) Bacillus amyloliquefaciens strain MBI 600 (available as SUBTILEX from BASF SE); (B1.8) Bacillus subtilis strain GB03 (available as Kodiak® from Bayer AG, DE); (B1.9) Bacillus subtilis var. amyloliquefaciens strain FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); (B1.10) Bacillus mycoides, isolate J (available as BmJ TGAI or WG from Certis USA); (B1.11) Bacillus licheniformis, in particular strain SB3086 (available as EcoGuard™ Biofungicide and Green Releaf from Novozymes); (B1.12) a Paenibacillus sp. strain having Accession No. NRRL B-50972 or Accession No. NRRL B-67129 and described in International Patent Publication No. WO 2016/154297.

In some embodiments, the biological control agent is a Bacillus subtilis or Bacillus amyloliquefaciens strain that produces a fengycin or plipastatin-type compound, an iturin-type compound, and/or a surfactin-type compound. For background, see the following review article: Ongena, M., et al., “Bacillus Lipopeptides: Versatile Weapons for Plant Disease Biocontrol,” Trends in Microbiology, Vol 16, No. 3, March 2008, pp. 115-125. Bacillus strains capable of producing lipopeptides include Bacillus subtilis QST713 (available as SERENADE OPTI or SERENADE ASO from Bayer CropScience LP, US, having NRRL Accession No. B21661 and described in U.S. Pat. No. 6,060,051), Bacillus amyloliquefaciens strain D747 (available as Double Nickel™ from Certis, US, having accession number FERM BP-8234 and disclosed in U.S. Pat. No. 7,094,592); Bacillus subtilis MBI600 (available as SUBTILEX® from Becker Underwood, US EPA Reg. No. 71840-8); Bacillus subtilis Y1336 (available as BIOBAC® WP from Bion-Tech, Taiwan, registered as a biological fungicide in Taiwan under Registration Nos. 4764, 5454, 5096 and 5277); Bacillus amyloliquefaciens, in particular strain FZB42 (available as RHIZOVITAL® from ABiTEP, DE); and Bacillus subtilis var. amyloliquefaciens FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); and (B2) fungi, for example: (B2.1) Coniothyrium minitans, in particular strain CON/M/91-8 (Accession No. DSM-9660; e.g. Contans® from Bayer); (B2.2) Metschnikowia fructicola, in particular strain NRRL Y-30752 (e.g. Shemer®); (B2.3) Microsphaeropsis ochracea (e.g. Microx® from Prophyta); (B2.5) Trichoderma spp., including Trichoderma atroviride, strain SC1 described in International Application No. PCT/IT2008/000196); (B2.6) Trichoderma harzianum rifaistrain KRL-AG2 (also known as strain T-22, /ATCC 208479, e.g. PLANTSHIELD T-22G, Rootshield®, and TurfShield from BioWorks, US); (B2.14) Gliocladium roseum, strain 321U from W.F. Stoneman Company LLC; (B2.35) Talaromyces flavus, strain V117b; (B2.36) Trichoderma asperellum, strain ICC 012 from Isagro; (B2.37) Trichoderma asperellum, strain SKT-1 (e.g. ECO-HOPE® from Kumiai Chemical Industry); (B2.38) Trichoderma atroviride, strain CNCM 1-1237 (e.g. Esquive® WP from Agrauxine, FR); (B2.39) Trichoderma atroviride, strain no. V08/002387; (B2.40) Trichoderma atroviride, strain NMI no. V08/002388; (B2.41) Trichoderma atroviride, strain NMI no. V08/002389; (B2.42) Trichoderma atroviride, strain NMI no. V08/002390; (B2.43) Trichoderma atroviride, strain LC52 (e.g. Tenet by Agrimm Technologies Limited); (B2.44) Trichoderma atroviride, strain ATCC 20476 (IMI 206040); (B2.45) Trichoderma atroviride, strain T11 (IM1352941/CECT20498); (B2.46) Trichoderma harmatum; (B2.47) Trichoderma harzianum; (B2.48) Trichoderma harzianum rifai T39 (e.g. Trichodex® from Makhteshim, US); (B2.49) Trichoderma harzianum, in particular, strain KD (e.g. Trichoplus from Biological Control Products, SA (acquired by Becker Underwood)); (B2.50) Trichoderma harzianum, strain ITEM 908 (e.g. Trianum-P from Koppert); (B2.51) Trichoderma harzianum, strain TH35 (e.g. Root-Pro by Mycontrol); (B2.52) Trichoderma virens (also known as Gliocladium virens), in particular strain GL-21 (e.g. SoilGard 12G by Certis, US); (B2.53) Trichoderma viride, strain TV1 (e.g. Trianum-P by Koppert); (B2.54) Ampelomyces quisqualis, in particular strain AQ 10 (e.g. AQ 10® by IntrachemBio Italia); (B2.56) Aureobasidium pullulans, in particular blastospores of strain DSM14940; (B2.57) Aureobasidium pullulans, in particular blastospores of strain DSM 14941; (B2.58) Aureobasidium pullulans, in particular mixtures of blastospores of strains DSM14940 and DSM 14941 (e.g. Botector® by bio-ferm, CH); (B2.64) Cladosporium cladosporioides, strain H39 (by Stichting Dienst Landbouwkundig Onderzoek); (B2.69) Gliocladium catenulatum (Synonym: Clonostachys rosea f. catenulate) strain J1446 (e.g. Prestop® by AgBio Inc. and also e.g. Primastop® by Kemira Agro Oy); (B2.70) Lecanicillium lecanii (formerly known as Verticillium lecanii) conidia of strain KV01 (e.g. Vertalec® by Koppert/Arysta); (B2.71) Penicillium vermiculatum; (B2.72) Pichia anomala, strain WRL-076 (NRRL Y-30842); (B2.75) Trichoderma atroviride, strain SKT-1 (FERM P-16510); (B2.76) Trichoderma atroviride, strain SKT-2 (FERM P-16511); (B2.77) Trichoderma atroviride, strain SKT-3 (FERM P-17021); (B2.78) Trichoderma gamsii(formerly T. viride), strain ICC080 (IMI CC 392151 CABI, e.g. BioDerma by AGROBIOSOL DE MEXICO, S.A. DE C.V.); (B2.79) Trichoderma harzianum, strain DB 103 (e.g., T-Gro 7456 by Dagutat Biolab); (B2.80) Trichoderma polysporum, strain IMI 206039 (e.g. Binab TF WP by BINAB Bio-Innovation AB, Sweden); (B2.81) Trichoderma stromaticum (e.g. Tricovab by Ceplac, Brazil); (B2.83) Ulocladium oudemansii, in particular strain HRU3 (e.g. Botry-Zen® by Botry-Zen Ltd, NZ); (B2.84) Verticillium albo-atrum (formerly V. dahliae), strain WCS850 (CBS 276.92; e.g. Dutch Trig by Tree Care Innovations); (B2.86) Verticillium chlamydosporium; (1B2.87) mixtures of Trichoderma asperellum strain ICC 012 and Trichoderma gamsii strain ICC 080 (product known as e.g. BIO-TAM™ from Bayer CropScience LP, US).

Further examples of biological control agents which may be combined with the compound of formula (I) and composition comprising thereof are:

bacteria selected from the group consisting of Bacillus cereus, in particular B. cereus strain CNCM I-1562 and Bacillus firmus, strain 1-1582 (Accession number CNCM I-1582), Bacillus subtilis strain OST 30002 (Accession No. NRRL B-50421), Bacillus thuringiensis, in particular B. thuringiensis subspecies israelensis (serotype H-14), strain AM65-52 (Accession No. ATCC 1276), B. thuringiensis subsp. aizawai, in particular strain ABTS-1857 (SD-1372), B. thuringiensis subsp. kurstaki strain HD-1, B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), and Streptomyces galbus strain AQ 6047 (Acession Number NRRL 30232);

fungi and yeasts selected from the group consisting of Beauveria bassiana, in particular strain ATCC 74040, Lecanicillium spp., in particular strain HRO LEC 12, Metarhizium anisopliae, in particular strain F52 (DSM3884 or ATCC 90448), Paecilomyces fumosoroseus (now: Isaria fumosorosea), in particular strain IFPC 200613, or strain Apopka 97 (Accesion No. ATCC 20874), and Paecilomyces lilacinus, in particular P. lilacinus strain 251 (AGAL 89/030550);

viruses selected from the group consisting of Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, and Spodoptera littoralis (African cotton leafworm) NPV. bacteria and fungi which can be added as ‘inoculant’ to plants or plant parts or plant organs and which, by virtue of their particular properties, promote plant growth and plant health. Examples are: Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium trifolii, Rhizopogon spp., Scleroderma spp., Suillus spp., and Streptomyces spp. plant extracts and products formed by microorganisms including proteins and secondary metabolites which can be used as biological control agents, such as Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, in particular oilseed rape powder or mustard powder.

Examples of insecticides, acaricides and nematicides, respectively, which could be mixed with the compound of formula (I) and composition comprising thereof are:

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel blockers, such as, for example, cyclodiene-organochlorines, for example chlordane and endosulfan or phenylpyrazoles (fiproles), for example ethiprole and fipronil.

(3) Sodium channel modulators, such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (Pyrethrum), resmethrin, silafluofen, tefluthrin, tetramethrin, tetramethrin [(1R)-isomer)], tralomethrin and transfluthrin or DDT or methoxychlor.

(4) Nicotinic acetylcholine receptor (nAChR) competitive modulators, such as, for example, neonicotinoids, e.g. acetamiprid, clothianidin, dinotefuran, imidacloprid, nitenpyram, thiacloprid and thiamethoxam or nicotine or sulfoxaflor or flupyradifurone.

(5) Nicotinic acetylcholine receptor (nAChR) allosteric modulators, such as, for example, spinosyns, e.g. spinetoram and spinosad.

(6) Glutamate-gated chloride channel (GluCI) allosteric modulators, such as, for example, avermectins/milbemycins, for example abamectin, emamectin benzoate, lepimectin and milbemectin.

(7) Juvenile hormone mimics, such as, for example, juvenile hormone analogues, e.g. hydroprene, kinoprene and methoprene or fenoxycarb or pyriproxyfen.

(8) Miscellaneous non-specific (multi-site) inhibitors, such as, for example, alkyl halides, e.g. methyl bromide and other alkyl halides; or chloropicrine or sulfuryl fluoride or borax or tartar emetic or methyl isocyanate generators, e.g. diazomet and metam.

(9) Modulators of Chordotonal Organs, such as, for example pymetrozine or flonicamid.

(10) Mite growth inhibitors, such as, for example clofentezine, hexythiazox and diflovidazin or etoxazole.

(11) Microbial disruptors of the insect gut membrane, such as, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins: Cry1Ab, Cry1Ac, Cry1Fa, Cry1A.105, Cry2Ab, Vip3A, mCry3A, Cry3Ab, Cry3Bb, Cry34Ab1/35Ab1.

(12) Inhibitors of mitochondrial ATP synthase, such as, ATP disruptors such as, for example, diafenthiuron or organotin compounds, for example azocyclotin, cyhexatin and fenbutatin oxide or propargite or tetradifon.

(13) Uncouplers of oxidative phosphorylation via disruption of the proton gradient, such as, for example, chlorfenapyr, DNOC and sulfluramid.

(14) Nicotinic acetylcholine receptor channel blockers, such as, for example, bensultap, cartap hydrochloride, thiocylam, and thiosultap-sodium.

(15) Inhibitors of chitin biosynthesis, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, for example buprofezin.

(17) Moulting disruptor (in particular for Diptera, i.e. dipterans), such as, for example, cyromazine.

(18) Ecdysone receptor agonists, such as, for example, chromafenozide, halofenozide, methoxyfenozide and tebufenozide.

(19) Octopamine receptor agonists, such as, for example, amitraz.

(20) Mitochondrial complex III electron transport inhibitors, such as, for example, hydramethylnone or acequinocyl or fluacrypyrim.

(21) Mitochondrial complex I electron transport inhibitors, such as, for example from the group of the METI acaricides, e.g. fenazaquin, fenpyroximate, pyrimidifen, pyridaben, tebufenpyrad and tolfenpyrad or rotenone (Derris).

(22) Voltage-dependent sodium channel blockers, such as, for example indoxacarb or metaflumizone.

(23) Inhibitors of acetyl CoA carboxylase, such as, for example, tetronic and tetramic acid derivatives, e.g. spirodiclofen, spiromesifen and spirotetramat.

(24) Mitochondrial complex IV electron transport inhibitors, such as, for example, phosphines, e.g. aluminium phosphide, calcium phosphide, phosphine and zinc phosphide or cyanides, e.g. calcium cyanide, potassium cyanide and sodium cyanide.

(25) Mitochondrial complex II electron transport inhibitors, such as, for example, beta-ketonitrile derivatives, e.g. cyenopyrafen and cyflumetofen and carboxanilides, such as, for example, pyflubumide.

(28) Ryanodine receptor modulators, such as, for example, diamides, e.g. chlorantraniliprole, cyantraniliprole and flubendiamide, further active compounds such as, for example, Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram, Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tigolaner, Tioxazafen, Thiofluoximate, Triflumezopyrim and iodomethane; furthermore preparations based on Bacillus firmus (1-1582, BioNeem, Votivo), and also the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3, 3,3-trifluoropropyl)sulfinyl]-propanamide, (+)-N-[3-chloro-1-(3-pyridinyl)-1H-pyrazol-4-yl]-N-ethyl-3-[(3, 3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl) [4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H-pyridinylidene]-2,2,2-trifluoro-acetamide (known from DE 3639877 A1, WO 2012029672 A1) (CAS 1363400-41-2), [N(E)]-N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]—N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1).

Examples of safeners which could be mixed with the compound of formula (I) and composition comprising thereof are, for example, benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}-sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

Examples of herbicides which could be mixed with the compound of formula (I) and composition comprising thereof are:

Acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate, and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, -2-ethylhexyl, -isobutyl, -isooctyl, -isopropylammonium, -potassium, -triisopropanolammonium, and -trolamine, 2,4-DB, 2,4-DB-butyl, -dimethylammonium, -isooctyl, -potassium, and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-{2-chloro-4-fluoro-5-[4-(3-fluoropropyl)-5-oxo-4,5-dihydro-1H-tetrazol-1-yl]phenyl}ethanesulfonamide, F-7967, i. e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium, and -trimesium, H-9201, i.e. O-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl) ethyl-(2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium, and -sodium, MCPB, MCPB-methyl, -ethy, l and -sodium, mecoprop, mecoprop-sodium, and -butotyl, mecoprop-P, mecoprop-P-butotyl, -dimethylammonium, -2-ethylhexyl, and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-(3-chloro-4-isopropylphenyl)-2-methylpentan amide, NGGC-011, napropamide, NC-310, i.e. [5-(benzyloxy)-1-methyl-1H-pyrazol-4-yl](2,4-dichlorophenyl)methanone, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxo-imidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

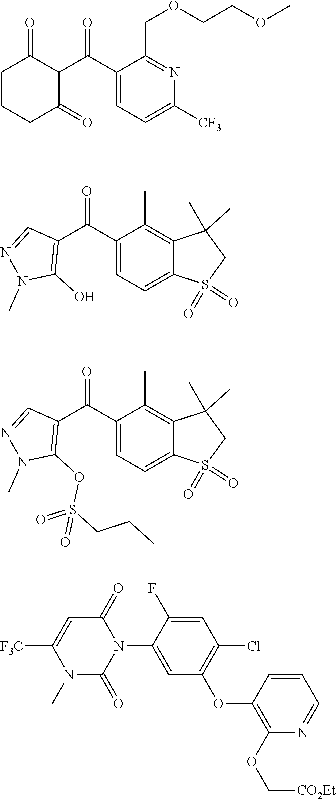

Examples for plant growth regulators are:

Acibenzolar, acibenzolar-S-methyl, 5-aminolevulinic acid, ancymidol, 6-benzylaminopurine, Brassinolid, catechine, chlormequat chloride, cloprop, cyclanilide, 3-(cycloprop-1-enyl) propionic acid, daminozide, dazomet, n-decanol, dikegulac, dikegulac-sodium, endothal, endothal-dipotassium, -disodium, and -mono(N,N-dimethylalkylammonium), ethephon, flumetralin, flurenol, flurenol-butyl, flurprimidol, forchlorfenuron, gibberellic acid, inabenfide, indol-3-acetic acid (IAA), 4-indol-3-ylbutyric acid, isoprothiolane, probenazole, jasmonic acid, maleic hydrazide, mepiquat chloride, 1-methyl-cyclopropene, methyl jasmonate, 2-(1-naphthyl)acetamide, 1-naphthylacetic acid, 2-naphthyloxyacetic acid, nitrophenolate-mixture, paclobutrazol, N-(2-phenylethyl)-beta-alanine, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Methods and Uses

The compound of formula (I) and composition comprising thereof comprising thereof have potent microbicidal activity and/or plant defense modulating potential. They can be used for controlling unwanted microorganisms, such as unwanted fungi and bacteria. They can be particularly useful in crop protection (they control microorganisms that cause plants diseases) or for protecting materials (e.g. industrial materials, timber, storage goods) as described in more details herein below. More specifically, the compound of formula (I) and composition comprising thereof can be used to protect seeds, germinating seeds, emerged seedlings, plants, plant parts, fruits, harvest goods and/or the soil in which the plants grow from unwanted microorganisms.

Control or controlling as used herein encompasses protective, curative and eradicative treatment of unwanted microorganisms. Unwanted microorganisms may be pathogenic bacteria, pathogenic virus, pathogenic oomycetes or pathogenic fungi, more specifically phytopathogenic bacteria, phytopathogenic virus, phytopathogenic oomycetes or phytopathogenic fungi. As detailed herein below, these phytopathogenic microorganims are the causal agents of a broad spectrum of plants diseases.

More specifically, the compound of formula (I) and composition comprising thereof can be used as fungicides. For the purpose of the specification, the term “fungicide” refers to a compound or composition that can be used in crop protection for the control of unwanted fungi, such as Plasmodiophoromycetes, Chytridiomycetes, Zygomycetes, Ascomycetes, Basidiomycetes and Deuteromycetes and/or for the control of Oomycetes.

The compound of formula (I) and composition comprising thereof may also be used as antibacterial agent. In particular, they may be used in crop protection, for example for the control of unwanted bacteria, such as Pseudomonadaceae, Rhizobiaceae, Xanthomonadaceae, Enterobacteriaceae, Corynebacteriaceae and Streptomycetaceae.

The compound of formula (I) and composition comprising thereof may also be used as antiviral agent in crop protection. For example the compound of formula (I) and composition comprising thereof may have effects on diseases from plant viruses, such as the tobacco mosaic virus (TMV), tobacco rattle virus, tobacco stunt virus (TStuV), tobacco leaf curl virus (VLCV), tobacco nervilia mosaic virus (TVBMV), tobacco necrotic dwarf virus (TNDV), tobacco streak virus (TSV), potato virus X (PVX), potato viruses Y, S, M, and A, potato acuba mosaic virus (PAMV), potato mop-top virus (PMTV), potato leaf-roll virus (PLRV), alfalfa mosaic virus (AMV), cucumber mosaic virus (CMV), cucumber green mottlemosaic virus (CGMMV), cucumber yellows virus (CuYV), watermelon mosaic virus (WMV), tomato spotted wilt virus (TSWV), tomato ringspot virus (TomRSV), sugarcane mosaic virus (SCMV), rice drawf virus, rice stripe virus, rice black-streaked drawf virus, strawberry mottle virus (SMoV), strawberry vein banding virus (SVBV), strawberry mild yellow edge virus (SMYEV), strawberry crinkle virus (SCrV), broad beanwilt virus (BBWV), and melon necrotic spot virus (MNSV).

The present invention also relates to a method for controlling phytopathogenic fungi, preferably selected from the group consisting of the Puccinia species, for example Puccinia recondita, Puccinia graminis or Puccinia striiformis; the Uromyces species, for example Uromyces appendiculatus; and the rust disease pathogens, in particular selected from the group consisting of the Gymnosporangium species, for example Gymnosporangium sabinae; Hemileia species, for example Hemileia vastatrix, and Phakopsora species, for example Phakopsora pachyrhizi or Phakopsora meibomiae, comprising the step of applying at least one compound of formula (I) or at least one composition according to the invention to the microorganisms and/or their habitat (to the plants, plant parts, seeds, fruits or to the soil in which the plants grow).

Typically, when the compound of formula (I) and composition comprising thereof are used in curative or protective methods for controlling phytopathogenic fungi, an effective and plant-compatible amount thereof is applied to the plants, plant parts, fruits, seeds or to the soil or substrates in which the plants grow. Suitable substrates that may be used for cultivating plants include inorganic based substrates, such as mineral wool, in particular stone wool, perlite, sand or gravel; organic substrates, such as peat, pine bark or sawdust; and petroleum based substrates such as polymeric foams or plastic beads.

Effective and plant-compatible amount means an amount that is sufficient to control or destroy the fungi present or liable to appear on the cropland and that does not entail any appreciable symptom of phytotoxicity for said crops. Such an amount can vary within a wide range depending on the fungus to be controlled, the type of crop, the crop growth stage, the climatic conditions and the respective compound of formula (I) or composition used. This amount can be determined by systematic field trials that are within the capabilities of a person skilled in the art.

Plants and Plant Parts

The compound of formula (I) and composition comprising thereof may be applied to any plants or plant parts.

Plants mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the genetically modified plants (GMO or transgenic plants) and the plant cultivars which are protectable and non-protectable by plant breeders' rights.

Genetically Modified Plants (GMO)

Genetically modified plants (GMO or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome. This gene gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plant cultivars are understood to mean plants which have new properties (“traits”) and have been obtained by conventional breeding, by mutagenesis or by recombinant DNA techniques. They can be cultivars, varieties, bio- or genotypes.

Plant parts are understood to mean all parts and organs of plants above and below the ground, such as shoots, leaves, needles, stalks, stems, flowers, fruit bodies, fruits, seeds, roots, tubers and rhizomes.

The plant parts also include harvested material and vegetative and generative propagation material, for example cuttings, tubers, rhizomes, slips and seeds.

Plants which may be treated in accordance with the methods described herein include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

Plants and plant cultivars which may be treated by the above disclosed methods include plants and plant cultivars which are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may be treated by the above disclosed methods include those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may be treated by the above disclosed methods include those plants characterized by enhanced yield characteristics. Increased yield in said plants may be the result of, for example, improved plant physiology, growth and development, such as water use efficiency, water retention efficiency, improved nitrogen use, enhanced carbon assimilation, improved photosynthesis, increased germination efficiency and accelerated maturation. Yield may furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants and plant cultivars which may be treated by the above disclosed methods include plants and plant cultivars which are hybrid plants that already express the characteristic of heterosis or hybrid vigor which results in generally higher yield, vigor, health and resistance towards biotic and abiotic stresses.

Plants and plant cultivars (obtained by plant biotechnology methods such as genetic engineering) which may be treated by the above disclosed methods include plants and plant cultivars which are herbicide-tolerant plants, i.e. plants made tolerant to one or more given herbicides. Such plants can be obtained either by genetic transformation, or by selection of plants containing a mutation imparting such herbicide tolerance.

Plants and plant cultivars (obtained by plant biotechnology methods such as genetic engineering) which may be treated by the above disclosed methods include plants and plant cultivars which are insect-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which may be treated by the above disclosed methods include plants and plant cultivars which are disease-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which may be treated by the above disclosed methods include plants and plant cultivars which are tolerant to abiotic stresses. Such plants can be obtained by genetic transformation, or by selection of plants containing a mutation imparting such stress resistance.

Plants and plant cultivars (obtained by plant biotechnology methods such as genetic engineering) which may be treated by the above disclosed methods include plants and plant cultivars which show altered quantity, quality and/or storage-stability of the harvested product and/or altered properties of specific ingredients of the harvested product.

Plants and plant cultivars (obtained by plant biotechnology methods such as genetic engineering) which may be treated by the above disclosed methods include plants and plant cultivars, such as cotton plants, with altered fiber characteristics. Such plants can be obtained by genetic transformation, or by selection of plants contain a mutation imparting such altered fiber characteristics.

Plants and plant cultivars (obtained by plant biotechnology methods such as genetic engineering) which may be treated by the above disclosed methods include plants and plant cultivars, such as oilseed rape or related Brassica plants, with altered oil profile characteristics. Such plants can be obtained by genetic transformation, or by selection of plants contain a mutation imparting such altered oil profile characteristics.

Plants and plant cultivars (obtained by plant biotechnology methods such as genetic engineering) which may be treated by the above disclosed methods include plants and plant cultivars, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants and plant cultivars (obtained by plant biotechnology methods such as genetic engineering) which may be treated by the above disclosed methods include plants and plant cultivars, such as Tobacco plants, with altered post-translational protein modification patterns.

Pathogens

Non-limiting examples of pathogens of fungal diseases which may be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Corynespora species, for example Corynespora cassiicola; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum; diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

fruit rot caused, for example, by Aspergillus species, for example Aspergillus flavus; Botrytis species, for example Botrytis cinerea; Monilinia species, for example Monilinia laxa; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum;

seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;

cancers, galls and witches' broom caused, for example, by Nectria species, for example Nectria galligena;

wilt diseases caused, for example, by Verticillium species, for example Verticillium longisporum; Fusarium species, for example Fusarium oxysporum;

deformations of leaves, flowers and fruits caused, for example, by Exobasidium species, for example Exobasidium vexans; Taphrina species, for example Taphrina deformans;

degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

diseases of plant tubers caused, for example, by Rhizoctonia species, for example Rhizoctonia solani; Helminthosporium species, for example Helminthosporium solani;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora; Liberibacter species, for example Liberibacter asiaticus; Xyella species, for example Xylella fastidiosa; Ralstonia species, for example Ralstonia solanacearum; Dickeya species, for example Dickeya solani; Clavibacter species, for example Clavibacter michiganensis; Streptomyces species, for example Streptomyces scabies.

diseases of soya beans:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), Cercospora leaf spot and blight (Cercospora kikuchii), Choanephora leaf blight (Choanephora infundibulifera trispora (Syn.)), Dactuliophora leaf spot (Dactuliophora glycines), downy mildew (Peronospora manshurica), Drechslera blight (Drechslera Glycini), frogeye leaf spot (Cercospora sojina), Leptosphaerulina leaf spot (Leptosphaerulina trifolii), Phyllostica leaf spot (Phyllosticta sojaecola), pod and stem blight (Phomopsis sojae), powdery mildew (Microsphaera diffusa), Pyrenochaeta leaf spot (Pyrenochaeta glycines), Rhizoctonia aerial, foliage, and web blight (Rhizoctonia solani), rust (Phakopsora pachyrhizi, Phakopsora meibomiae), scab (Sphaceloma glycines), Stemphylium leaf blight (Stemphylium botryosum), sudden death syndrome (Fusarium virguliforme), target spot (Corynespora cassiicola).

Fungal diseases on roots and the stem base caused, for example, by black root rot (Calonectria crotalariae), charcoal rot (Macrophomina phaseolina), Fusarium blight or wilt, root rot, and pod and collar rot (Fusarium oxysporum, Fusarium orthoceras, Fusarium semitectum, Fusarium equiseti), Mycoleptodiscus root rot (Mycoleptodiscus terrestris), Neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), Pythium rot (Pythium aphanidermatum, Pythium irregulare, Pythium debaryanum, Pythium myriotylum, Pythium ultimum), Rhizoctonia root rot, stem decay, and damping-off (Rhizoctonia solani), Sclerotinia stem decay (Sclerotinia sclerotiorum), Sclerotinia southern blight (Sclerotinia rolfsii), Thielaviopsis root rot (Thielaviopsis basicola).

Mycotoxins

In addition, the compound of formula (I) and composition comprising thereof may reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

Material Protection

The compound of formula (I) and composition comprising thereof may also be used in the protection of materials, especially for the protection of industrial materials against attack and destruction by phytopathogenic fungi.

In addition, the compound of formula (I) and composition comprising thereof may be used as antifouling compositions, alone or in combinations with other active ingredients.

Industrial materials in the present context are understood to mean inanimate materials which have been prepared for use in industry. For example, industrial materials which are to be protected from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compound of formula (I) and composition comprising thereof may prevent adverse effects, such as rotting, decay, discoloration, decoloration or formation of mould.

In the case of treatment of wood the compound of formula (I) and composition comprising thereof may also be used against fungal diseases liable to grow on or inside timber.

Timber means all types of species of wood, and all types of working of this wood intended for construction, for example solid wood, high-density wood, laminated wood, and plywood. In addition, the compound of formula (I) and composition comprising thereof may be used to protect objects which come into contact with saltwater or brackish water, especially hulls, screens, nets, buildings, moorings and signalling systems, from fouling.

The compound of formula (I) and composition comprising thereof may also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, may be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound of formula (I) and composition comprising thereof may prevent adverse effects, such as rotting, decay, discoloration, decoloration or formation of mould.

Microorganisms capable of degrading or altering industrial materials include, for example, bacteria, fungi, yeasts, algae and slime organisms. The compound of formula (I) and composition comprising thereof preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Seed Treatment

The compound of formula (I) and composition comprising thereof may also be used to protect seeds from unwanted microorganisms, such as phytopathogenic microorganisms, for instance phytopathogenic fungi or phytopathogenic oomycetes. The term seed(s) as used herein include dormant seeds, primed seeds, pregerminated seeds and seeds with emerged roots and leaves.

Thus, the present invention also relates to a method for protecting seeds from unwanted microorganisms which comprises the step of treating the seeds with the compound of formula (I) or the composition.

The treatment of seeds with the compound of formula (I) or the composition protects the seeds from phytopathogenic microorganisms, but also protects the germinating seeds, the emerging seedlings and the plants after emergence from the treated seeds. Therefore, the present invention also relates to a method for protecting seeds, germinating seeds and emerging seedlings.

The seeds treatment may be performed prior to sowing, at the time of sowing or shortly thereafter.

When the seeds treatment is performed prior to sowing (e.g. so-called on-seed applications), the seeds treatment may be performed as follows: the seeds may be placed into a mixer with a desired amount of the compound of formula (I) or the composition, the seeds and the compound of formula (I) or the composition are mixed until an homogeneous distribution on seeds is achieved. If appropriate, the seeds may then be dried.

The invention also relates to seeds coated with the compound of formula (I) or composition comprising thereof.

Preferably, the seeds are treated in a state in which it is sufficiently stable for no damage to occur in the course of treatment. In general, seeds can be treated at any time between harvest and shortly after sowing. It is customary to use seeds which have been separated from the plant and freed from cobs, shells, stalks, coats, hairs or the flesh of the fruits. For example, it is possible to use seeds which have been harvested, cleaned and dried down to a moisture content of less than 15% by weight. Alternatively, it is also possible to use seeds which, after drying, for example, have been treated with water and then dried again, or seeds just after priming, or seeds stored in primed conditions or pre-germinated seeds, or seeds sown on nursery trays, tapes or paper.

The amount of the compound of formula (I) or composition comprising thereof applied to the seeds is typically such that the germination of the seed is not impaired, or that the resulting plant is not damaged.

This must be ensured particularly in case the the compound of formula (I) would exhibit phytotoxic effects at certain application rates. The intrinsic phenotypes of transgenic plants should also be taken into consideration when determining the amount of the compound of formula (I) to be applied to the seed in order to achieve optimum seed and germinating plant protection with a minimum amount of compound being employed.

The compound of formula (I) can be applied as such, directly to the seeds, i.e. without the use of any other components and without having been diluted. Also the composition comprising thereof can be applied to the seeds.

The compound of formula (I) and composition comprising thereof are suitable for protecting seeds of any plant variety. Preferred seeds are that of cereals (such as wheat, barley, rye, millet, triticale, and oats), oilseed rape, maize, cotton, soybean, rice, potatoes, sunflower, beans, coffee, peas, beet (e.g. sugar beet and fodder beet), peanut, vegetables (such as tomato, cucumber, onions and lettuce), lawns and ornamental plants. More preferred are seeds of wheat, soybean, oilseed rape, maize and rice.

The compound of formula (I) and composition comprising thereof may be used for treating transgenic seeds, in particular seeds of plants capable of expressing a polypeptide or protein which acts against pests, herbicidal damage or abiotic stress, thereby increasing the protective effect. Seeds of plants capable of expressing a polypeptide or protein which acts against pests, herbicidal damage or abiotic stress may contain at least one heterologous gene which allows the expression of said polypeptide or protein. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

Application

The compound of formula (I) can be applied as such, or for example in the form of as ready-to-use solutions, emulsions, water- or oil-based suspensions, powders, wettable powders, pastes, soluble powders, dusts, soluble granules, granules for broadcasting, suspoemulsion concentrates, natural products impregnated with the compound of formula (I), synthetic substances impregnated with the compound of formula (I), fertilizers or microencapsulations in polymeric substances.

Application is accomplished in a customary manner, for example by watering, spraying, atomizing, broadcasting, dusting, foaming, spreading-on and the like. It is also possible to deploy the compound of formula (I) by the ultra-low volume method, via a drip irrigation system or drench application, to apply it in-furrow or to inject it into the soil stem or trunk. It is further possible to apply the compound of formula (I) by means of a wound seal, paint or other wound dressing.

The effective and plant-compatible amount of the compound of formula (I) which is applied to the plants, plant parts, fruits, seeds or soil will depend on various factors, such as the compound/composition employed, the subject of the treatment (plant, plant part, fruit, seed or soil), the type of treatment (dusting, spraying, seed dressing), the purpose of the treatment (curative and protective), the type of microorganisms, the development stage of the microorganisms, the sensitivity of the microorganisms, the crop growth stage and the environmental conditions.

When the compound of formula (I) is used as a fungicide, the application rates can vary within a relatively wide range, depending on the kind of application. For the treatment of plant parts, such as leaves, the application rate may range from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used). For the treatment of seeds, the application rate may range from 0.1 to 200 g per 100 kg of seeds, preferably from 1 to 150 g per 100 kg of seeds, more preferably from 2.5 to 25 g per 100 kg of seeds, even more preferably from 2.5 to 12.5 g per 100 kg of seeds. For the treatment of soil, the application rate may range from 0.1 to 10 000 g/ha, preferably from 1 to 5000 g/ha.

These application rates are merely examples and are not intended to limit the scope of the present invention.

Aspects of the present teaching may be further understood in light of the following examples, which should not be construed as limiting the scope of the present teaching in any way.

### EXAMPLES

Generality

Measurement of Log P values

Measurement of Log P values as provided herein was performed according to EEC directive 79/831

Annex V.A8 by HPLC (High Performance Liquid Chromatography) on reversed phase columns with the following methods:
^{[a]} Log P value is determined by measurement of LC-UV, in an acidic range, with 0.1% formic acid in water and acetonitrile as eluent (linear gradient from 10% acetonitrile to 95% acetonitrile).
^{[b]} Log P value is determined by measurement of LC-UV, in a neutral range, with 0.001 molar ammonium acetate solution in water and acetonitrile as eluent (linear gradient from 10% acetonitrile to 95% acetonitrile).
^{[c]} Log P value is determined by measurement of LC-UV, in an acidic range, with 0.1% phosphoric acid and acetonitrile as eluent (linear gradient from 10% acetonitrile to 95% acetonitrile).

If more than one Log P value is available within the same method, all the values are given and separated by “+”.

Calibration was done with straight-chain alkan2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals

¹H-NMR Data

¹H-NMR data of selected examples as provided herein are written in form of ¹H-NMR-peak lists. To each signal peak are listed the δ-value in ppm and the signal intensity in round brackets. Between the δ-value
signal intensity pairs are semicolons as delimiters.

The peak list of an example has therefore the form:

δ₁ (intensity₁); δ₂ (intensity₂); . . . δ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for ¹H spectra, we use tetramethylsilane and/or the chemical shift of the solvent used, especially in the case of spectra measured in DMSO. Therefore in NMR peak lists, tetramethylsilane peak can occur but not necessarily.

The ¹H-NMR peak lists are similar to classical ¹H-NMR prints and contains therefore usually all peaks, which are listed at classical NMR-interpretation.

Additionally they can show like classical ¹H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our ¹H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists you find in the publication “Citation of NMR Peaklist Data within patent Applications” of the Research Disclosure Database Number 564025.

The following examples illustrate in a non-limiting manner the preparation and biological activity of the compounds of formula (I) according to the invention.

### SYNTHESIS OF COMPOUNDS OF FORMULA (I) AND INTERMEDIATES

Table 1 illustrates in a non-limiting manner examples of compounds of formula (I) according to the invention:

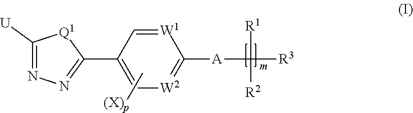

The compounds of formula (I) which are mentioned in table 1 herein below were prepared in accordance with the procedures detailed herein below in connection with specific examples and with the general description of the processes herein disclosed.

In table 1, the log P values were determined according to method [^{a}].

In table 1, “*” denotes the point of attachment of the diradical A to the aromatic group, “#” denotes the point of attachment of the diradical A to the diradical —[CR¹R²]ₘ—, and “˜” denotes the point of attachment of the diradical —[CR¹R²]ₘ— to the R³ group.

TABLE 1

| Example | U | Q¹ | W¹ | W² | (X)ₚ | A | —[CR¹R²]ₘ— | R³ | M + H | logP |
|---|---|---|---|---|---|---|---|---|---|---|
| I-001 | CHF₂ | O | CH | CH | — | *-(C═O)—O-# | — | H | | 1.66 |
| I-002 | CHF₂ | O | CH | CH | — | S | — | methyl | 243 | 2.75 |
| I-003 | CHF₂ | O | CH | CH | — | *-(C═O—O-# | — | methyl | | 2.35 |
| I-004 | CF₃ | O | CH | CH | — | *-(C═O)—O-# | — | H | | 2.20 |
| I-005 | CHF₂ | O | CH | CH | — | — | — | 1H-1,2,3-triazol-1-yl | 264 | 1.73 |
| I-006 | CF₃ | O | CH | CH | — | *-(C═O—O-# | — | methyl | | 2.97 |
| I-007 | CHF₂ | O | CH | CH | — | — | — | phenyl | 273 | 3.57 |
| I-008 | CF₃ | S | CH | CH | — | *-(C═O)—O-# | — | H | | 2.41 |
| I-009 | CHF₂ | O | CH | CH | — | — | — | bromo | 275 | 2.89 |
| I-010 | CHF₂ | O | CH | CH | — | — | CH₂ | 1H-imidazol-1-yl | 277 | 0.60 |
| I-011 | CF₃ | S | CH | CH | — | *-(C═O—O-# | — | methyl | | 3.23 |
| I-012 | CHF₂ | O | CH | CH | — | *-NH—(O═O)-# | — | difluoromethyl | 290 | 2.01 |
| I-013 | CHF₂ | O | CH | CH | — | NH | — | pyrimidin-2-yl | | 2.23 |
| I-014 | CF₃ | O | CH | CH | — | — | — | phenyl | | |
| I-015 | CHF₂ | O | CH | CH | — | — | — | 3-ethyl-1,2,4-oxadiazol-5-yl | 293 | 2.94 |
| I-016 | CHF₂ | O | CH | CH | — | *-(C═O)—NH-# | — | 2-methoxyethyl | | 1.58 |
| I-017 | CHF₂ | O | CH | CH | — | NH | — | 2-fluorophenyl | | 3.19 |
| I-018 | CF₃ | O | CH | CH | — | — | — | 3-ethyl-1,2,4-oxadiazol-5-yl | 311 | 3.59 |
| I-019 | CHF₂ | O | CH | CH | — | — | — | 5-(difluoromethyl)-1,3,4- | 315 | 2.44 |
| | | | | | | | | oxadiazol-2-yl | | |
| I-020 | CF₃ | O | CH | CH | — | *-(C═O)—NH-# | — | 2-methoxyethyl | | 2.04 |
| I-021 | CHF₂ | O | CH | CH | — | — | — | 4,4,5,5-tetramethyl-1,3,2- | 323 | 3.84 |
| | | | | | | | | dioxaborolan-2-yl | | |
| I-022 | CHF₂ | O | CH | CH | — | *-(C═O)—N(Me)-# | — | phenyl | 330 | 2.46 |
| I-023 | CF₂ | S | CH | CH | — | *-(C═O)—NH-# | — | 2-methoxyethyl | | 2.18 |
| I-024 | CHF₂ | O | CH | CH | — | — | CH₂ | 4-chlorophenoxy | 337 | 4.03 |
| I-025 | CHF₂ | O | CH | CH | — | NH | 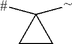 | 2-fluorophenyl | | 3.57 |
| I-026 | CF₃ | O | CH | CH | — | *-(C═O)—N(Me)-# | — | phenyl | 348 | 3.04 |
| I-027 | CHF₂ | O | CH | CH | — | *-(C═O)—NH-# | — | 2,4-difluorophenyl | | 2.57 |
| I-028 | CF₃ | O | CH | CH | — | *-(C═O)—NH-# | — | 2,4-difluorophenyl | | 3.12 |
| I-029 | CF₃ | S | CH | CH | — | *-(C═O)—NH-# | — | 2,4-difluorophenyl | | 3.29 |
| I-030 | CHF₂ | O | N | CH | — | NH | — | H | 213 | 0.11 |
| I-031 | CHF₂ | O | N | CH | — | — | — | chloro | 232 | 1.92 |
| I-032 | CHF₂ | O | N | CH | — | *-(C═O)—O-# | — | H | 240² | 0.83 |
| I-033 | CHF₂ | O | N | CH | — | *-(C═O)—NH-# | — | methyl | 255 | 1.32 |
| I-034 | CHF₂ | O | N | CH | — | *-NH—(O═O)-# | — | methyl | 255 | 1.32 |
| I-035 | CHF₂ | O | N | CH | — | — | — | 2-furyl | 264 | 2.33 |
| I-036 | CHF₂ | O | N | CH | — | — | — | 1H-pyrazol-1-yl | 264 | 2.31 |
| I-037 | CHF₂ | O | N | CH | — | — | — | trifluoromethyl | 266 | 2.39 |
| I-038 | CHF₂ | O | N | CH | — | *-NH—(C═O)—O-# | — | methyl | 271 | 1.66 |
| I-039 | CHF₂ | O | N | CH | — | *-(C═O)—NH-# | CH(C≡CH) | H | 279 | 1.68 |
| I-040 | CHF₂ | O | N | CH | — | — | — | piperidin-1-yl | 281 | 2.72 |
| I-041 | CHF₂ | O | N | CH | — | — | — | morpholin-4-yl | 283 | 1.91 |
| I-042 | CHF₂ | O | N | CH | — | *-(C═O)—NH—NMe-# | — | methyl | 284 | 1.14 |
| I-043 | CHF₂ | O | N | CH | — | O | — | phenyl | 290 | 2.90 |
| I-044 | CHF₂ | O | N | CH | — | *-(C═O)—NH-# | — | 1-methylcyclopropyl | 295 | 2.02 |
| I-045 | CHF₂ | O | N | CH | — | *-NH—(O═O)-# | — | 1-methylcyclopropyl | 295 | 2.28 |
| I-046 | CHF₂ | O | N | CH | — | *-(C═O)—NH-# | CH₂ | cyclopropyl | 295 | 2.16 |
| I-047 | CHF₂ | O | N | CH | — | *-(C═O)—NH-# | — | 2-methoxyethyl | 299 | 1.55 |
| I-048 | CHF₂ | O | N | CH | — | *-(C═O)—NH-# | — | 4-methylphenyl | 331 | 3.14 |
| I-049 | CHF₂ | O | N | CH | — | *-(C═O)—NMe-# | — | phenyl | 331 | 2.05 |
| I-050 | CHF₂ | O | N | CH | — | *-NH—(O═O)-# | CH₂ | phenyl | 331 | 2.54 |
| I-051 | CHF₂ | O | N | CH | — | *-NH—(C═O)—NH-# | — | phenyl | 332 | 2.57 |
| I-052 | CHF₂ | O | N | CH | — | NH | CH(Me) | 4-fluorophenyl | 335 | 2.95 |
| I-053 | CClF₂ | O | N | CH | — | NH | — | 2-fluorophenyl | | 3.54 |
| I-054 | CHF₂ | O | N | CH | — | NH | 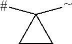 | 4-fluorophenyl | 347 | 2.79 |
| I-055 | CHF₂ | O | N | CH | — | *-NH—(O═O)-# | — | 2,4-difluorophenyl | 353 | 2.77 |
| I-056 | CHF₂ | O | N | CH | — | *-(C═O)—NH-# | 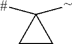 | phenyl | 371 | 3.20 |
| I-057 | CClF₂ | O | N | CH | — | NH | 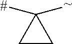 | 2-fluorophenyl | | 3.81 |
| I-058 | CHF₂ | O | N | CH | — | — | — | 4-(tert- | 382 | 3.25 |
| | | | | | | | | butoxycarbonyl)piperazin-1-yl | | |
| I-059 | CHF₂ | O | N | N | — | NH | — | H | 214 | 0.75 |
| I-060 | CHF₂ | O | N | N | — | NH | — | methyl | 228 | 1.23 |
| I-061 | CHF₂ | O | N | N | — | — | — | chloro | 233 | 1.55 |
| I-062 | CHF₂ | O | N | N | — | NMe | — | methyl | 242 | 1.87 |
| I-063 | CHF₂ | O | N | N | — | NH | CH(C≡CH) | H | 252 | 1.59 |
| I-064 | CHF₂ | O | N | N | — | *-NH—NMe-# | — | methyl | 257 | 0.76 |
| I-065 | CHF₂ | O | N | N | — | — | — | cyclopent-1-en-1-yl | 265 | 2.70 |
| I-066 | CHF₂ | O | N | N | — | NH | — | 1-methylcyclopropyl | 268 | 1.90 |
| I-067 | CHF₂ | O | N | N | — | NH | CH₂ | cyclopropyl | 268 | 2.16 |
| I-068 | CHF₂ | O | N | N | — | NH | — | 2-methoxyethyl | 272 | 1.48 |
| I-069 | CHF₂ | O | N | N | — | — | — | phenyl | 275 | 2.98 |
| I-070 | CHF₂ | O | N | N | — | — | — | pyridin-3-yl | | 2.03 |
| I-071 | CHF₂ | O | N | N | — | — | — | 2-thienyl | 281 | 2.64 |
| I-072 | CHF₂ | O | N | N | — | — | — | 3-thienyl | 281 | 2.64 |
| I-073 | CHF₂ | O | N | N | — | NH | CH(Me) | cyclopropyl | 282 | 2.51 |
| I-074 | CHF₂ | O | N | N | — | — | — | morpholin-4-yl | 284 | 1.87 |
| I-075 | CHF₂ | O | N | N | — | — | CH₂ | phenyl | 289 | 2.49 |
| I-076 | CHF₂ | O | N | N | — | NH | — | phenyl | 290 | 2.54 |
| I-077 | CHF₂ | O | N | N | — | — | — | 4-fluorophenyl | 293 | 3.70 |
| I-078 | CHF₂ | O | N | N | — | — | — | 1-ethyl-1H-pyrazol-5-yl | 293 | 2.18 |
| I-079 | CHF₂ | O | N | N | — | — | — | 2-fluoropyridin-4-yl | 294 | 2.33 |
| I-080 | CHF₂ | O | N | N | — | — | — | 6-fluoropyridin-3-yl | 294 | 2.31 |
| I-081 | CHF₂ | O | N | N | — | — | — | 3,5-dimethyl-1,2-oxazol-4-yl | 294 | 2.57 |
| I-082 | CHF₂ | O | N | N | — | NH | 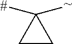 | cyclopropyl | 294 | 2.33 |
| I-083 | CHF₂ | O | N | N | — | — | CH₂ | cyclohexyl | 295 | 3.40 |
| I-084 | CHF₂ | O | N | N | — | NH | C(Me)₂ | cyclopropyl | 296 | 3.26 |
| I-085 | CHF₂ | O | N | N | — | — | — | 4-cyanophenyl | 299³ | 2.72 |
| I-086 | CHF₂ | O | N | N | — | NH | — | 2-methylphenyl | 304 | 2.51 |
| I-087 | CHF₂ | O | N | N | — | NMe | — | phenyl | 304 | 2.80 |
| I-088 | CHF₂ | O | N | N | — | NH | CH₂ | phenyl | 304 | 2.57 |
| I-089 | CHF₂ | O | N | N | — | — | — | 4-methoxyphenyl | 305 | 3.01 |
| I-090 | CHF₂ | O | N | N | — | O | CH₂ | phenyl | 305 | 2.75 |
| I-091 | CHF₂ | O | N | N | — | — | CH₂ | 4-fluorophenyl | 307 | 2.59 |
| I-092 | CHF₂ | O | N | N | — | S | — | phenyl | 307 | 3.04 |
| I-093 | CHF₂ | O | N | N | — | NH | — | 2-fluorophenyl | | 2.43 |
| I-094 | CHF₂ | O | N | N | — | NH | — | 3-fluorophenyl | 308 | 2.77 |
| I-095 | CHF₂ | O | N | N | — | NH | — | 4-fluorophenyl | 308 | 2.64 |
| I-096 | CHF₂ | O | N | N | — | NH | — | 1-ethyl-1H-pyrazol-5-yl | 308 | 1.49 |
| I-097 | CHF₂ | O | N | N | — | NH | 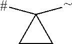 | 1-methylcyclopropyl | 308 | 2.89 |
| I-098 | CHF₂ | O | N | N | — | O | — | 4-fluorophenyl | 309 | 2.51 |
| I-099 | CHF₂ | O | N | N | — | — | — | 2-chlorophenyl | 309 | 3.32 |
| I.100 | CHF₂ | O | N | N | — | — | — | 3-chlorophenyl | 309 | 4.21 |
| I.101 | CHF₂ | O | N | N | — | — | — | 4-chlorophenyl | 309 | 3.60 |
| I.102 | CHF₂ | O | N | N | — | — | — | 2-chloropyridin-4-yl | 310 | 2.59 |
| I.103 | CHF₂ | O | N | N | — | — | — | 5-chloropyridin-3-yl | 310 | 2.59 |
| I.104 | CHF₂ | O | N | N | — | NH | — | 1- | 312 | 1.64 |
| | | | | | | | | (methoxycarbonyl)cyclopropyl | | |
| I.105 | CHF₂ | O | N | N | — | NH | CH₂ | tetrahydro-2H-pyran-4-yl | 312 | 1.73 |
| I.106 | CHF₂ | O | N | N | — | — | — | 1,3-dihydro-2H-isoindol-2-yl | 316 | 3.10 |
| I.107 | CHF₂ | O | N | N | — | — | CH₂ | 2-methoxyphenyl | 319 | 2.51 |
| I.108 | CHF₂ | O | N | N | — | *-NH—NMe-# | — | phenyl | 319 | 2.23 |
| I.109 | CHF₂ | O | N | N | — | NH | — | 2-methoxyphenyl | 320 | 2.80 |
| I.110 | CHF₂ | O | N | N | — | *-N(OH)-# | CH₂ | phenyl | 320 | 2.23 |
| I.111 | CHF₂ | O | N | N | — | NH | CH₂ | 2-fluorophenyl | 322 | 2.62 |
| I.112 | CHF₂ | O | N | N | — | NH | CH₂ | 4-fluorophenyl | 322 | 2.64 |
| I.113 | CHF₂ | S | N | N | — | NH | — | 2-fluorophenyl | | 2.77 |
| I.114 | CHF₂ | O | N | N | — | NH | — | 2-chlorophenyl | 324 | 2.92 |
| I.115 | CHF₂ | O | N | N | — | — | CH₂ | 2,4-difluorophenyl | 325 | 2.70 |
| I.116 | CF₃ | O | N | N | — | NH | — | 2-fluorophenyl | | 2.99 |
| I.117 | CHF₂ | O | N | N | — | NH | — | 1-(ethoxycarbonyl)cyclopropyl | 326 | 1.94 |
| I.118 | CHF₂ | O | N | N | — | NH | CH(C≡CH) | phenyl | 328 | 2.86 |
| I.119 | CHF₂ | O | N | N | — | NH | — | 2-cyclopropylphenyl | 330 | 3.10 |
| I.120 | CHF₂ | O | N | N | — | O | 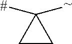 | phenyl | 331 | 2.95 |
| I.121 | CHF₂ | O | N | N | — | NH | 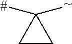 | pyridin-2-yl | 331 | 0.93 |
| I.122 | CHF₂ | O | N | N | — | NH | CH(Et) | phenyl | 332 | 3.16 |
| I.123 | CHF₂ | O | N | N | — | S | CH(Me) | phenyl | 335 | 3.72 |
| I.124 | CHF₂ | O | N | N | — | NH | CH(Me) | 2-fluorophenyl | 336 | 2.89 |
| I.125 | CHF₂ | O | N | N | — | NH | CH(Me) | 4-fluorophenyl | 336 | 2.89 |
| I.125a | CHF₂ | O | N | N | — | NH | (R)-CH(Me)¹ | 4-fluorophenyl | 336 | 2.86 |
| I.125b | CHF₂ | O | N | N | — | NH | (S)-CH(Me)¹ | 4-fluorophenyl | 336 | 2.85 |
| I.126 | CHF₂ | O | N | N | — | NH | 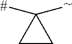 | cyclohexyl | 336 | 3.57 |
| I.127 | CHF₂ | O | N | N | — | O | CH(Me) | 4-fluorophenyl | 337 | 3.13 |
| I.128 | CHF₂ | O | N | N | — | NH | CH₂ | 2-chlorophenyl | 338 | 2.95 |
| I.129 | CHF₂ | O | N | N | — | NH | CH₂ | 2,6-difluorophenyl | 340 | 2.67 |
| I.130 | CF₃ | S | N | N | — | NH | — | 2-fluorophenyl | | 3.30 |
| I.131 | CClF₂ | O | N | N | — | NH | — | 2-fluorophenyl | | 3.26 |
| I.132 | CHF₂ | O | N | N | — | NH | 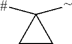 | 2-methylphenyl | 344 | 3.23 |
| I.133 | CHF₂ | O | N | N | — | NH | 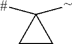 | 4-methylphenyl | 344 | 3.00 |
| I.134 | CHF₂ | O | N | N | — | NH | 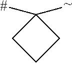 | phenyl | | 3.18 |
| I.135 | CHF₂ | O | N | N | — | NH | 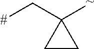 | phenyl | 344 | 3.25 |
| I.136 | CHF₂ | O | N | N | — | NH | CH(cPr) | phenyl | 344 | 3.14 |
| I.137 | CHF₂ | O | N | N | — | NH | 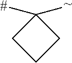 | pyridin-2-yl | 345 | 1.20 |
| I.138 | CHF₂ | O | N | N | — | NH | C(Me)(Et) | phenyl | 346 | 3.50 |
| I.139 | CHF₂ | O | N | N | — | NH | CH(iPr) | phenyl | 346 | 3.57 |
| I.140 | CHF₂ | O | N | N | — | NH | CH(Pr) | phenyl | 346 | 3.54 |
| I.141 | CHF₂ | O | N | N | — | NH | CH(CN) | 4-fluorophenyl | 347 | 2.65 |
| I.142 | CHF₂ | O | N | N | — | O | 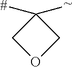 | phenyl | 347 | 2.51 |
| I.143 | CHF₂ | O | N | N | — | O | 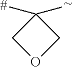 | pyridin-2-yl | 348 | 1.66 |
| I.144 | CHF₂ | O | N | N | — | NH | 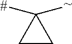 | 2-fluorophenyl | | 2.85 |
| I.145 | CHF₂ | O | N | N | — | NH | 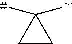 | 3-fluorophenyl | 348 | 2.72 |
| I.146 | CHF₂ | O | N | N | — | NH | 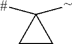 | 4-fluorophenyl | 348 | 2.75 |
| I.147 | CHF₂ | O | N | N | — | *-N(OMe)-# | CH(Me) | phenyl | 348 | 3.37 |
| I.148 | CHF₂ | O | N | N | — | *-N(OEt)-# | CH₂ | phenyl | 348 | 3.28 |
| I.149 | CHF₂ | O | N | N | — | O | 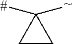 | 4-fluorophenyl | 349 | 3.19 |
| I.150 | CHF₂ | O | N | N | — | NH | C(Me)₂ | 4-fluorophenyl | 350 | 3.26 |
| I.151 | CHF₂ | O | N | N | — | NH | CH(Me) | 2,6-difluorophenyl | 354 | 3.10 |
| I.152 | CHF₂ | O | N | N | — | NH | 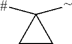 | 4-cyanophenyl | 355 | 2.33 |
| I.153 | CHF₂ | O | N | N | — | NH | 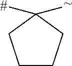 | phenyl | 358 | 3.57 |
| I.155 | CHF₂ | O | N | N | — | NH | 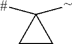 | 2-methoxyphenyl | 360 | 3.06 |
| I.156 | CHF₂ | O | N | N | — | NH | 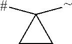 | 3-methoxyphenyl | 360 | 2.62 |
| I.157 | CHF₂ | O | N | N | — | NH | 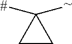 | 4-methoxyphenyl | 360 | 2.62 |
| I.158 | CHF₂ | O | N | N | — | NH | 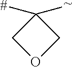 | 4-methylphenyl | 360 | 2.57 |
| I.159 | CHF₂ | S | N | N | — | NH | 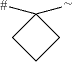 | phenyl | | 3.54 |
| I.160 | CF₃ | O | N | N | — | NH | 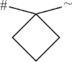 | phenyl | | 3.84 |
| I.161 | CHF₂ | O | N | N | — | *-N(OMe)-# | CH₂ | 2-methoxyphenyl | 364 | 2.97 |
| I.162 | CHF₂ | S | N | N | — | NH | 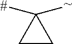 | 2-fluorophenyl | | 3.22 |
| I.163 | CHF₂ | O | N | N | — | NH | 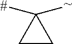 | 2-chlorophenyl | 364 | 3.34 |
| I.164 | CHF₂ | O | N | N | — | NH | 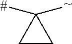 | 2,6-difluorophenyl | 366 | 2.95 |
| I.165 | CF₃ | O | N | N | — | NH | 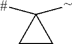 | 2-fluorophenyl | | 3.47 |
| I.166 | CHF₂ | O | N | N | — | NH | — | (biphenyl)—2-yl | 366 | 3.37 |
| I.167 | CHF₂ | O | N | N | — | O | 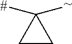 | 2,6-difluorophenyl | 367 | 3.25 |
| I.168 | CHF₂ | O | N | N | — | NH | C(Me)₂ | 2,6-difluorophenyl | 368 | 3.34 |
| I.169 | CHF₂ | O | N | N | — | *-N(OMe)-# | CH₂ | 4-chlorophenyl | 368 | 3.31 |
| I.170 | CHF₂ | O | N | N | — | NH | 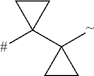 | phenyl | 370 | 3.64 |
| I.171 | CHF₂ | O | N | N | — | NH | 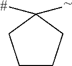 | 2-fluorophenyl | 376 | 3.74 |
| I.172 | CHF₂ | O | N | N | — | NH | 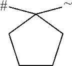 | 3-fluorophenyl | 376 | 3.60 |
| I.173 | CF₃ | S | N | N | — | NH | 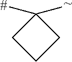 | phenyl | | 4.12 |
| I.174 | CHF₂ | O | N | N | — | NH | 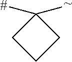 | 2-chlorophenyl | 378 | 3.81 |
| I.175 | CHF₂ | O | N | N | — | NH | 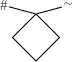 | 3-chlorophenyl | 378 | 3.67 |
| I.176 | CClF₂ | O | N | N | — | NH | 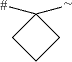 | phenyl | | 4.08 |
| I.177 | CHF₂ | O | N | N | — | NH | 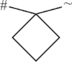 | 2,6-difluorophenyl | 380 | 3.50 |
| I.178 | CF₃ | S | N | N | — | NH | 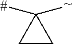 | 2-fluorophenyl | | 3.79 |
| I.179 | CClF₂ | O | N | N | — | NH | 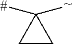 | 2-fluorophenyl | | 3.76 |
| I.180 | CHF₂ | O | N | N | — | NH | 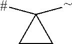 | 4-(methoxycarbonyl)phenyl | 388 | 2.51 |
| I.181 | CHF₂ | O | N | N | — | NH | CH(CF₃) | 4-fluorophenyl | 390 | 3.22 |
| I.182 | CHF₂ | O | N | N | — | NH | 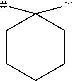 | 4-chlorophenyl | 406 | 4.40 |
| I.183 | CHF₂ | O | N | N | — | NH | 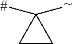 | 2-bromophenyl | 408 | 3.49 |
| I.184 | CHF₂ | O | N | N | — | *-N(OMe)-# | CH(Me) | 3-(trifluoromethyl)phenyl | 416 | 3.94 |
| I.185 | CHF₂ | O | N | N | — | *-N(2,4-difluoro- | — | 2,4-difluorophenyl | 494 | 3.59 |
| | | | | | | benzoyl)-C(═O)-# | | | | |
| I.186 | CHF₂ | O | N | N | — | NH | CH(Me) | 1-methylcyclopropyl | 296 | 2.91 |
| I.187 | CHF₂ | O | N | N | — | NH | — | 1-isopropylcyclopropyl | 296 | 2.65 |
| I.188 | CHF₂ | O | N | N | — | NH | CH(Me) | pyridin-3-yl | 319 | 0.74 |
| I.189 | CHF₂ | O | N | N | — | NH | CH(Me) | pyridin-2-yl | 319 | 1.00 |
| I.190 | CHF₂ | O | N | N | — | NH | CH(Me) | pyridin-4-yl | 319 | 0.65 |
| I.191 | CHF₂ | O | N | N | — | NH | CH(Me) | pyrimidin-2-yl | 320 | 1.49 |
| I.192 | CHF₂ | O | N | N | — | NH | CH(Me) | 2-thienyl | 324 | 2.70 |
| I.193 | CHF₂ | O | N | N | — | NH | CH(Me) | cyclohexyl | 324 | 3.74 |
| I.194 | CHF₂ | O | N | N | — | NH | CH(Me) | 1,3-thiazol-4-yl | 325 | 1.80 |
| I.195 | CHF₂ | O | N | N | — | NH | 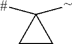 | phenyl | 330 | 2.62 |
| I.196 | CHF₂ | O | N | N | — | NH | 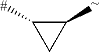 | phenyl | 330 | 2.94 |
| I.197 | CHF₂ | O | N | N | — | NH | C(Me)₂ | phenyl | 332 | 3.08 |
| I.198 | CHF₂ | O | N | N | — | NH | C(Me)₂ | pyridin-2-yl | 333 | 1.11 |
| I.199 | CHF₂ | O | N | N | — | NH | C(Me)₂ | pyridin-3-yl | 333 | 0.91 |
| I.200 | CHF₂ | O | N | N | — | NH | #-CH₂CH₂— | 4-fluorophenyl | 336 | 2.84 |
| I.201 | CHF₂ | O | N | N | — | NH | 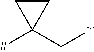 | phenyl | 344 | 2.99 |
| I.202 | CHF₂ | O | N | N | — | NH | 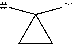 | 3-methylphenyl | 344 | 2.95 |
| I.203 | CHF₂ | O | N | N | — | NH | 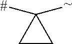 | 4-methylpyridin-2-yl | 345 | 0.93 |
| I.204 | CHF₂ | O | N | N | — | NH | C(Me)₂ | 4-methylphenyl | 346 | 3.46 |
| I.205 | CHF₂ | O | N | N | — | NH | C(Me)₂ | 2-methylphenyl | 346 | 3.28 |
| I.206 | CHF₂ | O | N | N | — | NH | 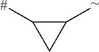 | 4-fluorophenyl | 348 | 2.97 |
| I.207 | CHF₂ | O | N | N | — | NH | CH(Me) | 4-methoxyphenyl | 348 | 2.75 |
| I.208 | CHF₂ | O | N | N | — | NH | 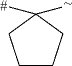 | 2-furyl | 348 | 3.18 |
| I.209 | CHF₂ | O | N | N | — | NH | CH(Me) | 2-methoxyphenyl | 348 | 2.95 |
| I.210 | CHF₂ | O | N | N | — | NH | #-CH(Me)CH₂-~ | 4-fluorophenyl | 350 | 3.12 |
| I.211 | CHF₂ | O | N | N | — | NH | #-CH₂CH(Me)-~ | 4-fluorophenyl | 350 | 3.13 |
| I.212 | CHF₂ | O | N | N | — | NH | C(Me)₂ | 2-fluorophenyl | 350 | 3.19 |
| I.213 | CHF₂ | O | N | N | — | NH | CH(Me) | 3-chlorophenyl | 352 | 3.20 |
| I.214 | CHF₂ | O | N | N | — | NH | CH(Me) | 6-chloropyridin-3-yl | 353 | 2.27 |
| I.215 | CHF₂ | O | N | N | — | NH | CH(Me) | 2,4-difluorophenyl | 354 | 3.01 |
| I.216 | CHF₂ | O | N | N | — | NH | 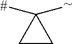 | 2-6-dimethylphenyl | 358 | 3.80 |
| I.217 | CHF₂ | O | N | N | — | NH | 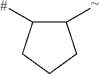 | phenyl | 358 | 3.44 |
| I.218 | CHF₂ | O | N | N | — | NH | 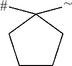 | pyridin-3-yl | 359 | 1.29 |
| I.219 | CHF₂ | O | N | N | — | NH | 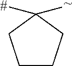 | pyridin-2-yl | 359 | 1.38 |
| I.220 | CHF₂ | O | N | N | — | NH | 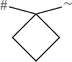 | 4-fluorophenyl | 362 | 3.33 |
| I.221 | CHF₂ | O | N | N | — | NMe | 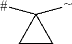 | 2-fluorophenyl | 362 | 3.98 |
| I.222 | CHF₂ | O | N | N | — | NH | — | 2-methoxy-2-oxo-1-(pyridin-2- | 363 | 1.92 |
| | | | | | | | | yl)ethyl | | |
| I.223 | CHF₂ | O | N | N | — | NH | #-C(Me)CH₂-~ | 4-fluorophenyl | 364 | 3.79 |
| I.224 | CHF₂ | O | N | N | — | NH | #-CH₂C(Me)₂-~ | 4-fluorophenyl | 364 | 3.44 |
| I.225 | CHF₂ | O | N | N | — | NH | 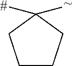 | 2-thienyl | 364 | 3.45 |
| I.226 | CHF₂ | O | N | N | — | NH | 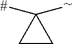 | 4-chlorophenyl | 364 | 3.16 |
| I.227 | CHF₂ | O | N | N | — | NH | 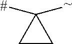 | 3-chlorophenyl | 364 | 3.11 |
| I.228 | CHF₂ | O | N | N | — | NH | 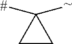 | 4-chloropyridin-2-yl | 365 | 2.31 |
| I.229 | CHF₂ | O | N | N | — | NH | 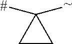 | 3,4-difluorophenyl | 366 | 2.84 |
| I.230 | CHF₂ | O | N | N | — | NH | CH(Et) | 4-chlorophenyl | 366 | 3.58 |
| I.231 | CHF₂ | O | N | N | — | NH | C(Me)₂ | 4-chlorophenyl | 366 | 3.55 |
| I.232 | CHF₂ | O | N | N | — | NH | CH(Me) | 2-naphthyl | 368 | 3.45 |
| I.233 | CHF₂ | O | N | N | — | NH | CH(Me) | guinolin-2-yl | 369 | 2.04 |
| I.234 | CHF₂ | O | N | N | — | NH | #-CH₂CF₂-~ | 4-fluorophenyl | 372 | 2.90 |
| I.235 | CHF₂ | O | N | N | — | NH | C(Me)(CH₂OH) | 3,4-dimethylphenyl | 376 | 2.68 |
| I.236 | CHF₂ | O | N | N | — | NH | 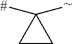 | 2-fluoro-3-methoxyphenyl | 378 | 2.81 |
| I.237 | CHF₂ | O | N | N | — | NH | 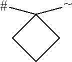 | 4-chlorophenyl | 378 | 3.71 |
| I.238 | CHF₂ | O | N | N | — | NH | CH(Phe) | phenyl | 380 | 3.52 |
| I.239 | CHF₂ | O | N | N | — | NH | CH(Me) | 3-(trifluoromethyl)phenyl | 386 | 3.37 |
| I.240 | CHF₂ | O | N | N | — | NH | CH(Me) | 2-(trifluoromethyl)phenyl | 386 | 3.42 |
| I.241 | CHF₂ | O | N | N | — | N(CH₂C≡CH) | 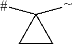 | 2-fluorophenyl | 386 | 4.09 |
| I.242 | CHF₂ | O | N | N | — | NH | 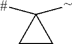 | 4-tert-butylphenyl | 386 | 4.05 |
| I.243 | CHF₂ | O | N | N | — | NH | 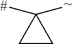 | 3,4-dimethoxyphenyl | 390 | 2.39 |
| I.244 | CHF₂ | O | N | N | — | NH | CH(cPent) | 4-fluorophenyl | 390 | 4.05 |
| I.245 | CHF₂ | O | N | N | — | NH | #-CH(Phe)CH₂-~ | phenyl | 394 | 3.68 |
| I.246 | CHF₂ | O | N | N | — | NH | 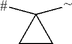 | 3-(difluoromethoxy)phenyl | 396 | 2.91 |
| I.247 | CHF₂ | O | N | N | — | NH | 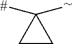 | 4-(difluoromethoxy)phenyl | 396 | 2.91 |
| I.248 | CHF₂ | O | N | N | — | NH | 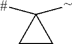 | 2-(difluoromethoxy)phenyl | 396 | 3.21 |
| I.249 | CHF₂ | O | N | N | — | NH | 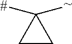 | 3-(trifluoromethyl)phenyl | 398 | 3.24 |
| I.250 | CHF₂ | O | N | N | — | NH | 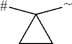 | 4-(trifluoromethyl)phenyl | 398 | 3.25 |
| I.251 | CHF₂ | O | N | N | — | NH | 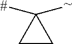 | 2,5-dichlorophenyl | 398 | 3.84 |
| I.252 | CHF₂ | O | N | N | — | NH | 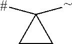 | 3,4-dichlorophenyl | 398 | 3.43 |
| I.253 | CHF₂ | O | N | N | — | NH | 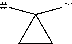 | 2,3-dichlorophenyl | 398 | 3.74 |
| I.254 | CHF₂ | O | N | N | — | NH | 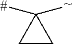 | 3,5-dichlorophenyl | 398 | 3.63 |
| I.255 | CHF₂ | O | N | N | — | NH | 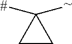 | 2,4-dichlorophenyl | 398 | 4.05 |
| I.256 | CHF₂ | O | N | N | — | NH | C(Me)₂ | 3-(trifluoromethyl)phenyl | 400 | 3.63 |
| I.257 | CHF₂ | O | N | N | — | NH | CH(Me) | 3-(trifluoromethoxy)phenyl | 402 | 3.53 |
| I.258 | CHF₂ | O | N | N | — | NH | 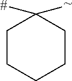 | 3-chlorophenyl | 406 | 4.35 |
| I.259 | CHF₂ | O | N | N | — | NH | 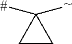 | 4-chloro-3-nitrophenyl | 409 | 2.96 |
| I.260 | CHF₂ | O | N | N | — | NH | CH(Et) | 2-bromophenyl | 410 | 3.64 |
| I.261 | CHF₂ | O | N | N | — | N(CH₂CHF₂) | 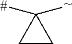 | 2-fluorophenyl | 412 | 4.40 |
| I.262 | CHF₂ | O | N | N | — | NH | 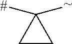 | 3-(trifluoromethoxy)phenyl | 414 | 3.37 |
| I.263 | CHF₂ | O | N | N | — | NH | 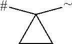 | 2-fluoro-4-(trifluoromethyl)phenyl | 416 | 3.59 |
| I.264 | CHF₂ | O | N | N | — | NH | 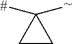 | 4,5-dichloro-2-fluorophenyl | 416 | 386 |
| I.265 | CHF₂ | O | N | N | — | NH | 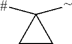 | 3-phenoxyphenyl | 422 | 3.62 |
| I.266 | CHF₂ | O | N | N | — | NH | 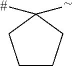 | 4-(trifluoromethyl)phenyl | 426 | 4.16 |
| I.267 | CHF₂ | O | N | N | — | N(COCF₃) | 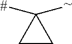 | 2-fluorophenyl | 444 | 3.91 |
| I.268 | CHF₂ | O | N | N | — | NH | 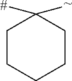 | 2-bromophenyl | 450 | 4.38 |

Note:
Me: methyl; Et: ethyl; Pr: propyl; iPr: isopropyl; cPr: cyclopropyl; cPent: cyclopentyl; Phe: phenyl
Note¹:
the two enantiomers were prepared from the enantiopure amines, respectively (1R)-1-(4-fluorophenyl)ethanamine and (1S)-1-(4-fluorophenyl)ethanamine
Note^{2:}
M-H ion by negative ionization
Note^{3:}
M ion by GC mass analysis

Table 2 illustrates in a non-limiting manner examples of compounds of formula (IIa) according to the invention as well as their acceptable salts

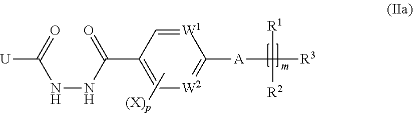

In table 2, the log P values were determined according to method [^{a}].

TABLE 2

| Example | U | W¹ | W² | (X)ₚ | A | —[CR¹R²]ₘ— | R³ | M + H | [symbol] |
|---|---|---|---|---|---|---|---|---|---|
| IIa.01 | CHF₂ | CH | CH | — | S | — | methyl | | [symbol] |
| IIa.02 | CHF₂ | CH | CH | — | N(COCH₃) | — | H | 272 | [symbol] |
| IIa.03 | CHF₂ | CH | CH | — | *—(C═O)—O—# | — | methyl | | [symbol] |
| IIa.04 | CF₃ | CH | CH | — | *—(C═O)—O—# | — | H | | [symbol] |
| IIa.05 | CHF₂ | CH | CH | — | — | — | 1H-1,2,3-triazol-1-yl | 282 | [symbol] |
| IIa.06 | CF₃ | CH | CH | — | *—(C═O)—O—# | — | methyl | | [symbol] |
| IIa.07 | CHF₂ | CH | CH | — | — | — | phenyl | 291 | [symbol] |
| IIa.08 | CHF₂ | CH | CH | — | — | — | Br | 293 | [symbol] |
| IIa.09 | CHF₂ | CH | CH | — | — | CH₂ | 1H-imidazol-1-yl | 295 | [symbol] |
| IIa.10 | CHF₂ | CH | CH | — | NH | — | pyrimidin-2-yl | | [symbol] |
| IIa.11 | CHF₂ | CH | CH | — | — | — | 3-ethyl-1,2,4- | 311 | [symbol] |
| | | | | | | | oxadiazol-5-yl | | |
| IIa.12 | CHF₂ | CH | CH | — | *—(C═O)—NH—# | — | 2-methoxyethyl | | [symbol] |
| IIa.13 | CHF₂ | CH | CH | — | NH | — | 2-fluorophenyl | | [symbol] |
| IIa.14 | CF₃ | CH | CH | — | — | — | 3-ethyl-1,2,4- | 329 | [symbol] |
| | | | | | | | oxadiazol-5-yl | | |
| IIa.15 | CF₃ | CH | CH | — | *—(C═O)—NH—# | — | 2-methoxyethyl | | [symbol] |
| IIa.16 | CHF₂ | CH | CH | — | *—(C═O)—NMe—# | — | phenyl | 348 | [symbol] |
| IIa.17 | CHF₂ | CH | CH | — | *—(C═O)—NH—N(COCHF₂)—# | — | H | | [symbol] |
| IIa.18 | CHF₂ | CH | CH | — | — | CH₂ | 4-chlorophenoxy | 355 | [symbol] |
| IIa.19 | CF₃ | CH | CH | — | *—(C═O)—NMe—# | — | phenyl | 366 | [symbol] |
| IIa.20 | CHF₂ | CH | CH | — | *—(C═O)—NH—# | — | 2,4-difluorophenyl | | [symbol] |
| IIa.21 | CF₃ | CH | CH | — | *—(C═O)—NH—# | — | 2,4-difluorophenyl | | [symbol] |
| IIa.22 | CHF₂ | N | CH | — | — | — | 2-furyl | 282 | [symbol] |
| IIa.23 | CHF₂ | N | CH | — | — | — | trifluoromethyl | 284 | [symbol] |
| IIa.24 | CHF₂ | N | CH | — | — | — | piperidin-1-yl | 299 | [symbol] |
| IIa.25 | CHF₂ | N | CH | — | O | — | phenyl | | [symbol] |
| IIa.26 | CHF₂ | N | CH | — | — | — | 4-(tert-butoxycarbonyl) | | [symbol] |
| | | | | | | | piperazin-1-yl | | |
| IIa.27 | CHF₂ | N | N | — | — | — | morpholin-4-yl | 302 | [symbol] |
| IIa.28 | CHF₂ | N | N | — | NH | — | 2-fluorophenyl | | [symbol] |
| IIa.29 | CF₃ | N | N | — | NH | — | 2-fluorophenyl | | [symbol] |
| IIa.30 | CHF₂ | N | N | — | NH | — | 1-(methoxycarbonyl) | 344 | [symbol] |
| | | | | | | | cyclopropyl | | |
| IIa.31 | CHF₂ | N | N | — | S | CH(Me) | phenyl | 353 | [symbol] |

Note:
Me: methyl
In table 2, “*” denotes the point of attachement of the diradical A to the aromatic group, “#” denotes the point of attachement of the diradical A to the —[CR¹R²]ₘR³ group. [symbol]
indicates data missing or illegible when filed

Table 3 provides the NMR data (¹H) of a selected number of compounds from tables 1 and 2

TABLE 3
NMR peak lists

I.001: ¹H-NMR(300.1 MHz, d₆-DMSO):
δ = 13.5177 (0.4); 13.4787 (0.4); 13.4175 (0.4); 13.3665 (0.4); 13.3423 (0.3); 8.1769 (16.0); 7.7478 (1.2); 7.5760 (2.4);
7.4069 (1.2); 3.3349 (3.2); 3.1400 (0.4); 2.5065 (13.3); 1.2307 (0.7); −0.0025 (1.6)
I.002: ¹H-NMR(400.1 MHz, d₆-DMSO):
δ = 7.9833 (14.6); 7.9622 (15.7); 7.6703 (4.4); 7.5418 (9.4); 7.5006 (16.0); 7.4795 (14.4); 7.4134 (4.7); 3.3179 (103.9);
2.8974 (1.3); 2.7384 (1.2); 2.5712 (58.9); 2.5469 (1.0); 2.5079 (21.6); 2.3933 (0.3)
I.003: ¹H-NMR(300.1 MHz, d₆-DMSO):
δ = 8.2244 (0.9); 8.1960 (6.3); 8.1913 (6.8); 8.1612 (0.9); 7.7582 (1.7); 7.5873 (3.9); 7.4165 (1.9); 3.9182 (16.0); 3.3466
(7.5); 2.5265 (0.7); 2.5207 (1.3); 2.5148 (1.7); 2.5090 (1.2); −0.0002 (0.5)
I.004: ¹H-NMR(300.1 MHz, d₆-DMSO):
δ = 8.2285 (3.3); 8.2213 (1.7); 8.1994 (16.0); 8.1836 (15.7); 8.1616 (1.7); 8.1570 (2.8); 8.1545 (3.4); 3.3467 (0.7);
3.3327 (0.7); 2.5147 (7.0); 2.5089 (13.7); 2.5029 (18.1); 2.4970 (12.6); 2.4913 (5.9); 1.2329 (0.5); 0.0104 (0.5); −0.0005
(12.2); −0.0114 (0.4)
I.005: ¹H-NMR(400.1 MHz, d₆-DMSO):
δ = 9.0172 (12.3); 8.3085 (8.5); 8.2868 (15.8); 8.2408 (16.0); 8.2191 (8.2); 8.0951 (0.3); 8.0582 (11.9); 7.7155 (3.5);
7.5872 (7.4); 7.4589 (3.7); 3.3194 (83.6); 2.5080 (17.0)
I.006: ¹H-NMR(300.1 MHz, d₆-DMSO):
δ = 8.2574 (1.7); 8.2550 (1.4); 8.2504 (0.9); 8.2358 (1.4); 8.2284 (6.6); 8.2074 (6.5); 8.1999 (1.3); 8.1854 (0.8); 8.1808
(1.3); 8.1783 (1.7); 3.9180 (16.0); 3.3347 (21.1); 2.5160 (1.8); 2.5101 (3.4); 2.5042 (4.4); 2.4982 (3.0); 2.4925 (1.4); −0.0002
(1.8)
I.007: ¹H-NMR(400.1 MHz, d₆-DMSO):
δ = 8.1659 (13.0); 8.1452 (15.7); 7.9729 (16.0); 7.9522 (13.0); 7.8018 (10.9); 7.7832 (13.2); 7.7065 (4.2); 7.5781 (9.1);
7.5583 (5.8); 7.5397 (12.9); 7.5204 (8.3); 7.4780 (5.0); 7.4602 (6.4); 7.4498 (5.1); 7.4432 (2.3); 3.3197 (103.7); 2.8967
(0.4); 2.7383 (0.4); 2.5443 (0.5); 2.5080 (21.6)
I.008: ¹H-NMR(300.1 MHz, d₆-DMSO):
δ = 13.3839 (0.6); 13.3502 (0.5); 8.2463 (8.4); 8.2242 (4.8); 8.2178 (16.0); 8.1729 (0.8); 8.1532 (15.8); 8.1246 (8.4);
3.3292 (8.0); 2.5151 (20.2); 2.5093 (39.0); 2.5034 (50.9); 2.4976 (35.3); 1.2318 (2.2); 0.8528 (0.4); −0.0004 (4.6)
I.009: ¹H-NMR(300.2 MHz, d₆-DMSO):
δ = 8.0368 (1.0); 8.0302 (0.3); 8.0146 (0.4); 8.0078 (1.4); 7.8961 (1.5); 7.8892 (0.4); 7.8736 (0.4); 7.8670 (0.9); 7.7457
(0.4); 7.5747 (0.9); 7.4038 (0.4); 3.3389 (16.0); 2.5348 (1.2); 2.5288 (2.6); 2.5227 (3.5); 2.5166 (2.5); 2.5106 (1.2);
0.0210 (5.5)
I.010: ¹H-NMR(400.1 MHz, d₆-DMSO):
δ = 8.4714 (5.8); 8.0992 (8.2); 8.0787 (8.6); 7.9594 (1.0); 7.6838 (2.5); 7.5551 (13.5); 7.5345 (7.6); 7.5062 (6.2); 7.4270
(2.7); 7.3076 (6.0); 5.4535 (16.0); 5.4225 (0.3); 3.8548 (0.4); 3.7500 (0.3); 3.7215 (0.3); 3.7134 (0.4); 3.6907 (0.4);
3.6367 (0.7); 3.4737 (2.4); 3.3940 (0.8); 3.3773 (0.9); 3.1501 (0.4); 3.1252 (0.4); 2.8973 (5.5); 2.7380 (5.1); 2.5440
(0.5); 2.5079 (16.4)
I.011: ¹H-NMR(300.1 MHz, d₆-DMSO):
δ = 8.2688 (3.0); 8.2627 (1.3); 8.2466 (1.7); 8.2402 (5.5); 8.1707 (5.5); 8.1643 (1.7); 8.1482 (1.3); 8.1421 (3.0); 3.9148
(16.0); 3.3296 (3.0); 2.5161 (3.6); 2.5105 (6.9); 2.5046 (9.0); 2.4988 (6.4); 1.2283 (0.6); −0.0005 (0.8)
I.012: ¹H-NMR(300.2 MHz, d₆-DMSO):
δ = 11.1483 (1.9); 8.1328 (0.5); 8.1253 (3.3); 8.1190 (1.1); 8.1024 (1.4); 8.0959 (4.8); 7.9692 (4.7); 7.9627 (1.4); 7.9458
(1.1); 7.9397 (3.1); 7.7391 (1.5); 7.5679 (3.3); 7.3969 (1.6); 6.6292 (1.2); 6.4507 (3.1); 6.2726 (1.4); 3.3466 (16.0);
2.5348 (3.1); 2.5288 (6.6); 2.5227 (9.0); 2.5166 (6.4); 2.5107 (2.9); 0.0199 (8.2)
I.013: ¹H-NMR(400.2 MHz, d₆-DMSO):
δ = 10.2168 (7.2); 8.5959 (15.6); 8.5839 (16.0); 8.5656 (0.6); 8.5534 (0.4); 8.0620 (6.9); 8.0396 (14.5); 7.9993 (14.2);
7.9770 (6.9); 7.8918 (0.3); 7.6648 (2.9); 7.5362 (6.9); 7.4078 (3.3); 6.9916 (4.2); 6.9796 (7.9); 6.9676 (4.2); 4.6368
(0.5); 4.4519 (0.9); 4.4167 (0.9); 4.3811 (0.9); 4.2084 (0.5); 4.1588 (0.4); 2.6724 (0.6); 2.5078 (76.0); 2.5036 (99.7);
2.4994 (74.5); 2.3304 (0.6); 0.0000 (10.0)
I.015: ¹H-NMR(300.2 MHz, CDCl₃):
δ = 8.3808 (1.3); 8.3777 (1.0); 8.3727 (0.7); 8.3596 (1.2); 8.3514 (10.6); 8.3422 (10.4); 8.3337 (1.3); 8.3205 (0.7);
8.3154 (1.0); 8.3124 (1.3); 7.2987 (16.8); 7.1570 (1.8); 6.9848 (4.0); 6.8127 (2.0); 2.9495 (1.8); 2.9242 (5.7); 2.8989
(5.9); 2.8737 (2.0); 1.5908 (16.0); 1.4762 (6.1); 1.4510 (12.5); 1.4257 (5.7); 1.2927 (0.5); 0.0479 (0.7); 0.0371 (17.5);
0.0262 (0.6)
I.016: ¹H-NMR(300.1 MHz, d₆-DMSO):
δ = 8.8033 (1.7); 8.7865 (3.1); 8.1762 (7.7); 8.1477 (16.0); 8.0924 (15.7); 8.0638 (7.6); 7.7434 (4.0); 7.5724 (9.3);
7.4014 (4.4); 3.5142 (1.2); 3.5038 (2.2); 3.4869 (11.2); 3.4774 (18.7); 3.4648 (7.3); 3.4508 (4.7); 3.4341 (1.4); 3.4235
(0.6); 3.3279 (17.2); 3.3031 (1.1); 3.2822 (61.9); 3.0456 (0.3); 2.5171 (4.2); 2.5114 (7.9); 2.5055 (10.2); 2.4997
(7.1); −0.0002 (6.0)
I.017: ¹H-NMR(300.1 MHz, d₆-DMSO):
δ = 8.7130 (8.5); 8.6000 (0.6); 7.8927 (15.2); 7.8634 (16.0); 7.8077 (1.2); 7.7785 (1.2); 7.6849 (5.2); 7.5134 (13.3);
7.4383 (2.4); 7.4316 (2.6); 7.4107 (4.7); 7.4062 (5.0); 7.3843 (3.6); 7.3787 (3.4); 7.3497 (3.3); 7.3422 (8.3); 7.3241
(4.0); 7.3178 (4.2); 7.3120 (3.2); 7.3065 (3.0); 7.2854 (3.9); 7.2800 (4.0); 7.2629 (0.6); 7.2575 (0.6); 7.2402 (2.4);
7.2346 (2.8); 7.2153 (6.7); 7.2095 (5.7); 7.1900 (6.6); 7.1870 (5.5); 7.1840 (5.3); 7.1799 (4.1); 7.1710 (3.4); 7.1644
(4.2); 7.1546 (3.3); 7.1456 (3.1); 7.1391 (3.2); 7.1299 (1.3); 7.1199 (1.4); 7.1140 (1.2); 7.0496 (10.3); 7.0470 (10.4);
7.0203 (9.9); 6.9261 (0.8); 6.8999 (0.7); 3.7799 (4.6); 3.3628 (0.5); 3.3348 (52.8); 3.3113 (9.6); 2.5166 (18.7); 2.5107
(36.1); 2.5048 (47.7); 2.4989 (33.8); 2.4932 (17.1); 2.2744 (0.3); 0.0000 (3.5)
I.018: ¹H-NMR(300.2 MHz, CDCl₃):
δ = 8.3920 (1.0); 8.3850 (0.5); 8.3703 (0.9); 8.3628 (4.4); 8.3446 (4.4); 8.3370 (0.8); 8.3222 (0.5); 8.3153 (1.0); 7.2987
(14.6); 2.9520 (0.9); 2.9267 (2.9); 2.9014 (3.0); 2.8762 (1.0); 1.5844 (16.0); 1.4773 (3.1); 1.4521 (6.3); 1.4268 (2.9);
1.2937 (0.4); 0.0486 (0.6); 0.0377 (14.8); 0.0267 (0.6)
I.019: ¹H-NMR(400.1 MHz, d₆-DMSO):
δ = 8.3180 (16.0); 7.7227 (1.8); 7.5944 (3.9); 7.4662 (1.9); 3.3177 (26.6); 2.8977 (0.6); 2.7382 (0.6); 2.5079 (5.5)
I.020: ¹H-NMR(300.1 MHz, d₆-DMSO):
δ = 8.8209 (1.9); 8.8045 (3.2); 8.1999 (9.1); 8.1775 (5.0); 8.1714 (16.0); 8.0996 (15.7); 8.0933 (4.4); 8.0710 (8.4);
3.5133 (1.6); 3.5036 (2.6); 3.4867 (11.8); 3.4771 (19.0); 3.4643 (7.4); 3.4504 (4.9); 3.4337 (1.5); 3.4224 (0.7); 3.3283
(17.4); 3.2811 (66.1); 3.0445 (0.4); 2.5148 (6.2); 2.5089 (11.8); 2.5030 (15.3); 2.4971 (10.5); 0.0105 (0.4); −0.0003 (8.1)
I.021: ¹H-NMR(300.2 MHz, CDCl₃):
δ = 8.1606 (0.8); 8.1385 (0.4); 8.1327 (1.1); 8.0208 (1.1); 7.9930 (0.7); 7.2986 (2.1); 7.1292 (0.4); 6.9568 (0.8); 6.7845
(0.4); 1.5997 (1.8); 1.4106 (16.0); 1.3075 (0.6); 1.2981 (6.1); 0.0365 (3.0)
I.022: ¹H-NMR(300.2 MHz, CDCl₃):
δ = 7.9701 (3.0); 7.9474 (1.2); 7.9417 (3.4); 7.5032 (3.5); 7.4974 (1.3); 7.4805 (1.2); 7.4748 (3.0); 7.3041 (0.6); 7.2987
(2.5); 7.2938 (0.5); 7.2765 (2.5); 7.2708 (1.4); 7.2559 (1.0); 7.2510 (2.4); 7.2422 (0.4); 7.2271 (0.7); 7.2225 (1.4);
7.2179 (0.9); 7.2065 (0.4); 7.1988 (1.4); 7.1740 (0.4); 7.0968 (1.9); 7.0920 (2.8); 7.0860 (2.1); 7.0684 (1.9); 6.9137
(2.9); 6.7415 (1.4); 3.5604 (16.0); 1.7766 (0.6); 0.0300 (0.9)
I.023: ¹H-NMR(300.1 MHz, d₆-DMSO):
δ = 8.7969 (1.9); 8.7792 (3.3); 8.2183 (10.1); 8.1905 (15.4); 8.0730 (16.0); 8.0668 (5.0); 8.0504 (4.1); 8.0444 (10.9);
3.5125 (1.3); 3.5023 (2.3); 3.4853 (13.0); 3.4760 (20.6); 3.4633 (8.0); 3.4495 (5.4); 3.4331 (1.5); 3.3249 (33.0); 3.2806
(82.6); 3.2317 (0.8); 3.0441 (0.5); 2.7337 (0.4); 2.7281 (0.5); 2.5140 (29.7); 2.5080 (59.2); 2.5020 (79.3); 2.4960
(54.3); 2.4902 (24.8); 2.2778 (0.4); 2.2719 (0.4); 1.2323 (1.3); 1.1464 (0.4); 0.8552 (0.3); 0.8301 (0.4); −0.0004
(9.9); −0.0113 (0.5); −0.0623 (2.2)
I.024: ¹H-NMR(400.1 MHz, d₆-DMSO):
δ = 8.1086 (8.0); 8.0882 (8.6); 7.7127 (8.2); 7.6919 (8.0); 7.5599 (5.3); 7.4315 (2.7); 7.3704 (8.1); 7.3483 (9.2); 7.0867
(9.3); 7.0645 (7.9); 5.2551 (16.0); 3.3187 (76.7); 2.8972 (0.4); 2.7384 (0.4); 2.5079 (15.8)
I.025: ¹H-NMR(400.2 MHz, CDCl₃):
δ = 7.8595 (1.4); 7.8533 (11.8); 7.8487 (3.7); 7.8359 (3.6); 7.8312 (12.5); 7.8251 (1.5); 7.4297 (2.0); 7.4254 (2.2);
7.4103 (4.0); 7.4060 (4.2); 7.3909 (2.3); 7.3865 (2.3); 7.2615 (18.5); 7.2334 (0.9); 7.2290 (1.0); 7.2203 (1.0); 7.2148
(2.1); 7.2097 (1.9); 7.2008 (1.8); 7.1950 (2.5); 7.1901 (1.5); 7.1814 (1.5); 7.1770 (1.4); 7.0891 (2.8); 7.0861 (3.3);
7.0702 (4.6); 7.0672 (5.2); 7.0514 (2.1); 7.0484 (2.3); 7.0404 (2.8); 7.0376 (2.5); 7.0200 (2.3); 7.0172 (2.2); 7.0123
(2.8); 7.0094 (2.6); 6.9973 (0.3); 6.9919 (2.2); 6.9891 (2.1); 6.9756 (4.1); 6.8446 (16.0); 6.8396 (4.3); 6.8270 (3.8);
6.8222 (12.3); 6.8159 (1.6); 6.7165 (4.5); 5.2541 (0.8); 2.0091 (2.4); 1.5883 (1.6); 1.3880 (2.8); 1.3753 (7.7); 1.3696
(8.9); 1.3586 (4.2); 1.3186 (0.6); 1.2909 (0.5); 1.2508 (3.4); 1.2400 (8.3); 1.2343 (7.5); 1.2217 (2.2); 0.0081 (0.8);
0.0000 (25.0); −0.0083 (0.9)
I.026: ¹H-NMR(499.9 MHz, CDCl₃):
δ = 7.9348 (3.2); 7.9182 (3.6); 7.4771 (3.5); 7.4604 (3.4); 7.2610 (16.5); 7.2445 (3.4); 7.2288 (2.5); 7.1885 (1.4); 7.1738
(1.9); 7.1590 (0.6); 7.0552 (2.9); 7.0400 (2.6); 3.5315 (16.0); 1.5607 (17.0); 1.2548 (0.8); 0.0062 (0.4); −0.0002 (12.5)
I.027: ¹H-NMR(300.1 MHz, d₆-DMSO):
δ = 10.4165 (4.2); 8.2550 (2.8); 8.2265 (16.0); 8.2158 (14.3); 8.1874 (2.6); 7.7582 (2.2); 7.6681 (1.3); 7.6385 (2.6);
7.6176 (2.7); 7.6095 (1.9); 7.5873 (6.2); 7.4165 (3.6); 7.3895 (2.5); 7.3623 (1.4); 7.3540 (1.5); 7.1831 (1.4); 7.1564
(2.4); 7.1279 (1.3); 3.5238 (0.3); 3.4586 (0.5); 3.3579 (65.1); 2.5091 (11.6); 1.2372 (0.4)
I.028: ¹H-NMR(300.1 MHz, d₆-DMSO):
δ = 10.4358 (4.8); 8.2760 (5.1); 8.2473 (16.0); 8.2202 (13.6); 8.1917 (4.4); 7.6619 (1.5); 7.6411 (1.8); 7.6323 (3.1);
7.6116 (3.1); 7.6031 (2.0); 7.5822 (1.6); 7.4347 (1.8); 7.4253 (2.0); 7.4043 (2.3); 7.3989 (2.6); 7.3950 (2.6); 7.3899
(2.4); 7.3689 (1.8); 7.3594 (1.9); 7.1891 (1.3); 7.1846 (1.5); 7.1797 (1.4); 7.1560 (2.5); 7.1501 (2.4); 7.1315 (1.3);
7.1273 (1.4); 7.1225 (1.2); 3.3407 (83.4); 3.2484 (0.4); 2.5087 (24.9); 2.5028 (32.8); 2.4970 (23.2); 1.2334 (0.4);
0.0105 (0.8); −0.0002 (19.2); −0.0113 (0.8)
I.029: ¹H-NMR(300.1 MHz, d₆-DMSO):
δ = 10.3876 (8.0); 8.2955 (8.9); 8.2673 (16.0); 8.1933 (13.9); 8.1650 (7.9); 8.1423 (0.5); 7.6663 (1.8); 7.6458 (2.2);
7.6370 (3.6); 7.6163 (3.7); 7.6075 (2.1); 7.5872 (1.8); 7.4344 (2.3); 7.4247 (2.3); 7.4035 (2.7); 7.3942 (3.1); 7.3898
(2.7); 7.3682 (2.1); 7.3589 (2.1); 7.1886 (1.5); 7.1834 (1.7); 7.1557 (3.0); 7.1270 (1.5); 6.5337 (0.5); 3.3260 (109.9);
3.3027 (3.2); 2.7272 (0.7); 2.5075 (84.4); 2.5017 (109.7); 2.4959 (77.6); 2.2719 (0.7); 1.2984 (0.4); 1.2580 (0.6);
1.2336 (2.4); 1.1435 (0.6); −0.0006 (6.6)
I.030: ¹H-NMR(499.9 MHz, d₆-DMSO):
δ = 8.6678 (15.9); 8.6640 (16.0); 8.4944 (0.4); 8.4913 (0.4); 8.3185 (0.3); 8.1917 (9.6); 8.1873 (9.4); 8.1735 (9.6);
8.1691 (9.2); 8.0224 (0.4); 8.0041 (0.4); 7.6403 (6.2); 7.5376 (13.4); 7.4350 (6.7); 7.0062 (0.3); 6.9875 (0.4); 6.9117
(13.6); 6.8935 (13.0); 3.6204 (0.4); 3.6076 (0.8); 3.5951 (0.3); 2.5249 (3.0); 2.5220 (3.3); 2.1943 (0.4); 1.7784 (0.5);
1.7719 (0.6); 1.7656 (0.9); 1.3659 (2.2); −0.0002 (1.0)
I.031: ¹H-NMR(499.9 MHz, d₆-DMSO):
δ = 9.0735 (14.2); 9.0689 (14.7); 8.4952 (9.7); 8.4903 (9.9); 8.4785 (10.4); 8.4736 (10.4); 7.8199 (16.0); 7.8031 (15.3);
7.6950 (7.5); 7.5926 (16.0); 7.4901 (8.1); 3.3246 (8.2); 2.5134 (3.5); 2.5103 (4.6); 2.5071 (3.7); −0.0002 (2.0)
I.032: ¹H-NMR(499.9 MHz, d₆-DMSO):
δ = 13.5751 (1.0); 9.3240 (5.3); 8.6162 (4.5); 8.6025 (4.8); 8.2648 (5.2); 8.2498 (4.9); 7.7099 (7.2); 7.6074 (16.0);
7.5049 (8.0); 2.5078 (4.1); 2.5043 (5.7); 2.5008 (4.3); 0.5395 (0.6)
I.033: ¹H-NMR(300.2 MHz, CDCl₃):
δ = 9.3266 (3.4); 9.3240 (3.6); 9.3196 (3.8); 9.3170 (3.3); 8.6014 (2.6); 8.5943 (2.5); 8.5741 (3.6); 8.5669 (3.6); 8.4483
(4.4); 8.4457 (4.2); 8.4210 (3.1); 8.4183 (2.9); 8.0771 (0.7); 7.2984 (4.8); 7.1667 (2.6); 6.9948 (5.6); 6.8229 (2.8);
5.3328 (0.4); 3.1263 (16.0); 3.1091 (15.9); 2.0416 (1.0); 1.7091 (0.4); 0.0287 (4.7)
I.034: ¹H-NMR(300.2 MHz, CDCl₃):
δ = 9.0491 (1.6); 9.0459 (1.8); 9.0425 (1.8); 9.0393 (1.6); 8.4712 (0.5); 8.4438 (2.3); 8.4421 (2.3); 8.4309 (2.1); 8.4239
(2.0); 8.4014 (0.4); 8.3945 (0.5); 8.2631 (0.7); 7.2990 (4.2); 7.1393 (1.3); 6.9671 (2.7); 6.7950 (1.3); 5.3368 (0.6);
2.3138 (16.0); 1.6649 (2.2); 1.2886 (0.4); 0.0339 (5.4)
I.035: ¹H-NMR(300.2 MHz, d₆-DMSO):
δ = 9.2278 (1.7); 9.2251 (1.8); 9.2204 (1.9); 9.2176 (1.6); 8.5156 (1.7); 8.5080 (1.7); 8.4875 (1.9); 8.4800 (1.9); 8.0050
(2.2); 8.0024 (2.6); 7.9990 (4.4); 7.9965 (3.9); 7.9706 (2.0); 7.9680 (1.9); 7.7815 (1.4); 7.6108 (3.2); 7.4400 (1.6);
7.3786 (2.1); 7.3763 (2.1); 7.3671 (2.2); 7.3647 (2.0); 6.7813 (2.1); 6.7754 (2.1); 6.7697 (2.0); 6.7638 (2.0); 3.3512
(16.0); 2.5341 (3.2); 2.5280 (6.9); 2.5219 (9.6); 2.5158 (6.9); 2.5099 (3.2); 0.0296 (0.4); 0.0188 (13.4); 0.0112 (0.3);
0.0079 (0.5)
I.036: ¹H-NMR(300.2 MHz, CDCl₃):
δ = 9.1896 (1.7); 9.1875 (1.8); 9.1822 (1.9); 8.6884 (2.1); 8.6795 (2.2); 8.5604 (1.4); 8.5528 (1.3); 8.5314 (1.6); 8.5238
(1.6); 8.2387 (2.2); 8.2365 (2.0); 8.2097 (1.8); 8.2075 (1.7); 7.8502 (2.1); 7.8471 (2.2); 7.3649 (0.4); 7.2987 (32.7);
7.1585 (1.3); 6.9864 (2.8); 6.8144 (1.4); 6.5829 (1.5); 6.5774 (1.8); 6.5741 (1.8); 6.5686 (1.5); 2.4679 (0.6); 1.6447
(16.0); 1.4726 (0.5); 1.4483 (0.7); 1.4229 (0.3); 0.0477 (1.5); 0.0370 (41.9); 0.0261 (1.5)
I.037: ¹H-NMR(300.2 MHz, d₆-DMSO):
δ = 9.4359 (0.9); 9.4293 (0.9); 8.7643 (0.6); 8.7592 (0.6); 8.7369 (0.6); 8.7317 (0.7); 8.2232 (1.3); 8.1956 (1.2); 7.8150
(0.9); 7.6446 (2.0); 7.4742 (1.0); 3.3474 (16.0); 2.5343 (2.7); 2.5283 (5.6); 2.5222 (7.6); 2.5162 (5.5); 2.5102 (2.6);
0.0307 (0.3); 0.0200 (9.2); 0.0090 (0.3)
I.038: ¹H-NMR(300.2 MHz, CDCl₃):
δ = 9.2593 (0.4); 9.2539 (0.5); 9.0386 (1.3); 9.0363 (1.4); 9.0310 (1.5); 9.0287 (1.4); 8.5519 (0.4); 8.5439 (0.4); 8.5237
(0.4); 8.5159 (0.5); 8.4358 (0.8); 8.4279 (0.8); 8.4053 (1.1); 8.3987 (1.1); 8.2556 (1.7); 8.2531 (1.7); 8.2260 (1.2);
8.2237 (1.2); 7.9173 (0.6); 7.6093 (0.6); 7.6069 (0.6); 7.5813 (0.5); 7.5788 (0.5); 7.4024 (0.5); 7.2990 (31.4); 7.1591
(0.4); 7.1331 (1.1); 6.9871 (0.8); 6.9609 (2.5); 6.8151 (0.4); 6.7887 (1.2); 5.3386 (4.2); 3.8979 (16.0); 3.8877 (10.4);
2.9959 (0.3); 1.5969 (5.3); 0.0481 (1.5); 0.0374 (44.9); 0.0265 (1.6)
I.039: ¹H-NMR(300.2 MHz, CDCl₃):
δ = 9.3542 (10.2); 9.3496 (10.9); 8.6224 (6.9); 8.6153 (6.9); 8.5951 (9.5); 8.5880 (9.5); 8.4522 (12.2); 8.4502 (12.2);
8.4250 (8.9); 8.4229 (8.9); 8.2400 (3.1); 7.2989 (30.7); 7.1732 (7.1); 7.0013 (14.8); 6.8295 (7.4); 4.3646 (15.3); 4.3561
(16.0); 4.3459 (15.7); 4.3374 (15.5); 2.9949 (0.5); 2.9217 (0.4); 2.3558 (7.2); 2.3473 (14.9); 2.3388 (7.4); 1.6221
(12.1); 0.0448 (1.1); 0.0341 (33.3); 0.0234 (1.3)
I.040: ¹H-NMR(300.2 MHz, d₆-DMSO):
δ = 8.7345 (0.9); 8.7265 (0.9); 8.0589 (0.7); 8.0506 (0.7); 8.0284 (0.7); 8.0201 (0.7); 7.7027 (0.6); 7.5314 (1.4); 7.3602
(0.7); 7.0324 (0.9); 7.0018 (0.8); 3.7305 (1.4); 3.7117 (1.7); 3.6946 (1.3); 3.3425 (16.0); 2.5349 (2.1); 2.5289 (4.4);
2.5228 (6.0); 2.5168 (4.3); 2.5109 (2.0); 1.6801 (0.5); 1.6636 (0.6); 1.5888 (1.0); 1.5732 (1.3); 0.0209 (6.9)
I.041: ¹H-NMR(300.2 MHz, CDCl₃):
δ = 8.9079 (5.5); 8.9062 (5.2); 8.9000 (5.8); 8.1766 (4.2); 8.1685 (4.1); 8.1464 (4.4); 8.1383 (4.3); 7.2987 (12.1); 7.1008
(3.8); 6.9283 (8.3); 6.7556 (5.3); 6.7513 (6.4); 6.7209 (5.8); 3.8843 (9.3); 3.8691 (15.4); 3.8517 (16.0); 3.8109 (0.5);
3.7865 (0.4); 3.7452 (15.6); 3.7278 (14.9); 3.7128 (9.0); 2.0815 (1.3); 1.6310 (10.9); 1.3186 (0.4); 1.2948 (0.8); 1.2710
(0.4); 0.0451 (0.5); 0.0344 (15.5); 0.0251 (0.5); 0.0235 (0.6)
I.042: ¹H-NMR(300.2 MHz, CDCl₃):
δ = 9.3086 (1.0); 9.3062 (1.1); 9.3018 (1.2); 9.2993 (1.0); 8.6946 (0.9); 8.5944 (0.7); 8.5873 (0.7); 8.5671 (1.1); 8.5600
(1.1); 8.4700 (1.4); 8.4676 (1.3); 8.4427 (0.9); 8.4402 (0.8); 7.2985 (1.7); 7.1687 (0.7); 6.9968 (1.6); 6.8249 (0.8);
3.2090 (0.7); 3.1558 (0.7); 2.7857 (16.0); 0.0266 (1.7)
I.043: ¹H-NMR(499.9 MHz, CDCl₃):
δ = 8.9016 (11.5); 8.8974 (11.1); 8.3884 (8.5); 8.3836 (8.3); 8.3710 (8.4); 8.3662 (8.1); 7.6991 (3.2); 7.6826 (3.3);
7.4738 (9.3); 7.4700 (5.1); 7.4585 (15.6); 7.4571 (16.0); 7.4455 (5.1); 7.4419 (11.7); 7.3011 (6.3); 7.2862 (11.0);
7.2714 (6.5); 7.2601 (25.1); 7.1950 (14.0); 7.1929 (17.2); 7.1778 (14.1); 7.1762 (13.0); 7.0879 (12.2); 7.0705 (11.6);
7.0165 (6.3); 6.9132 (13.4); 6.8097 (6.7); 3.2459 (2.4); 3.2316 (7.0); 3.2174 (7.0); 3.2030 (2.2); 2.4151 (10.6); 1.5605
(20.4); 1.1374 (7.6); 1.1232 (14.5); 1.1089 (6.9); 0.0060 (2.9); −0.0002 (33.0); −0.0065 (1.6)
I.044: ¹H-NMR(300.2 MHz, CDCl₃):
δ = 9.3002 (2.3); 9.2978 (2.5); 9.2933 (2.6); 9.2908 (2.4); 8.5882 (1.8); 8.5811 (1.8); 8.5609 (2.6); 8.5538 (2.6); 8.4398
(3.0); 8.4374 (3.2); 8.4125 (2.1); 8.4100 (2.2); 8.3164 (1.0); 8.0546 (0.3); 7.2989 (10.2); 7.1668 (1.8); 6.9950 (4.0);
6.8231 (2.0); 2.9944 (2.9); 2.9203 (2.4); 1.6291 (5.8); 1.5512 (16.0); 0.9819 (0.9); 0.9585 (3.6); 0.9428 (1.6); 0.8896
(0.7); 0.8377 (1.9); 0.8219 (3.9); 0.7980 (1.1); 0.0448 (0.4); 0.0340 (11.4); 0.0232 (0.4)
I.045: ¹H-NMR(300.2 MHz, CDCl₃):
δ = 9.1463 (0.3); 9.0544 (1.6); 9.0517 (1.6); 9.0469 (1.7); 9.0443 (1.4); 8.5356 (0.3); 8.4895 (1.1); 8.4868 (1.0); 8.4601
(3.0); 8.4575 (2.4); 8.4308 (0.6); 8.4199 (2.4); 8.4124 (2.3); 8.3907 (0.8); 8.3830 (0.8); 7.2987 (18.5); 7.1317 (1.2);
6.9807 (0.5); 6.9594 (2.6); 6.7873 (1.3); 2.0446 (0.5); 1.6737 (16.0); 1.5641 (10.4); 1.4287 (0.8); 1.4154 (2.7); 1.4060
(2.8); 1.3926 (1.0); 0.8499 (1.1); 0.8362 (3.7); 0.8266 (3.6); 0.8135 (1.0); 0.0477 (0.8); 0.0369 (21.3); 0.0276 (0.6);
0.0260 (0.8)
I.046: ¹H-NMR(300.2 MHz, CDCl₃):
δ = 9.3479 (9.5); 9.3456 (11.1); 9.3411 (11.0); 9.3386 (10.6); 8.6020 (7.1); 8.5949 (7.1); 8.5747 (10.1); 8.5676 (10.1);
8.4525 (12.1); 8.4501 (13.1); 8.4252 (8.5); 8.4228 (9.1); 8.1741 (3.0); 7.2987 (15.6); 7.1707 (7.1); 6.9988 (15.2);
6.8269 (7.6); 3.4323 (12.4); 3.4124 (15.3); 3.4090 (16.0); 3.3892 (12.7); 2.0431 (0.5); 1.6852 (1.7); 1.2862 (0.4);
1.2110 (0.4); 1.1947 (0.8); 1.1851 (1.3); 1.1791 (0.8); 1.1689 (2.6); 1.1603 (2.3); 1.1551 (2.0); 1.1445 (4.2); 1.1341
(2.1); 1.1282 (2.5); 1.1183 (3.0); 1.1103 (1.0); 1.1023 (1.7); 1.0941 (1.1); 1.0780 (0.6); 0.6636 (3.4); 0.6469 (10.2);
0.6439 (11.9); 0.6285 (5.7); 0.6211 (11.3); 0.6177 (10.0); 0.6021 (4.4); 0.5789 (0.7); 0.5531 (0.5); 0.4363 (0.4); 0.4188
(0.5); 0.3863 (4.2); 0.3704 (13.7); 0.3514 (13.6); 0.3354 (3.2); 0.3030 (0.4); 0.2890 (0.3); 0.0417 (0.7); 0.0309 (15.9);
0.0202 (0.8)
I.047: ¹H-NMR(300.2 MHz, CDCl₃):
δ = 9.3491 (1.4); 9.3469 (1.6); 9.3423 (1.6); 9.3400 (1.5); 8.6042 (1.1); 8.5971 (1.1); 8.5769 (1.5); 8.5698 (1.5); 8.4457
(1.9); 8.4434 (1.9); 8.4184 (1.4); 8.4161 (1.4); 8.3708 (0.4); 7.2988 (5.6); 7.1698 (1.1); 6.9979 (2.4); 6.8260 (1.2);
3.7743 (0.6); 3.7582 (1.7); 3.7409 (2.2); 3.7226 (1.0); 3.7184 (0.8); 3.6620 (2.2); 3.6454 (2.6); 3.6297 (1.1); 3.4638
(16.0); 2.9949 (0.5); 2.9208 (0.4); 1.6451 (0.3); 0.0339 (6.2)
I.048: ¹H-NMR(300.2 MHz, CDCl₃):
δ = 9.9412 (1.6); 9.3985 (2.9); 9.3938 (3.0); 8.6645 (1.9); 8.6575 (1.8); 8.6371 (3.0); 8.6301 (2.9); 8.5508 (3.8); 8.5488
(3.6); 8.5235 (2.3); 8.5214 (2.3); 7.7382 (4.9); 7.7101 (5.6); 7.2987 (12.9); 7.2708 (4.4); 7.2432 (4.0); 7.1838 (2.0);
7.0119 (4.2); 6.8401 (2.1); 4.0418 (0.6); 2.4023 (16.0); 1.6226 (1.6); 1.5886 (0.4); 0.0471 (0.4); 0.0363 (12.3); 0.0253 (0.4)
I.049: ¹H-NMR(300.2 MHz, CDCl₃):
δ = 9.0658 (1.0); 8.3602 (0.8); 8.3347 (0.8); 7.6925 (0.8); 7.6661 (0.8); 7.2988 (13.1); 7.2618 (1.6); 7.2362 (1.6); 7.2148
(1.2); 7.1926 (1.0); 7.1702 (0.4); 7.1153 (2.8); 7.0951 (1.3); 6.9432 (2.7); 6.7713 (1.4); 3.5907 (16.0); 2.9946 (1.9);
2.9211 (1.6); 1.6187 (4.3); 0.0457 (0.5); 0.0348 (15.0); 0.0240 (0.5)
I.050: ¹H-NMR(300.2 MHz, CDCl₃):
δ = 8.9798 (3.6); 8.9779 (3.6); 8.9726 (3.8); 8.4906 (2.5); 8.4612 (5.3); 8.4110 (3.4); 8.4036 (3.3); 8.3817 (1.6); 8.3742
(1.6); 8.1701 (1.9); 8.0553 (0.7); 7.5022 (0.8); 7.4876 (1.0); 7.4818 (1.4); 7.4776 (0.8); 7.4637 (2.3); 7.4575 (2.8);
7.4547 (2.8); 7.4462 (1.6); 7.4355 (5.9); 7.4334 (5.3); 7.4120 (3.1); 7.3988 (8.3); 7.3748 (3.2); 7.2984 (6.1); 7.1255
(2.7); 6.9533 (5.8); 6.7812 (2.9); 3.8483 (16.0); 3.7647 (0.6); 2.9940 (5.3); 2.9218 (4.8); 0.0339 (7.1)
I.051: ¹H-NMR(300.2 MHz, CDCl₃):
δ = 11.5416 (0.4); 9.0896 (1.2); 9.0823 (1.4); 8.4067 (0.7); 8.3990 (0.7); 8.3776 (0.7); 8.3699 (0.7); 7.6707 (1.2); 7.6453
(1.5); 7.4595 (0.9); 7.4344 (1.5); 7.4069 (0.9); 7.2988 (9.1); 7.2198 (0.5); 7.1951 (0.8); 7.1703 (0.3); 7.1433 (1.1);
7.1130 (0.9); 6.9741 (1.3); 6.8021 (0.6); 1.5989 (16.0); 0.0486 (0.7); 0.0379 (12.6); 0.0270 (0.4)
I.052: ¹H-NMR(300.2 MHz, CDCl₃):
δ = 8.8328 (4.2); 8.8254 (4.4); 8.0494 (2.9); 8.0416 (2.9); 8.0199 (3.0); 8.0121 (3.0); 7.4030 (0.4); 7.3926 (3.4); 7.3862
(1.6); 7.3746 (3.8); 7.3638 (4.6); 7.3534 (1.8); 7.3462 (4.3); 7.3367 (0.5); 7.2985 (7.6); 7.1078 (0.6); 7.0978 (4.8);
7.0908 (1.5); 7.0785 (4.1); 7.0691 (8.4); 7.0470 (1.3); 7.0401 (3.8); 7.0301 (0.4); 7.0138 (0.4); 6.9060 (6.6); 6.7336
(3.2); 6.3658 (4.4); 6.3363 (4.2); 5.4982 (1.3); 5.4780 (1.4); 4.9767 (0.3); 4.9540 (1.2); 4.9320 (1.7); 4.9096 (1.2);
4.8877 (0.3); 1.6819 (4.0); 1.6372 (16.0); 1.6146 (16.0); 0.0349 (8.8)
I.053: ¹H-NMR(300.1 MHz, d₆-DMSO):
δ = 9.5031 (15.6); 8.7801 (15.5); 8.7726 (16.0); 8.1797 (9.5); 8.1716 (9.5); 8.1502 (10.1); 8.1421 (10.1); 8.0260 (0.3);
8.0008 (4.2); 7.9939 (4.5); 7.9735 (7.7); 7.9680 (8.6); 7.9466 (4.6); 7.9410 (4.6); 7.3269 (3.8); 7.3205 (3.8); 7.3017
(5.4); 7.2950 (6.5); 7.2897 (4.8); 7.2837 (4.6); 7.2630 (5.5); 7.2573 (6.7); 7.2391 (3.2); 7.2333 (3.8); 7.2140 (9.5);
7.2080 (8.5); 7.1888 (12.7); 7.1815 (10.5); 7.1725 (5.3); 7.1652 (6.6); 7.1560 (4.6); 7.1468 (4.5); 7.1400 (4.8); 7.1212
(1.6); 7.1154 (1.4); 7.0761 (13.8); 7.0465 (13.3); 3.3265 (39.8); 2.7287 (0.4); 2.5088 (45.1); 2.5031 (59.3); 2.4975
(43.2); 2.2724 (0.4); 2.0762 (3.4); −0.0001 (29.1)
I.054: ¹H-NMR(300.2 MHz, CDCl₃):
δ = 8.8851 (0.3); 8.8660 (6.6); 8.8603 (6.9); 8.8584 (6.5); 8.4470 (0.4); 8.1054 (4.1); 8.0977 (4.0); 8.0759 (4.1); 8.0693
(4.2); 8.0440 (0.3); 7.5388 (0.4); 7.4051 (0.3); 7.3960 (0.4); 7.2987 (35.2); 7.2546 (0.5); 7.2316 (0.6); 7.2065 (0.9);
7.1960 (5.6); 7.1889 (2.6); 7.1789 (6.2); 7.1737 (4.2); 7.1662 (8.8); 7.1565 (3.8); 7.1492 (8.6); 7.1388 (1.6); 7.1231
(0.6); 7.1079 (0.5); 7.0894 (5.0); 7.0714 (0.5); 7.0569 (1.5); 7.0468 (8.8); 7.0394 (3.0); 7.0287 (2.2); 7.0241 (3.4);
7.0183 (12.5); 7.0114 (3.5); 6.9963 (2.5); 6.9889 (6.0); 6.9782 (1.1); 6.9644 (0.9); 6.9480 (0.4); 6.9169 (10.5); 6.8130
(0.4); 6.7924 (0.3); 6.7445 (5.1); 6.6368 (7.0); 6.6348 (7.1); 6.6073 (6.8); 6.6053 (6.9); 6.1147 (3.8); 5.3375 (3.4);
3.6021 (0.4); 3.3878 (0.3); 2.0460 (0.4); 1.6956 (0.5); 1.6722 (0.9); 1.6348 (16.0); 1.5622 (0.4); 1.4869 (1.7); 1.4545
(10.0); 1.4460 (7.5); 1.4227 (7.6); 1.4142 (9.9); 1.3822 (2.4); 1.3417 (0.6); 1.3279 (0.8); 1.3021 (1.9); 1.2772 (0.7);
1.2158 (0.4); 1.1932 (0.5); 1.0768 (0.3); 0.9391 (0.5); 0.9182 (1.4); 0.8944 (0.8); 0.0471 (1.6); 0.0362 (44.2); 0.0253 (1.8)
I.055: ¹H-NMR(300.2 MHz, CDCl₃):
δ = 9.2777 (0.8); 9.2308 (0.8); 9.1114 (2.8); 9.1059 (2.6); 9.1039 (2.7); 8.6408 (2.4); 8.6114 (3.5); 8.5009 (2.2); 8.4933
(2.2); 8.4715 (1.5); 8.4639 (1.4); 8.3026 (0.9); 8.2811 (1.0); 8.2728 (1.8); 8.2513 (1.8); 8.2431 (1.0); 8.2215 (0.9);
7.2992 (8.2); 7.1643 (0.8); 7.1564 (1.0); 7.1471 (2.0); 7.1371 (1.2); 7.1337 (1.4); 7.1098 (0.7); 7.1018 (0.8); 7.0696
(1.0); 7.0618 (0.9); 7.0418 (1.1); 7.0336 (1.0); 7.0296 (1.2); 7.0213 (0.9); 7.0015 (1.0); 6.9936 (0.9); 6.9750 (4.1);
6.8028 (2.0); 2.0450 (0.5); 1.6195 (16.0); 0.0469 (0.4); 0.0361 (9.6); 0.0253 (0.4)
I.056: ¹H-NMR(300.2 MHz, CDCl₃):
δ = 9.2939 (6.2); 9.2895 (5.8); 9.2870 (6.1); 8.5826 (3.7); 8.5755 (3.7); 8.5553 (5.0); 8.5483 (5.0); 8.4087 (7.5); 8.3814
(5.4); 8.1556 (1.9); 7.4257 (2.8); 7.4196 (4.2); 7.3972 (11.1); 7.3927 (10.6); 7.3832 (8.4); 7.3596 (10.0); 7.3334 (4.1);
7.2993 (26.5); 7.2881 (2.3); 7.2794 (1.5); 7.2702 (3.8); 7.2617 (1.0); 7.2479 (1.2); 7.1620 (3.6); 6.9901 (7.6); 6.8182
(3.8); 3.7409 (12.4); 3.7211 (12.2); 2.9961 (1.8); 2.9226 (1.6); 2.5216 (0.4); 1.6129 (16.0); 1.0728 (2.0); 1.0477 (10.0);
1.0379 (8.1); 1.0085 (7.7); 0.9990 (9.8); 0.9756 (1.7); 0.0470 (1.2); 0.0369 (27.7); 0.0266 (1.2)
I.057: ¹H-NMR(300.1 MHz, d₆-DMSO):
δ = 8.6576 (11.2); 8.6506 (11.0); 8.4474 (12.8); 7.9940 (2.9); 7.9675 (3.0); 7.6593 (2.1); 7.6337 (3.8); 7.6080 (2.1);
7.3024 (1.1); 7.2970 (1.3); 7.2789 (2.8); 7.2735 (3.0); 7.2596 (3.2); 7.2524 (3.8); 7.2458 (2.7); 7.2338 (2.3); 7.2282
(2.0); 7.1580 (5.2); 7.1504 (5.1); 7.1229 (11.4); 7.0968 (5.4); 6.7864 (2.5); 6.7574 (2.4); 3.3394 (58.5); 3.3164 (0.9);
2.5190 (7.5); 2.5132 (14.2); 2.5074 (18.4); 2.5015 (12.8); 2.0798 (16.0); 1.3392 (7.7); 1.2711 (0.6); 1.2462 (0.7);
1.1773 (9.8); 1.1556 (2.9); 0.0000 (3.5)
I.058: ¹H-NMR(300.2 MHz, d₆-DMSO):
δ = 8.7729 (0.5); 8.7651 (0.5); 8.1052 (0.3); 7.5399 (0.7); 7.0557 (0.4); 7.0248 (0.4); 3.7309 (0.5); 3.7142 (0.8); 3.6959
(0.7); 3.4834 (0.6); 3.4655 (0.7); 3.4497 (0.5); 3.3484 (16.0); 2.5346 (3.4); 2.5286 (7.2); 2.5226 (10.0); 2.5165 (7.2);
2.5106 (3.3); 1.4501 (9.6); 0.0314 (0.4); 0.0205 (12.7); 0.0096 (0.5)
I.059: ¹H-NMR(499.9 MHz, d₆-DMSO):
δ = 8.8296 (16.0); 7.6492 (5.6); 7.6201 (1.8); 7.5174 (3.8); 7.4147 (1.8); 3.3287 (0.6); 2.5080 (0.5); 2.5045 (0.7); 2.5011 (0.5)
I.060: ¹H-NMR(300.2 MHz, CDCl₃):
δ = 9.0294 (1.4); 8.9400 (1.3); 7.2985 (4.1); 7.1113 (2.4); 6.9391 (5.0); 6.7668 (2.5); 5.8584 (0.7); 3.1588 (16.0); 3.1417
(15.7); 1.7015 (2.7); 0.1023 (1.1); 0.0309 (4.9)
I.061: ¹H-NMR(499.9 MHz, d₆-DMSO):
δ = 9.4099 (16.0); 7.7218 (1.8); 7.6196 (4.0); 7.5174 (2.0); 2.5080 (0.5); 2.5044 (0.7); 2.5009 (0.5)
I.062: ¹H-NMR(499.9 MHz, d₆-DMSO):
δ = 8.9175 (16.0); 7.6302 (1.7); 7.5276 (3.8); 7.4249 (1.9); 3.3692 (1.2); 3.3289 (58.0); 3.2330 (50.6); 2.5073 (2.5);
2.5039 (3.2); 2.5006 (2.4)
I.063: ¹H-NMR(300.2 MHz, d₆-DMSO):
δ = 8.9808 (2.4); 8.9314 (2.4); 8.6029 (1.4); 8.5832 (2.7); 8.5637 (1.3); 7.7295 (2.5); 7.5586 (5.6); 7.3878 (2.8); 4.1970
(4.8); 4.1890 (5.1); 4.1772 (5.0); 4.1692 (4.7); 3.3520 (16.0); 3.1423 (2.4); 3.1343 (5.2); 3.1264 (2.3); 2.5277 (4.2);
2.5218 (5.5); 2.5160 (4.0); 0.0168 (5.5)
I.064: ¹H-NMR(300.2 MHz, CDCl₃):
δ = 9.0455 (0.5); 7.2982 (1.3); 7.1145 (0.7); 6.9423 (1.5); 6.7701 (0.8); 6.4438 (0.8); 2.7518 (16.0); 0.0243 (1.6)
I.065: ¹H-NMR(300.2 MHz, d₆-DMSO):
δ = 9.4255 (0.6); 9.3757 (12.1); 7.7971 (1.2); 7.6265 (2.7); 7.4561 (1.3); 7.2076 (1.2); 7.2013 (1.8); 3.3468 (16.0);
2.8653 (0.8); 2.8585 (0.9); 2.8343 (1.6); 2.8150 (1.0); 2.8081 (0.9); 2.6746 (0.8); 2.6658 (0.9); 2.6502 (1.5); 2.6424
(1.5); 2.6255 (1.0); 2.6166 (0.9); 2.5284 (7.7); 2.5225 (10.2); 2.5166 (7.4); 2.1057 (0.6); 2.0800 (1.6); 2.0552 (2.1);
2.0300 (1.4); 2.0049 (0.4); 0.0305 (0.5); 0.0198 (10.7); 0.0089 (0.4)
I.066: ¹H-NMR(300.2 MHz, CDCl₃):
δ = 9.0913 (0.9); 8.9312 (0.9); 7.2988 (3.2); 7.1128 (1.7); 6.9405 (3.5); 6.7683 (1.8); 6.2022 (1.2); 1.6860 (1.8); 1.5303
(16.0); 0.9236 (0.8); 0.8969 (3.8); 0.8849 (3.4); 0.8491 (3.7); 0.8358 (3.9); 0.8105 (0.9); 0.0316 (4.0)
I.067: ¹H-NMR(300.2 MHz, CDCl3):
δ = 8.9890 (5.4); 8.9505 (4.5); 7.2989 (20.0); 7.1085 (7.6); 6.9476 (0.4); 6.9363 (16.0); 6.7640 (8.0); 5.9097 (2.8);
3.4364 (12.8); 3.4178 (14.0); 3.4127 (14.9); 3.3941 (13.0); 1.6580 (9.5); 1.2878 (1.4); 1.2183 (0.5); 1.2022 (0.9);
1.1923 (1.2); 1.1864 (0.7); 1.1762 (2.5); 1.1676 (2.2); 1.1622 (1.8); 1.1518 (4.2); 1.1416 (1.9); 1.1356 (2.4); 1.1257
(2.9); 1.1173 (0.8); 1.1097 (1.6); 1.1013 (1.1); 1.0852 (0.6); 1.0312 (0.4); 1.0069 (0.8); 0.9825 (0.3); 0.8886 (0.4);
0.6538 (3.4); 0.6381 (9.5); 0.6340 (11.2); 0.6187 (5.2); 0.6114 (10.8); 0.6073 (9.5); 0.5922 (4.3); 0.5692 (0.5); 0.5434
(0.5); 0.4123 (0.4); 0.3946 (0.5); 0.3621 (4.1); 0.3464 (13.2); 0.3271 (13.2); 0.3112 (3.0); 0.1045 (0.5); 0.0443 (0.8);
0.0334 (25.1); 0.0226 (1.1)
I.068: ¹H-NMR(300.2 MHz, CDCl₃):
δ = 8.9935 (1.0); 8.9640 (0.9); 7.2984 (2.6); 7.1088 (1.2); 6.9365 (2.4); 6.7643 (1.2); 6.2312 (0.5); 3.7917 (0.8); 3.7747
(2.2); 3.7571 (2.8); 3.7389 (1.5); 3.6516 (2.9); 3.6342 (3.3); 3.6180 (1.5); 3.4401 (16.0); 1.6728 (1.8); 0.0322 (3.2)
I.069: ¹H-NMR(300.2 MHz, CDCl3):
δ = 9.5014 (16.0); 8.6258 (0.4); 8.6162 (3.2); 8.6109 (3.7); 8.6053 (1.9); 8.5915 (3.2); 8.5839 (3.6); 7.6203 (1.3); 7.6037
(7.1); 7.5799 (3.9); 7.5688 (0.8); 7.5614 (1.0); 7.5503 (0.6); 7.2987 (8.2); 7.1847 (2.0); 7.0128 (4.3); 6.8409 (2.2);
1.6054 (9.9); 0.0370 (8.0)
I.070: ¹H-NMR(499.9 MHz, d₆-DMSO):
δ = 10.0771 (0.6); 10.0707 (0.6); 9.6715 (8.0); 9.3686 (0.5); 9.3117 (1.8); 9.3026 (0.4); 9.2676 (16.0); 8.8304 (0.7);
8.7587 (3.9); 7.9526 (1.4); 7.7254 (1.7); 7.7192 (1.7); 7.6507 (0.4); 7.6360 (0.5); 7.6229 (4.2); 7.6164 (4.0); 7.6117
(0.8); 7.5334 (0.6); 7.5205 (2.0); 7.5137 (1.8); 6.5191 (0.4); 3.3138 (37.9); 2.8912 (9.7); 2.7313 (8.2); 2.5090 (7.6);
2.5055 (14.2); 2.5019 (18.8); 2.4983 (13.4); 2.4948 (6.3); 0.0063 (0.4); −0.0002 (7.9)
I.071: ¹H-NMR(300.2 MHz, CDCl₃):
δ = 9.3801 (16.0); 8.2186 (2.6); 8.2146 (2.6); 8.2060 (2.7); 8.2020 (2.5); 7.6819 (2.5); 7.6779 (2.4); 7.6653 (2.7); 7.6613
(2.5); 7.2987 (13.0); 7.2721 (2.5); 7.2595 (2.8); 7.2556 (2.6); 7.2429 (2.3); 7.1719 (2.0); 6.9999 (4.3); 6.8280 (2.2);
1.5971 (8.1); 1.2916 (0.4); 0.0478 (0.5); 0.0370 (17.3); 0.0279 (0.6); 0.0261 (0.7)
I.072: ¹H-NMR(300.2 MHz, CDCl₃):
δ = 9.4257 (16.0); 8.5327 (2.6); 8.5289 (2.6); 8.5224 (2.7); 8.5186 (2.4); 8.0096 (2.7); 8.0058 (2.6); 7.9926 (2.9); 7.9888
(2.6); 7.4848 (2.5); 7.4745 (2.5); 7.4679 (2.4); 7.4576 (2.3); 7.2986 (5.7); 7.1758 (2.0); 7.0039 (4.3); 6.8320 (2.1);
1.6234 (8.6); 0.0356 (7.1)
I.073: ¹H-NMR(400.1 MHz, d₆-DMSO):
δ = 8.6313 (4.3); 8.0582 (1.1); 8.0370 (1.1); 7.4529 (0.9); 7.3246 (1.9); 7.1964 (1.0); 3.4215 (0.4); 3.4011 (0.8); 3.3840
(0.8); 3.3639 (0.4); 3.1288 (16.0); 2.3026 (5.0); 1.0366 (5.2); 1.0201 (5.1); 0.8339 (0.4); 0.8217 (0.7); 0.8102 (0.6);
0.8013 (0.7); 0.7891 (0.4); 0.2663 (0.6); 0.2542 (0.7); 0.2442 (0.8); 0.2326 (0.4); 0.2225 (0.4); 0.2128 (0.4); 0.2015
(0.5); 0.1898 (0.7); 0.1804 (0.7); 0.1688 (0.6); 0.1423 (0.3); 0.1295 (0.6); 0.1190 (0.8); 0.1068 (0.9); 0.0955 (0.6);
0.0228 (0.3); 0.0107 (0.7); −0.0002 (0.9); −0.0124 (0.8); −0.0227 (0.5)
I.074: ¹H-NMR(300.2 MHz, d₆-DMSO):
δ = 8.9824 (3.0); 7.5615 (0.7); 7.3906 (0.4); 3.8991 (0.8); 3.8840 (1.2); 3.8666 (1.1); 3.7270 (1.1); 3.7099 (1.2); 3.6948
(0.7); 3.3478 (16.0); 2.5344 (3.3); 2.5284 (7.1); 2.5224 (9.7); 2.5163 (7.1); 2.5103 (3.3); 0.0314 (0.4); 0.0205 (12.4);
0.0096 (0.5)
I.075: ¹H-NMR(300.2 MHz, CDCl₃):
δ = 9.3826 (16.0); 7.4368 (1.5); 7.4312 (2.2); 7.4088 (5.1); 7.3928 (2.7); 7.3893 (3.0); 7.3824 (0.8); 7.3661 (4.9); 7.3604
(1.7); 7.3419 (2.2); 7.3190 (1.2); 7.3136 (1.9); 7.3077 (1.1); 7.2985 (8.3); 7.2910 (2.0); 7.2805 (0.4); 7.2718 (0.4);
7.2672 (0.6); 7.1513 (2.2); 6.9794 (4.7); 6.8076 (2.3); 5.3368 (0.6); 4.4472 (13.1); 1.6246 (1.5); 0.0483 (0.4); 0.0375
(10.0); 0.0266 (0.4)
I.076: ¹H-NMR(400.1 MHz, CDCl₃):
δ = 9.2806 (0.6); 9.0781 (16.0); 7.6628 (7.2); 7.6431 (8.5); 7.6211 (0.5); 7.5809 (0.4); 7.5196 (3.8); 7.4203 (4.2); 7.4018
(8.0); 7.3830 (4.2); 7.3570 (0.4); 7.3365 (0.4); 7.3104 (0.4); 7.2588 (26.9); 7.2236 (0.6); 7.2082 (0.4); 7.1806 (2.6);
7.1621 (4.0); 7.1435 (1.8); 7.0461 (2.3); 6.9425 (0.3); 6.9168 (4.3); 6.7873 (2.2); 2.9540 (1.2); 2.8832 (1.2); 2.3771
(0.8); 1.5455 (11.0); 1.4018 (0.6); 1.2534 (0.5); −0.0002 (31.4)
I.077: ¹H-NMR(300.2 MHz, CDCl₃):
δ = 9.4762 (16.0); 8.6523 (0.3); 8.6427 (3.0); 8.6356 (1.2); 8.6242 (3.2); 8.6128 (3.4); 8.6015 (1.2); 8.5943 (3.1); 8.5847
(0.4); 7.2988 (5.2); 7.2903 (0.5); 7.2805 (3.2); 7.2737 (1.0); 7.2517 (5.6); 7.2292 (1.0); 7.2224 (3.0); 7.2129 (0.4);
7.1835 (2.0); 7.0116 (4.1); 6.8397 (2.1); 1.6134 (6.1); 0.0358 (4.8)
I.078: ¹H-NMR(300.2 MHz, CDCl₃):
δ = 9.4867 (16.0); 7.6342 (4.1); 7.6276 (4.2); 7.2989 (7.0); 7.2928 (4.8); 7.2861 (4.4); 7.1825 (1.9); 7.0107 (4.1); 6.8389
(2.0); 4.9578 (1.6); 4.9340 (5.1); 4.9103 (5.2); 4.8866 (1.7); 2.9940 (0.6); 2.9209 (0.5); 1.6361 (4.2); 1.5488 (5.5);
1.5251 (11.9); 1.5013 (5.4); 0.0458 (0.3); 0.0350 (8.5)
I.079: ¹H-NMR(300.2 MHz, d₆-DMSO):
δ = 9.6497 (16.0); 8.5492 (2.2); 8.5319 (2.6); 8.3382 (1.1); 8.3332 (1.6); 8.3275 (1.2); 8.3210 (1.1); 8.3161 (1.4); 8.3102
(0.9); 8.0366 (2.9); 7.8407 (1.4); 7.6704 (3.3); 7.5002 (1.6); 3.3461 (13.9); 2.9111 (0.7); 2.7519 (0.6); 2.5346 (3.0);
2.5287 (6.4); 2.5226 (8.8); 2.5166 (6.4); 2.5106 (3.0); 0.0193 (7.8)
I.080: ¹H-NMR(300.2 MHz, d₆-DMSO):
δ = 9.5745 (5.6); 9.5446 (0.4); 9.2917 (1.2); 9.2850 (1.2); 8.9899 (0.4); 8.9817 (0.4); 8.9622 (0.6); 8.9548 (0.6); 8.9349
(0.4); 8.9271 (0.3); 7.8307 (0.5); 7.6603 (1.1); 7.4899 (0.6); 7.4761 (0.7); 7.4675 (0.7); 7.4471 (0.6); 7.4384 (0.6);
6.5687 (0.4); 3.3496 (16.0); 2.5282 (7.2); 2.5226 (9.3); 2.5171 (7.1); 0.0197 (5.0)
I.081: ¹H-NMR(300.2 MHz, CDCl₃):
δ = 9.4501 (10.4); 7.2986 (5.3); 7.1756 (1.3); 7.0037 (2.7); 6.8319 (1.4); 2.8969 (16.0); 2.6814 (15.9); 1.6100 (3.0);
0.0353 (6.8)
I.082: ¹H-NMR(400.1 MHz, d₆-DMSO):
δ = 8.7656 (1.3); 8.6608 (1.4); 8.3828 (2.3); 7.4820 (0.9); 7.3537 (1.9); 7.2255 (1.0); 3.1477 (16.0); 2.7112 (0.4); 2.5515
(0.4); 2.3226 (5.3); 1.3484 (0.5); 1.3403 (0.6); 1.3280 (1.0); 1.3153 (0.6); 1.3077 (0.5); 0.5028 (0.5); 0.4812 (3.1);
0.4464 (3.0); 0.4245 (0.5); 0.1938 (0.6); 0.1792 (1.9); 0.1682 (1.1); 0.1620 (1.8); 0.1588 (1.8); 0.1485 (0.7); 0.0126
(0.7); −0.0002 (2.4); −0.0115 (2.2); −0.0250 (0.6)
I.083: ¹H-NMR(300.2 MHz, CDCl₃):
δ = 9.3723 (16.0); 7.2991 (7.5); 7.1616 (2.1); 6.9897 (4.6); 6.8179 (2.3); 3.0137 (6.7); 2.9897 (7.0); 2.0813 (0.4); 2.0699
(0.6); 2.0570 (0.6); 2.0452 (0.7); 2.0336 (0.6); 2.0206 (0.5); 2.0092 (0.5); 1.7668 (1.2); 1.7572 (1.2); 1.7243 (4.2);
1.6880 (2.3); 1.6821 (2.4); 1.6231 (3.6); 1.3623 (0.3); 1.3307 (0.8); 1.3219 (1.0); 1.2833 (1.8); 1.2542 (2.0); 1.2356
(1.2); 1.1936 (0.8); 1.1783 (0.9); 1.1434 (1.5); 1.1050 (1.2); 1.0682 (0.4); 1.0591 (0.4); 0.0455 (0.4); 0.0347 (9.6);
0.0238 (0.4)
I.084: ¹H-NMR(300.2 MHz, CDCl₃):
δ = 8.9405 (0.6); 7.2987 (2.1); 7.1024 (0.7); 6.9301 (1.5); 6.7577 (0.7); 5.8753 (0.4); 1.6348 (4.3); 1.4406 (0.4); 1.4308
(0.4); 1.4139 (16.0); 1.3940 (0.4); 0.5233 (1.0); 0.5174 (0.8); 0.5112 (0.7); 0.4912 (1.5); 0.4843 (1.4); 0.4791 (1.0);
0.4716 (0.8); 0.4664 (1.0); 0.4600 (0.8); 0.0344 (2.8)
I.085: ¹H-NMR(499.9 MHz, CDCl₃):
δ = 9.5174 (16.0); 8.6993 (6.7); 8.6827 (7.0); 7.8442 (7.0); 7.8276 (6.8); 7.7918 (1.8); 7.7755 (2.4); 7.7031 (2.4); 7.6868
(1.8); 7.5797 (0.3); 7.5650 (0.3); 7.4855 (0.4); 7.4713 (0.3); 7.4018 (0.4); 7.2642 (4.9); 7.0916 (1.2); 6.9884 (2.5);
6.8853 (1.2); 4.1286 (0.8); 4.1143 (0.8); 2.0444 (3.3); 1.2736 (0.9); 1.2593 (1.9); 1.2451 (1.0); −0.0002 (5.2)
I.086: ¹H-NMR(400.1 MHz, d₆-DMSO):
δ = 9.8095 (3.7); 8.9489 (8.2); 7.9585 (1.0); 7.6782 (1.4); 7.5499 (3.2); 7.4218 (1.6); 7.3877 (2.0); 7.3687 (2.5); 7.2901
(1.7); 7.2716 (2.3); 7.2462 (0.9); 7.2282 (2.0); 7.2093 (1.3); 7.1839 (1.7); 7.1661 (2.1); 7.1473 (0.7); 3.3350 (28.6);
2.8958 (5.8); 2.7364 (5.4); 2.5075 (8.3); 2.2197 (16.0)
I.087: ¹H-NMR(300.2 MHz, CDCl₃):
δ = 9.0060 (4.9); 7.5375 (1.1); 7.5317 (0.6); 7.5110 (2.2); 7.4969 (0.5); 7.4859 (2.3); 7.3941 (1.1); 7.3776 (3.8); 7.3714
(2.6); 7.3510 (2.8); 7.2990 (3.4); 7.1069 (1.2); 6.9345 (2.5); 6.7622 (1.2); 3.6657 (16.0); 1.6148 (2.4); 0.0380 (4.3)
I.088: ¹H-NMR(499.9 MHz, CDCl₃):
δ = 8.9202 (4.6); 8.4736 (4.1); 7.3038 (23.2); 7.2960 (36.1); 7.2674 (4.5); 7.2595 (5.7); 7.2512 (5.4); 7.1866 (3.8);
6.9370 (3.1); 6.8335 (6.4); 6.7301 (3.3); 6.6590 (4.3); 5.2221 (0.3); 4.6515 (13.9); 4.6407 (16.0); 3.5905 (0.6); 1.5049
(0.5); 1.3993 (0.3); 1.3502 (0.6); 1.2610 (1.0); 1.2112 (2.2); 1.1820 (4.5); 0.9883 (0.5); 0.9705 (0.5); 0.9518 (0.5);
0.8053 (1.1); 0.7664 (3.4); −0.0002 (3.6); −0.0737 (2.1)
I.089: ¹H-NMR(300.2 MHz, CDCl₃):
δ = 9.4285 (10.1); 8.5808 (0.5); 8.5714 (3.6); 8.5645 (1.2); 8.5482 (1.2); 8.5412 (3.7); 8.5318 (0.4); 7.2987 (6.8); 7.1739
(1.2); 7.0988 (0.5); 7.0893 (3.7); 7.0824 (1.2); 7.0661 (1.2); 7.0592 (3.5); 7.0496 (0.4); 7.0019 (2.6); 6.8300 (1.3);
3.9509 (16.0); 1.6048 (8.2); 0.0365 (6.4)
I.090: ¹H-NMR(300.2 MHz, CDCl₃):
δ = 9.2921 (0.4); 9.2565 (16.0); 7.5603 (2.0); 7.5546 (2.4); 7.5333 (3.3); 7.5302 (3.1); 7.4590 (1.4); 7.4507 (1.5); 7.4454
(1.0); 7.4301 (4.0); 7.4248 (1.9); 7.4057 (3.9); 7.3960 (1.7); 7.3901 (0.8); 7.3808 (1.2); 7.3659 (0.4); 7.3549 (0.4);
7.3384 (0.4); 7.3106 (0.8); 7.2986 (35.5); 7.2317 (0.4); 7.1428 (2.0); 6.9709 (4.4); 6.7990 (2.2); 5.6052 (11.5); 5.3375
(0.5); 2.9955 (2.5); 2.9224 (2.2); 2.0456 (1.4); 1.6201 (11.4); 0.0472 (1.6); 0.0455 (1.0); 0.0364 (46.6); 0.0287 (1.3);
0.0271 (1.2); 0.0255 (1.6); −0.0306 (0.4)
I.091: ¹H-NMR(300.2 MHz, CDCl₃):
δ = 9.3825 (16.0); 7.4090 (2.2); 7.4018 (0.9); 7.3912 (2.4); 7.3798 (2.6); 7.3693 (1.1); 7.3619 (2.5); 7.2988 (8.9); 7.1538
(2.2); 7.0896 (0.4); 7.0796 (3.1); 7.0724 (1.0); 7.0574 (1.0); 7.0505 (5.5); 7.0435 (1.1); 7.0285 (0.9); 7.0214 (2.5);
6.9819 (4.8); 6.8101 (2.4); 4.4108 (10.0); 1.6202 (1.7); 0.0475 (0.4); 0.0367 (11.7); 0.0258 (0.4)
I.092: ¹H-NMR(300.2 MHz, CDCl₃):
δ = 9.1549 (16.0); 9.1444 (0.3); 7.6979 (2.1); 7.6922 (2.4); 7.6906 (2.7); 7.6795 (2.8); 7.6770 (2.5); 7.6735 (1.7); 7.6658
(3.3); 7.6592 (0.6); 7.5532 (0.8); 7.5515 (0.8); 7.5472 (1.0); 7.5397 (6.9); 7.5327 (5.8); 7.5228 (3.2); 7.5170 (3.1);
7.5108 (0.7); 7.5087 (0.6); 7.5038 (0.8); 7.2987 (3.4); 7.1325 (2.0); 6.9606 (4.2); 6.7886 (2.1); 1.6188 (1.8); 0.0380 (4.5)
I.093: ¹H-NMR(300.1 MHz, d₆-DMSO):
δ = 10.0530 (3.3); 9.0012 (16.0); 7.7251 (1.7); 7.6374 (0.9); 7.6321 (0.8); 7.6122 (1.6); 7.6064 (1.7); 7.5876 (1.1);
7.5799 (0.9); 7.5542 (4.1); 7.3835 (1.9); 7.3355 (0.4); 7.3284 (0.6); 7.3085 (1.4); 7.3025 (2.3); 7.2769 (2.4); 7.2689
(2.3); 7.2624 (2.5); 7.2519 (2.8); 7.2437 (1.3); 7.2386 (1.0); 7.2273 (1.8); 7.2187 (1.3); 7.2063 (0.5); 7.2032 (0.5);
7.1962 (0.5); 3.3348 (25.0); 2.5098 (5.4); 2.5040 (7.0); 2.4982 (4.9); −0.0002 (3.5)
I.094: ¹H-NMR(400.1 MHz, CDCl₃):
δ = 9.2782 (0.3); 9.1113 (16.0); 9.0610 (0.6); 8.0193 (0.6); 7.7381 (3.0); 7.7074 (3.0); 7.6780 (0.6); 7.6575 (0.5); 7.6138
(0.4); 7.5491 (3.5); 7.5182 (0.7); 7.3836 (0.3); 7.3560 (1.2); 7.3382 (3.1); 7.3183 (3.7); 7.3009 (2.4); 7.2597 (59.9);
7.1174 (0.3); 7.0973 (0.4); 7.0526 (2.4); 6.9962 (0.4); 6.9241 (4.5); 6.9018 (0.4); 6.8665 (1.9); 6.8457 (3.3); 6.8259
(2.0); 6.7943 (2.6); 2.9548 (2.0); 2.8836 (2.0); 2.3898 (1.5); 1.5383 (27.7); 1.4048 (0.5); 1.3559 (0.4); 1.2591 (0.6);
0.1460 (0.4); −0.0002 (69.0); −0.1487 (0.4); −0.1518 (0.5)
I.095: ¹H-NMR(300.2 MHz, CDCl₃):
δ = 9.1044 (16.0); 7.6609 (3.0); 7.6538 (1.2); 7.6452 (3.2); 7.6381 (2.0); 7.6308 (3.5); 7.6224 (1.3); 7.6151 (3.4); 7.6034
(0.4); 7.5515 (1.8); 7.2987 (12.9); 7.1769 (0.3); 7.1654 (3.4); 7.1580 (1.1); 7.1499 (0.6); 7.1353 (6.0); 7.1151 (1.0);
7.1079 (3.0); 6.9615 (4.6); 6.7894 (2.3); 5.3383 (5.3); 4.1713 (0.8); 4.1475 (0.8); 4.1237 (0.3); 2.0844 (4.0); 2.0476
(3.3); 1.6183 (7.8); 1.3208 (1.1); 1.2969 (1.9); 1.2731 (1.0); 0.0469 (0.6); 0.0361 (14.5); 0.0255 (0.6)
I.096: ¹H-NMR(400.1 MHz, CDCl₃):
δ = 9.3349 (0.6); 9.2740 (0.6); 9.1577 (0.6); 9.0849 (16.0); 8.0192 (1.9); 7.5582 (7.4); 7.2614 (11.9); 7.1773 (4.2);
7.0488 (2.2); 6.9198 (4.5); 6.7908 (2.2); 6.3849 (0.4); 6.3571 (7.5); 4.1723 (0.3); 4.1289 (2.4); 4.1110 (6.9); 4.0929
(6.9); 4.0750 (2.4); 2.9561 (8.9); 2.8829 (8.6); 1.6048 (8.4); 1.5372 (0.4); 1.5193 (0.7); 1.4845 (7.2); 1.4664 (13.9);
1.4484 (6.9); 1.3169 (0.4); −0.0002 (15.1)
I.097: ¹H-NMR(400.1 MHz, d₆-DMSO):
δ = 8.7093 (0.5); 8.6611 (0.5); 8.4548 (0.9); 7.4947 (0.3); 7.3664 (0.8); 7.2383 (0.4); 3.1661 (16.0); 2.3410 (5.2); 0.9769
(3.9); 0.5507 (1.5); 0.5334 (1.5); 0.4409 (0.4); 0.4291 (1.3); 0.4177 (0.4); 0.0104 (0.5); −0.0002 (1.3); −0.0029
(1.3); −0.0138 (0.4)
I.098: ¹H-NMR(300.2 MHz, CDCl₃):
δ = 9.2736 (16.0); 7.2989 (12.7); 7.2680 (0.7); 7.2584 (0.6); 7.2524 (1.2); 7.2468 (0.9); 7.2431 (0.9); 7.2370 (4.5);
7.2296 (2.0); 7.2208 (8.2); 7.2117 (1.3); 7.2037 (0.9); 7.1946 (4.3); 7.1726 (0.6); 7.1633 (1.1); 7.1528 (2.0); 6.9810
(4.1); 6.8091 (2.0); 2.9957 (0.6); 2.9228 (0.6); 2.0467 (0.5); 1.6040 (11.7); 0.0470 (0.5); 0.0363 (14.7); 0.0253 (0.5)
I.099: ¹H-NMR(300.2 MHz, CDCl₃):
δ = 9.5893 (16.0); 7.9401 (1.9); 7.9332 (1.3); 7.9279 (1.2); 7.9180 (1.7); 7.9087 (2.2); 7.6105 (1.4); 7.6025 (1.3); 7.5903
(1.2); 7.5850 (1.6); 7.5796 (2.7); 7.5278 (0.5); 7.5202 (0.9); 7.5033 (2.4); 7.4936 (3.1); 7.4825 (4.0); 7.4696 (3.1);
7.4629 (1.9); 7.4445 (0.6); 7.4383 (0.4); 7.2989 (3.2); 7.1940 (2.0); 7.0222 (4.2); 6.8504 (2.1); 1.6379 (3.4); 0.0335 (3.1)
I.100: ¹H-NMR(300.2 MHz, CDCl₃):
δ = 9.5105 (16.0); 8.6104 (1.8); 8.6048 (3.3); 8.5987 (2.1); 8.5105 (1.3); 8.5058 (2.1); 8.5008 (1.2); 8.4851 (1.3); 8.4805
(2.3); 8.4756 (1.3); 7.6024 (0.7); 7.5978 (1.0); 7.5911 (0.8); 7.5760 (1.8); 7.5697 (2.3); 7.5647 (1.7); 7.5436 (2.7);
7.5179 (3.3); 7.4917 (1.2); 7.2989 (11.6); 7.1888 (2.0); 7.0170 (4.1); 6.8452 (2.1); 1.5974 (14.4); 0.0475 (0.4); 0.0367
(11.5); 0.0258 (0.4)
I.101: ¹H-NMR(300.2 MHz, d₆-DMSO):
δ = 9.5346 (6.6); 8.5311 (0.4); 8.5228 (2.4); 8.5166 (0.8); 8.5003 (0.9); 8.4940 (2.6); 8.4857 (0.3); 7.8244 (0.7); 7.7138
(0.4); 7.7056 (2.6); 7.6993 (0.8); 7.6829 (0.8); 7.6767 (2.4); 7.6684 (0.4); 7.6539 (1.6); 7.4836 (0.8); 3.3493 (16.0);
2.5339 (1.2); 2.5282 (2.4); 2.5222 (3.2); 2.5163 (2.3); 2.5106 (1.1); 0.0186 (1.6)
I.102: ¹H-NMR(300.2 MHz, d₆-DMSO):
δ = 9.6444 (12.4); 8.7211 (2.2); 8.7136 (0.7); 8.7096 (0.7); 8.7022 (2.4); 8.3704 (4.4); 8.3649 (2.9); 8.3586 (2.2); 8.3538
(1.0); 7.8398 (1.1); 7.6694 (2.6); 7.4992 (1.2); 3.3477 (16.0); 2.9111 (0.4); 2.7505 (0.3); 2.5346 (2.4); 2.5287 (5.1);
2.5226 (7.1); 2.5166 (5.1); 2.5107 (2.4); 0.0192 (5.5)
I.103: ¹H-NMR(499.9 MHz, d₆-DMSO):
δ = 9.5838 (16.0); 9.5264 (3.9); 9.5230 (3.9); 8.8950 (3.7); 8.8902 (3.8); 8.7714 (2.5); 8.7671 (3.5); 8.7631 (2.2); 7.7458
(1.6); 7.6435 (3.6); 7.5412 (1.7); 3.3176 (58.4); 2.5940 (0.3); 2.5403 (0.6); 2.5080 (12.2); 2.5044 (16.0); 2.5009 (11.8)
I.104: ¹H-NMR(300.2 MHz, d₆-DMSO):
δ = 8.9860 (0.6); 8.9762 (0.9); 8.9363 (1.0); 8.9264 (0.5); 8.8620 (1.0); 7.7308 (0.5); 7.5598 (1.2); 7.3891 (0.5); 3.5934
(6.5); 3.3445 (16.0); 2.5344 (2.9); 2.5285 (6.3); 2.5224 (8.7); 2.5164 (6.2); 2.5105 (2.9); 1.5582 (0.4); 1.5422 (1.1);
1.5311 (1.2); 1.5170 (0.5); 1.2315 (0.5); 1.2172 (1.2); 1.2060 (1.1); 1.1901 (0.4); 0.0314 (0.3); 0.0206 (10.4); 0.0095 (0.4)
I.105: ¹H-NMR(300.2 MHz, CDCl₃):
δ = 8.9963 (4.1); 8.9361 (4.0); 7.2988 (9.0); 7.1114 (4.8); 6.9392 (10.2); 6.7669 (5.2); 5.9289 (1.6); 5.9107 (2.7); 5.8918
(1.6); 5.3343 (1.1); 4.0613 (4.4); 4.0491 (4.5); 4.0242 (5.0); 4.0115 (5.0); 3.5035 (8.6); 3.4814 (16.0); 3.4733 (6.2);
3.4654 (6.4); 3.4597 (9.6); 3.4331 (8.7); 3.4270 (8.8); 3.3942 (4.5); 3.3878 (4.3); 2.0055 (0.4); 1.9940 (0.6); 1.9816
(1.2); 1.9677 (1.4); 1.9562 (1.6); 1.9438 (2.3); 1.9308 (1.8); 1.9196 (1.5); 1.9057 (1.6); 1.8930 (0.9); 1.8822 (0.6);
1.8694 (0.4); 1.7564 (4.4); 1.7506 (4.2); 1.7165 (10.7); 1.4952 (2.2); 1.4803 (2.2); 1.4552 (4.4); 1.4403 (4.7); 1.4120
(3.8); 1.3972 (3.6); 1.3719 (1.5); 1.3570 (1.4); 1.2855 (0.8); 0.1027 (0.5); 0.0412 (0.4); 0.0304 (11.0)
I.106: ¹H-NMR(300.2 MHz, CDCl₃):
δ = 9.0788 (7.9); 7.4315 (0.5); 7.4162 (0.9); 7.4004 (3.8); 7.3917 (4.2); 7.3755 (0.8); 7.3608 (0.5); 7.2988 (37.4); 7.1228
(0.9); 6.9505 (2.1); 6.7782 (1.0); 5.3384 (4.6); 5.0607 (12.3); 5.0277 (0.4); 2.0465 (13.0); 1.5970 (16.0); 0.0483 (1.6);
0.0375 (50.6); 0.0282 (1.6); 0.0267 (1.8)
I.107: ¹H-NMR(300.2 MHz, CDCl₃):
δ = 9.3636 (9.1); 7.3522 (0.4); 7.3465 (0.6); 7.3209 (2.6); 7.2988 (7.2); 7.1517 (1.3); 7.0269 (0.8); 7.0236 (0.9); 7.0022
(1.4); 6.9993 (1.3); 6.9798 (3.0); 6.9471 (1.4); 6.9202 (1.2); 6.8079 (1.4); 4.4776 (7.0); 3.7859 (16.0); 1.6162 (4.8);
0.0370 (7.8)
I.108: ¹H-NMR(300.2 MHz, CDCl₃):
δ = 9.0732 (8.0); 9.0616 (0.5); 8.9650 (0.8); 7.4650 (1.5); 7.3410 (1.5); 7.3339 (0.6); 7.3175 (2.3); 7.3115 (2.3); 7.3050
(0.6); 7.2989 (3.6); 7.2874 (2.0); 7.1154 (1.2); 6.9668 (3.8); 6.9430 (4.8); 6.9408 (4.7); 6.9377 (3.7); 6.9170 (0.6);
6.9139 (0.7); 6.7711 (1.3); 4.0340 (0.3); 3.6696 (0.4); 3.3678 (16.0); 2.8332 (0.3); 2.6494 (0.4); 2.0812 (1.1); 1.6448
(4.9); 1.3193 (0.5); 1.3032 (0.7); 1.2956 (1.1); 1.2717 (0.3); 0.9188 (0.8); 0.0369 (4.5)
I.109: ¹H-NMR(400.1 MHz, d₆-DMSO):
δ = 9.2802 (3.2); 8.9825 (10.2); 7.7353 (1.7); 7.7156 (1.8); 7.6838 (1.2); 7.5555 (2.6); 7.4274 (1.3); 7.2149 (0.7); 7.2116
(0.7); 7.1943 (1.7); 7.1760 (1.2); 7.1135 (2.4); 7.0938 (1.5); 7.0057 (1.1); 6.9865 (1.9); 6.9679 (0.9); 3.8164 (16.0);
3.3345 (22.0); 2.5074 (7.2)
I.110: ¹H-NMR(300.2 MHz, CDCl₃):
δ = 10.4470 (1.1); 8.5201 (2.6); 7.4838 (2.1); 7.4761 (2.4); 7.4735 (2.4); 7.4575 (5.4); 7.4514 (5.9); 7.4418 (2.4); 7.4273
(2.6); 7.4114 (5.8); 7.4043 (3.3); 7.4018 (3.4); 7.3932 (7.0); 7.3873 (6.4); 7.3776 (1.5); 7.3719 (0.9); 7.2988 (10.4);
7.1503 (2.5); 6.9782 (5.6); 6.8062 (2.7); 5.1928 (16.0); 5.1024 (0.8); 2.0463 (0.4); 1.6133 (1.4); 0.0483 (0.5); 0.0375
(13.8); 0.0283 (0.4); 0.0267 (0.5)
I.111: ¹H-NMR(300.2 MHz, CDCl₃):
δ = 9.1263 (0.4); 9.0352 (4.0); 8.9028 (3.8); 7.4653 (3.1); 7.4447 (5.5); 7.4398 (6.2); 7.4198 (3.4); 7.4146 (3.4); 7.3721
(1.6); 7.3659 (1.8); 7.3468 (4.1); 7.3404 (3.4); 7.3280 (3.7); 7.3210 (4.7); 7.3141 (3.3); 7.2987 (42.1); 7.2336 (0.3);
7.1852 (4.7); 7.1817 (5.6); 7.1569 (13.4); 7.1308 (6.9); 7.1237 (6.0); 7.1100 (7.3); 7.0957 (3.6); 6.9378 (14.1); 6.7655
(7.0); 6.2358 (3.3); 4.8760 (0.5); 4.8391 (16.0); 4.8184 (15.4); 4.0707 (0.6); 3.9753 (0.6); 2.9949 (2.2); 2.9214 (1.9);
1.6122 (14.6); 1.2914 (0.6); 0.1074 (0.3); 0.0477 (2.8); 0.0370 (54.3); 0.0262 (2.6)
I.112: ¹H-NMR(300.2 MHz, CDCl₃):
δ = 9.0302 (2.7); 8.7661 (2.6); 7.4099 (0.8); 7.4001 (6.8); 7.3931 (3.3); 7.3824 (7.7); 7.3710 (9.0); 7.3606 (3.8); 7.3534
(8.4); 7.2986 (12.9); 7.1230 (1.6); 7.1139 (12.3); 7.1065 (3.7); 7.0913 (3.7); 7.0847 (18.0); 7.0779 (4.0); 7.0627 (2.9);
7.0558 (8.2); 7.0460 (1.0); 6.9438 (14.8); 6.7716 (7.3); 6.4974 (2.5); 5.3354 (1.1); 4.7416 (16.0); 4.7219 (15.7); 1.6772
(9.7); 0.0458 (0.5); 0.0349 (14.9); 0.0239 (0.6)
I.113: ¹H-NMR(400.2 MHz, d₆-DMSO):
δ = 9.9551 (4.3); 9.0392 (16.0); 7.7791 (1.7); 7.6460 (5.1); 7.6307 (2.2); 7.6269 (2.0); 7.6125 (1.2); 7.6067 (1.1); 7.5132
(1.9); 7.3146 (0.7); 7.3101 (0.8); 7.2945 (1.7); 7.2903 (2.1); 7.2803 (1.1); 7.2628 (3.2); 7.2572 (1.7); 7.2492 (1.9);
7.2432 (2.0); 7.2380 (2.9); 7.2318 (2.2); 7.2195 (2.3); 7.2143 (1.9); 7.2015 (0.8); 7.1967 (0.6); 3.3290 (8.2); 2.5057
(14.6); 2.5017 (18.4); 1.9883 (0.3); −0.0022 (1.2)
I.114: ¹H-NMR(300.2 MHz, CDCl₃):
δ = 9.1615 (6.1); 8.5577 (0.8); 8.5530 (0.8); 8.5300 (0.8); 8.5252 (0.8); 8.0216 (0.6); 7.5026 (0.8); 7.4979 (0.8); 7.4759
(0.9); 7.4711 (0.9); 7.4209 (0.4); 7.4160 (0.4); 7.3916 (0.7); 7.3685 (0.4); 7.3636 (0.4); 7.2985 (7.5); 7.1544 (0.6);
7.1494 (0.6); 7.1412 (0.8); 7.1295 (0.8); 7.1279 (0.8); 7.1245 (0.8); 7.1030 (0.4); 7.0979 (0.4); 6.9690 (1.7); 6.7969
(0.9); 1.5993 (16.0); 0.0475 (0.4); 0.0367 (9.7); 0.0258 (0.3)
I.115: ¹H-NMR(300.2 MHz, CDCl₃):
δ = 9.3821 (16.0); 7.3938 (0.6); 7.3643 (1.6); 7.3430 (1.5); 7.3376 (1.2); 7.3152 (1.1); 7.2995 (12.9); 7.1566 (2.2);
6.9848 (4.6); 6.9442 (0.7); 6.9354 (1.1); 6.9187 (1.9); 6.9112 (3.4); 6.8839 (3.7); 6.8558 (1.2); 6.8475 (0.8); 6.8130
(2.3); 4.4647 (9.9); 2.9957 (0.5); 2.9219 (0.5); 1.6042 (5.8); 0.0481 (0.6); 0.0373 (15.7); 0.0266 (0.7)
I.116: ¹ H-NMR(300.1 MHz, d₆-DMSO):
δ = 10.1154 (3.7); 9.0262 (16.0); 7.6287 (0.9); 7.6236 (0.8); 7.6036 (1.6); 7.5988 (1.8); 7.5785 (1.0); 7.5715 (0.8);
7.3320 (0.5); 7.3116 (1.6); 7.3050 (2.2); 7.2869 (2.4); 7.2815 (2.7); 7.2686 (2.6); 7.2621 (2.1); 7.2552 (2.6); 7.2465
(1.1); 7.2305 (1.6); 7.2222 (1.1); 7.2110 (0.4); 7.2068 (0.5); 7.1998 (0.5); 3.3318 (55.0); 2.5144 (3.4); 2.5086 (6.6);
2.5027 (8.5); 2.4968 (5.9); −0.0004 (4.2)
I.117: ¹H-NMR(300.2 MHz, d₆-DMSO):
δ = 8.9851 (0.9); 8.9750 (1.6); 8.9381 (1.7); 8.9280 (0.9); 8.8440 (1.6); 7.7289 (0.7); 7.5579 (1.9); 7.3872 (0.9); 4.0902
(0.8); 4.0666 (2.6); 4.0429 (2.6); 4.0194 (0.8); 3.3479 (16.0); 2.5343 (2.7); 2.5283 (5.8); 2.5222 (8.1); 2.5161 (5.8);
2.5102 (2.7); 2.0096 (0.4); 1.5448 (0.6); 1.5288 (1.6); 1.5179 (1.8); 1.5036 (0.7); 1.2174 (0.8); 1.2027 (1.7); 1.1917
(1.7); 1.1757 (0.6); 1.1219 (2.8); 1.0983 (5.9); 1.0747 (2.6); 0.0199 (6.1)
I.118: ¹H-NMR(300.2 MHz, CDCl₃):
δ = 9.0759 (1.2); 8.7434 (1.2); 7.9680 (0.4); 7.6572 (7.2); 7.6515 (8.6); 7.6319 (10.3); 7.6298 (9.9); 7.6272 (10.1);
7.6180 (1.4); 7.5985 (0.3); 7.5881 (0.3); 7.5831 (0.4); 7.5597 (0.4); 7.5151 (0.4); 7.4892 (0.6); 7.4781 (3.2); 7.4692
(3.6); 7.4635 (2.3); 7.4583 (2.1); 7.4488 (12.8); 7.4436 (5.5); 7.4296 (6.4); 7.4245 (13.6); 7.4204 (11.0); 7.4147 (6.1);
7.4089 (2.5); 7.3996 (3.8); 7.3970 (3.8); 7.3834 (0.9); 7.3790 (1.0); 7.3739 (1.3); 7.3693 (0.9); 7.2988 (22.7); 7.1243
(6.1); 6.9521 (14.1); 6.9219 (3.3); 6.8942 (3.6); 6.7800 (6.8); 6.2795 (4.5); 6.2716 (4.7); 6.2517 (4.2); 6.2438 (4.3);
5.3373 (4.6); 4.1711 (0.4); 4.1473 (0.4); 3.2599 (0.6); 2.5909 (15.9); 2.5829 (16.0); 2.5498 (0.4); 2.5422 (0.4); 2.0832
(1.7); 2.0452 (1.2); 1.6256 (11.3); 1.3442 (1.3); 1.3222 (1.6); 1.3033 (2.4); 1.2970 (2.9); 1.2731 (0.7); 0.9409 (0.9);
0.9192 (2.9); 0.8960 (1.0); 0.0482 (0.8); 0.0374 (28.5); 0.0265 (1.1)
I.119: ¹H-NMR(300.2 MHz, CDCl₃):
δ = 9.3616 (1.0); 9.1196 (10.6); 8.2546 (1.3); 8.2511 (1.3); 8.2274 (1.4); 8.2240 (1.3); 8.0233 (1.0); 7.3735 (0.5); 7.3679
(0.6); 7.3437 (1.1); 7.3222 (0.6); 7.3166 (0.7); 7.2987 (7.9); 7.2545 (0.8); 7.2320 (1.4); 7.1719 (1.0); 7.1682 (1.0);
7.1470 (1.3); 7.1434 (1.3); 7.1334 (1.4); 7.1220 (0.5); 7.1184 (0.5); 6.9612 (3.0); 6.7890 (1.5); 1.9600 (0.4); 1.9507
(0.4); 1.9326 (0.8); 1.9147 (0.4); 1.9052 (0.4); 1.6117 (16.0); 1.1308 (0.7); 1.1168 (1.9); 1.1105 (2.1); 1.1031 (0.9);
1.0968 (1.0); 1.0886 (2.1); 1.0826 (1.9); 1.0690 (0.8); 0.7934 (0.9); 0.7797 (2.0); 0.7743 (2.5); 0.7616 (2.1); 0.7559
(2.1); 0.7415 (0.7); 0.0481 (0.3); 0.0373 (10.0); 0.0265 (0.4)
I.120: ¹H-NMR(300.2 MHz, CDCl₃):
δ = 9.1898 (16.0); 7.4387 (1.9); 7.4334 (2.8); 7.4264 (1.0); 7.4102 (4.3); 7.4062 (3.9); 7.3989 (0.7); 7.3680 (1.5); 7.3639
(2.4); 7.3572 (0.9); 7.3405 (4.5); 7.3349 (1.8); 7.3201 (1.2); 7.3152 (2.3); 7.3078 (0.7); 7.2987 (13.9); 7.2907 (1.4);
7.2855 (2.0); 7.2805 (1.1); 7.2704 (0.7); 7.2617 (2.0); 7.2524 (0.4); 7.2425 (0.4); 7.2380 (0.6); 7.2331 (0.3); 7.1162
(1.8); 6.9443 (4.0); 6.7723 (2.0); 3.3375 (0.9); 1.5955 (10.9); 1.5847 (1.3); 1.5726 (1.0); 1.5611 (2.0); 1.5554 (3.5);
1.5525 (3.1); 1.5476 (2.3); 1.5370 (2.0); 1.5295 (0.6); 1.5223 (1.2); 1.4870 (1.2); 1.4744 (2.0); 1.4617 (2.3); 1.4538
(3.4); 1.4483 (2.0); 1.4368 (0.9); 1.4266 (0.9); 1.2927 (0.7); 0.0480 (0.6); 0.0371 (17.2); 0.0263 (0.7)
I.121: ¹H-NMR(300.2 MHz, CDCl₃):
δ = 9.0198 (7.2); 9.0115 (7.2); 8.5502 (3.4); 8.5472 (4.1); 8.5445 (4.3); 8.5415 (3.7); 8.5342 (3.7); 8.5312 (4.3); 8.5284
(4.2); 8.5254 (3.6); 7.6136 (2.6); 7.6076 (2.7); 7.5872 (4.8); 7.5813 (4.7); 7.5620 (3.6); 7.5560 (3.5); 7.3589 (7.3);
7.3559 (4.7); 7.3354 (3.7); 7.3323 (5.9); 7.2987 (24.6); 7.1333 (3.6); 7.1298 (3.6); 7.1172 (3.7); 7.1136 (3.9); 7.1083
(4.2); 7.1038 (7.1); 7.0923 (3.4); 7.0888 (3.2); 6.9312 (10.7); 6.7590 (5.3); 6.5542 (5.0); 4.1699 (0.5); 4.1461 (0.5);
2.0824 (2.5); 1.8991 (4.3); 1.8833 (12.9); 1.8734 (13.3); 1.8586 (5.0); 1.8055 (0.5); 1.6461 (16.0); 1.4909 (0.5); 1.4374
(5.1); 1.4229 (13.1); 1.4129 (13.1); 1.3973 (4.4); 1.3458 (0.4); 1.3195 (0.8); 1.2957 (1.7); 1.2719 (0.7); 0.9174 (0.5);
0.0463 (1.1); 0.0355 (30.3); 0.0247 (1.3)
I.122: ¹H-NMR(300.2 MHz, CDCl₃):
δ = 8.9424 (1.3); 8.8930 (1.3); 7.4061 (0.6); 7.3919 (14.7); 7.3771 (17.6); 7.3588 (0.7); 7.3516 (0.7); 7.3470 (0.8);
7.3432 (0.8); 7.3298 (1.8); 7.3163 (2.4); 7.3111 (1.1); 7.2987 (4.2); 7.2881 (1.0); 7.2727 (0.4); 7.0956 (2.9); 6.9233
(6.4); 6.7510 (3.1); 6.3075 (1.4); 6.2803 (1.5); 5.3334 (0.3); 5.1484 (1.1); 5.1241 (2.7); 5.0973 (2.6); 5.0728 (1.1);
2.0410 (0.7); 2.0380 (0.7); 2.0268 (0.6); 2.0166 (2.2); 2.0025 (2.1); 1.9919 (3.3); 1.9785 (3.2); 1.9672 (2.3); 1.9545
(2.4); 1.9426 (0.6); 1.9316 (0.8); 1.9086 (0.3); 1.7334 (6.9); 1.3038 (1.7); 1.0335 (7.7); 1.0089 (16.0); 0.9842 (7.0);
0.9406 (0.7); 0.9187 (2.0); 0.8955 (0.7); 0.0373 (4.0)
I.123: ¹H-NMR(300.2 MHz, CDCl₃):
δ = 9.1842 (13.8); 7.5311 (4.0); 7.5069 (5.4); 7.4457 (0.4); 7.4023 (2.5); 7.3788 (5.4); 7.3532 (3.5); 7.3236 (2.6); 7.2989
(9.6); 7.2754 (1.0); 7.1414 (1.8); 6.9695 (3.7); 6.7976 (1.8); 5.2111 (0.8); 5.1877 (2.4); 5.1640 (2.4); 5.1403 (0.8);
1.9253 (0.3); 1.8586 (11.3); 1.8348 (11.0); 1.6029 (16.0); 0.0379 (8.7)
I.124: ¹H-NMR(400.1 MHz, d₆-DMSO):
δ = 8.8910 (4.8); 8.8498 (6.4); 8.8319 (5.6); 7.9581 (1.4); 7.6492 (3.0); 7.5209 (6.8); 7.4823 (2.1); 7.4628 (4.2); 7.4436
(2.2); 7.3928 (3.4); 7.3071 (0.9); 7.2893 (2.4); 7.2738 (3.0); 7.2590 (1.7); 7.1879 (5.0); 7.1658 (9.0); 7.1455 (3.7);
5.5160 (0.6); 5.4982 (2.4); 5.4799 (3.5); 5.4613 (2.4); 5.4436 (0.6); 3.3347 (71.6); 2.8956 (7.9); 2.7361 (7.3); 2.5075
(22.6); 1.4984 (16.0); 1.4809 (15.9)
I.125: ¹H-NMR(300.2 MHz, CDCl₃):
δ = 8.9585 (0.8); 8.8259 (0.8); 7.4250 (0.4); 7.4149 (3.6); 7.4088 (1.6); 7.3973 (3.9); 7.3919 (2.5); 7.3865 (4.6); 7.3753
(1.8); 7.3689 (4.2); 7.3591 (0.5); 7.2985 (2.7); 7.1049 (3.2); 7.0914 (4.9); 7.0843 (1.6); 7.0690 (1.7); 7.0625 (8.6);
7.0558 (1.8); 7.0405 (1.3); 7.0335 (4.0); 7.0234 (0.5); 6.9326 (6.7); 6.7604 (3.3); 6.4716 (1.5); 6.4461 (1.6); 5.3638
(0.4); 5.3404 (1.7); 5.3165 (2.4); 5.2926 (1.7); 5.2691 (0.4); 2.0783 (1.0); 1.7848 (3.0); 1.6555 (16.0); 1.6325 (15.8);
1.3151 (0.6); 1.2979 (1.5); 1.2915 (1.7); 1.2676 (0.4); 0.9348 (0.5); 0.9131 (1.7); 0.8898 (0.6); 0.0322 (2.5)
I.125a: ¹H-NMR(400.2 MHz, d₆-DMSO):
δ = 8.8682 (4.7); 8.8392 (4.7); 8.7912 (3.8); 8.7706 (3.9); 7.9557 (1.5); 7.6487 (3.2); 7.5204 (8.0); 7.4545 (5.2); 7.4497
(2.5); 7.4405 (6.0); 7.4329 (6.7); 7.4241 (2.7); 7.4190 (6.0); 7.3923 (3.8); 7.1686 (0.8); 7.1609 (6.4); 7.1559 (2.3);
7.1386 (11.5); 7.1214 (2.0); 7.1164 (5.7); 7.1090 (0.7); 5.2670 (0.6); 5.2494 (2.2); 5.2306 (3.0); 5.2118 (2.2); 5.1941
(0.6); 3.3422 (31.7); 2.8936 (9.5); 2.7344 (8.4); 2.5290 (0.9); 2.5153 (18.3); 2.5113 (35.9); 2.5068 (46.9); 2.5023
(35.3); 2.4982 (18.0); 1.4867 (16.0); 1.4692 (15.9)
I.125b: ¹H-NMR(400.2 MHz, d₆-DMSO):
δ = 8.8681 (4.5); 8.8392 (4.5); 8.7911 (3.7); 8.7703 (3.8); 7.9557 (1.4); 7.6486 (3.3); 7.5203 (8.4); 7.4543 (5.2); 7.4494
(2.4); 7.4404 (6.0); 7.4328 (6.7); 7.4240 (2.8); 7.4189 (6.0); 7.3921 (3.9); 7.1684 (0.8); 7.1610 (6.5); 7.1558 (2.2);
7.1387 (11.6); 7.1214 (2.1); 7.1164 (5.7); 7.1090 (0.7); 5.2668 (0.5); 5.2493 (2.1); 5.2307 (3.0); 5.2117 (2.2); 5.1939
(0.6); 3.3409 (34.0); 2.8935 (9.5); 2.7349 (8.2); 2.7340 (8.0); 2.5290 (0.9); 2.5154 (18.6); 2.5110 (36.5); 2.5065 (47.5);
2.5020 (35.1); 2.4976 (17.4); 1.4865 (16.0); 1.4690 (16.0)
I.126: ¹H-NMR(400.1 MHz, d₆-DMSO):
δ = 8.9085 (7.7); 8.9016 (9.1); 8.8130 (9.4); 8.8060 (7.6); 8.5119 (13.3); 7.9594 (0.5); 7.6610 (4.7); 7.5326 (10.8);
7.4045 (5.3); 3.3328 (100.4); 2.8959 (2.6); 2.7364 (2.4); 2.5074 (34.1); 1.7882 (6.7); 1.7574 (7.1); 1.6957 (6.1); 1.6653
(7.1); 1.5907 (3.5); 1.5626 (3.4); 1.4818 (2.0); 1.4519 (4.3); 1.4226 (2.5); 1.1792 (1.4); 1.1472 (4.6); 1.1149 (6.2);
1.0824 (4.5); 1.0437 (3.2); 1.0129 (2.3); 0.9610 (3.0); 0.9296 (6.4); 0.9038 (5.3); 0.8723 (1.8); 0.8203 (0.4); 0.7861
(4.4); 0.7689 (15.9); 0.7572 (6.8); 0.7182 (1.1); 0.7078 (1.1); 0.6683 (7.0); 0.6567 (16.0); 0.6399 (4.5); 0.6052 (0.3)
I.127: ¹H-NMR(300.2 MHz, CDCl₃):
δ = 9.1937 (16.0); 8.9715 (0.9); 7.5229 (2.3); 7.5166 (1.0); 7.5050 (2.6); 7.4944 (2.9); 7.4837 (1.1); 7.4767 (2.7); 7.2984
(4.0); 7.1248 (2.0); 7.1082 (0.4); 7.0985 (3.0); 7.0915 (0.9); 7.0695 (5.2); 7.0628 (1.1); 7.0471 (0.9); 7.0404 (2.4);
6.9529 (4.1); 6.7809 (2.0); 6.3206 (0.6); 6.2986 (2.1); 6.2767 (2.1); 6.2548 (0.7); 3.3335 (3.2); 1.7788 (10.8); 1.7568
(10.7); 1.6350 (3.6); 1.2896 (0.5); 0.0334 (4.1)
I.128: ¹H-NMR(400.1 MHz, d₆-DMSO):
δ = 8.9829 (0.4); 8.9279 (9.0); 8.8981 (8.8); 8.7571 (3.2); 8.7421 (6.3); 8.7268 (3.1); 7.9582 (0.3); 7.6644 (4.5); 7.5361
(10.1); 7.4824 (5.0); 7.4764 (3.5); 7.4699 (5.7); 7.4601 (6.1); 7.4080 (5.0); 7.3558 (1.9); 7.3444 (4.1); 7.3313 (12.3);
7.3197 (16.0); 7.3070 (7.7); 7.3028 (6.5); 7.2962 (6.2); 7.2851 (1.3); 4.6832 (14.6); 4.6679 (14.5); 3.8163 (0.5); 3.3350
(94.4); 2.8956 (1.9); 2.7362 (1.7); 2.5074 (29.7)
I.129: ¹H-NMR(300.2 MHz, CDCl₃):
δ = 9.0620 (1.8); 8.9986 (0.5); 8.9191 (1.9); 7.4385 (0.6); 7.4177 (0.3); 7.3676 (1.3); 7.3461 (2.5); 7.3398 (2.3); 7.3241
(1.9); 7.3182 (5.0); 7.3120 (2.6); 7.2987 (50.6); 7.2905 (3.6); 7.2686 (1.3); 7.1806 (0.5); 7.1557 (1.0); 7.1296 (0.6);
7.1225 (0.6); 7.1077 (5.2); 7.0950 (0.4); 7.0026 (1.1); 6.9890 (6.3); 6.9753 (1.3); 6.9631 (8.6); 6.9501 (1.7); 6.9355
(16.0); 6.9218 (0.9); 6.7632 (5.5); 6.1713 (0.4); 6.1094 (2.1); 4.8966 (10.8); 4.8767 (10.7); 4.8408 (1.3); 4.8197 (1.2);
2.9953 (1.5); 2.9231 (1.2); 1.6044 (15.5); 1.2915 (0.7); 0.1134 (0.3); 0.1036 (0.4); 0.0477 (2.5); 0.0369 (67.5); 0.0260 (2.6)
I.130: ¹H-NMR(400.2 MHz, d₆-DMSO):
δ = 10.0345 (4.2); 9.0628 (16.0); 7.6393 (1.1); 7.6355 (1.2); 7.6196 (2.2); 7.6162 (2.2); 7.6012 (1.3); 7.5961 (1.2);
7.3209 (0.6); 7.3165 (0.8); 7.3007 (1.8); 7.2964 (2.2); 7.2896 (1.5); 7.2853 (1.2); 7.2751 (2.5); 7.2709 (4.0); 7.2589
(2.1); 7.2541 (1.9); 7.2435 (2.6); 7.2375 (2.0); 7.2247 (2.2); 7.2195 (2.1); 7.2066 (0.8); 7.2019 (0.8); 3.3323 (10.6);
2.5057 (6.5); 2.5018 (8.4); 2.4979 (6.4); −0.0020 (6.6)
I.131: ¹H-NMR(400.2 MHz, d₆-DMSO):
δ = 10.1178 (4.8); 9.0163 (16.0); 7.6217 (1.2); 7.6180 (1.2); 7.6019 (2.2); 7.5992 (2.3); 7.5831 (1.3); 7.5789 (1.1);
7.3297 (0.6); 7.3251 (0.8); 7.3095 (2.1); 7.3048 (2.5); 7.2983 (1.7); 7.2846 (3.8); 7.2798 (3.8); 7.2712 (2.1); 7.2666
(2.0); 7.2601 (0.9); 7.2512 (2.8); 7.2452 (1.9); 7.2322 (2.3); 7.2268 (2.0); 7.2142 (0.8); 7.2095 (0.8); 3.3293 (14.4);
2.6725 (0.4); 2.5077 (42.2); 2.5035 (53.8); 2.4992 (38.2); 0.0014 (3.5)
I.132: ¹H-NMR(400.1 MHz, CDCl₃):
δ = 8.9987 (0.9); 8.8353 (0.9); 7.7639 (1.9); 7.7493 (1.8); 7.2582 (8.3); 7.1549 (5.7); 7.1454 (7.0); 6.9993 (1.3); 6.8704
(2.6); 6.7412 (1.3); 6.3689 (2.5); 2.9537 (1.4); 2.8827 (1.4); 2.5502 (16.0); 1.5543 (8.0); 1.2923 (14.0); 1.2555
(0.4); −0.0002 (10.5)
I.133: ¹H-NMR(400.1 MHz, CDCl₃):
δ = 8.9713 (2.0); 8.9292 (2.0); 8.0183 (1.3); 7.2587 (8.2); 7.1736 (4.0); 7.1537 (6.2); 7.0987 (5.9); 7.0789 (3.5); 7.0097
(1.4); 6.8805 (2.8); 6.7513 (1.4); 6.4179 (2.8); 2.9535 (6.6); 2.8822 (6.3); 2.3454 (0.6); 2.3308 (0.5); 2.2980 (16.0);
1.5707 (7.4); 1.4186 (1.2); 1.3964 (5.8); 1.3592 (5.1); 1.3528 (5.5); 1.3305 (0.9); −0.0002 (9.9)
I.134: ¹H-NMR(300.1 MHz, d₆-DMSO):
δ = 9.0620 (9.7); 8.8448 (3.3); 8.6954 (3.3); 7.6684 (4.7); 7.4972 (16.0); 7.4904 (10.8); 7.4665 (12.2); 7.4641 (11.5);
7.3261 (9.9); 7.2992 (12.9); 7.2730 (7.4); 7.1934 (4.8); 7.1895 (3.0); 7.1752 (2.2); 7.1691 (6.5); 7.1449 (2.3); 3.3311
(130.2); 2.6524 (0.5); 2.6058 (5.9); 2.5845 (15.0); 2.5586 (9.5); 2.5406 (1.4); 2.5161 (7.6); 2.5102 (14.2); 2.5042
(18.6); 2.4983 (12.8); 2.1064 (0.4); 2.0845 (1.1); 2.0749 (7.8); 2.0637 (1.0); 2.0544 (1.6); 2.0473 (1.9); 2.0334 (1.2);
2.0251 (1.7); 2.0183 (2.2); 1.9952 (1.3); 1.9677 (0.5); 1.9501 (0.6); 1.9234 (1.9); 1.8980 (2.6); 1.8859 (1.4); 1.8712
(1.9); 1.8615 (1.7); 1.8458 (0.7); 1.8336 (1.0); 0.0105 (0.4); −0.0004 (10.9); −0.0115 (0.3)
I.135: ¹H-NMR(300.2 MHz, CDCl₃):
δ = 8.9322 (4.1); 8.8175 (3.8); 7.3935 (3.1); 7.3874 (4.9); 7.3807 (2.2); 7.3651 (13.6); 7.3603 (13.0); 7.3533 (11.2);
7.3293 (11.3); 7.2992 (9.5); 7.2659 (2.4); 7.2604 (4.0); 7.2543 (2.3); 7.2461 (1.7); 7.2372 (4.3); 7.2286 (1.2); 7.2195
(1.0); 7.2145 (1.5); 7.2093 (0.8); 7.1050 (4.3); 6.9328 (9.1); 6.7605 (4.6); 5.9569 (2.6); 5.3358 (0.7); 4.1697 (0.7);
4.1458 (0.8); 4.1221 (0.3); 3.7697 (15.0); 3.7508 (14.7); 2.0827 (3.8); 1.6784 (9.2); 1.3647 (0.4); 1.3419 (0.7); 1.3195
(1.9); 1.2960 (5.6); 1.2721 (1.3); 1.0361 (1.3); 1.0064 (16.0); 1.0013 (11.3); 0.9844 (15.4); 0.9546 (1.4); 0.9391 (1.6);
0.9176 (4.1); 0.8944 (1.7); 0.0361 (6.0)
I.136: ¹H-NMR(300.2 MHz, CDCl₃):
δ = 8.9116 (14.1); 7.4625 (1.5); 7.4568 (2.1); 7.4330 (4.1); 7.4308 (4.0); 7.4082 (1.7); 7.4041 (2.3); 7.3970 (0.7); 7.3810
(4.2); 7.3754 (1.6); 7.3566 (2.1); 7.3365 (1.3); 7.3313 (1.8); 7.3261 (1.0); 7.3170 (1.0); 7.3081 (1.9); 7.2985 (6.2);
7.2923 (0.4); 7.2892 (0.5); 7.2849 (0.7); 7.2054 (0.4); 7.0901 (1.8); 6.9178 (4.0); 6.9046 (0.4); 6.7455 (2.0); 6.3704
(0.9); 6.3446 (0.9); 5.3359 (0.4); 4.7163 (1.2); 4.6887 (2.3); 4.6612 (1.2); 2.0827 (0.6); 1.6489 (16.0); 1.3804 (0.4);
1.3644 (0.9); 1.3372 (2.0); 1.3203 (2.7); 1.3048 (7.7); 1.2825 (1.3); 1.2657 (0.6); 0.9411 (2.7); 0.9193 (8.3); 0.8962
(3.0); 0.7207 (0.4); 0.7035 (2.3); 0.6969 (2.9); 0.6764 (2.5); 0.6706 (2.4); 0.6516 (0.5); 0.5641 (0.3); 0.5339 (1.6);
0.5298 (1.4); 0.5170 (2.3); 0.5110 (2.5); 0.5008 (1.0); 0.4943 (1.4); 0.0373 (5.0)
I.137: ¹H-NMR(400.1 MHz, CDCl₃):
δ = 8.9226 (2.5); 8.8383 (2.5); 8.6090 (6.4); 8.5980 (6.4); 8.0184 (3.1); 7.6503 (3.0); 7.6309 (6.5); 7.6115 (4.0); 7.4974
(8.0); 7.4772 (6.0); 7.2603 (25.4); 7.1650 (4.1); 7.1507 (5.5); 7.1351 (3.6); 6.9970 (3.6); 6.8685 (7.1); 6.7974 (7.0);
6.7387 (3.6); 2.9546 (14.5); 2.8835 (16.0); 2.8628 (5.4); 2.8403 (6.1); 2.8204 (3.6); 2.7056 (0.4); 2.6796 (3.6); 2.6565
(6.0); 2.6368 (5.7); 2.6090 (2.9); 2.2851 (0.6); 2.2579 (1.6); 2.2428 (2.8); 2.2191 (2.9); 2.2028 (1.6); 2.1764 (0.6);
2.1337 (0.8); 2.1143 (1.8); 2.1084 (1.8); 2.0932 (3.0); 2.0814 (2.3); 2.0697 (2.6); 2.0299 (0.4); 1.5941 (8.4); −0.0002 (32.6)
I.138: ¹H-NMR(300.2 MHz, CDCl₃):
δ = 8.9294 (0.5); 8.7793 (0.5); 7.4137 (1.4); 7.4081 (2.2); 7.4009 (0.8); 7.3846 (4.3); 7.3806 (3.8); 7.3735 (0.9); 7.3622
(1.8); 7.3593 (2.5); 7.3522 (0.8); 7.3359 (3.8); 7.3301 (1.5); 7.3154 (1.0); 7.3099 (1.8); 7.2988 (3.9); 7.2792 (1.0);
7.2741 (1.6); 7.2687 (0.9); 7.2592 (0.6); 7.2505 (1.6); 7.2420 (0.4); 7.2323 (0.4); 7.2276 (0.5); 7.0709 (1.6); 6.8986
(3.6); 6.7263 (1.8); 6.1632 (1.4); 2.2756 (0.7); 2.2508 (0.9); 2.2303 (1.2); 2.2055 (1.1); 2.1807 (0.4); 2.0866 (0.4);
2.0617 (1.1); 2.0444 (0.6); 2.0370 (1.2); 2.0163 (0.9); 1.9918 (0.8); 1.8761 (16.0); 1.6619 (0.6); 0.9130 (4.1); 0.8883
(8.8); 0.8635 (3.8); 0.0374 (4.9)
I.139: ¹H-NMR(400.2 MHz, d₆-DMSO):
δ = 8.8436 (3.3); 8.8364 (8.9); 8.8231 (11.8); 8.7994 (3.6); 7.9553 (1.6); 7.6422 (3.1); 7.5139 (7.9); 7.4182 (5.1); 7.4152
(6.6); 7.3973 (9.5); 7.3858 (4.1); 7.3324 (5.2); 7.3141 (9.8); 7.2947 (5.1); 7.2329 (3.4); 7.2146 (4.7); 7.1965 (1.7);
4.8256 (2.4); 4.8028 (4.9); 4.7800 (2.5); 3.3425 (54.6); 2.8925 (10.8); 2.7335 (9.2); 2.5285 (0.9); 2.5150 (19.1); 2.5107
(37.2); 2.5062 (48.2); 2.5017 (35.6); 2.4974 (17.7); 2.1560 (0.4); 2.1392 (1.2); 2.1225 (1.8); 2.1169 (1.4); 2.1056 (1.5);
2.1002 (1.8); 2.0835 (1.2); 2.0667 (0.5); 1.0130 (16.0); 0.9965 (15.5); 0.7419 (15.1); 0.7251 (14.8)
I.140: ¹H-NMR(300.2 MHz, CDCl₃):
δ = 8.9417 (1.4); 8.9014 (1.4); 7.4066 (0.8); 7.3908 (14.2); 7.3788 (10.7); 7.3747 (10.8); 7.3550 (0.8); 7.3488 (0.9);
7.3458 (1.0); 7.3396 (0.6); 7.3248 (1.6); 7.3145 (1.7); 7.3096 (1.4); 7.3034 (1.6); 7.2984 (5.5); 7.2878 (1.0); 7.2823
(0.8); 7.2774 (0.5); 7.2677 (0.4); 7.0938 (2.8); 6.9214 (6.2); 6.7492 (3.0); 6.2414 (1.4); 6.2138 (1.5); 5.2354 (1.1);
5.2102 (2.2); 5.1849 (2.3); 5.1589 (1.0); 1.9719 (1.1); 1.9661 (0.5); 1.9608 (0.4); 1.9504 (1.6); 1.9408 (1.6); 1.9270
(2.7); 1.9200 (2.3); 1.9047 (1.5); 1.8967 (2.4); 1.8751 (1.3); 1.8523 (0.5); 1.7047 (7.2); 1.5227 (0.5); 1.5188 (0.5);
1.4983 (1.0); 1.4732 (1.3); 1.4478 (1.6); 1.4243 (1.7); 1.4009 (1.3); 1.3935 (1.0); 1.3780 (0.8); 1.3700 (0.9); 1.3554
(0.4); 1.3472 (0.6); 1.3245 (0.3); 1.0158 (7.7); 0.9914 (16.0); 0.9670 (6.1); 0.0372 (5.6)
I.141: ¹H-NMR(300.2 MHz, CDCl₃):
δ = 7.6569 (1.8); 7.6509 (0.9); 7.6402 (2.0); 7.6343 (1.3); 7.6283 (2.3); 7.6180 (0.9); 7.6117 (2.2); 7.2989 (12.5); 7.2603
(0.4); 7.2501 (2.6); 7.2428 (0.9); 7.2220 (4.1); 7.2002 (0.8); 7.1932 (2.2); 7.1826 (0.3); 7.1401 (1.7); 6.9679 (3.5);
6.7959 (1.7); 6.4640 (0.8); 6.4377 (1.1); 6.3126 (2.2); 6.2858 (1.6); 1.6114 (16.0); 1.2965 (0.6); 0.9192 (0.6); 0.0479
(0.5); 0.0371 (13.4); 0.0263 (0.4)
I.142: ¹H-NMR(300.2 MHz, CDCl₃):
δ = 9.3526 (0.4); 9.1420 (16.0); 7.6150 (2.6); 7.6100 (3.8); 7.5867 (4.5); 7.5838 (4.2); 7.4407 (1.2); 7.4357 (1.8); 7.4296
(0.9); 7.4125 (4.6); 7.3873 (3.3); 7.3709 (1.4); 7.3663 (2.4); 7.3621 (1.5); 7.3512 (0.7); 7.3429 (2.1); 7.3325 (0.4);
7.3225 (0.4); 7.3185 (0.6); 7.2989 (4.6); 7.1096 (1.9); 6.9377 (4.0); 6.7659 (2.0); 5.2997 (5.1); 5.2727 (6.7); 5.1129
(6.3); 5.0859 (5.1); 1.6414 (2.2); 0.0347 (5.6)
I.143: ¹H-NMR(300.2 MHz, CDCl₃):
δ = 9.4060 (1.6); 9.1487 (0.5); 9.1397 (16.0); 8.7182 (1.4); 8.7151 (1.6); 8.7126 (1.6); 8.7096 (1.3); 8.7021 (1.4); 8.6990
(1.7); 8.6964 (1.6); 8.6936 (1.2); 7.7134 (0.9); 7.7074 (0.9); 7.6880 (1.8); 7.6821 (1.8); 7.6617 (1.3); 7.6557 (1.3);
7.4419 (2.6); 7.4153 (2.2); 7.2984 (3.4); 7.2919 (1.5); 7.2884 (1.3); 7.2756 (1.4); 7.2721 (1.3); 7.2668 (1.4); 7.2633
(1.2); 7.2506 (1.2); 7.2471 (1.1); 7.1134 (1.8); 6.9976 (0.4); 6.9416 (3.9); 6.7698 (1.9); 5.2869 (2.6); 5.2615 (7.6);
5.2407 (8.3); 5.2153 (2.8); 2.2024 (0.5); 2.0402 (3.1); 0.0270 (3.5)
I.144: ¹H-NMR(499.9 MHz, d₆-DMSO):
δ = 9.0173 (15.3); 8.8773 (6.5); 8.8347 (6.6); 7.6487 (3.7); 7.6329 (7.2); 7.6167 (8.3); 7.5137 (10.3); 7.4110 (5.1);
7.2569 (1.8); 7.2427 (4.2); 7.2305 (5.0); 7.2187 (2.8); 7.1084 (10.8); 7.0927 (14.3); 7.0777 (5.6); 7.0706 (4.8); 3.3425
(20.2); 2.5038 (2.1); 1.3040 (4.2); 1.2886 (16.0); 1.2797 (8.0); 1.2493 (2.5); 1.2189 (7.0); 1.2093 (15.3); 1.1951 (5.0);
1.1641 (0.4)
I.145: ¹H-NMR(400.1 MHz, CDCl₃):
δ = 8.9750 (9.1); 8.0189 (3.3); 7.2597 (32.7); 7.2298 (5.2); 7.2116 (2.2); 7.0445 (0.4); 7.0068 (7.6); 6.9872 (6.3); 6.9602
(5.4); 6.9359 (5.2); 6.9044 (3.5); 6.8864 (12.1); 6.8628 (2.7); 6.7583 (3.8); 6.3628 (7.0); 2.9545 (16.0); 2.8830 (15.4);
1.5544 (33.7); 1.4702 (1.9); 1.4437 (19.6); 1.4225 (19.2); 1.3956 (1.5); −0.0002 (40.4)
I.146: ¹H-NMR(400.1 MHz, CDCl₃):
δ = 9.1792 (0.5); 8.9829 (3.6); 8.9384 (3.5); 8.0166 (2.1); 7.3465 (0.4); 7.3415 (0.4); 7.2943 (5.1); 7.2797 (7.3); 7.2769
(7.2); 7.2608 (17.8); 7.0152 (2.9); 6.9843 (5.3); 6.9640 (9.4); 6.9430 (4.6); 6.8857 (5.3); 6.7564 (2.7); 6.4167 (5.3);
2.9542 (9.8); 2.8825 (9.5); 1.5784 (23.2); 1.4085 (1.4); 1.3829 (16.0); 1.3653 (15.5); 1.3395 (1.0); 1.3218
(0.4); −0.0002 (17.2)
I.147: ¹H-NMR(499.9 MHz, CDCl₃):
δ = 9.0725 (16.0); 8.9256 (0.4); 7.4877 (3.7); 7.4729 (4.5); 7.3409 (1.9); 7.3267 (4.6); 7.3114 (3.0); 7.2873 (1.9); 7.2729
(2.2); 7.2607 (12.8); 7.0096 (1.8); 6.9062 (3.8); 6.8886 (0.5); 6.8028 (1.9); 6.0301 (0.7); 6.0160 (2.2); 6.0020 (2.2);
5.9879 (0.7); 3.7065 (4.6); 3.5905 (0.7); 3.5765 (0.7); 1.7323 (8.6); 1.7182 (8.7); 1.5635 (7.8); 1.2705 (3.1); 1.2568
(3.2); 1.2523 (2.0); 1.2382 (0.7); 0.0061 (0.6); −0.0002 (14.8)
I.148: ¹H-NMR(300.2 MHz, CDCl₃):
δ = 9.0992 (16.0); 7.4519 (1.7); 7.4452 (2.1); 7.4253 (3.7); 7.4203 (3.9); 7.3948 (1.4); 7.3854 (4.2); 7.3750 (2.6); 7.3666
(5.9); 7.3481 (3.2); 7.3417 (5.7); 7.3197 (1.4); 7.3085 (0.6); 7.2993 (9.2); 7.1184 (1.8); 6.9462 (4.0); 6.7740 (2.0);
5.1655 (11.3); 4.0979 (1.7); 4.0743 (5.5); 4.0508 (5.6); 4.0272 (1.8); 3.8548 (3.3); 2.0463 (0.3); 1.6288 (7.5); 1.3329
(5.7); 1.3094 (12.0); 1.2858 (5.6); 0.0485 (0.4); 0.0469 (0.4); 0.0454 (0.4); 0.0378 (11.7); 0.0270 (0.5)
I.149: ¹H-NMR(300.2 MHz, CDCl₃):
δ = 9.1909 (16.0); 9.0812 (0.4); 7.5218 (0.3); 7.5117 (2.6); 7.5045 (1.2); 7.4941 (2.8); 7.4892 (1.6); 7.4870 (1.6); 7.4821
(3.2); 7.4718 (1.3); 7.4645 (3.0); 7.4546 (0.4); 7.2990 (10.1); 7.1196 (1.9); 7.0638 (0.4); 7.0537 (3.0); 7.0466 (1.0);
7.0311 (1.1); 7.0246 (5.2); 7.0181 (1.3); 7.0026 (0.9); 6.9955 (2.6); 6.9853 (0.4); 6.9477 (4.1); 6.7758 (2.0); 5.3376
(0.8); 3.3366 (0.4); 2.0462 (0.5); 1.6154 (2.8); 1.5576 (1.2); 1.5412 (1.9); 1.5376 (2.4); 1.5312 (3.8); 1.5241 (2.4);
1.5121 (2.0); 1.4708 (0.7); 1.4569 (0.7); 1.4174 (2.0); 1.4035 (2.4); 1.3965 (3.7); 1.3901 (2.4); 1.3697 (1.1); 1.3226
(0.4); 1.3021 (1.0); 0.9401 (0.4); 0.9181 (1.1); 0.8947 (0.4); 0.0471 (0.3); 0.0361 (8.2); 0.0253 (0.4)
I.150: ¹H-NMR(300.2 MHz, CDCl₃):
δ = 7.4352 (1.1); 7.4281 (0.5); 7.4178 (1.2); 7.4126 (0.6); 7.4104 (0.6); 7.4054 (1.3); 7.3952 (0.5); 7.3880 (1.2); 7.2983
(1.8); 7.0763 (0.8); 7.0419 (1.3); 7.0345 (0.4); 7.0192 (0.4); 7.0131 (2.2); 7.0062 (0.5); 6.9909 (0.4); 6.9836 (1.1);
6.9040 (1.8); 6.7318 (0.9); 6.2161 (0.7); 2.0446 (0.5); 1.8419 (16.0); 1.6616 (0.5); 0.0353 (2.1)
I.151: ¹H-NMR(300.2 MHz, d₆-DMSO):
δ = 8.8759 (1.6); 8.8388 (1.6); 8.7928 (1.3); 8.7690 (1.3); 7.7025 (1.4); 7.5315 (3.6); 7.3851 (0.3); 7.3607 (2.1); 7.3416
(0.5); 7.3355 (1.4); 7.3295 (0.6); 7.3124 (0.6); 7.3077 (0.9); 7.2859 (0.4); 7.0847 (2.0); 7.0563 (3.5); 7.0408 (0.4);
7.0287 (1.6); 5.4990 (0.8); 5.4751 (1.3); 5.4513 (0.8); 3.3462 (16.0); 2.5346 (3.6); 2.5286 (7.6); 2.5226 (10.4); 2.5165
(7.5); 2.5105 (3.5); 1.6195 (5.6); 1.5956 (5.5); 0.0304 (0.3); 0.0195 (8.8); 0.0085 (0.3)
I.152: ¹H-NMR(400.1 MHz, CDCl₃):
δ = 9.0094 (0.4); 8.9694 (16.0); 8.0183 (1.5); 7.6488 (0.6); 7.6291 (0.6); 7.5795 (12.0); 7.5603 (13.4); 7.5163 (0.3);
7.3070 (14.4); 7.2873 (12.6); 7.2594 (52.0); 7.0200 (3.6); 6.9947 (0.3); 6.8906 (7.1); 6.7611 (3.6); 6.3297 (7.8); 2.9552
(7.1); 2.8830 (6.8); 1.6893 (0.3); 1.6585 (0.6); 1.5451 (52.5); 1.5070 (28.1); 1.4752 (1.3); 0.1445 (0.5); 0.0676 (0.4);
0.0424 (0.5); −0.0002 (61.8); −0.1490 (0.4)
I.153: ¹H-NMR(400.1 MHz, CDCl₃):
δ = 8.8669 (2.4); 8.7735 (2.3); 8.0172 (1.8); 7.4389 (10.6); 7.4196 (13.5); 7.3018 (6.2); 7.2830 (12.5); 7.2603 (22.9);
7.2143 (4.9); 7.1972 (5.6); 7.1784 (2.0); 6.9824 (3.4); 6.8529 (6.8); 6.7240 (3.4); 6.1169 (6.5); 2.9537 (8.5); 2.8826
(8.2); 2.4577 (4.8); 2.4253 (6.8); 2.2404 (6.1); 2.2226 (4.3); 2.2101 (4.3); 1.8901 (16.0); 1.5656 (30.5); −0.0002 (25.0)
I.155: ¹H-NMR(400.1 MHz, CDCl₃):
δ = 8.9769 (0.8); 8.8267 (0.8); 7.5868 (1.9); 7.5682 (1.9); 7.2588 (6.2); 7.2436 (1.0); 7.2238 (1.8); 7.2043 (1.0); 6.9950
(1.0); 6.9030 (1.2); 6.8845 (2.2); 6.8635 (4.0); 6.8388 (1.9); 6.7336 (1.7); 6.7247 (2.1); 3.9078 (12.8); 2.9535 (0.6);
2.8827 (0.6); 1.5526 (5.5); 1.2722 (0.4); 1.2313 (16.0); −0.0002 (7.9)
I.156: ¹H-NMR(400.1 MHz, CDCl₃):
δ = 8.9782 (2.0); 8.9426 (1.9); 8.0170 (1.2); 7.2594 (8.6); 7.2216 (1.2); 7.2024 (2.5); 7.1823 (1.4); 7.0133 (1.3); 6.8838
(2.6); 6.8362 (2.6); 6.8160 (6.2); 6.7505 (2.5); 6.7260 (2.0); 6.3929 (2.8); 3.7879 (0.6); 3.7657 (16.0); 2.9539 (5.6);
2.8823 (5.4); 1.5620 (8.5); 1.4583 (1.3); 1.4393 (5.2); 1.3822 (5.1); 1.3630 (1.2); −0.0002 (10.8)
I.157: ¹H-NMR(400.1 MHz, CDCl₃):
δ = 8.9885 (1.5); 8.9198 (1.5); 8.0177 (1.0); 7.2611 (11.2); 7.2444 (4.7); 7.0110 (1.2); 6.8822 (2.6); 6.8301 (4.7); 6.8100
(4.2); 6.7529 (1.3); 6.3950 (2.7); 3.7675 (16.0); 2.9541 (4.7); 2.8828 (4.5); 1.5631 (8.5); 1.3804 (0.8); 1.3565 (5.9);
1.3258 (5.8); 1.3014 (0.6); −0.0002 (10.5)
I.158: ¹H-NMR(300.2 MHz, CDCl₃):
δ = 8.9945 (0.3); 8.9810 (0.3); 8.9256 (0.3); 8.9090 (0.3); 7.5041 (3.3); 7.4767 (4.1); 7.2992 (13.0); 7.2404 (3.2); 7.2139
(2.6); 7.0914 (1.3); 6.9192 (2.9); 6.7470 (1.4); 6.4737 (1.7); 5.1258 (3.4); 5.1034 (5.9); 5.0497 (5.8); 5.0273 (3.5);
2.3843 (11.7); 1.6046 (16.0); 1.2925 (0.4); 0.1075 (0.9); 0.0488 (0.6); 0.0380 (18.0); 0.0273 (1.1)
I.159: ¹H-NMR(300.1 MHz, d₆-DMSO):
δ = 8.9917 (11.5); 8.9089 (3.4); 8.7318 (3.4); 7.7722 (4.9); 7.5947 (13.9); 7.4968 (8.8); 7.4923 (11.9); 7.4685 (14.2);
7.4657 (13.4); 7.4174 (6.0); 7.3257 (7.9); 7.3012 (15.0); 7.2751 (8.7); 7.1921 (5.6); 7.1882 (3.6); 7.1741 (2.7); 7.1679
(7.7); 7.1438 (2.8); 3.3334 (98.5); 3.3095 (1.2); 2.7281 (0.8); 2.7221 (0.6); 2.6449 (0.8); 2.6205 (1.9); 2.6073 (6.1);
2.5975 (7.1); 2.5771 (16.0); 2.5512 (10.6); 2.5140 (45.4); 2.5082 (87.2); 2.5022 (113.8); 2.4963 (78.1); 2.4907 (35.6);
2.2786 (0.6); 2.2722 (0.7); 2.1059 (0.5); 2.0766 (1.8); 2.0695 (1.2); 2.0467 (2.2); 2.0339 (1.6); 2.0256 (2.0); 2.0178
(2.5); 1.9956 (1.6); 1.9680 (0.6); 1.9535 (0.8); 1.9275 (2.2); 1.9022 (3.0); 1.8749 (2.2); 1.8657 (2.0); 1.8491 (0.8);
1.8379 (1.1); 1.8114 (0.3); 0.0105 (3.1); −0.0005 (74.6); −0.0116 (2.3)
I.160: ¹H-NMR(300.1 MHz, d₆-DMSO):
δ = 9.1364 (9.4); 8.8675 (3.7); 8.7200 (3.7); 7.4933 (7.2); 7.4888 (10.0); 7.4650 (11.8); 7.4621 (11.2); 7.3239 (6.6);
7.3179 (2.5); 7.2995 (12.7); 7.2733 (7.3); 7.1981 (2.7); 7.1942 (4.8); 7.1902 (3.0); 7.1763 (2.2); 7.1699 (6.4); 7.1635
(1.8); 7.1497 (1.4); 7.1457 (2.3); 7.1418 (1.3); 3.3288 (155.7); 2.6543 (0.4); 2.6106 (6.0); 2.5879 (16.0); 2.5619 (10.2);
2.5451 (1.2);2.5151 (7.7);2.5091 (14.8);2.5031 (19.7);2.4971 (13.6); 2.4913 (6.2);2.1066 (0.4); 2.0848 (1.0); 2.0739
(5.0); 2.0633 (0.9); 2.0549 (1.5); 2.0471 (1.9); 2.0339 (1.2); 2.0252 (1.6); 2.0183 (2.1); 1.9959 (1.3); 1.9670 (0.4);
1.9511 (0.6); 1.9247 (1.9); 1.8991 (2.6); 1.8878 (1.4); 1.8724 (1.8); 1.8627 (1.7); 1.8474 (0.6); 1.8351 (0.9); 0.0105
(0.5); −0.0004 (12.6); −0.0114 (0.4)
I.161: ¹H-NMR(300.2 MHz, CDCl₃):
δ = 9.1011 (10.3); 7.3270 (0.5); 7.3216 (0.6); 7.2984 (1.7); 7.2727 (1.6); 7.2698 (1.7); 7.2466 (1.3); 7.1149 (1.2); 6.9427
(4.4); 6.9255 (1.7); 6.9159 (1.6); 6.9012 (0.8); 6.7705 (1.3); 5.2254 (6.7); 3.8801 (15.0); 3.8368 (16.0); 1.7854 (0.5);
0.0292 (0.8)
I.162: ¹H-NMR(400.2 MHz, d₆-DMSO):
δ = 8.9196 (16.0); 8.8992 (12.1); 7.7375 (5.1); 7.6446 (3.8); 7.6280 (6.4); 7.6247 (7.0); 7.6044 (15.9); 7.4714 (5.8);
7.2748 (1.6); 7.2707 (1.7); 7.2573 (3.6); 7.2532 (3.9); 7.2425 (3.8); 7.2377 (4.7); 7.2322 (2.9); 7.2234 (2.6); 7.2193
(2.3); 7.1248 (11.6); 7.1052 (14.0); 7.0987 (6.8); 7.0879 (4.4); 7.0856 (4.4); 7.0783 (4.1); 3.3192 (81.2); 3.2958 (0.5);
2.5054 (26.4); 2.5011 (33.9); 2.4968 (24.5); 2.0737 (0.4); 1.3109 (4.6); 1.2911 (14.6); 1.2801 (7.0); 1.2526 (1.7);
1.2426 (1.9); 1.2146 (7.3); 1.2037 (13.9); 1.1843 (3.6); −0.0019 (22.0)
I.163: ¹H-NMR(400.1 MHz, d₆-DMSO):
δ = 8.9041 (7.3); 8.8856 (3.3); 8.8233 (3.1); 7.9580 (2.9); 7.8526 (2.9); 7.8471 (3.0); 7.8342 (2.8); 7.8295 (3.1); 7.6471
(2.3); 7.5187 (5.1); 7.3906 (5.3); 7.3729 (3.6); 7.3685 (3.8); 7.2977 (1.2); 7.2830 (3.3); 7.2794 (3.3); 7.2654 (5.4);
7.2517 (3.0); 7.2470 (2.6); 7.2334 (0.9); 3.3342 (55.4); 2.8955 (16.0); 2.7361 (14.9); 2.5074 (17.5); 1.2805 (1.6);
1.2593 (7.0); 1.2497 (5.8); 1.2211 (5.9); 1.2117 (7.1); 1.1902 (1.8)
I.164: ¹H-NMR(400.1 MHz, d₆-DMSO):
δ = 8.9978 (5.5); 8.8278 (16.0); 7.9581 (2.3); 7.6455 (1.9); 7.5171 (4.2); 7.3890 (2.0); 7.3494 (0.5); 7.3332 (1.2); 7.3289
(1.2); 7.3131 (2.1); 7.2964 (1.3); 7.2929 (1.3); 7.2766 (0.6); 7.0236 (3.4); 7.0030 (6.1); 6.9825 (2.9); 3.3340 (48.8);
2.8956 (12.8); 2.7361 (11.9); 2.5074 (15.5); 1.3153 (1.4); 1.2952 (5.5); 1.2848 (3.2); 1.2448 (3.2); 1.2342 (5.3); 1.2140 (1.4)
I.165: ¹H-NMR(300.1 MHz, d₆-DMSO):
δ = 9.0948 (16.0); 8.9013 (8.9); 8.8651 (8.9); 7.6617 (3.4); 7.6565 (4.1); 7.6360 (6.6); 7.6303 (7.8); 7.6104 (3.8); 7.6041
(4.4); 7.2912 (1.7); 7.2851 (1.8); 7.2737 (2.1); 7.2678 (4.1); 7.2621 (3.9); 7.2580 (3.4); 7.2487 (4.0); 7.2416 (5.4);
7.2335 (3.5); 7.2223 (3.3); 7.2163 (3.0); 7.1330 (8.8); 7.1068 (13.9); 7.1028 (13.7); 7.0817 (4.8); 7.0774 (5.4); 7.0734
(5.1); 7.0695 (3.8); 3.3299 (291.6); 3.2849 (0.5); 2.5158 (12.0); 2.5098 (24.0); 2.5039 (32.1); 2.4979 (22.0); 2.4921
(10.0); 2.0753 (1.3); 1.3243 (4.2); 1.2965 (14.6); 1.2828 (8.7); 1.2719 (3.4); 1.2374 (3.6); 1.2257 (8.0); 1.2119 (13.9);
1.1852 (3.7); 0.0106 (0.7); −0.0004 (19.1); −0.0114 (0.6)
I.166: ¹H-NMR(300.2 MHz, CDCl₃):
δ = 9.0646 (2.8); 8.3915 (0.5); 8.3654 (0.6); 7.5242 (0.7); 7.5202 (0.8); 7.5116 (0.8); 7.5025 (1.4); 7.4950 (0.6); 7.4890
(0.6); 7.4726 (0.8); 7.4644 (1.6); 7.4620 (1.5); 7.4529 (0.7); 7.4443 (0.4); 7.4391 (0.7); 7.4339 (0.4); 7.3908 (0.3);
7.3860 (0.4); 7.3656 (0.7); 7.3606 (0.6); 7.2985 (8.2); 7.2821 (0.6); 7.1234 (0.4); 6.9511 (1.0); 6.7790 (0.5); 5.3379
(1.4); 1.5934 (16.0); 0.0483 (0.4); 0.0375 (9.8); 0.0267 (0.3)
I.167: ¹H-NMR(300.2 MHz, CDCl₃):
δ = 9.2139 (0.4); 9.2048 (16.0); 9.1942 (0.4); 7.3359 (0.4); 7.3155 (0.9); 7.3087 (0.8); 7.2986 (6.8); 7.2876 (1.8); 7.2801
(0.6); 7.2677 (0.8); 7.2596 (1.1); 7.2392 (0.5); 7.1148 (1.8); 6.9428 (4.2); 6.9347 (0.6); 6.9244 (2.9); 6.9115 (0.4);
6.9067 (0.6); 6.8967 (5.1); 6.8868 (0.6); 6.8813 (0.4); 6.8688 (2.2); 6.8584 (0.4); 6.7709 (2.0); 5.3368 (0.3); 1.6161
(9.3); 1.5907 (3.2); 1.5708 (1.5); 1.5176 (0.5); 1.4660 (1.5); 1.4445 (3.1); 1.4168 (0.8); 1.3013 (0.7); 1.2949 (0.7);
0.9165 (0.8); 0.0347 (7.2)
I.168: ¹H-NMR(300.2 MHz, d₆-DMSO):
δ = 8.8562 (0.5); 8.7954 (1.5); 8.6418 (0.5); 7.6820 (0.7); 7.5110 (1.8); 7.3403 (0.9); 7.2950 (0.4); 7.2672 (0.7); 7.2474
(0.3); 7.2397 (0.4); 6.9932 (1.0); 6.9641 (1.0); 6.9592 (1.1); 6.9306 (0.8); 3.3441 (16.0); 2.5344 (2.7); 2.5284 (5.7);
2.5224 (7.7); 2.5163 (5.6); 2.5104 (2.6); 2.0957 (0.5); 1.8540 (6.3); 0.0200 (7.5)
I.169: ¹H-NMR(300.2 MHz, CDCl₃):
δ = 9.1153 (10.3); 7.3997 (1.3); 7.3923 (0.6); 7.3710 (4.6); 7.3480 (5.6); 7.3399 (1.2); 7.3264 (0.7); 7.3202 (1.3); 7.3188
(1.4); 7.2984 (2.5); 7.1234 (1.2); 6.9513 (2.5); 6.7791 (1.2); 5.1189 (7.0); 3.8467 (16.0); 1.6400 (2.7); 0.0337 (3.5)
I.170: ¹H-NMR(300.2 MHz, CDCl₃):
δ = 9.3595 (1.2); 8.9732 (2.6); 8.6415 (2.6); 7.8958 (0.5); 7.3558 (4.6); 7.3505 (6.8); 7.3436 (2.2); 7.3274 (11.3); 7.3236
(10.1); 7.2984 (11.2); 7.2917 (6.6); 7.2849 (2.0); 7.2683 (11.7); 7.2629 (4.8); 7.2545 (2.2); 7.2437 (5.8); 7.2146 (2.8);
7.2095 (4.8); 7.2047 (2.5); 7.1946 (1.7); 7.1861 (4.8); 7.1768 (1.3); 7.1669 (1.0); 7.1624 (1.5); 7.1574 (0.8); 7.1244
(4.5); 7.0054 (0.4); 6.9521 (10.1); 6.7798 (5.0); 6.6094 (5.3); 5.3358 (0.4); 1.6553 (16.0); 1.0718 (4.1); 1.0564 (11.9);
1.0502 (13.5); 1.0360 (7.6); 1.0230 (9.4); 1.0072 (5.4); 0.9874 (2.2); 0.9589 (1.9); 0.9392 (5.5); 0.9237 (9.3); 0.9105
(2.8); 0.8986 (2.7); 0.8635 (1.0); 0.8110 (5.2); 0.7967 (11.6); 0.7909 (11.7); 0.7752 (4.0); 0.0474 (0.4); 0.0366 (9.6)
I.171: ¹H-NMR(400.1 MHz, CDCl₃):
δ = 8.8122 (3.8); 8.8032 (3.8); 8.0171 (3.4); 7.5293 (4.5); 7.5093 (8.9); 7.4895 (4.6); 7.2597 (25.0); 7.2145 (2.2); 7.1968
(5.3); 7.1813 (5.7); 7.1640 (3.0); 7.0820 (6.0); 7.0631 (9.4); 7.0445 (4.1); 6.9805 (7.6); 6.9640 (5.2); 6.9552 (5.7);
6.9343 (4.4); 6.8495 (9.6); 6.7200 (4.9); 6.1639 (9.4); 2.9541 (16.0); 2.8825 (15.5); 2.6222 (7.0); 2.5901 (8.4); 2.2891
(7.6); 2.2742 (6.8); 2.2577 (6.1); 1.8656 (14.2); 1.8524 (14.4); 1.5711 (45.8); −0.0002 (30.8)
I.172: ¹H-NMR(400.1 MHz, CDCl₃):
δ = 9.2036 (0.4); 8.8880 (2.2); 8.7816 (2.2); 8.0175 (2.6); 7.2598 (20.1); 7.2297 (5.0); 7.2038 (8.3); 7.1844 (3.3); 7.1419
(4.6); 7.1150 (4.5); 6.9887 (3.2); 6.9092 (2.8); 6.8888 (4.9); 6.8599 (7.2); 6.7302 (3.2); 6.0996 (6.4); 2.9542 (12.0);
2.8826 (11.6); 2.4238 (4.6); 2.3916 (6.5); 2.2164 (5.8); 2.1972 (4.1); 2.1851 (4.1); 1.8920 (16.0); 1.5631 (20.6); 1.2711
(1.2); 1.2532 (1.4); −0.0002 (23.2)
I.173: ¹H-NMR(300.1 MHz, d₆-DMSO):
δ = 9.0785 (10.4); 8.9283 (3.8); 8.8256 (0.4); 8.7628 (3.8); 7.4975 (8.2); 7.4931 (11.1); 7.4692 (13.2); 7.4665 (12.3);
7.3279 (7.4); 7.3035 (13.8); 7.2773 (8.0); 7.1946 (5.1); 7.1907 (3.4); 7.1765 (2.6); 7.1704 (6.9); 7.1460 (2.6); 3.3313
(124.0); 3.3082 (1.5); 2.7279 (1.0); 2.7218 (0.7); 2.6481 (0.7); 2.6029 (6.7); 2.5818 (16.0); 2.5559 (10.8); 2.5138
(61.5); 2.5079 (118.5); 2.5019 (155.7); 2.4961 (107.5); 2.4905 (49.8); 2.2776 (0.7); 2.2716 (1.0); 2.2660 (0.7); 2.1053
(0.4); 2.0834 (1.0); 2.0692 (1.0); 2.0465 (2.0); 2.0326 (1.5); 2.0170 (2.3); 1.9950 (1.5); 1.9658 (0.6); 1.9551 (0.7);
1.9279 (2.0); 1.9027 (2.9); 1.8759 (2.0); 1.8668 (1.8); 1.8380 (1.0); 1.8140 (0.4); 0.1945 (0.5); 0.0105 (4.6); −0.0004
(113.1); −0.0115 (3.7); −0.1992 (0.5)
I.174: ¹H-NMR(400.1 MHz, d₆-DMSO):
δ = 9.0629 (5.8); 8.8105 (2.5); 8.6738 (2.6); 7.9582 (2.6); 7.7725 (3.0); 7.7538 (3.2); 7.6286 (2.0); 7.5004 (4.6); 7.3722
(2.3); 7.3228 (1.8); 7.3083 (5.0); 7.2899 (5.7); 7.2280 (2.4); 7.2251 (2.4); 7.2068 (2.8); 7.1901 (1.2); 3.3342 (63.8);
2.8955 (14.7); 2.7585 (2.9); 2.7361 (16.0); 2.7256 (3.5); 2.7006 (3.6); 2.6793 (2.4); 2.6496 (0.9); 2.5074 (18.8); 2.1226
(0.9); 2.1007 (1.4); 2.0751 (1.5); 2.0541 (0.9); 1.8131 (0.6); 1.8026 (0.9); 1.7919 (1.2); 1.7793 (1.2); 1.7657 (1.1);
1.7542 (0.8); 1.7435 (0.5)
I.175: ¹H-NMR(400.1 MHz, CDCl₃):
δ = 8.9589 (0.4); 8.8661 (6.0); 8.0172 (3.4); 7.4813 (14.4); 7.3786 (7.2); 7.3593 (9.3); 7.3055 (0.5); 7.2597 (29.4);
7.2489 (10.8); 7.2288 (6.3); 7.2041 (10.5); 7.1842 (4.9); 6.9948 (4.8); 6.8656 (9.2); 6.7366 (4.6); 6.3235 (9.6); 2.9540
(16.0); 2.8824 (15.6); 2.7773 (0.4); 2.7483 (3.4); 2.7198 (7.1); 2.6986 (8.8); 2.6794 (5.2); 2.5922 (4.8); 2.5727 (8.5);
2.5514 (7.5); 2.5245 (3.8); 2.2387 (0.8); 2.2191 (2.1); 2.1964 (3.7); 2.1737 (3.8); 2.1521 (2.0); 2.1342 (0.7); 2.0493
(1.0); 2.0305 (2.5); 2.0086 (3.9); 1.9923 (3.3); 1.9862 (3.3); 1.9464 (0.5); 1.6145 (0.4); 1.5623 (29.8); −0.0002 (34.0)
I.176: ¹H-NMR(400.2 MHz, d₆-DMSO):
δ = 9.1486 (12.8); 8.8615 (5.9); 8.7096 (6.0); 7.4861 (13.6); 7.4678 (15.3); 7.3172 (8.7); 7.2984 (15.8); 7.2789 (9.2);
7.1887 (5.6); 7.1704 (8.0); 7.1521 (3.1); 3.3339 (5.8); 2.6397 (1.5); 2.6109 (7.8); 2.6015 (9.4); 2.5877 (16.0); 2.5688
(9.9); 2.5534 (2.3); 2.5357 (1.4); 2.5090 (9.4); 2.5048 (12.1); 2.5009 (9.4); 2.0891 (0.8); 2.0758 (2.7); 2.0679 (1.5);
2.0614 (1.5); 2.0568 (1.6); 2.0494 (2.6); 2.0448 (2.8); 2.0346 (2.0); 2.0282 (2.5); 2.0224 (2.9); 2.0123 (1.2); 2.0058
(1.7); 1.9844 (0.6); 1.9337 (0.8); 1.9135 (2.4); 1.8948 (3.4); 1.8741 (2.6); 1.8676 (2.5); 1.8553 (1.0); 1.8467 (1.3);
1.8278 (0.4); −0.0017 (4.8)
I.177: ¹H-NMR(400.1 MHz, CDCl₃):
δ = 9.2056 (0.3); 8.8920 (16.0); 8.0172 (1.9); 7.2601 (14.1); 7.1638 (0.6); 7.1461 (1.8); 7.1294 (2.6); 7.1100 (1.9);
7.0930 (0.7); 6.9898 (2.1); 6.8603 (4.6); 6.8099 (4.2); 6.7897 (7.1); 6.7693 (3.5); 6.7308 (2.2); 6.4545 (4.0); 2.9546
(8.9); 2.9036 (1.5); 2.8823 (11.7); 2.8524 (4.1); 2.8272 (1.8); 2.6699 (2.8); 2.6467 (4.3); 2.6206 (2.5); 2.3822 (0.4);
2.3592 (1.3); 2.3345 (2.2); 2.3114 (2.1); 2.2880 (1.0); 2.0304 (0.9); 2.0077 (2.1); 1.9851 (1.9); 1.9624 (0.6); 1.5661
(24.2); −0.0002 (17.9)
I.178: ¹H-NMR(400.2 MHz, d₆-DMSO):
δ = 9.0211 (16.0); 8.9347 (9.7); 8.9174 (9.4); 8.3191 (1.5); 7.6483 (3.5); 7.6452 (4.0); 7.6293 (6.8); 7.6255 (7.3); 7.6102
(3.9); 7.6058 (3.8); 7.2794 (1.8); 7.2750 (2.0); 7.2662 (2.4); 7.2618 (4.2); 7.2573 (4.2); 7.2472 (4.1); 7.2422 (5.0);
7.2363 (3.0); 7.2278 (2.8); 7.2235 (2.4); 7.1291 (11.8); 7.1097 (15.2); 7.0915 (4.9); 7.0888 (5.0); 7.0834 (4.5); 3.3295
(17.5); 3.3058 (0.4); 2.6714 (0.4); 2.5067 (43.3); 2.5023 (55.2); 2.4979 (38.4); 2.3295 (0.5); 2.0763 (11.1); 1.3153
(5.2); 1.2955 (15.4); 1.2843 (7.5); 1.2560 (1.8); 1.2467 (2.0); 1.2181 (8.0); 1.2072 (14.9); 1.1876 (3.9); 0.0065
(1.8); −0.0014 (27.2); −0.0098 (0.9)
I.179: ¹H-NMR(400.2 MHz, d₆-DMSO):
δ = 9.0877 (15.9); 8.8888 (8.1); 8.8493 (8.1); 7.6513 (3.6); 7.6479 (4.0); 7.6320 (6.8); 7.6283 (7.6); 7.6129 (3.8); 7.6085
(4.1); 7.2784 (1.9); 7.2740 (2.0); 7.2605 (4.2); 7.2563 (4.2); 7.2460 (4.1); 7.2411 (5.2); 7.2354 (3.3); 7.2266 (2.9);
7.2224 (2.7); 7.1231 (9.4); 7.1037 (14.5); 7.1014 (14.2); 7.0848 (5.0); 7.0822 (5.8); 7.0791 (5.4); 3.3164 (283.4);
3.2930 (2.2); 2.6686 (0.8); 2.5043 (72.6); 2.4999 (96.2); 2.4956 (70.6); 2.3268 (0.7); 2.0726 (1.3); 1.3127 (5.2); 1.2928
(16.0); 1.2817 (8.0); 1.2549 (2.1); 1.2440 (2.3); 1.2169 (8.1); 1.2058 (15.2); 1.1862 (4.2); 0.1447 (0.3); 0.0063
(3.6); −0.0016 (65.7); −0.0098 (2.8)
I.180: ¹H-NMR(300.2 MHz, CDCl₃):
δ = 9.0008 (5.0); 8.0519 (0.9); 8.0002 (0.5); 7.9937 (3.4); 7.9874 (1.2); 7.9711 (1.2); 7.9648 (3.6); 7.3077 (0.7); 7.3006
(5.4); 7.2990 (6.0); 7.2785 (1.2); 7.2723 (3.3); 7.0999 (1.2); 6.9277 (2.6); 6.7555 (1.3); 6.5838 (1.7); 3.9459 (0.6);
3.9205 (16.0); 2.9921 (6.8); 2.9189 (6.1); 2.9174 (5.5); 1.7007 (2.6); 1.5871 (0.4); 1.5525 (2.4); 1.5442 (2.0); 1.5206
(2.1); 1.5136 (2.4); 1.4784 (0.4); 0.0323 (4.8)
I.181: ¹H-NMR(300.2 MHz, CDCl₃):
δ = 9.0428 (16.0); 7.5317 (1.9); 7.5142 (2.1); 7.5030 (2.4); 7.4858 (2.2); 7.2987 (16.2); 7.1993 (0.4); 7.1892 (3.3);
7.1821 (1.0); 7.1665 (1.2); 7.1606 (5.5); 7.1544 (1.2); 7.1389 (0.9); 7.1318 (2.8); 7.1162 (2.1); 6.9441 (4.5); 6.7720
(2.2); 6.3544 (0.9); 6.3227 (1.2); 6.0918 (0.4); 6.0661 (1.2); 6.0404 (1.3); 6.0342 (1.1); 6.0084 (1.0); 2.0472 (0.5);
1.6034 (11.3); 1.3045 (0.6); 0.9191 (0.7); 0.8963 (0.3); 0.0480 (0.8); 0.0371 (22.5); 0.0259 (0.8)
I.182: ¹H-NMR(300.2 MHz, CDCl₃):
δ = 8.9349 (3.1); 8.7484 (3.1); 7.4418 (0.3); 7.4055 (17.4); 7.3990 (6.9); 7.3833 (8.9); 7.3764 (29.8); 7.3683 (4.6);
7.3315 (0.6); 7.2984 (46.2); 7.2911 (9.1); 7.2752 (6.6); 7.2687 (16.7); 7.0708 (7.4); 6.8985 (16.0); 6.7263 (8.0); 6.0921
(9.6); 5.3367 (7.4); 2.5410 (6.9); 2.4971 (7.9); 1.9559 (3.8); 1.9427 (4.2); 1.9120 (7.3); 1.9012 (7.6); 1.8689 (4.8);
1.8571 (5.0); 1.8203 (3.1); 1.8091 (2.9); 1.7748 (7.6); 1.7390 (7.8); 1.7263 (9.7); 1.7151 (8.1); 1.6689 (7.3); 1.6519
(12.2); 1.6274 (5.3); 1.5934 (1.4); 1.5823 (1.9); 1.4524 (1.2); 1.4379 (1.9); 1.4118 (2.2); 1.3985 (3.3); 1.3845 (2.3);
1.3727 (2.1); 1.3598 (2.8); 1.3473 (1.6); 1.3347 (1.3); 1.3185 (1.3); 1.2916 (3.1); 1.2585 (0.4); 1.2452 (0.4); 0.9158
(0.5); 0.8887 (0.6); 0.8774 (0.5); 0.8685 (0.5); 0.1142 (0.4); 0.0470 (0.9); 0.0364 (18.8); 0.0256 (0.8)
I.183: ¹H-NMR(300.2 MHz, CDCl₃):
δ = 9.0357 (1.0); 8.8971 (1.0); 7.8602 (2.1); 7.8545 (2.2); 7.8347 (2.3); 7.8290 (2.3); 7.5543 (2.2); 7.5506 (2.4); 7.5279
(2.6); 7.5242 (2.6); 7.3127 (1.2); 7.3087 (1.2); 7.2984 (0.7); 7.2878 (2.5); 7.2838 (2.4); 7.2626 (1.5); 7.2586 (1.4);
7.1601 (1.5); 7.1543 (1.5); 7.1346 (2.0); 7.1291 (2.1); 7.1090 (1.1); 7.1031 (1.1); 7.0970 (2.0); 7.0149 (2.6); 6.9247
(4.2); 6.7525 (2.1); 2.0302 (3.5); 1.3751 (16.0); 0.0299 (0.5)
I.184: ¹H-NMR(300.2 MHz, CDCl₃):
δ = 9.1260 (16.0); 7.7786 (2.6); 7.7290 (1.3); 7.7032 (1.6); 7.5906 (1.1); 7.5646 (1.8); 7.5047 (1.5); 7.4791 (1.9); 7.4531
(0.7); 7.2989 (11.6); 7.1212 (1.8); 6.9490 (4.1); 6.7769 (2.0); 6.1084 (0.6); 6.0848 (1.8); 6.0614 (1.8); 6.0378 (0.6);
3.8037 (5.2); 1.8003 (7.9); 1.7768 (7.8); 1.6051 (2.9); 0.0472 (0.5); 0.0365 (15.1); 0.0271 (0.4); 0.0256 (0.5)
I.185: ¹H-NMR(300.2 MHz, CDCl₃):
δ = 9.2938 (16.0); 8.1121 (0.5); 8.0904 (0.5); 7.9626 (0.8); 7.9534 (0.5); 7.9417 (1.3); 7.9342 (2.0); 7.9133 (2.0); 7.9058
(1.4); 7.8944 (0.5); 7.8848 (0.9); 7.2987 (12.4); 7.1491 (1.7); 7.0548 (1.3); 7.0463 (1.7); 7.0266 (2.2); 7.0190 (2.6);
7.0088 (0.7); 6.9998 (1.4); 6.9909 (1.8); 6.9774 (4.0); 6.9608 (0.4); 6.9536 (0.5); 6.9465 (0.3); 6.9252 (0.3); 6.8445
(1.0); 6.8365 (1.1); 6.8157 (1.5); 6.8059 (3.4); 6.7803 (1.2); 6.7724 (0.9); 4.1725 (0.4); 4.1487 (0.5); 2.0838 (2.1);
1.3445 (0.4); 1.3210 (1.1); 1.2973 (3.0); 1.2734 (0.7); 0.9413 (0.7); 0.9196 (2.2); 0.8964 (0.8); 0.0483 (0.6); 0.0466
(0.4); 0.0450 (0.5); 0.0375 (16.9); 0.0282 (0.5); 0.0266 (0.6)
I.186: ¹H-NMR(400.2 MHz, d₆-DMSO):
δ = 8.8375 (9.6); 8.2215 (1.5); 8.1993 (1.5); 7.6559 (1.5); 7.5276 (3.7); 7.3994 (1.8); 3.8059 (1.2); 3.7887 (1.3); 3.7837
(1.2); 3.7665 (1.2); 3.3399 (18.2); 2.8943 (0.9); 2.7347 (0.8); 2.5288 (0.4); 2.5152 (7.6); 2.5110 (14.5); 2.5065 (18.6);
2.5020 (13.8); 2.4978 (6.9); 1.1994 (8.6); 1.1823 (8.5); 1.0667 (16.0); 0.6422 (1.0); 0.6307 (0.8); 0.6193 (1.0); 0.6109
(0.7); 0.3962 (0.6); 0.3859 (1.1); 0.3754 (0.9); 0.3617 (1.0); 0.2471 (0.5); 0.2352 (0.4); 0.2247 (1.3); 0.2083 (3.8);
0.1929 (1.2); 0.1824 (0.4); 0.1787 (0.4); 0.1705 (0.4)
I.187: ¹H-NMR(400.2 MHz, d₆-DMSO):
δ = 8.9070 (1.8); 8.8997 (2.3); 8.8199 (2.3); 8.8124 (1.8); 8.5234 (3.1); 7.6582 (1.5); 7.5299 (3.8); 7.4017 (1.8); 3.3387
(11.6); 2.8947 (0.6); 2.7348 (0.5); 2.5292 (0.4); 2.5157 (7.7); 2.5114 (14.9); 2.5069 (19.3); 2.5024 (14.2); 2.4980 (7.0);
1.9042 (0.4); 1.8872 (1.1); 1.8702 (1.5); 1.8532 (1.1); 1.8362 (0.4); 0.8838 (16.0); 0.8668 (15.5); 0.7837 (1.2); 0.7654
(3.9); 0.7541 (1.9); 0.7202 (0.5); 0.7169 (0.5); 0.6825 (1.9); 0.6714 (3.9); 0.6534 (1.1)
I.188: ¹H-NMR(400.2 MHz, d₆-DMSO):
δ = 8.8823 (4.0); 8.8426 (6.2); 8.8218 (3.8); 8.6227 (3.9); 8.4419 (2.7); 8.4324 (2.8); 7.9533 (1.6); 7.8176 (2.3); 7.8133
(3.4); 7.8091 (2.3); 7.7979 (2.6); 7.7936 (3.7); 7.7893 (2.4); 7.6470 (3.1); 7.5188 (7.4); 7.3906 (3.6); 7.3690 (2.8);
7.3570 (2.9); 7.3495 (2.8); 7.3376 (2.5); 5.2970 (0.6); 5.2795 (2.2); 5.2609 (3.2); 5.2423 (2.2); 5.2243 (0.6); 3.3359
(79.1); 2.8915 (10.5); 2.7324 (9.0); 2.7315 (9.2); 2.6767 (0.7); 2.6725 (0.9); 2.6681 (0.7); 2.5256 (3.1); 2.5079 (121.6);
2.5035 (155.8); 2.4990 (117.1); 2.3349 (0.7); 2.3303 (0.9); 2.3259 (0.7); 1.5302 (16.0); 1.5126 (16.0); −0.0002 (0.7)
I.189: ¹H-NMR(400.2 MHz, d₆-DMSO):
δ = 8.9009 (3.1); 8.8943 (4.2); 8.8328 (4.2); 8.8264 (3.1); 8.7034 (3.3); 8.6835 (3.3); 8.5269 (3.5); 8.5252 (3.6); 8.5151
(3.7); 8.5133 (3.6); 7.9553 (1.2); 7.7690 (2.0); 7.7646 (2.0); 7.7498 (4.2); 7.7454 (4.1); 7.7306 (2.6); 7.7262 (2.5);
7.6507 (2.9); 7.5224 (6.9); 7.4099 (5.2); 7.3935 (5.3); 7.3904 (5.2); 7.2665 (2.6); 7.2644 (2.6); 7.2545 (2.8); 7.2521
(2.9); 7.2480 (2.9); 7.2358 (2.6); 7.2336 (2.4); 5.2920 (0.6); 5.2744 (2.4); 5.2558 (3.4); 5.2373 (2.4); 5.2196 (0.6);
3.3442 (6.0); 2.8933 (7.7); 2.7339 (6.7); 2.5106 (36.6); 2.5062 (47.0); 2.5017 (35.0); 1.5302 (16.0); 1.5126 (15.9);
1.4837 (0.4)
I.190: ¹H-NMR(400.2 MHz, d₆-DMSO):
δ = 9.2375 (1.0); 8.9023 (3.1); 8.8655 (3.2); 8.8455 (3.4); 8.8360 (1.4); 8.8260 (3.3); 8.5022 (2.5); 7.9549 (2.5); 7.8250
(0.4); 7.8133 (0.4); 7.8099 (0.4); 7.6489 (2.7); 7.5207 (6.8); 7.5020 (0.4); 7.4331 (0.5); 7.4252 (0.4); 7.3925 (8.5);
7.3792 (5.4); 5.2373 (0.4); 5.2196 (1.8); 5.2012 (2.7); 5.1826 (1.9); 5.1648 (0.5); 3.3477 (4.1); 2.8929 (16.0); 2.7341
(13.5); 2.7331 (13.8); 2.6746 (0.4); 2.6702 (0.4); 2.6310 (2.1); 2.5281 (1.2); 2.5145 (26.0); 2.5102 (51.2); 2.5057
(66.7); 2.5012 (49.6); 2.4968 (25.0); 2.3370 (0.3); 2.3326 (0.4); 1.6953 (0.4); 1.6782 (0.4); 1.5010 (13.6); 1.4833
(13.7); 1.4583 (0.5); −0.0002 (0.4)
I.191: ¹H-NMR(400.2 MHz, d₆-DMSO):
δ = 8.8931 (2.8); 8.8857 (3.5); 8.7794 (3.6); 8.7709 (16.0); 8.7587 (14.8); 8.7465 (1.0); 8.6298 (2.4); 8.6110 (2.4);
7.9548 (1.3); 7.6449 (2.2); 7.5167 (5.8); 7.3889 (5.4); 7.3771 (6.1); 7.3648 (3.2); 5.3027 (0.4); 5.2849 (1.9); 5.2667
(2.9); 5.2485 (1.9); 5.2307 (0.5); 3.3407 (49.3); 2.8931 (8.6); 2.7341 (7.4); 2.7329 (7.3); 2.5279 (0.8); 2.5144 (17.0);
2.5101 (33.5); 2.5056 (43.5); 2.5010 (32.1); 2.4967 (15.9); 1.5762 (12.0); 1.5584 (11.9); 1.5319 (0.6)
I.192: ¹H-NMR(400.2 MHz, d₆-DMSO):
δ = 8.9082 (6.0); 8.8992 (6.1); 8.7949 (3.3); 8.7736 (3.4); 7.6602 (3.0); 7.5320 (7.5); 7.4038 (3.6); 7.3646 (5.0); 7.3617
(5.2); 7.3520 (5.4); 7.3491 (5.3); 7.0358 (3.0); 7.0333 (4.8); 7.0306 (3.6); 7.0272 (4.3); 7.0246 (6.1); 7.0220 (3.9);
6.9716 (5.7); 6.9628 (4.8); 6.9590 (5.6); 6.9502 (4.3); 5.5649 (0.5); 5.5473 (2.0); 5.5290 (2.7); 5.5107 (2.0); 5.4929
(0.5); 3.3394 (48.3); 2.8921 (1.8); 2.7325 (1.5); 2.5269 (1.0); 2.5134 (18.8); 2.5090 (36.9); 2.5045 (48.2); 2.4999
(35.7); 2.4955 (17.7); 1.6040 (16.0); 1.5866 (15.9)
I.193: ¹H-NMR(400.2 MHz, d₆-DMSO):
δ = 8.8572 (3.6); 8.8496 (6.1); 8.8363 (0.5); 8.8209 (6.6); 8.8132 (3.6); 8.1308 (2.8); 8.1085 (2.9); 7.6551 (2.8); 7.5268
(7.0); 7.3986 (3.3); 3.9854 (1.1); 3.9681 (1.8); 3.9639 (1.4); 3.9507 (1.5); 3.9463 (1.8); 3.9291 (1.1); 3.3391 (38.0);
2.8942 (0.9); 2.7342 (0.8); 2.5287 (0.7); 2.5151 (15.4); 2.5107 (30.3); 2.5062 (39.6); 2.5017 (29.4); 2.4973 (14.6);
1.7450 (2.2); 1.7252 (2.9); 1.6927 (2.4); 1.6876 (2.4); 1.6175 (1.6); 1.5925 (1.3); 1.4775 (0.5); 1.4688 (0.5); 1.4570
(1.0); 1.4495 (1.2); 1.4404 (1.1); 1.4310 (1.3); 1.4226 (1.0); 1.4116 (0.7); 1.4028 (0.6); 1.3956 (0.3); 1.2257 (0.4);
1.1958 (1.1); 1.1864 (1.2); 1.1719 (1.6); 1.1653 (1.7); 1.1555 (1.8); 1.1475 (2.2); 1.1218 (16.0); 1.1 050 (15.7); 1.0804
(1.0); 1.0029 (1.3); 0.9729 (2.8); 0.9430 (2.3); 0.9183 (0.8)
I.194: ¹H-NMR(400.2 MHz, d₆-DMSO):
δ = 9.4146 (3.9); 9.1599 (0.8); 9.0522 (9.4); 9.0473 (9.4); 9.0366 (0.5); 8.8907 (10.2); 8.8864 (9.8); 8.6982 (3.4); 8.6771
(3.4); 7.9542 (1.9); 7.7541 (0.4); 7.6574 (3.1); 7.6264 (0.9); 7.5291 (8.2); 7.4988 (0.4); 7.4562 (6.6); 7.4541 (7.0);
7.4513 (7.0); 7.4493 (6.1); 7.4087 (0.4); 7.4009 (3.7); 5.4878 (0.5); 5.4697 (2.0); 5.4513 (2.7); 5.4328 (2.0); 5.4147
(0.6); 3.3446 (101.0); 3.2359 (0.5); 2.8931 (13.1); 2.7340 (11.3); 2.6744 (0.4); 2.5277 (1.2); 2.5144 (22.6); 2.5099
(44.0); 2.5054 (56.7); 2.5008 (41.3); 2.4965 (20.2); 2.3322 (0.3); 1.5689 (16.0); 1.5514 (15.9); 1.5273 (0.6); −0.0002 (0.5)
I.195: ¹H-NMR(400.2 MHz, d₆-DMSO):
δ = 9.0353 (5.6); 8.9066 (3.2); 8.8992 (4.9); 8.8598 (4.8); 8.8523 (3.1); 7.9541 (2.5); 7.6516 (2.3); 7.5233 (5.6); 7.3952
(2.7); 7.2717 (2.7); 7.2521 (6.2); 7.2339 (5.7); 7.1814 (5.9); 7.1779 (8.5); 7.1598 (5.8); 7.1573 (6.1); 7.1364 (3.7);
7.1216 (1.0); 7.1185 (1.4); 3.3406 (32.8); 2.8920 (16.0); 2.7328 (13.8); 2.5133 (14.6); 2.5092 (28.0); 2.5047 (35.9);
2.5002 (26.6); 2.4961 (13.3); 1.3514 (1.1); 1.3280 (6.0); 1.3226 (4.8); 1.2971 (5.3); 1.2907 (6.0); 1.2679 (1.1)
I.196: ¹H-NMR(300.2 MHz, CDCl₃):
δ = 9.0418 (0.4); 7.3840 (0.6); 7.3690 (0.4); 7.3560 (1.6); 7.3461 (0.4); 7.3355 (2.0); 7.2989 (10.4); 7.2851 (3.2); 7.2759
(0.6); 7.2708 (0.6); 7.2631 (1.8); 7.2399 (0.3); 7.1147 (1.0); 6.9425 (2.2); 6.7702 (1.1); 6.0025 (0.6); 3.1483 (0.4);
3.1446 (0.4); 3.1341 (0.7); 3.1235 (0.7); 3.1131 (0.4); 3.1094 (0.5); 2.2180 (0.4); 2.2068 (0.6); 2.1955 (0.4); 2.1856
(0.4); 2.1747 (0.3); 1.6044 (16.0); 1.4836 (0.5); 1.4633 (0.9); 1.4592 (0.6); 1.4426 (0.7); 1.4390 (1.0); 1.4182 (0.6);
1.3556 (0.6); 1.3410 (0.6); 1.3359 (0.5); 1.3223 (0.8); 1.3087 (0.6); 1.3038 (0.5); 1.2891 (0.4); 0.0487 (0.4); 0.0378
(12.9); 0.0269 (0.4)
I.197: ¹H-NMR(400.2 MHz, d₆-DMSO):
δ = 8.8659 (0.6); 8.6031 (2.2); 7.6250 (0.9); 7.4968 (2.4); 7.3686 (2.6); 7.3653 (2.2); 7.3471 (2.9); 7.3449 (2.7); 7.2802
(1.7); 7.2754 (0.6); 7.2617 (2.8); 7.2417 (1.6); 7.1673 (1.0); 7.1644 (0.6); 7.1491 (1.5); 7.1338 (0.4); 7.1310 (0.6);
3.3398 (14.0); 2.8921 (0.5); 2.7336 (0.4); 2.5139 (6.0); 2.5095 (11.6); 2.5050 (15.0); 2.5005 (11.0); 2.4960 (5.3);
1.7118 (16.0)
I.198: ¹H-NMR(400.2 MHz, d₆-DMSO):
δ = 8.8852 (0.6); 8.6428 (2.5); 8.5012 (1.0); 8.4990 (1.1); 8.4969 (0.9); 8.4913 (1.0); 8.4892 (1.1); 8.4870 (1.0); 8.4850
(0.9); 7.7146 (0.6); 7.7100 (0.6); 7.6948 (1.0); 7.6911 (1.0); 7.6759 (0.8); 7.6714 (0.7); 7.6296 (0.9); 7.5013 (2.1);
7.4021 (1.6); 7.3820 (1.5); 7.3732 (1.1); 7.2219 (0.8); 7.2195 (0.8); 7.2098 (0.8); 7.2074 (0.9); 7.2034 (0.9); 7.2010
(0.8); 7.1913 (0.8); 7.1889 (0.7); 3.3665 (61.0); 2.8940 (0.9); 2.7349 (0.7); 2.5295 (0.3); 2.5162 (6.4); 2.5119 (12.5);
2.5074 (16.1); 2.5029 (11.8); 2.4986 (5.8); 1.7245 (16.0); 1.6979 (0.4)
I.199: ¹H-NMR(400.2 MHz, d₆-DMSO):
δ = 8.8869 (0.5); 8.6808 (2.0); 8.6243 (0.5); 8.5911 (1.5); 8.5860 (1.5); 8.3848 (1.0); 8.3819 (1.1); 8.3734 (1.1); 8.3703
(1.0); 7.9538 (0.8); 7.7386 (0.7); 7.7345 (0.9); 7.7286 (0.7); 7.7186 (0.8); 7.7143 (1.0); 7.7128 (1.0); 7.7086 (0.7);
7.6273 (0.9); 7.4990 (2.2); 7.3709 (1.0); 7.3074 (0.9); 7.2960 (0.9); 7.2878 (0.9); 7.2761 (0.8); 3.3536 (5.9); 2.8921
(5.5); 2.7325 (4.7); 2.5266 (0.5); 2.5130 (11.7); 2.5088 (23.3); 2.5043 (30.5); 2.4998 (22.9); 2.4956 (11.7); 1.7384
(16.0); 1.7119 (0.4)
I.200: ¹H-NMR(400.2 MHz, d₆-DMSO):
δ = 8.8979 (6.2); 8.8904 (8.9); 8.8416 (9.3); 8.8341 (6.4); 8.3193 (3.0); 8.3169 (2.9); 8.3022 (5.4); 8.2879 (2.7); 7.6594
(5.0); 7.5311 (12.4); 7.4029 (5.8); 7.2977 (7.0); 7.2926 (3.5); 7.2835 (8.5); 7.2761 (10.1); 7.2674 (4.0); 7.2620 (8.9);
7.1375 (1.3); 7.1301 (9.9); 7.1250 (3.5); 7.1135 (3.9); 7.1077 (16.0); 7.1023 (4.2); 7.0906 (3.0); 7.0856 (7.9); 7.0781
(1.0); 3.6171 (3.6); 3.6005 (7.8); 3.5820 (7.7); 3.5662 (4.0); 3.3420 (51.1); 3.3182 (0.5); 2.8881 (6.7); 2.8697 (11.3);
2.8518 (6.1); 2.6756 (0.4); 2.5290 (1.2); 2.5154 (24.9); 2.5111 (49.6); 2.5066 (64.9); 2.5020 (48.4); 2.4977 (24.1);
2.3334 (0.4); −0.0002 (0.4)
I.201: ¹H-NMR(400.2 MHz, d₆-DMSO):
δ = 9.0164 (4.5); 9.0090 (5.1); 8.8325 (5.2); 8.8250 (4.6); 8.3835 (8.0); 7.9537 (0.9); 7.6711 (3.4); 7.5429 (8.6); 7.4147
(4.0); 7.2795 (3.6); 7.2622 (10.2); 7.2436 (8.8); 7.2072 (4.1); 7.2040 (3.0); 7.1946 (1.5); 7.1891 (5.3); 7.1828 (1.4);
7.1708 (1.9); 7.1609 (8.8); 7.1573 (10.3); 7.1403 (7.7); 3.3466 (110.3); 3.0089 (16.0); 2.8927 (6.2); 2.7340 (5.2);
2.7330 (5.1); 2.6742 (0.3); 2.5276 (1.0); 2.5141 (21.8); 2.5097 (43.6); 2.5052 (57.0); 2.5006 (42.1); 2.4962 (20.8);
2.3321 (0.3); 0.8780 (2.5); 0.8593 (9.0); 0.8490 (4.4); 0.8296 (1.2); 0.8125 (1.2); 0.7928 (4.3); 0.7826 (8.6); 0.7643
(2.4); −0.0002 (0.4)
I.202: ¹H-NMR(300.2 MHz, d₆-DMSO):
δ = 9.0276 (2.8); 8.9198 (1.3); 8.9098 (2.4); 8.8751 (2.3); 8.8651 (1.2); 7.7134 (1.2); 7.5424 (3.2); 7.3717 (1.5); 7.1738
(0.9); 7.1492 (1.4); 7.1464 (1.5); 7.1332 (0.4); 7.1216 (1.6); 7.0056 (1.8); 6.9942 (2.5); 6.9869 (2.6); 6.9837 (2.6);
6.9541 (1.1); 3.3495 (16.0); 2.5340 (2.4); 2.5281 (5.0); 2.5220 (6.8); 2.5159 (5.0); 2.5100 (2.4); 2.2620 (10.8); 2.0955
(0.8); 1.3568 (0.5); 1.3248 (2.9); 1.2804 (3.2); 1.2737 (2.9); 1.2660 (2.7); 0.8990 (0.6); 0.8772 (2.1); 0.8541 (0.7);
0.0191 (5.4)
I.203: ¹H-NMR(300.2 MHz, CDCl₃):
δ = 9.0230 (4.8); 8.3913 (2.3); 8.3750 (2.4); 7.2987 (20.2); 7.1580 (3.2); 7.1042 (2.1); 6.9427 (1.9); 6.9319 (5.7); 6.7597
(2.3); 6.5252 (1.9); 2.4826 (0.4); 2.4710 (0.8); 2.3700 (0.4); 2.3533 (0.6); 2.3015 (16.0); 2.0819 (0.8); 2.0717 (0.7);
2.0450 (1.0); 1.8669 (1.8); 1.8511 (5.3); 1.8413 (5.4); 1.8265 (2.2); 1.8065 (0.3); 1.7983 (0.4); 1.7823 (0.4); 1.7731
(0.5); 1.7085 (1.9); 1.6739 (3.8); 1.4129 (2.1); 1.3984 (5.2); 1.3886 (5.2); 1.3727 (1.8); 1.3361 (0.7); 1.3142 (0.8);
1.2888 (6.0); 0.9360 (0.4); 0.9166 (0.9); 0.8931 (0.4); 0.7717 (0.4); 0.1057 (0.5); 0.0455 (0.8); 0.0349 (19.0); 0.0240 (0.6)
I.204: ¹H-NMR(400.2 MHz, d₆-DMSO):
δ = 8.8553 (0.7); 8.8363 (1.1); 8.6209 (0.7); 8.5577 (2.2); 7.9550 (0.8); 7.6267 (1.0); 7.4984 (2.4); 7.3703 (1.1); 7.2399
(2.9); 7.2194 (3.7); 7.0661 (3.0); 7.0461 (2.4); 3.3411 (14.9); 2.8928 (5.1); 2.7344 (4.4); 2.7334 (4.2); 2.5146 (5.7);
2.5103 (11.2); 2.5058 (14.5); 2.5013 (10.7); 2.4970 (5.3); 2.2358 (9.5); 1.6883 (16.0)
I.205: ¹H-NMR(400.2 MHz, d₆-DMSO):
δ = 8.8405 (1.0); 8.8343 (1.1); 8.6912 (2.2); 8.5187 (1.1); 8.5126 (1.0); 7.9552 (1.2); 7.6136 (1.0); 7.4853 (2.6); 7.4149
(1.2); 7.3970 (1.4); 7.3572 (1.2); 7.1670 (0.6); 7.1491 (1.2); 7.1319 (0.7); 7.1293 (0.7); 7.0786 (0.7); 7.0759 (0.7);
7.0601 (1.5); 7.0575 (1.5); 7.0419 (0.8); 7.0393 (0.8); 6.9904 (1.5); 6.9735 (1.0); 3.3398 (11.5); 2.8929 (7.8); 2.7344
(6.7); 2.5148 (5.9); 2.5105 (11.5); 2.5060 (15.0); 2.5015 (11.1); 2.4971 (5.5); 2.3206 (10.1); 1.7509 (16.0)
I.206: ¹H-NMR(400.2 MHz, d₆-DMSO):
δ = 8.9283 (4.8); 8.8800 (4.9); 8.6131 (6.3); 8.6028 (6.2); 7.6613 (4.6); 7.5330 (11.0); 7.4048 (5.3); 7.2547 (7.0); 7.2496
(3.5); 7.2408 (8.4); 7.2329 (10.7); 7.2246 (4.3); 7.2191 (9.6); 7.1322 (1.3); 7.1249 (9.6); 7.1026 (16.0); 7.0806 (7.2);
3.3424 (67.1); 3.0360 (1.2); 3.0252 (3.1); 3.0166 (4.3); 3.0061 (4.4); 2.9975 (3.3); 2.9865 (1.4); 2.8930 (1.1); 2.7339
(0.9); 2.6750 (0.4); 2.5281 (1.2); 2.5144 (25.2); 2.5104 (48.6); 2.5060 (63.0); 2.5015 (47.0); 2.4973 (23.6); 2.3327
(0.4); 2.1099 (2.0); 2.1017 (2.2); 2.0943 (2.8); 2.0864 (4.6); 2.0785 (2.7); 2.0709 (2.5); 2.0627 (2.3); 1.3580 (2.0);
1.3443 (4.1); 1.3329 (4.2); 1.3211 (3.8); 1.3089 (2.9); 1.2996 (3.0); 1.2840 (4.9); 1.2808 (4.5); 1.2650 (4.7); 1.2504 (1.8)
I.207: ¹H-NMR(400.2 MHz, d₆-DMSO):
δ = 8.8509 (2.0); 8.8386 (1.9); 8.7202 (1.1); 8.6993 (1.1); 7.9544 (0.5); 7.6459 (1.0); 7.5176 (2.4); 7.3894 (1.1); 7.3283
(3.1); 7.3066 (3.4); 7.2997 (0.4); 6.8894 (0.5); 6.8821 (3.7); 6.8772 (1.3); 6.8651 (1.2); 6.8603 (3.4); 6.8530 (0.4);
5.2051 (0.6); 5.1859 (0.8); 5.1671 (0.6); 3.7139 (16.0); 3.3449 (17.6); 2.8923 (3.4); 2.7332 (2.9); 2.5145 (5.5); 2.5102
(10.7); 2.5056 (13.8); 2.5011 (10.2); 2.4967 (5.0); 1.4692 (4.5); 1.4517 (4.5)
I.208: ¹H-NMR(400.2 MHz, d₆-DMSO):
δ = 8.8555 (2.7); 8.8362 (9.9); 8.7480 (2.6); 8.5478 (9.3); 7.9549 (2.4); 7.6736 (2.4); 7.6563 (1.1); 7.6427 (4.1); 7.5281
(2.2); 7.5145 (9.9); 7.4601 (9.8); 7.4578 (9.8); 7.4558 (9.0); 7.4101 (0.3); 7.3999 (1.1); 7.3863 (4.7); 6.3135 (6.9);
6.3089 (7.6); 6.3054 (8.2); 6.3009 (7.5); 6.1489 (8.8); 6.1470 (9.2); 6.1409 (8.3); 6.1390 (8.2); 3.3429 (65.3); 2.8935
(16.0); 2.7339 (13.8); 2.6753 (0.4); 2.5150 (24.9); 2.5108 (47.8); 2.5063 (61.6); 2.5018 (45.8); 2.4975 (22.9); 2.4242
(1.9); 2.4128 (3.3); 2.3951 (3.6); 2.3812 (4.0); 2.3687 (3.2); 2.3378 (0.4); 2.3333 (0.5); 2.3290 (0.3); 2.1649 (2.2);
2.1582 (2.4); 2.1491 (3.9); 2.1327 (3.9); 2.1168 (3.2); 2.1035 (2.0); 1.8025 (0.6); 1.7879 (1.5); 1.7780 (2.0); 1.7697
(3.2); 1.7595 (3.8); 1.7503 (4.1); 1.7426 (4.5); 1.7385 (4.5); 1.7322 (3.5); 1.7156 (4.3); 1.7103 (4.5); 1.7038 (3.9);
1.6929 (4.5); 1.6845 (2.9); 1.6732 (1.9); 1.6544 (0.6)
I.209: ¹H-NMR(400.2 MHz, d₆-DMSO):
δ = 8.8742 (1.2); 8.8673 (1.9); 8.8244 (1.9); 8.8176 (1.2); 8.6955 (1.3); 8.6743 (1.3); 7.9545 (1.0); 7.6433 (1.2); 7.5150
(2.9); 7.3868 (1.4); 7.3388 (1.4); 7.3350 (1.5); 7.3199 (1.6); 7.3162 (1.6); 7.2237 (0.7); 7.2196 (0.8); 7.2033 (1.4);
7.2013 (1.4); 7.1848 (1.0); 7.1807 (1.0); 6.9892 (2.1); 6.9703 (1.7); 6.9687 (1.7); 6.9080 (1.1); 6.9061 (1.1); 6.8894
(1.9); 6.8875 (1.9); 6.8708 (0.9); 6.8688 (0.9); 5.5379 (0.8); 5.5200 (1.0); 5.4999 (0.8); 3.9362 (0.4); 3.8841 (0.4);
3.8431 (16.0); 3.3445 (25.5); 2.8923 (6.9); 2.7342 (5.8); 2.7331 (5.9); 2.5278 (0.4); 2.5144 (7.1); 2.5101 (13.6); 2.5056
(17.4); 2.5011 (12.9); 2.4967 (6.4); 1.4164 (6.0); 1.3992 (6.0)
I.210: ¹H-NMR(400.2 MHz, d₆-DMSO):
δ = 8.8393 (3.0); 8.8322 (6.3); 8.8117 (6.6); 8.8045 (3.0); 8.2379 (3.3); 8.2169 (3.4); 7.6500 (3.1); 7.5216 (7.9); 7.3934
(3.6); 7.2921 (4.4); 7.2870 (2.2); 7.2779 (5.4); 7.2705 (6.2); 7.2618 (2.5); 7.2564 (5.4); 7.1049 (0.8); 7.0976 (6.0);
7.0926 (2.1); 7.0809 (2.5); 7.0753 (9.8); 7.0581 (1.9); 7.0531 (4.9); 7.0457 (0.6); 4.3150 (0.7); 4.2962 (1.4); 4.2791
(2.0); 4.2621 (1.5); 4.2432 (0.7); 3.3450 (60.4); 2.8934 (2.1); 2.8736 (1.6); 2.8593 (2.8); 2.8401 (2.7); 2.7805 (2.8);
2.7648 (2.9); 2.7469 (1.7); 2.7312 (1.6); 2.5295 (0.9); 2.5160 (17.2); 2.5117 (34.3); 2.5071 (44.9); 2.5026 (33.1);
2.4982 (16.4); 1.1802 (16.0); 1.1638 (15.8)
I.211: ¹H-NMR(400.2 MHz, d₆-DMSO):
δ = 8.8763 (4.3); 8.8688 (6.0); 8.8168 (6.4); 8.8092 (4.4); 8.2954 (1.7); 8.2811 (3.3); 8.2662 (1.7); 7.9554 (1.1); 7.6561
(3.1); 7.5278 (7.9); 7.3996 (3.6); 7.3096 (4.5); 7.3045 (2.2); 7.2955 (5.4); 7.2880 (6.3); 7.2792 (2.6); 7.2740 (5.5);
7.1358 (0.8); 7.1283 (6.0); 7.1233 (2.1); 7.1060 (10.1); 7.0888 (1.9); 7.0838 (4.8); 7.0761 (0.6); 3.5938 (0.9); 3.5771
(1.4); 3.5601 (2.4); 3.5425 (2.6); 3.5262 (1.7); 3.5011 (1.6); 3.4866 (2.2); 3.4831 (2.2); 3.4684 (2.6); 3.4536 (1.1);
3.4497 (1.2); 3.4355 (0.9); 3.3426 (40.9); 3.1656 (0.4); 3.1426 (1.3); 3.1247 (2.6); 3.1068 (2.6); 3.0890 (1.2); 2.8935
(7.1); 2.7349 (6.0); 2.7338 (6.1); 2.5286 (1.0); 2.5152 (17.7); 2.5109 (34.2); 2.5064 (44.0); 2.5018 (32.4); 2.4975
(15.9); 1.2427 (16.0); 1.2253 (15.7)
I.212: ¹H-NMR(400.2 MHz, d₆-DMSO):
δ = 8.8642 (0.8); 8.6327 (2.9); 8.3185 (0.8); 7.6270 (1.2); 7.4988 (3.0); 7.3773 (0.7); 7.3708 (1.7); 7.3538 (1.3); 7.3363
(0.8); 7.3321 (0.8); 7.2566 (0.3); 7.2485 (0.4); 7.2421 (0.8); 7.2379 (0.7); 7.2297 (0.7); 7.2235 (0.8); 7.2181 (0.6);
7.2101 (0.6); 7.2060 (0.5); 7.1231 (1.0); 7.1201 (1.2); 7.1040 (1.6); 7.1011 (1.8); 7.0824 (1.6); 7.0635 (0.8); 7.0608
(0.8); 7.0518 (1.0); 7.0491 (0.9); 7.0316 (0.8); 7.0289 (0.7); 3.3483 (30.2); 3.3248 (0.4); 2.8937 (0.5); 2.7351 (0.4);
2.5159 (6.1); 2.5115 (12.1); 2.5070 (15.7); 2.5024 (11.5); 2.4980 (5.6); 1.7779 (16.0)
I.213: ¹H-NMR(400.2 MHz, d₆-DMSO):
δ = 8.8849 (4.5); 8.8432 (4.5); 8.8116 (3.9); 8.7909 (3.9); 7.9557 (1.9); 7.6498 (3.2); 7.5215 (7.5); 7.4648 (7.2); 7.3934
(3.7); 7.3810 (1.1); 7.3773 (1.1); 7.3655 (10.4); 7.3586 (5.1); 7.3500 (7.6); 7.3309 (2.0); 7.3076 (0.4); 7.2939 (3.2);
7.2884 (4.8); 7.2818 (2.5); 7.2774 (2.3); 7.2721 (2.9); 7.2663 (1.7); 5.2631 (0.6); 5.2456 (2.4); 5.2268 (3.2); 5.2080
(2.4); 5.1905 (0.6); 3.3412 (35.3); 3.1796 (0.4); 3.1666 (0.4); 2.8935 (11.9); 2.7344 (10.7); 2.5112 (35.9); 2.5069
(45.6); 2.5024 (34.2); 1.4931 (16.0); 1.4755 (16.0)
I.214: ¹H-NMR(400.2 MHz, d₆-DMSO):
δ = 8.8904 (3.5); 8.8514 (3.6); 8.8359 (4.5); 8.8157 (3.9); 8.4536 (7.1); 8.4474 (7.0); 7.9534 (2.0); 7.8947 (4.0); 7.8883
(3.9); 7.8738 (4.6); 7.8676 (4.4); 7.6494 (3.3); 7.5211 (8.5); 7.4998 (7.4); 7.4790 (6.8); 7.3930 (3.9); 5.2874 (0.5);
5.2701 (2.3); 5.2516 (3.3); 5.2331 (2.3); 5.2153 (0.6); 3.3364 (158.9); 3.1756 (1.1); 3.1626 (1.1); 2.8920 (14.1); 2.7329
(12.0); 2.7319 (11.6); 2.6773 (0.8); 2.6729 (1.1); 2.6682 (0.8); 2.5766 (0.9); 2.5262 (3.6); 2.5127 (70.9); 2.5083
(139.5); 2.5038 (181.4); 2.4992 (133.0); 2.4949 (65.0); 2.3395 (0.4); 2.3352 (0.8); 2.3307 (1.1); 2.3261 (0.8); 1.5225
(16.0); 1.5049 (15.9); 1.4747 (0.5); −0.0002 (0.9)
I.215: ¹H-NMR(400.2 MHz, d₆-DMSO):
δ = 8.8899 (4.4); 8.8470 (7.3); 8.8262 (4.0); 7.9561 (2.0); 7.6488 (3.3); 7.5206 (8.7); 7.5067 (2.2); 7.5016 (3.5); 7.4850
(3.5); 7.4800 (2.2); 7.4632 (1.7); 7.3924 (3.8); 7.2286 (1.6); 7.2223 (1.8); 7.1997 (2.9); 7.1785 (1.7); 7.1723 (1.8);
7.0868 (1.7); 7.0812 (1.6); 7.0655 (3.2); 7.0599 (2.9); 7.0442 (1.6); 7.0385 (1.4); 5.4664 (0.6); 5.4488 (2.3); 5.4304
(3.4); 5.4118 (2.3); 5.3944 (0.6); 3.3465 (77.3); 3.1797 (0.4); 3.1667 (0.3); 2.8948 (13.5); 2.7359 (11.5); 2.7350 (11.4);
2.5300 (1.0); 2.5166 (19.9); 2.5123 (38.1); 2.5078 (48.9); 2.5032 (35.8); 2.4988 (17.6); 1.4876 (16.0); 1.4701 (16.0)
I.216: ¹H-NMR(400.2 MHz, d₆-DMSO):
δ = 8.8331 (1.5); 8.8091 (2.1); 7.9537 (0.9); 7.6407 (0.8); 7.5124 (2.2); 7.3842 (1.0); 7.0246 (0.6); 7.0084 (0.9); 7.0037
(1.0); 6.9876 (1.5); 6.9411 (3.2); 6.9224 (1.6); 3.3443 (19.9); 2.8917 (5.9); 2.7336 (5.0); 2.7326 (5.1); 2.6811 (16.0);
2.5140 (4.7); 2.5096 (9.4); 2.5051 (12.3); 2.5006 (9.2); 2.4962 (4.6); 1.3922 (0.8); 1.3790 (2.1); 1.3736 (2.2); 1.3614
(0.9); 1.1217 (0.9); 1.1094 (2.1); 1.1041 (2.2); 1.0908 (0.8)
I.217: ¹H-NMR(400.2 MHz, d₆-DMSO):
δ = 8.7896 (0.6); 8.7827 (1.9); 8.7715 (2.1); 8.7640 (0.6); 8.7165 (4.9); 8.7091 (9.2); 8.6827 (9.7); 8.6752 (5.0); 8.4052
(0.8); 8.3841 (0.8); 7.9844 (4.5); 7.9624 (4.7); 7.9546 (2.9); 7.6362 (0.9); 7.6272 (4.5); 7.5080 (2.0); 7.4989 (11.3);
7.3797 (1.0); 7.3706 (5.3); 7.3195 (0.9); 7.3158 (1.3); 7.2983 (2.3); 7.2699 (1.5); 7.2513 (2.2); 7.2319 (1.0); 7.1882
(4.2); 7.1840 (6.1); 7.1669 (16.0); 7.1641 (14.3); 7.1540 (12.0); 7.1484 (3.1); 7.1359 (14.0); 7.1322 (5.5); 7.1163 (5.6);
7.0657 (2.8); 7.0618 (4.7); 7.0576 (2.6); 7.0499 (2.3); 7.0443 (5.7); 7.0386 (1.8); 7.0306 (1.5); 7.0269 (2.3); 7.0230
(1.2); 4.7484 (1.0); 4.7313 (2.4); 4.7134 (2.9); 4.7100 (2.7); 4.6949 (2.5); 4.6777 (1.0); 4.4578 (0.4); 4.4344 (0.6);
4.4141 (0.4); 3.3905 (1.9); 3.3720 (3.9); 3.3666 (3.1); 3.3445 (114.3); 3.3308 (3.6); 3.1782 (0.5); 3.1650 (0.3); 3.1545
(0.6); 3.1335 (0.5); 2.8920 (15.2); 2.7337 (12.7); 2.7327 (12.7); 2.6742 (0.4); 2.6696 (0.3); 2.5275 (1.3); 2.5141 (28.5);
2.5097 (56.6); 2.5052 (73.7); 2.5006 (53.9); 2.4962 (26.1); 2.3365 (0.3); 2.3320 (0.4); 2.3275 (0.3); 2.2210 (0.4);
2.2056 (0.4); 2.1875 (0.4); 2.1473 (1.0); 2.1368 (1.6); 2.1298 (1.3); 2.1241 (1.5); 2.1153 (3.8); 2.1057 (3.5); 2.0919
(3.7); 2.0836 (4.7); 2.0743 (1.7); 2.0694 (1.7); 2.0606 (4.2); 2.0531 (1.6); 2.0443 (1.9); 2.0376 (2.4); 2.0326 (2.7);
2.0246 (3.2); 2.0125 (2.2); 2.0045 (2.3); 1.9924 (1.9); 1.9807 (1.3); 1.9721 (1.7); 1.9614 (2.1); 1.9517 (2.0); 1.9408
(2.2); 1.9308 (2.2); 1.9208 (1.4); 1.9091 (1.0); 1.9000 (0.6); 1.8354 (1.8); 1.8210 (1.9); 1.8163 (2.7); 1.8026 (3.2);
1.7958 (2.3); 1.7891 (1.7); 1.7824 (3.0); 1.7697 (2.0); 1.7635 (1.5); 1.7496 (1.3); 1.7308 (0.4); 1.7178 (0.9); 1.6969
(2.0); 1.6877 (1.0); 1.6749 (2.3); 1.6665 (1.8); 1.6552 (1.6); 1.6465 (1.9); 1.6335 (0.7); 1.6244 (1.4); 1.6030 (0.8);
1.5828 (0.4); 1.5728 (0.4); 1.5515 (0.4); −0.0002 (0.5)
I.218: ¹H-NMR(400.2 MHz, d₆-DMSO):
δ = 9.2317 (0.5); 9.2219 (0.5); 8.8629 (2.9); 8.8335 (0.5); 8.7031 (7.2); 8.6678 (2.9); 8.6150 (5.7); 8.6100 (5.6); 8.3693
(4.1); 8.3657 (4.4); 8.3576 (4.3); 8.3539 (4.1); 8.3177 (0.6); 7.9530 (2.3); 7.7719 (2.4); 7.7677 (3.1); 7.7619 (2.4);
7.7519 (2.7); 7.7478 (3.3); 7.7460 (3.4); 7.7419 (2.5); 7.6274 (3.0); 7.4992 (7.8); 7.3711 (3.6); 7.3067 (3.1); 7.3054
(3.1); 7.2936 (3.2); 7.2867 (3.0); 7.2749 (2.8); 7.2736 (2.7); 4.1122 (0.5); 4.0985 (0.5); 3.3368 (297.7); 3.1748 (2.7);
3.1621 (2.6); 2.8914 (16.0); 2.7482 (0.4); 2.7323 (13.4); 2.7314 (13.6); 2.6768 (1.7); 2.6721 (2.0); 2.6676 (1.5); 2.5436
(3.4); 2.5255 (8.6); 2.5119 (129.4); 2.5076 (248.3); 2.5031 (321.4); 2.4986 (239.6); 2.4943 (120.0); 2.3345 (1.4);
2.3300 (1.9); 2.3255 (1.4); 2.0887 (2.6); 2.0724 (2.6); 2.0558 (2.4); 2.0423 (1.5); 1.8484 (1.1); 1.8281 (2.3); 1.8166
(2.6); 1.8093 (3.3); 1.7995 (3.6); 1.7809 (3.8); 1.7766 (3.9); 1.7688 (3.2); 1.7585 (3.2); 1.7390 (1.3); 1.2351
(0.6); −0.0001 (1.5)
I.219: ¹H-NMR(400.2 MHz, d₆-DMSO):
δ = 8.8903 (3.5); 8.8838 (3.8); 8.6955 (7.9); 8.6420 (3.8); 8.6351 (3.4); 8.5017 (3.3); 8.4997 (4.0); 8.4974 (4.2); 8.4951
(3.8); 8.4898 (3.7); 8.4877 (4.3); 8.4854 (4.1); 8.4832 (3.5); 8.3178 (0.5); 7.9528 (2.2); 7.6519 (2.5); 7.6473 (2.6);
7.6319 (5.2); 7.6285 (7.6); 7.6132 (3.2); 7.6086 (3.0); 7.5004 (9.5); 7.3723 (4.2); 7.3059 (6.3); 7.2858 (5.7); 7.1763
(3.3); 7.1738 (3.4); 7.1643 (3.3); 7.1617 (3.6); 7.1578 (3.5); 7.1552 (3.2); 7.1458 (3.2); 7.1432 (3.0); 3.3364 (282.0);
3.1750 (0.6); 3.1621 (0.6); 2.8913 (16.0); 2.7324 (12.9); 2.7313 (13.6); 2.6767 (1.1); 2.6722 (1.6); 2.6676 (1.2); 2.6631
(0.6); 2.5256 (5.0); 2.5121 (97.0); 2.5077 (193.0); 2.5032 (252.4); 2.4986 (186.2); 2.4942 (91.2); 2.3602 (1.3); 2.3299
(8.4); 2.3177 (5.4); 2.3095 (5.8); 2.2899 (2.3); 2.2769 (1.5); 2.2605 (0.7); 1.7985 (7.2); 1.7859 (6.9); 1.7745 (4.2);
1.7444 (0.5); 1.2347 (0.4); −0.0001 (1.2)
I.220: ¹H-NMR(400.2 MHz, d₆-DMSO):
δ = 9.0779 (9.0); 8.8514 (3.3); 8.7084 (3.3); 7.6329 (3.9); 7.5275 (0.8); 7.5196 (7.0); 7.5143 (3.5); 7.5048 (16.0); 7.4975
(8.8); 7.4891 (3.5); 7.4837 (7.6); 7.4758 (1.0); 7.3765 (4.6); 7.1414 (0.9); 7.1334 (7.5); 7.1283 (2.7); 7.1111 (14.1);
7.0940 (2.4); 7.0888 (6.8); 7.0810 (0.8); 3.3444 (83.8); 2.8937 (1.9); 2.7351 (1.7); 2.6760 (0.4); 2.6282 (0.9); 2.6093
(1.7); 2.5990 (4.4); 2.5814 (8.3); 2.5681 (7.1); 2.5636 (7.9); 2.5476 (4.5); 2.5357 (1.9); 2.5295 (1.7); 2.5157 (23.5);
2.5114 (45.4); 2.5069 (59.1); 2.5023 (43.8); 2.4980 (21.8); 2.3338 (0.4); 2.0800 (0.4); 2.0636 (0.9); 2.0578 (0.9);
2.0517 (0.9); 2.0477 (0.9); 2.0409 (1.7); 2.0359 (1.8); 2.0309 (1.4); 2.0256 (1.3); 2.0194 (1.7); 2.0133 (2.0); 2.0029
(0.8); 1.9971 (1.2); 1.9924 (0.9); 1.9756 (0.4); 1.9152 (0.5); 1.8945 (1.5); 1.8874 (0.8); 1.8766 (2.3); 1.8552 (1.7);
1.8489 (1.7); 1.8372 (0.7); 1.8276 (0.9)
I.221: ¹H-NMR(300.2 MHz, CDCl₃):
δ = 9.0611 (0.8); 9.0061 (0.8); 7.9060 (1.1); 7.9004 (1.3); 7.8807 (2.4); 7.8746 (2.6); 7.8549 (1.3); 7.8490 (1.3); 7.2987
(14.4); 7.2771 (0.6); 7.2696 (1.4); 7.2625 (1.3); 7.2517 (1.2); 7.2435 (1.6); 7.2364 (1.0); 7.2255 (1.0); 7.2194 (0.8);
7.1213 (1.7); 7.1176 (2.0); 7.0990 (3.4); 7.0924 (3.6); 7.0862 (2.2); 7.0711 (1.3); 7.0673 (1.5); 7.0591 (1.5); 7.0553
(1.3); 7.0496 (1.8); 7.0459 (1.6); 7.0222 (1.3); 7.0188 (1.2); 6.9275 (4.9); 6.7552 (2.5); 3.4347 (15.7); 3.4282 (16.0);
2.0841 (0.7); 1.5965 (13.6); 1.5456 (1.1); 1.5195 (4.2); 1.5030 (2.2); 1.4713 (0.6); 1.4523 (0.7); 1.4031 (4.9); 1.3786
(0.9); 1.3209 (0.4); 1.2977 (1.1); 0.9193 (0.7); 0.8958 (0.3); 0.0482 (0.7); 0.0373 (19.9); 0.0269 (0.9)
I.222: ¹H-NMR(400.1 MHz, d₆-DMSO):
δ = 8.9688 (6.0); 8.7453 (1.9); 8.7261 (2.0); 8.5875 (1.8); 8.5759 (1.8); 7.8969 (0.9); 7.8931 (0.8); 7.8777 (1.8); 7.8741
(1.7); 7.8585 (1.1); 7.8549 (1.0); 7.6719 (1.2); 7.6263 (2.3); 7.6068 (2.0); 7.5436 (2.6); 7.4285 (1.4); 7.4156 (2.7);
7.3978 (1.2); 5.9484 (2.6); 5.9293 (2.6); 3.6675 (16.0); 3.3742 (303.8); 3.3343 (0.4); 2.5072 (7.0)
I.223: ¹H-NMR(400.2 MHz, d₆-DMSO):
δ = 8.9824 (0.6); 8.8816 (0.6); 7.7381 (2.1); 7.6762 (1.0); 7.5479 (2.5); 7.4198 (1.2); 7.0560 (3.9); 7.0514 (3.7); 7.0353
(8.3); 3.3394 (10.8); 3.1860 (4.6); 2.8936 (1.1); 2.7349 (1.0); 2.7336 (1.0); 2.5148 (5.4); 2.5103 (10.7); 2.5058 (13.9);
2.5012 (10.2); 2.4967 (4.9); 1.3651 (16.0)
I.224: ¹H-NMR(400.2 MHz, d₆-DMSO):
δ = 8.8017 (7.5); 8.1089 (0.5); 8.0932 (1.0); 8.0775 (0.5); 7.6501 (1.0); 7.5218 (2.4); 7.4510 (1.6); 7.4456 (0.7); 7.4373
(1.9); 7.4286 (2.0); 7.4203 (0.8); 7.4149 (1.8); 7.3936 (1.1); 7.1165 (1.8); 7.1111 (0.6); 7.0941 (3.4); 7.0771 (0.6);
7.0718 (1.6); 3.6284 (2.8); 3.6124 (2.8); 3.3453 (16.5); 2.8940 (0.5); 2.7354 (0.4); 2.7342 (0.4); 2.5158 (5.0); 2.5115
(9.7); 2.5069 (12.6); 2.5024 (9.3); 2.4979 (4.5); 1.3234 (16.0)
I.225: ¹H-NMR(400.2 MHz, d₆-DMSO):
δ = 8.8704 (3.0); 8.8363 (5.9); 8.7516 (3.0); 8.7068 (8.7); 7.9546 (2.5); 7.6735 (1.6); 7.6557 (0.7); 7.6396 (3.7); 7.5275
(1.3); 7.5114 (8.8); 7.3993 (0.6); 7.3832 (4.2); 7.2529 (6.1); 7.2501 (6.6); 7.2403 (6.7); 7.2375 (6.8); 6.9563 (5.8);
6.9533 (6.3); 6.9475 (8.0); 6.9445 (7.6); 6.8933 (6.6); 6.8844 (5.9); 6.8807 (6.8); 6.8718 (5.0); 3.3485 (114.1); 2.8932
(16.0); 2.7337 (14.0); 2.6754 (0.3); 2.5864 (3.4); 2.5782 (2.8); 2.5716 (2.7); 2.5571 (4.3); 2.5446 (2.7); 2.5288 (1.4);
2.5108 (43.2); 2.5064 (55.9); 2.5019 (42.2); 2.3331 (0.4); 2.1234 (3.9); 2.1048 (2.9); 2.0915 (3.2); 2.0748 (1.6); 1.8102
(0.6); 1.7643 (10.0); 1.7549 (8.8)
I.226: ¹H-NMR(300.2 MHz, d₆-DMSO):
δ = 9.0664 (0.4); 7.5439 (0.5); 7.3406 (0.5); 7.3116 (0.9); 7.2195 (0.8); 7.1904 (0.5); 3.3469 (16.0); 2.5342 (1.6); 2.5283
(3.3); 2.5222 (4.5); 2.5161 (3.2); 2.5102 (1.5); 1.3549 (0.5); 1.3194 (0.4); 0.0203 (4.5)
I.227: ¹H-NMR(400.1 MHz, d₆-DMSO):
δ = 9.0538 (12.7); 8.9150 (8.9); 8.8773 (8.9); 7.6496 (4.1); 7.5211 (9.2); 7.3930 (4.6); 7.3124 (3.2); 7.2933 (8.5); 7.2739
(6.5); 7.2187 (7.7); 7.1978 (16.0); 7.1461 (7.1); 7.1260 (5.7); 3.5215 (0.4); 3.5073 (0.5); 3.4835 (0.7); 3.3775 (1903.0);
3.2969 (1.2); 3.2888 (1.0); 3.2559 (0.7); 3.2254 (0.4); 3.2107 (0.3); 3.0603 (0.4); 3.0410 (0.4); 3.0270 (0.4); 2.6747
(0.3); 2.5068 (40.5); 1.9908 (1.1); 1.4045 (3.5); 1.3852 (12.4); 1.3747 (6.2); 1.3571 (1.9); 1.3373 (2.0); 1.3185 (6.8);
1.3085 (12.8); 1.2887 (3.8); 1.2461 (6.3); 1.1953 (0.6); 1.1763 (0.7); 1.1591 (0.4); 0.8755 (2.5); 0.8588 (5.8); 0.8413 (2.6)
I.229: ¹H-NMR(300.2 MHz, d₆-DMSO):
δ = 9.0586 (3.0); 8.9259 (2.4); 8.9039 (2.3); 7.7174 (1.1); 7.5464 (2.5); 7.3756 (1.3); 7.3685 (0.7); 7.3395 (1.3); 7.3104
(0.9); 7.3041 (1.3); 7.2752 (0.7); 7.2396 (0.7); 7.2322 (0.8); 7.2139 (0.8); 7.2066 (0.8); 7.1985 (0.8); 7.1909 (0.8);
7.1723 (0.7); 7.1653 (0.7); 7.0699 (1.0); 7.0562 (0.9); 7.0413 (0.8); 3.3486 (16.0); 2.5273 (6.3); 2.5219 (7.9); 2.5164
(6.1); 2.0085 (0.4); 1.4118 (0.9); 1.3838 (3.0); 1.3699 (2.0); 1.3213 (2.1); 1.3084 (3.1); 1.2809 (1.0); 1.2645 (0.9);
0.8763 (0.5); 0.0183 (4.5)
I.230: ¹H-NMR(400.2 MHz, d₆-DMSO):
δ = 8.8690 (3.0); 8.8622 (4.6); 8.8196 (4.8); 8.8118 (3.8); 8.8065 (4.2); 8.7847 (3.4); 7.9561 (1.5); 7.6463 (2.9); 7.5180
(7.1); 7.4323 (5.6); 7.4273 (2.4); 7.4108 (15.1); 7.3894 (7.0); 7.3854 (16.0); 7.3802 (4.0); 7.3688 (2.5); 7.3639 (6.0);
5.0015 (1.1); 4.9853 (1.7); 4.9802 (2.4); 4.9645 (2.5); 4.9431 (1.1); 3.3484 (72.6); 2.8941 (10.6); 2.7349 (8.9); 2.5299
(0.8); 2.5165 (16.8); 2.5122 (32.6); 2.5077 (42.1); 2.5032 (31.0); 2.4988 (15.2); 1.8932 (0.7); 1.8750 (1.1); 1.8717
(1.0); 1.8590 (1.6); 1.8404 (1.8); 1.8380 (1.8); 1.8192 (1.5); 1.7968 (1.5); 1.7801 (2.1); 1.7624 (2.3); 1.7444 (1.4);
1.7284 (0.8); 0.9180 (7.2); 0.8998 (15.5); 0.8816 (6.7)
I.231: ¹H-NMR(400.2 MHz, d₆-DMSO):
δ = 8.8730 (0.7); 8.6402 (2.8); 7.9551 (0.5); 7.6290 (1.0); 7.5007 (2.4); 7.3801 (2.3); 7.3730 (1.7); 7.3635 (1.5); 7.3581
(5.1); 7.3235 (5.0); 7.3067 (1.0); 7.3016 (2.3); 3.3440 (25.4); 2.8934 (3.1); 2.7347 (2.6); 2.7338 (2.7); 2.5285 (0.3);
2.5108 (13.3); 2.5063 (17.1); 2.5019 (13.0); 1.6970 (16.0)
I.232: ¹H-NMR(400.2 MHz, d₆-DMSO):
δ = 8.9023 (2.9); 8.8824 (5.4); 8.8330 (2.9); 7.9562 (2.4); 7.8952 (4.0); 7.8819 (4.7); 7.8726 (8.7); 7.8687 (7.3); 7.8589
(5.3); 7.6405 (2.2); 7.6155 (3.0); 7.6113 (3.0); 7.5941 (2.6); 7.5900 (2.7); 7.5122 (6.4); 7.4953 (2.8); 7.4907 (2.5);
7.4845 (3.0); 7.4781 (5.2); 7.4715 (2.8); 7.4653 (2.4); 7.4610 (2.6); 7.4476 (0.8); 7.4440 (0.7); 7.3840 (2.6); 5.4376
(0.4); 5.4203 (1.5); 5.4014 (2.1); 5.3827 (1.5); 5.3649 (0.4); 3.3486 (39.7); 3.1814 (0.4); 3.1684 (0.4); 2.8921 (16.0);
2.7342 (13.7); 2.5293 (0.6); 2.5159 (13.0); 2.5116 (25.7); 2.5070 (33.5); 2.5025 (24.8); 2.4982 (12.3); 1.5945 (10.5);
1.5770 (10.5)
I.233: ¹H-NMR(400.2 MHz, d₆-DMSO):
δ = 8.9236 (3.4); 8.9170 (4.2); 8.8443 (4.0); 8.8256 (7.9); 8.3497 (5.5); 8.3283 (5.9); 8.0084 (4.3); 7.9873 (5.2); 7.9529
(4.2); 7.9325 (4.5); 7.7742 (2.3); 7.7708 (2.5); 7.7569 (3.1); 7.7533 (4.5); 7.7498 (2.7); 7.7359 (2.5); 7.7324 (2.5);
7.6457 (3.2); 7.6271 (8.0); 7.6058 (7.9); 7.5958 (3.0); 7.5931 (3.2); 7.5757 (4.9); 7.5731 (3.5); 7.5584 (2.3); 7.5557
(2.3); 7.5174 (8.1); 7.3893 (3.8); 5.4585 (0.6); 5.4410 (2.7); 5.4227 (4.0); 5.4044 (2.7); 5.3867 (0.6); 3.3476 (26.1);
2.8938 (1.5); 2.7359 (1.3); 2.7349 (1.3); 2.5307 (0.9); 2.5172 (19.4); 2.5129 (38.2); 2.5084 (49.9); 2.5038 (37.4);
2.4995 (19.0); 1.6241 (16.0); 1.6065 (16.0)
I.234: ¹H-NMR(400.2 MHz, d₆-DMSO):
δ = 8.8799 (7.7); 8.8386 (7.6); 8.6564 (3.2); 8.6404 (6.7); 8.6242 (3.2); 8.3199 (2.6); 7.6608 (5.9); 7.6268 (8.2); 7.6134
(9.4); 7.6048 (10.4); 7.5916 (9.3); 7.5326 (14.5); 7.4044 (6.9); 7.3378 (8.7); 7.3158 (16.0); 7.2937 (7.7); 4.2477 (3.2);
4.2316 (3.4); 4.2115 (6.9); 4.1953 (6.8); 4.1753 (3.6); 4.1590 (3.4); 3.3420 (61.6); 3.3182 (1.0); 2.8938 (0.6); 2.7353
(0.5); 2.6801 (0.4); 2.6758 (0.5); 2.6713 (0.4); 2.5291 (1.5); 2.5156 (30.8); 2.5113 (60.6); 2.5068 (78.8); 2.5023 (58.4);
2.4979 (29.0); 2.3382 (0.4); 2.3337 (0.5); 2.3291 (0.4); −0.0002 (0.4)
I.235: ¹H-NMR(400.2 MHz, d₆-DMSO):
δ = 8.8711 (1.2); 8.8353 (0.3); 8.6475 (1.1); 7.9461 (3.8); 7.6286 (1.5); 7.5004 (3.6); 7.3722 (1.7); 7.0848 (3.8); 7.0235
(0.3); 6.9999 (9.1); 6.9804 (0.4); 5.1020 (1.2); 5.0867 (2.6); 5.0714 (1.2); 3.7419 (1.0); 3.7269 (1.1); 3.7145 (1.4);
3.6996 (1.3); 3.5335 (1.4); 3.5177 (1.5); 3.5061 (1.2); 3.4903 (1.1); 3.3468 (43.4); 2.8919 (3.3); 2.7329 (2.9); 2.5272
(0.5); 2.5134 (9.7); 2.5094 (18.6); 2.5050 (24.1); 2.5005 (17.9); 2.4963 (8.9); 2.1607 (15.8); 2.1472 (16.0); 1.7471 (14.8)
I.236: ¹H-NMR(400.2 MHz, d₆-DMSO):
δ = 9.0278 (2.8); 8.8727 (1.4); 8.8368 (1.5); 7.6458 (1.1); 7.5174 (2.8); 7.3893 (1.3); 7.1908 (0.5); 7.1787 (1.0); 7.1746
(0.8); 7.1677 (1.2); 7.1567 (0.9); 7.1507 (0.7); 7.0378 (0.4); 7.0176 (3.9); 7.0038 (2.2); 3.7862 (16.0); 3.3458 (25.6);
2.5157 (6.2); 2.5114 (12.2); 2.5069 (15.7); 2.5023 (11.5); 2.4980 (5.7); 1.2971 (0.8); 1.2769 (2.8); 1.2660 (1.4); 1.2456
(0.4); 1.2293 (0.4); 1.2084 (1.4); 1.1976 (2.6); 1.1780 (0.7)
I.237: ¹H-NMR(400.2 MHz, d₆-DMSO):
δ = 9.1048 (8.4); 8.8586 (3.3); 8.7003 (3.3); 7.9556 (1.3); 7.6333 (3.5); 7.5049 (9.6); 7.4975 (11.9); 7.4929 (4.5); 7.4808
(5.1); 7.4761 (16.0); 7.4697 (2.3); 7.3768 (4.2); 7.3618 (15.0); 7.3574 (5.2); 7.3405 (11.2); 3.3440 (74.5); 2.8936 (9.0);
2.7345 (7.8); 2.6759 (0.4); 2.6207 (0.8); 2.6021 (1.7); 2.5917 (4.1); 2.5741 (7.7); 2.5609 (6.7); 2.5565 (7.3); 2.5406
(4.3); 2.5287 (2.7); 2.5154 (22.5); 2.5113 (43.0); 2.5068 (54.8); 2.5023 (40.6); 2.4980 (20.3); 2.3336 (0.3); 2.0878
(0.4); 2.0710 (0.8); 2.0649 (0.8); 2.0594 (0.9); 2.0552 (0.8); 2.0481 (1.5); 2.0431 (1.7); 2.0376 (1.3); 2.0331 (1.2);
2.0266 (1.6); 2.0206 (1.9); 2.0103 (0.7); 2.0044 (1.1); 1.9830 (0.4); 1.9256 (0.4); 1.9049 (1.4); 1.8978 (0.7); 1.8869
(2.0); 1.8656 (1.6); 1.8592 (1.6); 1.8474 (0.7); 1.8380 (0.8)
I.238: ¹H-NMR(400.2 MHz, d₆-DMSO):
δ = 9.2617 (3.1); 9.2385 (3.2); 8.9185 (3.7); 8.9031 (3.7); 8.3176 (0.6); 7.9526 (1.3); 7.6542 (2.4); 7.5259 (6.5); 7.4255
(10.0); 7.4074 (15.5); 7.3980 (4.1); 7.3555 (8.6); 7.3510 (3.1); 7.3373 (16.0); 7.3180 (8.3); 7.2685 (5.6); 7.2656 (3.5);
7.2553 (2.5); 7.2503 (7.4); 7.2452 (2.1); 7.2322 (2.5); 6.5280 (3.8); 6.5048 (3.7); 3.3393 (382.6); 2.8909 (9.0); 2.7321
(7.5); 2.7311 (7.7); 2.6809 (0.5); 2.6765 (1.0); 2.6720 (1.4); 2.6676 (1.0); 2.5254 (4.4); 2.5119 (86.1); 2.5075 (170.5);
2.5030 (223.0); 2.4984 (165.1); 2.4941 (82.2); 2.3343 (1.0); 2.3298 (1.3); 2.3253 (1.0); 1.2348 (0.4); −0.0001 (1.1)
I.239: ¹H-NMR(400.2 MHz, d₆-DMSO):
δ = 8.8928 (4.4); 8.8830 (4.9); 8.8622 (4.5); 8.8418 (4.1); 7.9578 (1.0); 7.7769 (6.7); 7.7307 (2.9); 7.7137 (3.9); 7.6492
(3.3); 7.6060 (1.3); 7.5860 (10.0); 7.5671 (4.3); 7.5478 (1.2); 7.5209 (8.2); 7.3928 (3.9); 5.3607 (0.6); 5.3431 (2.3);
5.3245 (3.1); 5.3055 (2.3); 5.2879 (0.6); 3.3451 (42.4); 2.8953 (6.5); 2.7369 (5.6); 2.5312 (1.0); 2.5178 (18.2); 2.5134
(35.3); 2.5089 (45.6); 2.5044 (33.5); 2.5000 (16.4); 1.5251 (16.0); 1.5075 (15.8)
I.240: ¹H-NMR(400.2 MHz, d₆-DMSO):
δ = 8.9762 (3.8); 8.9570 (3.8); 8.8785 (3.0); 8.8238 (0.4); 8.7767 (3.0); 8.3200 (0.8); 7.9560 (0.6); 7.8576 (4.1); 7.8381
(5.1); 7.6916 (6.1); 7.6717 (8.9); 7.6500 (2.6); 7.6397 (3.6); 7.5115 (8.3); 7.4598 (2.7); 7.4408 (4.4); 7.4216 (2.0);
7.3833 (3.9); 5.5863 (0.5); 5.5685 (2.1); 5.5507 (3.2); 5.5330 (2.1); 5.5155 (0.5); 3.3413 (57.3); 3.3185 (0.5); 2.8935
(3.7); 2.7351 (3.1); 2.7340 (3.3); 2.6755 (0.4); 2.5153 (27.1); 2.5111 (50.4); 2.5066 (63.5); 2.5021 (46.6); 2.4979
(23.0); 2.3334 (0.4); 1.5206 (0.5); 1.4974 (16.0); 1.4802 (15.8)
I.241: ¹H-NMR(300.2 MHz, CDCl₃):
δ = 9.1035 (6.8); 7.9275 (2.4); 7.9216 (2.7); 7.9020 (5.0); 7.8959 (5.4); 7.8763 (2.8); 7.8702 (2.8); 7.2987 (4.5); 7.2923
(1.3); 7.2861 (1.3); 7.2749 (1.3); 7.2677 (2.8); 7.2607 (2.4); 7.2500 (2.2); 7.2481 (2.2); 7.2410 (3.4); 7.2343 (2.1);
7.2232 (2.0); 7.2170 (1.9); 7.1207 (3.6); 7.1165 (4.5); 7.1051 (5.6); 7.0956 (5.7); 7.0913 (7.0); 7.0771 (4.0); 7.0728
(3.6); 7.0708 (3.8); 7.0662 (3.3); 7.0502 (2.9); 7.0462 (2.8); 7.0406 (3.7); 7.0367 (3.3); 7.0134 (2.7); 7.0095 (2.5);
6.9328 (11.2); 6.7606 (5.5); 4.7205 (15.7); 4.7127 (16.0); 2.1816 (4.8); 2.1737 (11.1); 2.1658 (4.9); 1.6855 (5.8);
1.6567 (6.1); 1.5859 (0.5); 1.5586 (0.4); 1.4881 (6.9); 1.3014 (1.4); 0.9387 (0.5); 0.9170 (1.4); 0.8938 (0.6); 0.1118
(0.7); 0.0363 (5.6)
I.242: ¹H-NMR(300.2 MHz, d₆-DMSO):
δ = 9.0325 (1.0); 8.9186 (0.4); 8.9086 (0.8); 8.8808 (0.8); 8.8709 (0.4); 7.7131 (0.4); 7.5420 (0.9); 7.3712 (0.4); 7.2965
(1.0); 7.2681 (1.4); 7.1253 (1.4); 7.0968 (1.0); 3.3516 (14.9); 2.5343 (0.9); 2.5283 (1.9); 2.5223 (2.6); 2.5162 (1.9);
2.5103 (0.9); 1.3004 (1.1); 1.2743 (1.4); 1.2527 (16.0); 0.0195 (2.4)
I.243: ¹H-NMR(300.2 MHz, d₆-DMSO):
δ = 9.0045 (1.2); 8.8953 (4.2); 7.7131 (0.5); 7.5420 (1.3); 7.3713 (0.6); 6.8274 (1.7); 6.8137 (2.6); 3.7177 (7.1); 3.7073
(7.1); 3.3503 (16.0); 2.5341 (1.1); 2.5282 (2.2); 2.5222 (3.0); 2.5161 (2.2); 2.5103 (1.0); 2.0091 (0.6); 1.2975 (1.1);
1.2841 (0.8); 1.2655 (0.5); 1.2401 (0.7); 1.2274 (1.0); 1.1942 (0.5); 0.8773 (0.4); 0.0192 (2.4)
I.244: ¹H-NMR(400.2 MHz, d₆-DMSO):
δ = 8.8759 (3.4); 8.8536 (3.5); 8.8377 (2.8); 8.8308 (9.7); 8.8217 (9.2); 8.8149 (2.6); 7.9567 (2.4); 7.6444 (2.9); 7.5161
(7.2); 7.4844 (4.6); 7.4794 (2.2); 7.4704 (5.4); 7.4627 (5.8); 7.4539 (2.5); 7.4488 (5.0); 7.3879 (3.4); 7.1526 (5.2);
7.1303 (9.4); 7.1082 (4.7); 4.8607 (2.2); 4.8360 (3.3); 4.8127 (2.3); 3.3444 (50.6); 2.8944 (16.0); 2.7351 (13.8); 2.5303
(0.8); 2.5168 (16.6); 2.5125 (32.8); 2.5080 (42.8); 2.5035 (31.8); 2.4991 (15.9); 2.4030 (0.9); 2.3823 (1.6); 2.3583
(1.6); 2.3373 (1.2); 1.8872 (1.0); 1.8760 (1.4); 1.8653 (1.0); 1.8572 (1.6); 1.8456 (1.0); 1.8270 (0.5); 1.6494 (0.4);
1.6119 (2.1); 1.6077 (2.2); 1.5981 (2.4); 1.5923 (2.4); 1.5809 (2.2); 1.5701 (2.1); 1.5586 (1.5); 1.5466 (1.8); 1.5254
(1.8); 1.5069 (1.5); 1.4875 (1.1); 1.4689 (1.0); 1.4565 (1.2); 1.4486 (1.4); 1.4406 (1.1); 1.4281 (1.1); 1.4220 (0.8);
1.4084 (0.4); 1.3910 (0.6); 1.3719 (1.2); 1.3518 (1.4); 1.3407 (1.4); 1.3204 (1.2); 1.3006 (0.8); 1.2825 (1.0); 1.2705
(1.3); 1.2643 (1.1); 1.2513 (1.6); 1.2328 (0.9); 1.1640 (0.6); 1.1438 (1.4); 1.1331 (0.9); 1.1229 (1.5); 1.1122 (1.2);
1.1024 (0.8); 1.0914 (0.9)
I.245: ¹H-NMR(400.2 MHz, d₆-DMSO):
δ = 8.9010 (3.1); 8.8788 (3.2); 8.8005 (3.2); 8.7932 (4.9); 8.7490 (4.8); 8.7417 (3.1); 7.9542 (2.4); 7.6240 (2.6); 7.4956
(6.7); 7.4812 (6.1); 7.4631 (7.7); 7.3674 (3.1); 7.3444 (4.2); 7.3265 (12.0); 7.3080 (10.9); 7.2595 (4.6); 7.2413 (10.0);
7.2256 (6.3); 7.2223 (5.5); 7.2077 (1.5); 7.1653 (2.8); 7.1471 (3.8); 7.1289 (1.4); 5.3923 (0.9); 5.3790 (1.2); 5.3686
(1.8); 5.3560 (1.9); 5.3454 (1.1); 5.3320 (1.0); 3.3488 (76.8); 3.1709 (1.3); 3.1457 (1.4); 3.1366 (2.6); 3.1116 (2.5);
3.0769 (2.4); 3.0635 (2.7); 3.0425 (1.4); 3.0293 (1.2); 2.8905 (16.0); 2.7331 (13.5); 2.7323 (13.8); 2.5276 (0.8); 2.5139
(16.3); 2.5098 (31.8); 2.5053 (41.0); 2.5008 (30.1); 2.4964 (14.8)
I.246: ¹H-NMR(400.2 MHz, d₆-DMSO):
δ = 9.0524 (7.8); 8.9231 (3.8); 8.9158 (5.8); 8.8756 (5.8); 8.8683 (3.7); 7.9545 (2.4); 7.6544 (3.0); 7.5261 (7.3); 7.3980
(3.4); 7.3703 (3.8); 7.3266 (3.1); 7.3067 (7.0); 7.2868 (4.2); 7.1847 (7.8); 7.0598 (3.9); 7.0583 (3.8); 7.0385 (3.5);
6.9992 (3.9); 6.9694 (2.9); 6.9643 (3.5); 6.9491 (2.4); 6.9443 (3.4); 6.9289 (5.0); 6.9244 (6.4); 3.3424 (59.0); 2.8930
(16.0); 2.7340 (13.8); 2.5142 (18.6); 2.5100 (35.2); 2.5055 (45.1); 2.5010 (33.4); 2.4968 (16.7); 1.3988 (1.9); 1.3776
(6.8); 1.3676 (3.9); 1.3278 (4.1); 1.3182 (7.0); 1.2977 (1.7)
I.247: ¹H-NMR(400.2 MHz, d₆-DMSO):
δ = 9.0540 (8.2); 8.9049 (3.9); 8.8978 (6.8); 8.8710 (6.8); 8.8640 (3.8); 7.9543 (2.7); 7.6523 (2.9); 7.5240 (6.7); 7.3958
(3.3); 7.3382 (3.4); 7.2455 (10.1); 7.2235 (13.2); 7.1525 (7.0); 7.0748 (11.0); 7.0531 (8.8); 6.9668 (3.5); 3.3470 (83.5);
2.8928 (16.0); 2.7336 (14.5); 2.5099 (36.3); 2.5056 (46.1); 2.5013 (35.4); 1.3430 (1.5); 1.3197 (8.3); 1.2881 (7.5);
1.2823 (8.3); 1.2596 (1.4)
I.248: ¹H-NMR(400.2 MHz, d₆-DMSO):
δ = 8.8716 (3.6); 8.8211 (3.5); 8.7564 (8.5); 7.9537 (2.5); 7.7538 (4.5); 7.7497 (4.9); 7.7348 (5.0); 7.7306 (5.1); 7.6409
(3.5); 7.5126 (8.4); 7.4318 (4.0); 7.3844 (4.0); 7.3058 (1.9); 7.3016 (2.0); 7.2860 (4.2); 7.2825 (4.4); 7.2670 (3.4);
7.2628 (3.3); 7.2468 (8.5); 7.1781 (3.1); 7.1756 (3.7); 7.1592 (5.2); 7.1567 (6.0); 7.1340 (6.1); 7.1134 (4.5); 7.0618
(4.2); 3.3477 (165.2); 2.8926 (16.0); 2.7334 (13.8); 2.6744 (0.4); 2.5139 (27.9); 2.5098 (53.9); 2.5053 (69.6); 2.5009
(52.3); 2.3365 (0.3); 2.3321 (0.4); 2.3275 (0.3); 1.2466 (1.1); 1.2202 (12.4); 1.2018 (12.2); 1.1753 (1.2)
I.249: ¹H-NMR(400.2 MHz, d₆-DMSO):
δ = 9.1165 (9.8); 8.9352 (4.7); 8.9280 (6.8); 8.8791 (6.7); 8.8719 (4.6); 7.9564 (0.5); 7.6538 (3.8); 7.5254 (13.4); 7.5210
(6.2); 7.5101 (16.0); 7.4980 (6.6); 7.4910 (4.3); 7.4833 (9.2); 7.3974 (4.3); 3.3441 (57.6); 2.8946 (3.6); 2.7355 (3.0);
2.6766 (0.3); 2.5164 (22.4); 2.5122 (42.8); 2.5077 (55.0); 2.5032 (40.6); 2.4989 (20.2); 2.3346 (0.3); 1.4595 (2.6);
1.4391 (8.9); 1.4280 (4.3); 1.4020 (1.1); 1.3899 (1.2); 1.3637 (4.4); 1.3528 (9.1); 1.3329 (2.3)
I.250: ¹H-NMR(400.2 MHz, d₆-DMSO):
δ = 9.1129 (14.6); 8.9369 (7.6); 8.9295 (10.2); 8.8638 (10.0); 8.8564 (7.5); 7.6544 (5.6); 7.6213 (13.7); 7.6004 (16.0);
7.5262 (14.1); 7.3980 (6.7); 7.3645 (15.3); 7.3440 (13.5); 3.3495 (174.8); 2.8940 (2.2); 2.7346 (1.8); 2.6806 (0.4);
2.6761 (0.5); 2.6715 (0.4); 2.5294 (1.6); 2.5159 (31.0); 2.5116 (61.5); 2.5071 (80.7); 2.5026 (60.1); 2.4982 (30.0);
2.3384 (0.3); 2.3339 (0.5); 2.3295 (0.4); 1.4668 (3.3); 1.4456 (12.2); 1.4357 (7.0); 1.4013 (3.9); 1.3943 (6.9); 1.3848
(12.6); 1.3643 (3.0); −0.0002 (0.3)
I.251: ¹H-NMR(400.2 MHz, d₆-DMSO):
δ = 8.9837 (4.0); 8.9145 (1.8); 8.8394 (1.8); 8.3175 (0.4); 7.9540 (1.6); 7.8357 (4.1); 7.8292 (4.3); 7.6482 (1.5); 7.5200
(3.6); 7.4262 (3.0); 7.4049 (5.1); 7.3918 (1.8); 7.3472 (2.7); 7.3409 (2.7); 7.3260 (1.6); 7.3198 (1.6); 3.3434 (82.1);
3.3193 (0.6); 2.8928 (9.6); 2.7332 (8.7); 2.5096 (37.0); 2.5053 (47.7); 2.5010 (36.7); 1.2551 (16.0)
I.252: ¹H-NMR(400.2 MHz, d₆-DMSO):
δ = 9.0676 (4.2); 8.9269 (1.8); 8.9200 (2.9); 8.8818 (2.9); 8.8749 (1.8); 7.9560 (2.5); 7.6569 (1.6); 7.5286 (4.1); 7.5197
(4.1); 7.4985 (4.5); 7.4005 (1.9); 7.3771 (4.7); 7.3715 (4.8); 7.1881 (2.7); 7.1824 (2.5); 7.1668 (2.4); 7.1612 (2.3);
3.3487 (52.0); 3.1796 (0.4); 3.1665 (0.4); 2.8947 (16.0); 2.7358 (14.0); 2.7348 (13.5); 2.5299 (0.5); 2.5163 (10.6);
2.5121 (19.9); 2.5076 (25.2); 2.5030 (18.4); 2.4987 (9.0); 1.4309 (1.1); 1.4107 (3.7); 1.3995 (1.8); 1.3677 (0.4); 1.3615
(0.5); 1.3289 (1.8); 1.3180 (3.9); 1.3136 (3.3); 1.2981 (1.0)
I.253: ¹H-NMR(400.2 MHz, d₆-DMSO):
δ = 9.0038 (14.4); 8.8799 (6.8); 8.8313 (6.8); 8.3188 (0.4); 7.9555 (1.8); 7.8287 (8.2); 7.8251 (8.6); 7.8094 (9.3); 7.8058
(9.0); 7.6455 (5.8); 7.5245 (8.7); 7.5209 (11.6); 7.5174 (16.0); 7.5046 (10.0); 7.5011 (9.7); 7.3891 (6.6); 7.3291 (8.3);
7.3093 (13.9); 7.2897 (6.6); 3.3445 (186.8); 2.8939 (11.5); 2.7344 (10.2); 2.6803 (0.6); 2.6759 (0.8); 2.6714 (0.6);
2.5113 (97.3); 2.5069 (122.1); 2.5024 (89.7); 2.3380 (0.6); 2.3337 (0.7); 2.3294 (0.5); 1.3043 (2.7); 1.2811 (15.9);
1.2504 (14.0); 1.2438 (15.3); 1.2211 (2.8); −0.0001 (0.5)
I.254: ¹H-NMR(300.2 MHz, d₆-DMSO):
δ = 9.0750 (0.5); 7.5488 (0.5); 7.4117 (0.5); 7.2092 (1.2); 7.2029 (1.2); 3.3464 (16.0); 2.5340 (2.7); 2.5280 (5.8); 2.5219
(8.0); 2.5158 (5.7); 2.5099 (2.6); 1.4736 (0.4); 1.3391 (0.4); 1.3322 (0.3); 0.0199 (7.4)
I.255: ¹H-NMR(300.2 MHz, d₆-DMSO):
δ = 8.9840 (1.8); 8.8891 (0.7); 8.8500 (0.7); 7.8714 (1.6); 7.8435 (1.8); 7.7085 (0.7); 7.5470 (1.9); 7.5394 (2.8); 7.3980
(1.2); 7.3907 (1.1); 7.3701 (1.2); 7.3667 (1.2); 7.3630 (1.1); 3.3467 (16.0); 2.5340 (3.6); 2.5280 (7.9); 2.5220 (10.9);
2.5159 (7.8); 2.5099 (3.6); 1.3028 (0.5); 1.2660 (3.5); 1.2315 (1.6); 0.8994 (0.6); 0.8775 (2.1); 0.8543 (0.7); 0.0301
(0.4); 0.0193 (12.8); 0.0083 (0.4)
I.256: ¹H-NMR(400.2 MHz, d₆-DMSO):
δ = 8.8995 (0.5); 8.7026 (2.3); 8.6277 (0.5); 7.9565 (0.6); 7.6884 (0.8); 7.6833 (0.6); 7.6768 (0.8); 7.6726 (1.1); 7.6667
(0.8); 7.6433 (2.1); 7.6248 (1.0); 7.5477 (0.3); 7.5352 (3.6); 7.5197 (1.3); 7.4966 (2.5); 7.3684 (1.1); 3.3475 (26.7);
2.8946 (4.2); 2.7360 (3.5); 2.7350 (3.6); 2.5304 (0.4); 2.5167 (6.4); 2.5124 (12.3); 2.5079 (15.9); 2.5034 (11.7); 2.4990
(5.8); 1.7470 (16.0)
I.257: ¹H-NMR(400.2 MHz, d₆-DMSO):
δ = 8.8915 (4.0); 8.8414 (7.7); 8.8204 (4.2); 7.6490 (3.4); 7.5207 (8.6); 7.4853 (1.7); 7.4662 (6.2); 7.4479 (13.4); 7.4333
(1.2); 7.4298 (1.4); 7.4250 (0.6); 7.3923 (7.1); 7.3859 (5.9); 7.2246 (2.9); 7.2224 (3.0); 7.2134 (1.7); 7.2051 (2.4);
5.3150 (0.6); 5.2974 (2.3); 5.2786 (3.1); 5.2597 (2.3); 5.2422 (0.6); 3.3513 (103.6); 3.3280 (0.5); 2.5311 (0.9); 2.5177
(18.5); 2.5133 (36.4); 2.5088 (47.3); 2.5042 (34.7); 2.4998 (17.0); 1.5098 (16.0); 1.4922 (15.8)
I.258: ¹H-NMR(400.2 MHz, d₆-DMSO):
δ = 8.8881 (1.8); 8.6509 (1.8); 8.3747 (4.0); 7.9560 (2.4); 7.6316 (1.8); 7.5033 (4.7); 7.4202 (2.5); 7.4158 (4.5); 7.4113
(3.0); 7.3850 (1.8); 7.3809 (1.3); 7.3752 (2.5); 7.3682 (2.4); 7.3650 (3.0); 7.3616 (2.0); 7.3291 (2.5); 7.3095 (4.4);
7.2899 (2.2); 7.2333 (2.0); 7.2308 (2.5); 7.2288 (2.4); 7.2259 (2.0); 7.2139 (1.4); 7.2113 (1.6); 7.2092 (1.6); 7.2066
(1.3); 3.3479 (53.8); 2.8941 (16.0); 2.7357 (13.7); 2.7345 (13.6); 2.5927 (1.3); 2.5615 (1.5); 2.5300 (0.7); 2.5164
(11.2); 2.5120 (21.9); 2.5075 (28.4); 2.5030 (21.0); 2.4986 (10.3); 1.7861 (0.7); 1.7741 (0.9); 1.7460 (1.6); 1.7260
(1.0); 1.7142 (1.0); 1.6615 (0.9); 1.6240 (1.5); 1.5697 (3.1); 1.3307 (0.5); 1.3182 (0.7); 1.3046 (0.6); 1.2903 (0.6);
1.2793 (0.4)
I.259: ¹H-NMR(400.2 MHz, d₆-DMSO):
δ = 9.1122 (5.4); 8.9419 (2.3); 8.9355 (3.2); 8.8844 (3.2); 8.8778 (2.3); 7.9556 (2.5); 7.7873 (5.8); 7.7816 (5.8); 7.6608
(5.0); 7.6396 (6.0); 7.5282 (4.7); 7.5084 (3.5); 7.5027 (3.3); 7.4871 (2.8); 7.4813 (2.7); 7.4002 (2.2); 3.3506 (73.2);
2.8950 (16.0); 2.7354 (13.8); 2.5299 (0.7); 2.5163 (13.7); 2.5122 (25.8); 2.5077 (32.8); 2.5032 (24.5); 1.4991 (1.4);
1.4789 (4.8); 1.4675 (2.2); 1.4294 (0.7); 1.3913 (2.2); 1.3800 (4.9); 1.3760 (4.4); 1.3602 (1.3)
I.260: ¹H-NMR(400.2 MHz, d₆-DMSO):
δ = 8.9101 (3.5); 8.8903 (6.6); 8.8858 (6.2); 8.8159 (4.7); 8.8088 (3.5); 7.9557 (2.4); 7.6443 (2.9); 7.5965 (4.4); 7.5941
(4.5); 7.5766 (5.0); 7.5742 (4.9); 7.5251 (3.8); 7.5210 (4.5); 7.5161 (7.3); 7.5057 (4.8); 7.5018 (4.6); 7.3878 (3.4);
7.3761 (2.4); 7.3739 (2.4); 7.3570 (4.4); 7.3386 (2.3); 7.3364 (2.2); 7.1882 (2.4); 7.1842 (2.5); 7.1687 (3.8); 7.1653
(3.6); 7.1501 (2.0); 7.1460 (1.9); 5.3372 (1.2); 5.3243 (1.4); 5.3153 (2.5); 5.3028 (2.3); 5.2941 (1.6); 5.2810 (1.2);
3.3515 (88.2); 2.8940 (15.8); 2.7350 (13.5); 2.5299 (0.8); 2.5163 (17.0); 2.5122 (32.1); 2.5078 (40.9); 2.5033 (30.1);
2.4991 (14.8); 1.8356 (0.3); 1.8202 (0.8); 1.8012 (2.2); 1.7883 (3.4); 1.7829 (2.6); 1.7700 (3.5); 1.7484 (2.2); 1.7358
(0.6); 1.7313 (0.9); 1.7137 (0.5); 1.0103 (7.5); 0.9922 (16.0); 0.9739 (6.9)
I.261: ¹H-NMR(300.2 MHz, CDCl₃):
δ = 9.8798 (0.6); 9.1130 (1.1); 9.0215 (1.1); 8.8341 (0.4); 7.9209 (2.4); 7.9151 (2.7); 7.8956 (5.1); 7.8895 (5.4); 7.8699
(2.8); 7.8638 (2.8); 7.2987 (17.5); 7.2876 (1.4); 7.2763 (1.4); 7.2692 (2.9); 7.2618 (2.6); 7.2512 (2.4); 7.2425 (3.4);
7.2358 (2.1); 7.2246 (2.0); 7.2184 (1.9); 7.1162 (3.7); 7.1119 (5.0); 7.1074 (5.9); 7.0911 (5.6); 7.0868 (7.2); 7.0776
(4.3); 7.0659 (3.0); 7.0617 (3.2); 7.0507 (3.1); 7.0466 (2.8); 7.0412 (3.8); 7.0372 (3.2); 7.0140 (2.7); 7.0100 (2.5);
6.9475 (0.3); 6.9351 (11.2); 6.7629 (5.5); 6.5173 (0.7); 6.5042 (1.4); 6.4887 (0.7); 6.3248 (1.4); 6.3121 (3.0); 6.3093
(2.8); 6.2966 (1.4); 6.1329 (0.7); 6.1200 (1.5); 6.1173 (1.4); 6.1045 (0.7); 4.3010 (3.1); 4.2856 (3.3); 4.2567 (6.4);
4.2413 (6.3); 4.2124 (3.6); 4.1969 (3.3); 1.6057 (16.0); 1.5545 (6.0); 1.4613 (4.7); 1.3940 (0.7); 1.3674 (0.5); 1.3554
(0.4); 1.3416 (0.4); 1.3317 (0.4); 1.2929 (2.0); 1.2724 (0.5); 1.2493 (0.7); 1.2261 (0.4); 0.9193 (0.7); 0.8959 (0.4);
0.1087 (4.7); 0.0487 (0.7); 0.0377 (21.8); 0.0266 (0.9)
I.262: ¹H-NMR(400.2 MHz, d₆-DMSO):
δ = 9.0766 (9.5); 8.9334 (4.4); 8.9263 (6.7); 8.8820 (6.5); 8.8749 (4.3); 7.9558 (2.4); 7.6545 (3.6); 7.5263 (9.0); 7.4166
(3.9); 7.3969 (11.9); 7.3765 (5.6); 7.1978 (4.9); 7.1964 (4.9); 7.1763 (4.3); 7.1524 (3.8); 7.1503 (3.7); 7.1320 (3.3);
7.1299 (3.3); 7.0987 (6.7); 3.3477 (111.1); 2.8940 (16.0); 2.7346 (14.0); 2.6759 (0.4); 2.5295 (1.1); 2.5157 (23.2);
2.5115 (45.4); 2.5070 (59.3); 2.5025 (44.1); 2.4983 (22.0); 2.3340 (0.4); 1.4242 (2.3); 1.4028 (8.4); 1.3927 (5.0);
1.3544 (5.0); 1.3445 (8.6); 1.3238 (2.1)
I.263: ¹H-NMR(400.2 MHz, d₆-DMSO):
δ = 9.1517 (11.5); 8.8774 (10.2); 8.8691 (10.3); 7.9581 (0.7); 7.8521 (2.9); 7.8323 (6.2); 7.8129 (3.4); 7.6504 (4.4);
7.5916 (4.8); 7.5642 (4.8); 7.5220 (16.0); 7.5000 (4.9); 7.3940 (5.2); 3.3551 (170.6); 2.8970 (4.6); 2.7380 (3.9); 2.6794
(0.4); 2.5326 (1.3); 2.5189 (27.8); 2.5148 (52.9); 2.5103 (67.3); 2.5058 (49.0); 2.5015 (23.9); 2.3373 (0.4); 1.4158
(3.5); 1.4008 (10.2); 1.3963 (11.3); 1.3843 (5.0); 1.3450 (0.7); 1.3200 (0.6); 1.2806 (4.8); 1.2682 (10.7); 1.2645 (10.4);
1.2496 (3.2)
I.264: ¹H-NMR(400.2 MHz, d₆-DMSO):
δ = 9.1032 (15.9); 8.9129 (6.8); 8.8649 (6.9); 8.3193 (2.8); 7.9576 (0.4); 7.8310 (13.9); 7.8125 (14.0); 7.6538 (6.3);
7.6123 (10.5); 7.5870 (10.7); 7.5255 (15.1); 7.3974 (7.3); 3.3512 (209.9); 3.3280 (2.2); 2.8964 (3.0); 2.7368 (2.6);
2.6830 (0.4); 2.6784 (0.6); 2.6743 (0.4); 2.5317 (2.0); 2.5183 (39.1); 2.5140 (75.7); 2.5095 (97.6); 2.5050 (71.5);
2.5007 (35.0); 2.3409 (0.4); 2.3364 (0.6); 2.3321 (0.4); 1.3687 (5.0); 1.3538 (14.4); 1.3493 (16.0); 1.3371 (7.1); 1.2976
(0.9); 1.2692 (0.9); 1.2293 (7.2); 1.2170 (15.4); 1.2128 (14.9); 1.1980 (4.6); −0.0001 (0.4)
I.265: ¹H-NMR(400.2 MHz, d₆-DMSO):
δ = 8.9973 (4.5); 8.8697 (16.0); 7.9545 (2.0); 7.6674 (1.6); 7.5390 (3.9); 7.4108 (1.9); 7.3328 (2.8); 7.3279 (1.2); 7.3140
(4.8); 7.3116 (4.7); 7.2973 (1.7); 7.2928 (3.9); 7.2880 (2.4); 7.2680 (3.9); 7.2482 (2.2); 7.0814 (1.8); 7.0629 (3.0);
7.0445 (1.3); 6.9374 (4.4); 6.9349 (5.4); 6.9157 (4.8); 6.9137 (4.4); 6.9056 (2.5); 6.9036 (2.4); 6.8856 (1.9); 6.8837
(2.1); 6.8219 (2.5); 6.8167 (4.0); 6.8120 (2.8); 6.7818 (2.2); 6.7774 (1.7); 6.7618 (1.9); 6.7574 (1.7); 3.3507 (63.5);
2.8928 (12.8); 2.7338 (11.1); 2.5104 (22.3); 2.5060 (28.4); 2.5016 (21.3); 1.3587 (0.8); 1.3357 (4.3); 1.3294 (3.4);
1.3026 (3.8); 1.2956 (4.3); 1.2732 (0.8)
I.266: ¹H-NMR(400.2 MHz, d₆-DMSO):
δ = 8.8785 (3.6); 8.8722 (3.9); 8.7606 (8.3); 8.6578 (3.9); 8.6519 (3.5); 8.3180 (1.2); 7.9548 (0.9); 7.6444 (4.6); 7.6229
(16.0); 7.6055 (14.1); 7.5840 (4.5); 7.4983 (8.6); 7.3703 (4.0); 3.3524 (270.5); 2.8936 (6.1); 2.7347 (5.2); 2.7337 (5.1);
2.6799 (0.5); 2.6755 (0.7); 2.6709 (0.5); 2.5286 (3.9); 2.5153 (43.6); 2.5110 (81.1); 2.5064 (104.0); 2.5019 (76.8);
2.4976 (38.6); 2.3377 (0.4); 2.3332 (0.6); 2.3287 (0.4); 2.0929 (2.9); 2.0765 (2.8); 2.0605 (2.6); 2.0471 (1.6); 1.8576
(1.1); 1.8264 (3.0); 1.8183 (3.9); 1.8093 (4.2); 1.7885 (4.7); 1.7809 (3.7); 1.7707 (3.6); 1.7519 (1.4); −0.0002 (0.4)
I.267: ¹H-NMR(300.2 MHz, CDCl₃):
δ = 9.4229 (16.0); 7.8677 (0.9); 7.8616 (0.9); 7.8420 (1.8); 7.8359 (1.9); 7.8165 (1.0); 7.8103 (1.0); 7.3152 (0.4); 7.3091
(0.5); 7.2988 (4.4); 7.2909 (1.0); 7.2843 (0.8); 7.2733 (0.8); 7.2669 (0.9); 7.2638 (1.1); 7.2571 (0.7); 7.2463 (0.7);
7.2401 (0.7); 7.1840 (1.4); 7.1795 (1.9); 7.1766 (2.2); 7.1586 (2.0); 7.1546 (2.1); 7.1336 (0.9); 7.1295 (0.9); 7.0047
(3.9); 6.9234 (1.1); 6.9195 (1.1); 6.8960 (1.0); 6.8921 (1.0); 6.8867 (1.2); 6.8827 (1.1); 6.8594 (1.0); 6.8555 (0.9);
6.8331 (2.0); 1.8696 (1.0); 1.8479 (3.2); 1.8437 (3.3); 1.8227 (1.4); 1.6138 (4.2); 1.5618 (1.4); 1.5418 (3.6); 1.5368
(3.4); 1.5154 (1.0); 1.2958 (0.3); 0.0360 (4.5)
I.268: ¹H-NMR(400.2 MHz, d₆-DMSO):
δ = 8.8622 (2.3); 8.8552 (2.4); 8.4850 (2.4); 8.4780 (2.3); 8.1420 (3.3); 7.9551 (2.6); 7.6629 (2.0); 7.6456 (2.2); 7.6429
(2.2); 7.6134 (1.6); 7.4850 (3.6); 7.4660 (2.3); 7.4635 (2.6); 7.4465 (2.7); 7.4439 (2.8); 7.3935 (1.2); 7.3913 (1.2);
7.3734 (2.4); 7.3565 (2.8); 7.1213 (1.3); 7.1185 (1.4); 7.1007 (2.3); 7.0835 (1.1); 7.0808 (1.1); 3.3494 (58.0); 2.8934
(16.0); 2.8470 (1.2); 2.8231 (1.3); 2.7342 (14.8); 2.5109 (23.2); 2.5067 (29.2); 2.5024 (21.8); 1.7845 (1.0); 1.6677
(1.0); 1.6366 (1.0); 1.5910 (1.8); 1.5679 (1.4); 1.2974 (0.7); 1.2678 (0.6)
IIa.01: ¹H-NMR(300.2 MHz, d₆-DMSO):
δ = 10.9458 (4.6); 10.5956 (5.9); 7.9012 (1.2); 7.8722 (1.7); 7.8575 (8.3); 7.8291 (9.2); 7.4029 (10.1); 7.3744 (9.0);
7.3466 (0.4); 7.3333 (0.4); 6.6372 (2.6); 6.4608 (6.2); 6.2847 (2.9); 4.3241 (0.8); 4.3005 (0.9); 3.8517 (1.2); 3.3459
(16.0); 2.6172 (0.3); 2.5860 (0.8); 2.5518 (47.8); 2.5344 (9.2); 2.5285 (17.4); 2.5224 (22.6); 2.5164 (16.2); 2.5104
(7.8); 2.4854 (0.5); 1.3762 (0.8); 1.3550 (1.0); 1.3312 (1.9); 1.3075 (1.0); 1.2566 (0.4); 0.0316 (0.6); 0.0208 (14.1);
0.0098 (0.6); −0.0398 (0.7)
IIa.02: ¹H-NMR(300.2 MHz, d₆-DMSO):
δ = 10.2416 (0.6); 7.8655 (0.8); 7.8362 (1.2); 7.7234 (1.2); 7.6943 (0.8); 6.4457 (0.7); 6.2695 (0.3); 3.3447 (16.0);
2.5346 (2.6); 2.5286 (5.6); 2.5225 (7.7); 2.5165 (5.6); 2.5105 (2.6); 2.1002 (4.1); 0.0211 (6.0)
IIa.03: ¹H-NMR(400.2 MHz, d₆-DMSO):
δ = 10.9418 (0.9); 8.0995 (3.4); 8.0784 (5.5); 8.0096 (5.4); 7.9885 (3.4); 6.5885 (1.1); 6.4561 (2.7); 6.3239 (1.3); 3.8936
(16.0); 3.3353 (3.3); 2.5073 (3.0); 2.5029 (4.0); 2.4986 (2.9); −0.0015 (2.0)
IIa.04: ¹H-NMR(400.2 MHz, d₆-DMSO):
δ = 10.9926 (6.8); 8.1366 (0.4); 8.0900 (10.2); 8.0688 (16.0); 8.0442 (4.2); 8.0354 (1.4); 8.0142 (1.9); 7.9905 (15.2);
7.9693 (10.1); 7.9411 (1.6); 7.9199 (1.1); 3.3685 (0.8); 2.8914 (0.4); 2.7326 (0.3); 2.5130 (6.3); 2.5089 (12.1); 2.5044
(16.0); 2.5000 (11.6); 2.0761 (2.4); 0.0081 (0.5); 0.0000 (9.5); −0.0082 (0.4)
IIa.05: ¹H-NMR(300.2 MHz, d₆-DMSO):
δ = 8.9811 (0.7); 8.9772 (0.7); 8.1271 (3.9); 8.0564 (0.7); 8.0526 (0.7); 6.4885 (0.5); 3.3454 (16.0); 2.5346 (1.0); 2.5286
(2.0); 2.5226 (2.8); 2.5165 (2.0); 2.5107 (1.0); 0.0206 (2.4)
IIa.06: ¹H-NMR(400.2 MHz, d₆-DMSO):
δ = 11.0278 (3.3); 8.1348 (0.8); 8.1149 (3.6); 8.0938 (5.5); 8.0125 (5.3); 7.9914 (3.5); 3.8964 (16.0); 3.3734 (32.0);
2.5085 (4.4); 2.5042 (5.9); 2.4998 (4.4); −0.0015 (2.9)
IIa.07: ¹H-NMR(300.2 MHz, d₆-DMSO):
δ = 10.9993 (0.6); 10.7085 (1.8); 8.0201 (5.7); 7.9920 (8.2); 7.8683 (8.4); 7.8402 (5.7); 7.7933 (3.9); 7.7886 (5.4);
7.7650 (6.2); 7.5551 (2.8); 7.5318 (6.5); 7.5063 (4.4); 7.4700 (2.8); 7.4535 (1.0); 7.4461 (3.2); 7.4377 (0.7); 7.4218
(0.9); 6.6574 (2.0); 6.4808 (5.1); 6.3048 (2.3); 3.3454 (16.0); 3.1199 (1.0); 3.0957 (1.0); 2.5344 (5.9); 2.5284 (12.1);
2.5224 (16.3); 2.5164 (11.7); 2.5106 (5.4); 1.2153 (1.0); 1.1911 (2.1); 1.1667 (1.0); 0.0314 (0.4); 0.0205 (11.2); 0.0097 (0.4)
IIa.08: ¹H-NMR(300.2 MHz, d₆-DMSO):
δ = 7.8569 (0.7); 7.8348 (0.4); 7.8279 (1.6); 7.8213 (0.3); 7.7817 (1.6); 7.7748 (0.4); 7.7527 (0.7); 6.6455 (0.3); 6.4692
(0.8); 6.2932 (0.4); 3.3473 (16.0); 2.5345 (1.0); 2.5285 (2.1); 2.5224 (2.9); 2.5163 (2.0); 2.5104 (0.9); 0.0205 (3.3)
IIa.09: ¹H-NMR(300.2 MHz, d₆-DMSO):
δ = 10.9870 (1.0); 10.9796 (1.0); 10.6380 (1.4); 7.8913 (7.7); 7.8638 (8.6); 7.8022 (7.0); 7.3883 (7.7); 7.3609 (6.9);
7.2284 (7.4); 6.9505 (7.4); 6.6349 (2.4); 6.4585 (5.7); 6.2824 (2.7); 5.3065 (15.2); 3.3465 (16.0); 2.5286 (19.9); 2.5226
(26.6); 2.5167 (19.7); 2.0104 (0.4); 0.0321 (0.9); 0.0214 (17.1)
IIa.10: ¹H-NMR(400.2 MHz, d₆-DMSO):
δ = 10.8848 (6.6); 10.4276 (6.5); 10.0238 (7.6); 8.5646 (15.3); 8.5527 (16.0); 7.9156 (6.7); 7.8934 (12.6); 7.8457
(11.3); 7.8236 (6.8); 6.9511 (4.4); 6.9392 (7.6); 6.9272 (4.4); 6.5744 (2.3); 6.4419 (5.7); 6.3097 (2.7); 3.7167 (0.4);
3.7006 (0.4); 3.6654 (0.8); 3.6490 (1.4); 3.6231 (2.5); 3.3736 (2.3); 2.5083 (20.7); 2.5040 (26.8); 2.4997 (20.7); 0.0000 (3.0)
IIa.11: ¹H-NMR(300.2 MHz, CDCl₃):
δ = 9.6674 (0.7); 9.5446 (0.4); 8.2710 (2.2); 8.2646 (1.8); 8.2595 (4.1); 8.2536 (2.0); 8.2488 (1.6); 8.2424 (3.2); 8.2369
(2.6); 8.2309 (5.2); 8.0500 (3.8); 8.0446 (5.8); 8.0215 (3.7); 8.0161 (4.3); 7.2986 (6.2); 6.3325 (1.3); 6.1766 (2.6);
6.1548 (3.1); 5.9997 (5.3); 5.9772 (1.5); 5.8227 (2.6); 2.9441 (2.2); 2.9188 (6.9); 2.8935 (7.2); 2.8683 (2.4); 1.4609
(7.8); 1.4356 (16.0); 1.4104 (7.3); 1.2889 (2.2); 0.0453 (0.4); 0.0346 (6.3)
IIa.12: ¹H-NMR(400.2 MHz, d₆-DMSO):
δ = 10.8403 (0.6); 8.7135 (1.4); 8.7005 (2.5); 8.6892 (1.2); 7.9810 (1.8); 7.9588 (16.0); 7.9545 (15.7); 7.9323 (1.7);
6.5920 (1.7); 6.4596 (4.2); 6.3274 (1.9); 3.4936 (1.0); 3.4868 (1.8); 3.4737 (7.0); 3.4637 (8.3); 3.4490 (5.5); 3.4360
(3.6); 3.3457 (33.2); 3.2739 (38.0); 2.5079 (9.7); 2.5035 (12.7); 2.4991 (9.2); 0.0082 (0.3); 0.0001 (7.1)
IIa.13: ¹H-NMR(300.1 MHz, d₆-DMSO):
δ = 10.8626 (0.9); 10.6901 (0.3); 10.4328 (0.4); 10.3176 (1.1); 10.2443 (0.4); 8.5063 (0.6); 8.4705 (11.8); 7.7689
(14.6); 7.7399 (16.0); 7.4098 (2.9); 7.4038 (3.1); 7.3827 (5.6); 7.3771 (5.9); 7.3552 (3.7); 7.3497 (3.6); 7.3155 (3.2);
7.3102 (3.3); 7.2890 (4.2); 7.2835 (4.6); 7.2774 (3.5); 7.2723 (3.5); 7.2504 (4.0); 7.2454 (4.5); 7.2026 (3.0); 7.1976
(3.2); 7.1775 (7.0); 7.1725 (6.2); 7.1519 (4.8); 7.1465 (4.0); 7.1263 (3.0); 7.1200 (3.2); 7.1102 (3.2); 7.1040 (3.9);
7.0945 (3.3); 7.0845 (3.2); 7.0788 (3.1); 7.0695 (1.3); 7.0585 (1.3); 7.0529 (1.2); 6.9535 (13.3); 6.9258 (12.4); 6.5922
(4.2); 6.4154 (11.2); 6.2390 (5.0); 3.3303 (14.9); 2.7279 (0.4); 2.5138 (23.4); 2.5079 (45.1); 2.5019 (59.5); 2.4960
(40.7); 2.4903 (18.6); 2.2784 (0.3); 2.2718 (0.4); 2.0754 (0.3); 0.0108 (1.6); −0.0001 (39.4); −0.0111 (1.2)
IIa.14: ¹H-NMR(300.2 MHz, CDCl₃):
δ = 9.3656 (1.0); 8.2856 (5.7); 8.2575 (6.9); 8.0537 (7.4); 8.0252 (5.6); 7.2985 (7.7); 5.3362 (0.4); 2.9502 (2.2); 2.9249
(7.0); 2.8997 (7.2); 2.8745 (2.4); 1.4656 (7.8); 1.4403 (16.0); 1.4150 (7.3); 1.2895 (2.3); 0.0462 (0.4); 0.0356 (8.3)
IIa.15: ¹H-NMR(300.1 MHz, d₆-DMSO):
δ = 10.9277 (5.2); 8.7130 (1.3); 8.6956 (2.6); 7.9975 (2.1); 7.9902 (1.6); 7.9684 (16.0); 7.9597 (15.8); 7.9307 (2.1);
7.8929 (0.9); 3.5042 (0.8); 3.4917 (1.5); 3.4751 (7.8); 3.4638 (12.8); 3.4549 (6.0); 3.4356 (3.5); 3.4201 (1.2); 3.4087
(0.5); 3.3296 (18.3); 3.2749 (40.2); 2.5074 (9.4); 2.5018 (12.3); 2.4961 (8.7); −0.0002 (6.8)
IIa.16: ¹H-NMR(300.2 MHz, CDCl₃):
δ = 7.6907 (1.4); 7.6632 (1.8); 7.4360 (2.2); 7.4081 (1.8); 7.3260 (0.6); 7.2987 (34.3); 7.2785 (2.1); 7.2660 (1.3); 7.2437
(0.9); 7.0959 (1.6); 7.0730 (1.3); 6.2956 (0.8); 6.2847 (0.4); 6.1779 (7.7); 6.1176 (2.7); 6.1100 (2.0); 6.0771 (2.6);
6.0613 (2.6); 6.0012 (16.0); 5.9400 (1.1); 5.9355 (0.8); 5.8245 (7.6); 3.5801 (10.0); 1.4070 (0.4); 1.2946 (0.6); 0.0488
(1.1); 0.0379 (34.5); 0.0269 (1.2)
IIa.17: ¹H-NMR(300.2 MHz, d₆-DMSO):
δ = 10.9168 (0.4); 10.8791 (0.4); 10.8716 (0.4); 8.0503 (0.5); 8.0205 (3.4); 7.8891 (0.8); 6.6656 (0.4); 6.4899 (0.9);
6.3132 (0.4); 3.3524 (16.0); 2.5273 (6.5); 2.5218 (7.1); 1.1831 (0.5); 0.0264 (2.1); 0.0133 (1.2)
IIa.18: ¹H-NMR(300.2 MHz, d₆-DMSO):
δ = 10.9778 (0.4); 10.6587 (0.4); 7.9313 (2.7); 7.9037 (3.2); 7.6062 (2.9); 7.5788 (2.3); 7.3796 (3.5); 7.3721 (1.1);
7.3570 (1.2); 7.3494 (4.2); 7.3377 (0.4); 7.0972 (0.4); 7.0856 (4.2); 7.0780 (1.2); 7.0628 (1.1); 7.0554 (3.2); 6.6422
(0.9); 6.4655 (2.3); 6.2894 (1.0); 5.2230 (5.3); 3.3416 (16.0); 2.5341 (4.3); 2.5281 (9.0); 2.5221 (12.2); 2.5160 (8.7);
2.5102 (4.0); 0.0315 (0.4); 0.0207 (11.6); 0.0130 (0.4); 0.0115 (0.3); 0.0098 (0.4)
IIa.19: ¹H-NMR(300.2 MHz, CDCl₃):
δ = 9.2538 (0.6); 7.6139 (3.8); 7.5880 (4.6); 7.5027 (0.6); 7.3648 (4.9); 7.3386 (4.4); 7.3135 (2.6); 7.2984 (24.4); 7.2661
(6.1); 7.2348 (3.1); 7.2100 (1.4); 7.1482 (0.6); 7.0823 (4.2); 7.0590 (3.9); 6.9474 (0.4); 6.9269 (0.3); 5.3382 (0.6);
3.5612 (16.0); 2.4525 (0.4); 2.4278 (0.7); 2.4023 (0.5); 2.3344 (0.5); 2.3017 (0.5); 2.2854 (0.4); 2.2725 (0.4); 2.2562
(0.5); 2.2285 (0.4); 2.0688 (0.4); 2.0582 (0.4); 2.0207 (0.4); 1.9535 (0.4); 1.8612 (0.4); 1.8048 (0.5); 1.7881 (0.5);
1.7321 (0.6); 1.6810 (0.7); 1.6557 (0.7); 1.6412 (0.6); 1.6156 (0.7); 1.6047 (1.2); 1.5713 (0.6); 1.5388 (0.7); 1.5155
(0.7); 1.4627 (1.0); 1.4388 (1.2); 1.4148 (1.2); 1.3598 (2.5); 1.3293 (2.6); 1.2920 (12.4); 1.2710 (2.7); 1.2584 (2.2);
1.2478 (2.3); 1.1306 (0.7); 1.0118 (0.8); 0.9961 (0.9); 0.9742 (0.9); 0.9162 (3.6); 0.8924 (3.3); 0.8685 (1.8); 0.0471
(1.0); 0.0364 (21.9); 0.0271 (0.8); 0.0255 (0.9)
IIa.20: ¹H-NMR(300.1 MHz, d₆-DMSO):
δ = 11.0348 (0.4); 10.9352 (2.9); 10.8385 (0.5); 10.3106 (9.9); 8.1441 (0.5); 8.1052 (7.3); 8.0773 (15.2); 8.0295 (16.0);
8.0016 (7.8); 7.6521 (1.8); 7.6226 (3.8); 7.6017 (3.8); 7.5721 (1.9); 7.4239 (2.1); 7.4145 (2.3); 7.3860 (3.6); 7.3576
(2.1); 7.3488 (2.3); 7.1749 (2.0); 7.1703 (1.9); 7.1465 (3.6); 7.1166 (1.8); 6.6443 (3.1); 6.4676 (7.8); 6.2914 (3.6);
3.4044 (0.8); 3.3395 (259.8); 2.5025 (43.2); −0.0002 (18.6)
IIa.21: ¹H-NMR(300.1 MHz, d₆-DMSO):
δ = 11.7134 (0.7); 11.6516 (0.4); 11.0015 (3.9); 10.3228 (9.8); 8.1204 (7.5); 8.0923 (14.8); 8.0729 (3.6); 8.0357 (16.0);
8.0074 (8.3); 7.6563 (1.8); 7.6353 (2.2); 7.6266 (4.0); 7.6059 (3.9); 7.5971 (2.5); 7.5764 (2.0); 7.4257 (2.2); 7.4163
(2.4); 7.3952 (2.8); 7.3900 (3.3); 7.3860 (3.3); 7.3811 (3.1); 7.3599 (2.3); 7.3506 (2.4); 7.1816 (1.7); 7.1772 (1.9);
7.1718 (1.8); 7.1480 (3.3); 7.1428 (3.2); 7.1244 (1.6); 7.1197 (1.7); 7.1146 (1.6); 3.3468 (128.2); 2.5150 (8.7); 2.5093
(16.8); 2.5034 (22.0); 2.4975 (15.4); 2.0755 (0.4); 0.0106 (0.5); −0.0002 (12.0); −0.0112 (0.5)
IIa.22: ¹H-NMR(300.2 MHz, d₆-DMSO):
δ = 9.0461 (0.6); 9.0411 (0.6); 8.3285 (0.5); 8.3209 (0.4); 8.3008 (0.5); 8.2931 (0.5); 7.9728 (0.4); 7.9619 (0.7); 7.9586
(0.8); 7.9071 (0.6); 7.8775 (0.5); 7.3129 (0.6); 7.3037 (0.6); 6.7513 (0.6); 6.7455 (0.6); 6.7400 (0.6); 6.7341 (0.5);
6.4915 (0.8); 6.3155 (0.3); 3.5786 (0.5); 3.3463 (14.7); 3.0999 (0.5); 2.9113 (2.1); 2.7522 (1.8); 2.7097 (16.0); 2.5345
(6.0); 2.5285 (13.1); 2.5224 (18.3); 2.5163 (13.3); 2.5103 (6.3); 1.2598 (9.5); 1.2377 (10.0); 1.2101 (1.9); 0.0315 (0.6);
0.0207 (18.5); 0.0097 (0.7)
IIa.23: ¹H-NMR(300.2 MHz, d₆-DMSO):
δ = 9.2015 (1.7); 9.1955 (1.6); 8.5404 (0.9); 8.5336 (0.8); 8.5129 (1.0); 8.5062 (1.0); 8.1578 (0.4); 8.1454 (1.9); 8.1180
(1.6); 6.6767 (1.0); 6.5004 (2.4); 6.3244 (1.1); 3.6189 (0.6); 3.5985 (0.8); 3.5753 (0.6); 3.3472 (14.7); 3.1538 (0.3);
3.1289 (0.8); 3.1044 (0.8); 3.0808 (0.3); 2.5343 (5.3); 2.5283 (11.1); 2.5223 (15.1); 2.5162 (10.8); 2.5103 (5.0); 1.2670
(16.0); 1.2449 (16.0); 1.2177 (2.0); 0.0311 (0.4); 0.0203 (11.8); 0.0093 (0.5)
IIa.24: ¹H-NMR(300.2 MHz, d₆-DMSO):
δ = 12.4653 (0.6); 8.6209 (8.7); 8.6136 (9.0); 7.9204 (6.2); 7.9123 (6.0); 7.8901 (6.6); 7.8821 (6.4); 6.8596 (7.8); 6.8293
(7.5); 3.6813 (13.7); 3.6626 (16.0); 3.6452 (12.4); 3.3475 (2.7); 2.5345 (10.4); 2.5285 (22.6); 2.5225 (31.4); 2.5164
(22.7); 2.5105 (10.6); 2.0959 (0.6); 1.6580 (5.0); 1.6419 (5.3); 1.6234 (3.3); 1.5792 (4.6); 1.5612 (9.5); 1.5447 (12.1);
1.5261 (6.5); 1.5074 (2.4); 0.0308 (1.3); 0.0200 (39.4); 0.0090 (1.5)
IIa.25: ¹H-NMR(300.2 MHz, d₆-DMSO):
δ = 8.6493 (0.8); 8.6430 (0.9); 8.3134 (0.6); 8.3052 (0.6); 8.2847 (0.6); 8.2764 (0.6); 7.5047 (0.5); 7.4800 (1.1); 7.4586
(0.4); 7.4524 (0.9); 7.3116 (0.5); 7.2869 (0.7); 7.2261 (1.0); 7.2222 (1.3); 7.1972 (1.0); 7.1942 (0.9); 7.1836 (0.9);
7.1545 (0.8); 6.6221 (0.4); 6.4452 (0.9); 6.2687 (0.4); 3.3453 (16.0); 2.5347 (3.7); 2.5287 (7.8); 2.5226 (10.8); 2.5165
(7.8); 2.5106 (3.6); 0.0319 (0.4); 0.0211 (11.8); 0.0101 (0.4)
IIa.26: ¹H-NMR(300.2 MHz, d₆-DMSO):
δ = 10.4272 (0.4); 8.6620 (0.7); 8.6545 (0.7); 8.0240 (0.4); 8.0161 (0.4); 7.9940 (0.4); 7.9859 (0.4); 6.9390 (0.5); 6.9090
(0.5); 6.4505 (0.8); 6.2744 (0.4); 4.0613 (0.4); 4.0376 (0.4); 3.6728 (0.9); 3.6582 (1.5); 3.6500 (1.3); 3.6389 (1.4);
3.4609 (1.1); 3.4435 (1.2); 3.4284 (0.8); 3.3488 (16.0); 2.7097 (3.3); 2.5346 (2.9); 2.5286 (6.0); 2.5226 (8.1); 2.5165
(5.8); 2.5106 (2.7); 2.0101 (1.7); 1.4466 (14.4); 1.2744 (4.3); 1.2525 (4.6); 1.2262 (0.8); 1.2191 (0.9); 1.1952 (1.1);
1.1715 (0.5); 0.0317 (0.3); 0.0209 (8.2)
IIa.27: ¹H-NMR(300.2 MHz, d₆-DMSO):
δ = 10.9606 (0.4); 10.5886 (0.7); 8.8686 (1.4); 8.8337 (11.2); 6.6426 (1.2); 6.4662 (2.8); 6.2903 (1.4); 3.8507 (5.6);
3.8367 (9.1); 3.8208 (7.3); 3.7541 (0.5); 3.6999 (7.8); 3.6843 (9.0); 3.6386 (0.8); 3.6179 (0.7); 3.3607 (16.0); 3.1446
(0.6); 3.1207 (0.6); 2.5215 (16.6); 1.2716 (7.7); 1.2495 (8.1); 1.2221 (1.4); 1.1918 (0.4); 0.0185 (11.1)
IIa.28: ¹H-NMR(400.1 MHz, d₆-DMSO):
δ = 10.9847 (0.3); 10.6487 (0.3); 9.8183 (4.9); 8.8350 (16.0); 8.8234 (0.7); 7.6376 (1.1); 7.6327 (1.0); 7.6183 (2.1);
7.6135 (1.8); 7.6003 (1.1); 7.5936 (1.2); 7.3029 (0.7); 7.2980 (0.9); 7.2827 (1.5); 7.2781 (2.2); 7.2701 (0.8); 7.2645
(0.9); 7.2600 (1.6); 7.2571 (1.4); 7.2519 (2.7); 7.2466 (2.4); 7.2417 (1.6); 7.2346 (2.1); 7.2263 (3.7); 7.2206 (2.1);
7.2181 (2.2); 7.2078 (2.8); 7.2023 (2.3); 7.1894 (0.8); 7.1844 (0.7); 6.5868 (1.6); 6.4543 (4.3); 6.3222 (1.9); 3.3397
(8.1); 2.5121 (4.4); 2.5077 (9.2); 2.5032 (12.5); 2.4987 (8.8); 2.4943 (4.0); 0.0081 (0.4); −0.0001 (9.6)
IIa.29: ¹H-NMR(400.1 MHz, d₆-DMSO):
δ = 10.7983 (2.2); 9.8591 (5.0); 8.8400 (16.0); 8.8199 (1.0); 7.6328 (1.1); 7.6281 (1.1); 7.6133 (2.1); 7.6088 (2.0);
7.5950 (1.2); 7.5889 (1.2); 7.3060 (0.7); 7.3013 (0.9); 7.2859 (1.6); 7.2813 (2.2); 7.2738 (1.0); 7.2704 (0.9); 7.2638
(1.3); 7.2601 (1.6); 7.2548 (2.8); 7.2526 (2.9); 7.2475 (1.8); 7.2401 (2.0); 7.2345 (2.0); 7.2291 (3.2); 7.2224 (2.4);
7.2150 (1.1); 7.2105 (2.5); 7.2051 (2.3); 7.1923 (0.8); 7.1874 (0.8); 3.3434 (5.8); 2.5126 (5.3); 2.5083 (10.9); 2.5038
(14.7); 2.4993 (10.4); 2.4950 (4.8); −0.0001 (4.7)
IIa.30: ¹H-NMR(499.9 MHz, d₆-DMSO):
δ = 10.9948 (3.4); 10.5766 (3.6); 8.8244 (4.5); 8.8121 (4.1); 8.6165 (5.2); 6.5997 (1.6); 6.4937 (3.9); 6.3879 (1.8);
4.1048 (2.8); 4.0907 (7.7); 4.0765 (7.6); 4.0624 (2.4); 3.3780 (11.5); 2.5648 (5.8); 2.5613 (7.5); 2.5578 (5.6); 2.0480
(1.1); 1.5468 (2.7); 1.5376 (6.0); 1.5311 (6.2); 1.5225 (2.3); 1.5008 (0.4); 1.2958 (0.6); 1.2485 (0.5); 1.2430 (0.4);
1.2343 (1.0); 1.2199 (1.1); 1.2118 (3.0); 1.2030 (6.3); 1.1965 (6.1); 1.1873 (2.3); 1.1461 (8.5); 1.1319 (16.0); 1.1177
(7.5); 1.1078 (0.5)
IIa.31: ¹H-NMR(300.2 MHz, CDCl₃):
δ = 9.9005 (0.6); 8.9595 (16.0); 7.4901 (2.0); 7.4850 (3.0); 7.4616 (4.1); 7.3747 (1.2); 7.3700 (1.8); 7.3639 (0.8); 7.3469
(4.2); 7.3415 (1.9); 7.3219 (2.7); 7.2986 (4.0); 7.2916 (1.2); 7.2809 (0.7); 7.2725 (2.0); 7.2627 (0.4); 7.2526 (0.4);
7.2483 (0.6); 6.2693 (1.4); 6.0914 (3.4); 5.9138 (1.6); 5.1611 (0.6); 5.1375 (2.0); 5.1138 (2.0); 5.0902 (0.6); 4.1677
(0.4); 4.1439 (0.4); 3.7396 (0.6); 2.0828 (2.0); 2.0209 (0.3); 1.9970 (0.3); 1.8076 (9.4); 1.7839 (9.3); 1.3184 (0.9);
1.3051 (1.2); 1.2948 (1.8); 1.2709 (0.6); 0.9421 (0.4); 0.9204 (1.3); 0.8971 (0.5); 0.0386 (3.0)

### PREPARATION EXAMPLES

### Preparation Example 1: 5-[5-(difluoromethyl)-1,3,4-oxadiazol-2-yl]-N-(1-phenylcyclobutyl)pyrimidin-2-amine (Compound I.134)

### Step 1: Preparation of ethyl 2-[(1-phenylcyclobutyl)amino]pyrimidine-5-carboxylate

To a solution of 2.53 g (13.6 mmol) of ethyl 2-chloropyrimidine-5-carboxylate and 2.00 g (13.6 mmol) of 1-phenylcyclobutanamine in 15 mL of 1,4-dioxane were added 5.27 g (40.8 mmol) of N,N-diisopropylethylamine at room temperature. The reaction mixture was stirred at 110° C. for 4 hours and concentrated under reduced pressure. The residue was purified by column chromatography on silica gel (petroleum ether/ethyl acetate) to yield 3.60 g (97% purity, 86% yield) of ethyl 2-[(1-phenylcyclobutyl)amino]pyrimidine-5-carboxylate as a white solid. Log P=3.53 [Method A]. Mass (M+H)=298.

### Step 2: Preparation of 2-[(1-phenylcyclobutyl)amino]pyrimidine-5-carbohydrazide

To solution of 700 mg (2.35 mmol) of ethyl 2-[(1-phenylcyclobutyl)amino]pyrimidine-5-carboxylate in 5 mL of ethanol were added 2.29 mL (98% purity, 46.3 mmol) of hydrazine monohydrate. The reaction mixture was heated under microwave at 120° C. for 1 hour and concentrated under reduced pressure. The resulting mixture was diluted by water (10 mL) and extracted with ethyl acetate (3×20 mL). The combined organic layers were washed with brine (10 mL), dried over anhydrous sodium sulfate, filtrated and concentrated under reduced pressure. The residue was purified by preparative HPLC (gradient acetonitrile/water+10 mmol/L NH₄HCO₃) to yield 650 mg (97% purity, 95% yield) of preparation of 2-[(1-phenylcyclobutyl)amino]pyrimidine-5-carbohydrazide as a light yellow solid. Log P=1.63 [Method A]. Mass (M+H)=284.

### Step 3: Preparation of N′-(difluoroacetyl)-2-[(1-phenylcyclobutyl)amino]pyrimidine-5-carbohydrazide

To a solution of 500 mg (1.76 mmol) of 2-[(1-phenylcyclobutyl)amino]pyrimidine-5-carbohydrazide and 268 mg (2.65 mmol) of triethylamine in 5 mL of tetrahydrofuran were added 369 mg (2.12 mmol) of difluoroacetic anhydride at room temperature. The reaction mixture was stirred at room temperature for 2 hours. The resulting mixture was diluted by water (10 mL) and extracted with ethyl acetate (3×15 mL). The combined organic extracts were washed with brine (10 mL), dried over anhydrous sodium sulfate, filtrated and concentrated under reduced pressure. The residue was purified by column chromatography on C18 silica gel (gradient water/acetonitrile) to yield 600 mg (99% purity, 93% yield) of N′-(difluoroacetyl)-2-[(1-phenylcyclobutyl)amino]pyrimidine-5-carbohydrazide as an off-white solid. Log P=1.99 [Method A]. Mass (M+H)=362.

### Step 4: 5-[5-(difluoromethyl)-1,3,4-oxadiazol-2-yl]-N-(1-phenylcyclobutyl)pyrimidin-2-amine (Compound I.134)

To a solution of 500 mg (1.38 mmol) of N′-(difluoroacetyl)-2-[(1-phenylcyclobutyl)amino]pyrimidine-5-carbohydrazide in 5 mL of tetrahydrofuran were added 659 mg (2.77 mmol) of Burgess reagent (CAS 29684-56-8) at room temperature. The reaction mixture was heated under microwave at 150° C. for 1 hour. The resulting mixture was diluted by water (10 mL) and extracted with ethyl acetate (3×15 mL).

The residue was purified by preparative HPLC (gradient acetonitrile/water+0.1% CF₃CO₂H) to yield 200 mg (99% purity, 42% yield) of N′-(difluoroacetyl)-2-[(1-phenylcyclobutyl)amino]pyrimidine-5-carbohydrazide. Log P=3.18 [Method A]. Mass (M+H)=344.

### Preparation Example 2: 3-ethyl-5-{4-[5-(trifluoromethyl)-1,3,4-oxadiazol-2-yl]phenyl}-1,2,4-oxadiazole (Compound I.018)

### Step 1: Preparation of tert-butyl 2-[4-(3-ethyl-1,2,4-oxadiazol-5-yl)benzoyl]hydrazinecarboxylate

To a solution of 85 mg (0.39 mmol) of 4-(3-ethyl-1,2,4-oxadiazol-5-yl)benzoic acid and 149 mg (0.39 mmol) of HATU (CAS 148893-10-1) in 3 mL of dichloromethane were added 62 mg (0.47 mmol) of tert-butyl carbazate and 101 mg (0.78 mmol) of N,N-diisopropylethylamine. The reaction mixture was stirred at 40° C. for 7 hours and concentrated under reduced pressure. The residue was purified by preparative HPLC (gradient acetonitrile/water+0.1% HCO₂H) to yield 114 mg (99% purity, 87% yield) of tert-butyl 2-[4-(3-ethyl-1,2,4-oxadiazol-5-yl)benzoyl]hydrazinecarboxylate as a light yellow solid. Log P=1.23 [Method A]. Mass (M+H)=333.

### Step 2: Preparation of 4-(3-ethyl-1,2,4-oxadiazol-5-yl)-N′-(trifluoroacetyl)benzohydrazide (Compound IIa.14)

To a solution of 312 mg (0.94 mmol) of tert-butyl 2-[4-(3-ethyl-1,2,4-oxadiazol-5-yl)benzoyl]hydrazine-carboxylate in 1.5 mL of dichloromethane were added 0.5 mL of trifluoroacetic acid (6.49 mmol). The reaction mixture was stirred at room temperature for 1 hour and concentrated under reduced pressure to afford a crude product. To a solution of half of the crude product obtained previously in 4 mL of dichloromethane was added 0.14 mL (0.99 mmol) of trifluoroacetic anhydride at room temperature. The reaction mixture was stirred at room temperature for 45 minutes and concentrated under reduced pressure to afford 138 mg (96% purity, 86% yield) of 4-(3-ethyl-1,2,4-oxadiazol-5-yl)-N′-(trifluoroacetyl)-benzohydrazide. Log P=1.93 [Method A]. Mass (M+H)=329.

### Step 3: Preparation of 3-ethyl-5-{4-[5-(trifluoromethyl)-1,3,4-oxadiazol-2-yl]phenyl}-1,2,4-oxadiazole (Compound I.018)

To a solution of 128 mg (0.39 mmol) of 4-(3-ethyl-1,2,4-oxadiazol-5-yl)-N′-(trifluoroacetyl)benzo-hydrazide in 1.5 mL of tetrahydrofuran were added 139 mg (0.59 mmol) of Burgess reagent (CAS 29684-56-8) at room temperature. The reaction mixture was stirred at 60° C. for 2 hours and concentrated under reduced pressure. The residue was purified by preparative HPLC (gradient acetonitrile/water+0.1% HCO₂H) to yield 35 mg (99% purity, 29% yield) of 3-ethyl-5-{4-[5-(trifluoro-methyl)-1,3,4-oxadiazol-2-yl]phenyl}-1,2,4-oxadiazole as an off-white solid. Log P=3.59 [Method A]. Mass (M+H)=311.

### Preparation Example 3: 4-[5-(trifluoromethyl)-1,3,4-thiadiazol-2-yl]benzoic acid (Compound I.008)

### Step 1: Preparation of methyl 4-[5-(trifluoromethyl)-1,3,4-thiadiazol-2-yl]benzoate (Compound I.011)

To a solution of 5.0 g (17.2 mmol) of methyl 4-{[2-(trifluoroacetyl)hydrazino]carbonyl}benzoate in 50 mL of toluene were added 7.67 g (18.9 mmol) of Lawesson reagent (CAS 19172-47-5) at room temperature under nitrogen atmosphere. The reaction mixture was stirred at 120° C. for 2 hours, cooled to room temperature and quenched by addition of a saturated aqueous sodium bicarbonate solution. The resulting mixture was extracted with ethyl acetate (2×100 mL). The combined organic layers were washed with brine (30 mL), dried over anhydrous sodium sulfate, filtrated and concentrated under reduced pressure. The residue was purified by column chromatography on silica gel (hexane/ethyl acetate) to yield 4.0 g (99% purity, 80% yield) of methyl 4-[5-(trifluoromethyl)-1,3,4-thiadiazol-2-yl]benzoate as a white solid. Log P=3.23 [Method A]. Mass (M+H)=289.

### Step 2: Preparation of 4-[5-(trifluoromethyl)-1,3,4-thiadiazol-2-yl]benzoic acid (Compound I.008)

To a solution of 1.10 g (3.82 mmol) of methyl 4-[5-(trifluoromethyl)-1,3,4-thiadiazol-2-yl]benzoate in 5 mL of a 1:1 mixture of tetrahydrofuran and methanol were added 2.86 mL of a solution of lithium hydroxide (2.0 M in tetrahydrofuran, 5.72 mmol). The reaction mixture was stirred at room temperature for 20 minutes and concentrated under reduced pressure. The resulting mixture was diluted by water (10 mL) and acidified to pH 4 with a 1 M aqueous hydrochloric acid solution. The precipitated solids were collected by filtration and washed with water (2×10 mL). The resulting solid was dried in an oven under reduced pressure to yield 600 mg (97% purity, 56% yield) of 4-[5-(trifluoromethyl)-1,3,4-thiadiazol-2-yl]benzoic acid as a white solid. Log P=1.41 [Method A]. Mass (M+H)=275.

### Preparation Example 4: 5-[5-(difluoromethyl)-1,3,4-oxadiazol-2-yl]-N-[2-(4-fluorophenyl)propan-2-yl]pyrimidin-2-amine (Compound I.150)

In a 2.5 mL oven-dried microwave tube, to a solution of 100 mg (0.65 mmol) of 2-(4-fluorophenyl)-propan-2-amine and 159 mg (0.68 mmol) of 2-chloro-5-[5-(difluoromethyl)-1,3,4-oxadiazol-2-yl]-pyrimidine in 1 mL of dry dioxane, were added 253 mg (1.95 mmol) of N,N-diisopropylethylamine. The reaction mixture was heated under microwave at 90° C. for 16 hours. The cooled reaction mixture was concentrated under reduced pressure and the residue was purified by preparative HPLC (gradient acetonitrile/water+0.1% HCO₂H) to yield 145 mg (98% purity, 62% yield) of 5-[5-(difluoromethyl)-1,3,4-oxadiazol-2-yl]-N-[2-(4-fluorophenyl)propan-2-yl]pyrimidin-2-amine as a solid. Log P=3.26 [Method A]. Mass (M+H)=350.

### BIOLOGICAL EXAMPLES

### Example A: Fungicidal Activity Against Phakosporra pachirrhizi

Cryo-conserved wild-type spores of the biotroph Phakosporra pachirrhizi were humidified in dedicated chamber overnight at 18° C. in the dark. The next day, a solution of spores at 7×10³ sp/mL was prepared in a water based growth medium (H₂O+0.2 mM MOPS at pH 7+0.01% Tween 20) and spores were distributed in a 96-MTPS (final volume of 200 μL per well) thanks to a dispenser robot. Each molecule was tested at 10 doses (from 30 to 0.002 ppm final concentration) and accordingly 1.2 μL of each dilution was transferred in dedicated well to end-up with a final concentration of 0.6% DMSO. Wild-type spores and molecules were incubated for 4 hours at 21° C., and 6 images par well were then acquired with Transmitted Light images (Image Xpress Micro microscope, Molecular Devices, Objective 10×, 6 images per well). Detection and quantification of the number of germinated spores per image was performed with a dedicated in-house developed algorithm (MetaXpress software, Molecular Devices).

Inhibition of the fungal germination was hence determined by comparing the number of germinated spores in wells containing the tested compounds to the ones without active ingredient.

In this test, the following compounds according to the invention showed efficacy of at least 70% at a concentration of 30 ppm of active ingredient: I.04; I.018 In this test, the following compounds according to the invention showed efficacy of at least 70% at a concentration of 20 ppm of active ingredient: I.008; I.134; I.144: I.160 In this test, the following compounds according to the invention showed efficacy of at least 70% at a concentration of 4 ppm of active ingredient: I.027

### Example B: In Vitro Cell Test on Colletotrichum lindemuthianum

Solvent: dimethyl sulfoxide

Culture medium: 14.6 g anhydrous D-glucose (VWR),
7.1 g mycological peptone (Oxoid),
1.4 g granulated yeast extract (Merck), QSP 1 liter

Inoculum: spore suspension

The tested compounds were solubilized in dimethyl sulfoxide and the solution used to prepare the required range of concentrations. The final concentration of dimethyl sulfoxide used in the assay was ≤1%.

A spore suspension of Colletotrichum lindemuthianum was prepared and diluted to the desired spore density.

The compounds were evaluated for their ability to inhibit spore germination and mycelium growth in liquid culture assay. The compounds were added in the desired concentration to the culture medium with spores. After 6 days incubation, fungi-toxicity of compounds was determined by spectrometric measurement of mycelium growth. Inhibition of fungal growth was determined by comparing the absorbance values in wells containing the tested compounds with the absorbance in control wells without tested compounds.

In this test, the following compound according to the invention showed efficacy between 70% and 79% at a concentration of 20 ppm of tested compound: I.051; I.112; I.120; I.132; I.141; I.146; I.171; I.172; I.176; I.182; I.196

In this test, the following compound according to the invention showed efficacy between 80% and 89% at a concentration of 20 ppm of tested compound: I.025; I.067; I.125; I.135; I.136; I.145; I.150; I.157; I.160; I.165; I.166; I.169; I.173; I.175; I.181; I.183

In this test, the following compounds according to the invention showed efficacy between 90% and 100% at a concentration of 20 ppm of tested compound: I.013; I.017; I.061; I.063; I.068; I.077; I.089; I.110; I.118; I.122; I.123; I.131; I.134; I.139; I.144; I.152; I.159; I.177

### Example C: In Vivo Preventive Test on Botrytis cinerea (Grey Mould)

Solvent: 5% by volume of dimethyl sulfoxide
10% by volume of acetone

Emulsifier: 1 μl of Tween® 80 per mg of active ingredient

The tested compounds were made soluble and homogenized in a mixture of dimethyl sulfoxide/acetone/Tween® 80 and then diluted in water to the desired concentration.

The young plants of gherkin or cabbage were treated by spraying the tested compound prepared as described above. Control plants were treated only with an aqueous solution of acetone/dimethyl sulfoxide/Tween® 80.

After 24 hours, the plants were contaminated by spraying the leaves with an aqueous suspension of Botrytis cinerea spores. The contaminated gherkin plants were incubated for 4 to 5 days at 17° C. and at 90% relative humidity. The contaminated cabbage plants were incubated for 4 to 5 days at 20° C. and at 100% relative humidity.

The test was evaluated 4 to 5 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I.077; I.135; I.153; I.161; I.170; I.181

In this test the following compounds according to the invention showed efficacy between 80% and 89% at a concentration of 500 ppm of active ingredient: I.030; I.034; I.050; I.053; I.116

In this test the following compounds according to the invention showed efficacy between 90% and 100% at a concentration of 500 ppm of active ingredient: I.045; I.076; I.113; I.123; I.146; I.196

### Example D: In Vivo Preventive Test on Puccinia recondita (Brown Rust on Wheat)

Solvent: 5% by volume of dimethyl sulfoxide
10% by volume of acetone

Emulsifier: 1 μL of Tween® 80 per mg of active ingredient

The tested compounds were made soluble and homogenized in a mixture of dimethyl sulfoxide/acetone/Tween® 80 and then diluted in water to the desired concentration.

The young plants of wheat were treated by spraying the tested compound prepared as described above. Control plants were treated only with an aqueous solution of acetone/dimethyl sulfoxide/Tween® 80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test, the following compound according to the invention showed efficacy between 70% and 79% at a concentration of 500 ppm of tested compound: I.108; I.120; I.121; I.127; I.134; I.180

In this test, the following compound according to the invention showed efficacy between 80% and 89% at a concentration of 500 ppm of tested compound: I.026; I.118; I.132; I.136; I.155

In this test, the following compounds according to the invention showed efficacy between 90% and 100% at a concentration of 500 ppm of tested compound: I.093; I.122; I.125; I.133; I.137; I.144; I.145; I.146; I.150; I.156; I.157; I.165; I.166; I.175; I.177; I.181

### Example E: In Vivo Preventive Test on Alternaria brassicae (Cabbage)

Solvent: 5% by volume of dimethyl sulfoxide
10% by volume of acetone

Emulsifier: 1 μL of Tween® 80 per mg of active ingredient

The tested compounds were made soluble and homogenized in a mixture of Dimethyl sulfoxide/Acetone/Tween® 80 and then diluted in water to the desired concentration.

The young plants of cabbage were treated by spraying the tested compound prepared as described above. Control plants were treated only with an aqueous solution of Acetone/Dimethyl sulfoxide/Tween® 80.

After 24 hours, the plants were contaminated by spraying the leaves with an aqueous suspension of Alternaria brassicae spores. The contaminated cabbage plants were incubated for 6 days at 20° C. and at 100% relative humidity.

The test was evaluated 6 days after the inoculation. 0% means an efficacy which corresponds to that of the control plants while an efficacy of 100% means that no disease was observed.

In this test, the following compound according to the invention showed efficacy between 70% and 79% at a concentration of 500 ppm of tested compound: I.002; I.040; I.068; I.072; I.133; I.150; I.157; I.166; I.175

In this test, the following compound according to the invention showed efficacy between 80% and 89% at a concentration of 500 ppm of tested compound: I.007; I.017; I.105; I.171; I.172; I.177

In this test, the following compounds according to the invention showed efficacy between 90% and 100% at a concentration of 500 ppm of tested compound: I.005; I.010; I.013; I.045; I.076; I.087; I.094; I.110; I.113; I.131; I.135; I.137; I.146; I.147; I.156; I.170; I.196

### Example F: In Vivo Preventive Test on Phakopsora pachyrhizi (Soybeans)

Solvent: 5% by volume of dimethyl sulfoxide
10% by volume of acetone

Emulsifier: 1 μL of Tween® 80 per mg of active ingredient

The tested compounds were made soluble and homogenized in a mixture of dimethyl sulfoxide/acetone/Tween® 80 and then diluted in water to the desired concentration.

The young plants of wheat were treated by spraying the tested compound prepared as described above. Control plants were treated only with an aqueous solution of acetone/dimethyl sulfoxide/Tween® 80.

After 24 hours, the plants were contaminated by spraying the leaves with an aqueous suspension of Phakospora pachyrhizi spores. The contaminated soybean plants were incubated for 24 hours at 24° C. and at 100% relative humidity and then for 11 days at 24° C. and at 70-80% relative humidity.

The test was evaluated 12 days after the inoculation. 0% means an efficacy which corresponds to that of the control plants while an efficacy of 100% means that no disease was observed.

In this test, the following compound according to the invention showed efficacy between 70% and 79% at a concentration of 500 ppm of tested compound: I.009; I.115; I.171

In this test, the following compound according to the invention showed efficacy between 80% and 89% at a concentration of 500 ppm of tested compound: I.147

In this test, the following compounds according to the invention showed efficacy between 90% and 100% at a concentration of 500 ppm of tested compound: I.019; I.024; I.059; I.083; I.088; I.093; I.110; I.118; I.120; I.121; I.122; I.125; I.127; I.132; I.133; I.134; I.136; I.137; I.144; I.145; I.146; I.150; I.155; I.156; I.157; I.160; I.165; I.166; I.177; I.181; I.196

### Example G: In Vivo Preventive Test on Phakopsora pachyrhizi (Soybeans)

Solvent: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous spore suspension of the causal agent of soybean rust (Phakopsora pachyrhizi) and stay for 24 hours without light in an incubation cabinet at approximately 24° C. and a relative atmospheric humidity of 95%.

The plants remained in the incubation cabinet at approximately 24° C. and a relative atmospheric humidity of approximately 80% and a day/night interval of 12 hours.

The test was evaluated 7 days after the inoculation. 0% means an efficacy which corresponds to that of the untreated control, while an efficacy of 100% means that no disease is observed.

In this test, the following compounds according to the invention showed efficacy between 90% and 100% at a concentration of 250 ppm of active ingredient: I.134; I.165.

1. A product comprising a compound of formula (I) or a salt, N-oxide or solvate thereof for controlling phytopathogenic fungi: 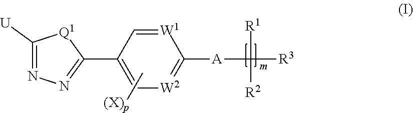
wherein
U is a C₁-C₃-haloalkyl comprising 2 to 7 halogen atoms that can be the same or different and selected from the group consisting of fluorine and chlorine;
Q¹ is O or S;
W¹ and W² are independently N, CH or CF;
A is selected from the group consisting of direct bond, O, S, S═O, S(═O)₂, NR⁴, —(C═O)—, —(C═S)—, —O—(C═O), —O—(C═S)—, —N(R⁴)—(C═O)—, —N(R⁴)—(C═S)—, —(C═O)—O—, —(C═S)—O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O— and —O—(C═S)═O—;
m=0, 1 or 2; wherein, if m is 2, the two [CR¹R²] groups may be the same or different;
p=0, 1 or 2;
X is fluorine;
each R¹ and each R² are independently selected from the group consisting of hydrogen, halogen, cyano, aminocarbonyl, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with, respectively, one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with, respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of O, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, hydroxyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted with, respectively one or more R^{4b} and R^{5b} substituents;
R¹, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
provided that compound of formula (I) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
2-isopropyl-5,6-dimethyl-3-{4-[5-(trifluoromethyl)-1,3,4-oxadiazol-2-yl]benzyl}pyridin-4-ol [2133324-02-2].

2. A product comprising the compound of formula (I) according to claim 1 or a salt, N-oxide or solvate thereof,
wherein
U is selected from CHF₂, CClF₂ and CF₃;
Q¹ is O or S;
W¹ and W² are independently N or CH;
A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
m is 0, 1 or 2; wherein, if m is 2, the two [CR¹R²] groups may be the same or different;
p is 0 or 1;
X is fluorine;
each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or
R¹ and R² may form, together with the carbon atom to which they are linked, a C₃-C₇-cycloalkyl or oxetanyl ring, or
two consecutive R¹, when m is 2, may form, together with the carbon atoms to which they are linked, a C₃-C₇-cycloalkyl ring;
R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;
R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms.
provided that compound of formula (I) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
as well as their salts, N-oxides, solvates and optically active isomers or geometric isomers.

3. A product comprising the compound of formula (I) according to claim 1, or a salt, N-oxide or solvate thereof, wherein
U is selected from CHF₂, CClF₂ and CF₃;
Q¹ is O or S;
W¹ and W² are independently N or CH;
A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
m is 0, 1 or 2; wherein, if m is 2, the two [CR¹R²] groups may be the same or different;
p is 0;
each R¹ is independently selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl,
or two consecutive R¹, when m is 2, may form, together with the carbon atoms to which they are linked, a cyclopropyl, cyclobutyl or cyclopentyl ring, and
each R² is independently selected from the group consisting of hydrogen, fluorine, chlorine, methyl, ethyl, trifluoromethyl and difluoromethyl,
or
R¹ and R² may form, together with the carbon atom to which they are linked, a cyclopropyl, cyclobutyl, cyclopentyl, cyclohexyl or oxetanyl ring;
R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
R⁴ and R⁵ are independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms;
provided that compound of formula (I) is not:
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6].

4. A product comprising the compound of formula (I) according to claim 1, or a salt, N-oxide or solvate thereof, wherein
U is CHF₂ or CF₃, optionally CHF₂.

5. A product comprising the compound of formula (I) according to claim 1, or a salt, N-oxide or solvate thereof, wherein
A is a direct bond, O, NR⁴, —N(R⁴)—(C═O)—, —(C═O)—O— or —(C═O)—N(R⁵)—.

6. A product comprising the compound of formula (I) according to claim 1, or a salt, N-oxide or solvate thereof, wherein
R⁴ and R⁵ are independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms.

7. A product comprising the compound of formula (I) according to claim 1, or a salt, N-oxide or solvate thereof, wherein W¹ and W² are N, or W¹ is N and W² is CH.

8. A product comprising the compound of formula (I) according to claim 1, or a salt, N-oxide or solvate thereof, wherein Q¹ is O.

9. A product comprising the compound of formula (I) according to claim 1, or a salt, N-oxide or solvate thereof, wherein A is O or NR⁴.

10. A product comprising the compound of formula (I) according to claim 1, or a salt, N-oxide or solvate thereof, wherein
U is CHF₂ or CF₃;
Q¹ is O;
p is 0;
W¹ and W² are N;
A is O or NH;
m is 1;
R¹ is selected from the group consisting of hydrogen, C₁-C₈-alkyl, C₁-C₈-halogenoalkyl, C₂-C₈-alkynyl, C₂-C₈-alkenyl, C₃-C₇-cycloalkyl and phenyl;
R² is hydrogen;
or
R¹ and R² form, together with the carbon atom to which they are linked, a C₃-C₇-cycloalkyl or oxetanyl ring, optionally a a cyclopropyl, cyclobutyl or oxetanyl ring; and
R³ is selected from the group consisting of C₃-C₇-cycloalkyl, C₃-C₈-cycloalkenyl, aryl, heterocyclyl and heteroaryl,
wherein C₃-C₇-cycloalkyl is selected from cyclopropyl, cyclobutyl, cyclopentyl and cyclohexyl;
wherein C₃-C₈-cycloalkenyl is selected from cyclopentenyl and cyclohexenyl;
wherein aryl is selected from phenyl and naphthyl;
wherein heterocyclyl is selected from piperidin-1-yl, piperazin-1-yl, tetrahydro-2H-pyran-4-yl, tetrahydrothiopyran-4-yl, morpholin-4-yl and 1,3-dihydro-2H-isoindol-2-yl;
wherein heteroaryl is selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl;
and wherein said C₃-C₇-cycloalkyl, C₃-C₈-cycloalkenyl, aryl, heterocyclyl and heteroaryl may be substituted with one to three substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

11. A product comprising the compound of formula (I) according to claim 10, or a salt, N-oxide or solvate thereof, wherein
R¹ is selected from the group consisting of hydrogen, methyl, ethyl, isopropyl, trifluoromethyl, ethenyl, ethynyl, phenyl and cyclopropyl, and
R² is hydrogen,
or
R¹ and R² form, together with the carbon atom to which they are linked, a cyclopropyl, cyclobutyl or oxetanyl ring; and
R³ is selected from phenyl and pyridine, wherein the phenyl and the pyridine may be substituted with one to three R^{3b} substituents independently selected from fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl.

12. A compound of formula (I): 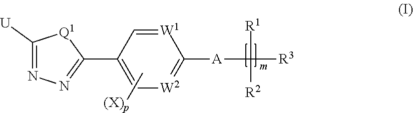
wherein
U is a C₁-C₃-haloalkyl comprising 2 to 7 halogen atoms that can be the same or different and selected from the group consisting of fluorine and chlorine;
Q¹ is O or S;
A is selected from the group consisting of direct bond, O, NR⁴, S, S═O, S(═O)₂, —(C═O)—, —(C═S)—, —O—(C═O)—, —O—(C═S)—, —N(R⁴)—(C═O)—, —N(R⁴)—(C═S)—, —(C═O)—O—, —(C═S)—O—, —(C═O)—N(R⁵)—, —(C═S)—N(R⁵)—, —(C═O)—N(R⁴)—N(R⁵)—, —(C═S)—N(R⁴)—N(R⁵)—, —O—N(R⁵)—, —N(R⁴)—O—, —N(R⁴)—N(R⁵)—, —O—(C═O)—N(R⁵)—, —O—(C═S)—N(R⁵)—, —N(R⁴)—(C═O)—O—, —N(R⁴)—(C═S)═O—, —N(R⁴)—(C═O)—N(R⁵)—, —N(R⁴)—(C═S)—N(R⁵)—, —O—(C═O)—O—, —O—(C═S)—O—;
W¹ and W² are independently N, CH or CF;
m=0, 1 or 2; wherein, if m is 2, the two [CR¹R²] groups may be the same or different;
p=0, 1 or 2;
X is fluorine;
each R¹ and each R² are independently selected from the group consisting of hydrogen, halogen, cyano, C₁-C₈-alkyl, C₁-C₈-halogenoalkyl, C₂-C₈-alkenyl, C₂-C₈-halogenoalkenyl, C₂-C₈-alkynyl, C₂-C₈-halogenoalkynyl, C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said C₁-C₈-alkyl, C₂-C₈-alkenyl and C₂-C₈-alkynyl may be substituted with respectively one or more R^{1a} and R^{2a} substituents and wherein said C₃-C₇-cycloalkyl, aryl, heterocyclyl, heteroaryl, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with respectively one or more R^{1b} and R^{2b} substituents; or
R¹ and R² may form, together with the carbon atom to which they are linked, a C₃-C₇-cycloalkyl or a 3- to 10-membered saturated or partially unsaturated heterocyclyl ring that contains 1 to 3 heteroatoms that can be the same or different and selected from the group consisting of O, S and NH, wherein said C₃-C₇-cycloalkyl and 3- to 10-membered saturated or partially unsaturated heterocyclyl ring may be substituted with one or more R^{1b} substituents; or
two consecutive R¹, when m is 2, may form, together with the carbon atoms to which they are linked, a C₃-C₇-cycloalkyl ring wherein said C₃-C₇-cycloalkyl ring may be substituted with one or more R^{1b} substituents;
R³ is hydrogen, halogen, borono, potassium (trifluoro)boryl, di-(C₁-C₈-alkoxy)boryl, 1,3,2-dioxaborolan-2-yl, 1,3,2-dioxaborinan-2-yl, C₁-C₈-alkyl, C₁-C₈-halogenoalkyl, C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl, wherein said 1,3,2-dioxaborolan-2-yl and 1,3,2-dioxaborinan-2-yl may be substituted with one to four C₁-C₃-alkyl substituents, and wherein said C₁-C₈-alkyl may be substituted with one or more R^{3a} substituents, and wherein said C₃-C₇-cycloalkyl, C₃-C₈-cycloalkenyl, aryl, heterocyclyl, heteroaryl, biphenyl, phenoxyphenyl, aryloxy, heterocyclyloxy, heteroaryloxy, aryl-C₁-C₈-alkyl, heterocyclyl-C₁-C₈-alkyl, heteroaryl-C₁-C₈-alkyl and C₃-C₇-cycloalkyl-C₁-C₈-alkyl may be substituted with one or more R^{3b} substituents;
R⁴ and R⁵ are independently selected from the group consisting of hydrogen atom, hydroxy, C₁-C₈-alkyl, C₁-C₈-halogenoalkyl, C₁-C₈-alkoxy, C₁-C₈-halogenoalkoxy, C₂-C₈-alkenyl, C₂-C₈-halogenoalkenyl, C₃-C₈-alkynyl, C₃-C₈-halogenoalkynyl, C₃-C₇-cycloalkyl, C₃-C₇-cycloalkyl-C₁-C₈-alkyl, formyl, C₁-C₈-alkylcarbonyl, C₁-C₈-halogenoalkyl-carbonyl, arylcarbonyl, C₁-C₈-alkoxycarbonyl, C₁-C₈-halogenoalkoxycarbonyl, C₁-C₈-alkylsulfonyl, C₁-C₈-halogenoalkylsulfonyl, aryl, heteroaryl, aryl-C₁-C₈-alkyl, heteroaryl-C₁-C₈-alkyl and phenylsulfonyl, wherein said C₁-C₈-alkyl, C₁-C₈-alkoxy, C₂-C₈-alkenyl, C₃-C₈-alkynyl, C₁-C₈-alkylcarbonyl, C₁-C₈-alkoxycarbonyl and C₁-C₈-alkylsulfonyl may be substituted with respectively one or more R^{4a} and R^{5a} substituents and wherein said C₃-C₇-cycloalkyl, C₃-C₇-cycloalkyl-C₁-C₈-alkyl, arylcarbonyl, aryl, heteroaryl, aryl-C₁-C₈-alkyl, heteroaryl-C₁-C₈-alkyl and phenylsulfonyl, may be substituted respectively with one or more R^{4b} and R^{5b} substituents;
R^{1a}, R^{2a}, R^{3a}, R^{4a} and R^{5a} are independently selected from the group consisting of halogen, nitro, hydroxyl, cyano, carboxyl, amino, sulfanyl, pentafluoro-λ⁶-sulfanyl, formyl, carbamoyl, carbamate, C₃-C₇-cycloalkyl, C₃-C₇-halogenocycloalkyl having 1 to 5 halogen atoms, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
R^{1b}, R^{2b}, R^{3b}, R^{4b} and R^{5b} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₇-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms; C₁-C₈-alkylsulfonylamino, C₁-C₈-halogenoalkylsulfonylamino having 1 to 5 halogen atoms; sulfamoyl; C₁-C₈-alkylsulfamoyl and di-C₁-C₈-alkylsulfamoyl;
provided that A is not NR⁴ when m is 1 or 2 and W¹ and W² are N;
provided U is not CCl₃ or CHCl₂ when W¹ and W² are CH;
provided that compound of formula (I) is not:
2,5-bis[5-(trichloromethyl)-1,3,4-oxadiazol-2-yl]pyridine [222190-08-1],
2-(bromomethyl)-5-[5-(difluoromethyl)-1,3,4-oxadiazol-2-yl]pyridine [2071232-31-8],
5-[5-(difluoromethyl)-1,3,4-oxadiazol-2-yl]-2-methylpyridine [2071232-29-4],
2-chloro-5-[5-(trifluoromethyl)-1,3,4-oxadiazol-2-yl]pyridine [2011795-38-1]
2-chloro-5-[5-(dichloromethyl)-1,3,4-oxadiazol-2-yl]pyridine [160152-11-4],
2-{5-[5-(trifluoromethyl)-1,3,4-oxadiazol-2-yl]pyridin-2-yl}-1H-benzimidazole-7-carboxamide [1103394-47-3],
5-[5-(difluoromethyl)-1,3,4-oxadiazol-2-yl]-N-{(4-fluorophenyl)[1-(methylsulfonyl)azetidin-3-yl]methyl}pyrimidin-2-amine [2243579-66-8],
5-[5-(difluoromethyl)-1,3,4-oxadiazol-2-yl]-2-(methylsulfonyl)pyrimidine [2095318-34-4],
5-[5-(difluoromethyl)-1,3,4-oxadiazol-2-yl]-2-(methylsulfanyl)pyrimidine [2095318-33-3].
N-{4-[5-(trifluoromethyl)-1,3,4-oxadiazol-2-yl]phenyl}cyclopropanecarboxamide [2376135-82-7],
tert-butyl {4-[5-(trifluoromethyl)-1,3,4-oxadiazol-2-yl]phenyl}carbamate [2376135-81-6],
N-[2-({5-[5-(difluoromethyl)-1,3,4-oxadiazol-2-yl]pyrimidin-2-yl}amino)-2-(tetrahydro-2H-pyran-4-yl)ethyl]methanesulfonamide [2243579-65-7],
1-[4-({5-[5-(difluoromethyl)-1,3,4-oxadiazol-2-yl]pyrimidin-2-yl}amino)-4-isopropylpiperidin-1-yl]ethanone [2243579-38-4],
N-{5-[5-(difluoromethyl)-1,3,4-oxadiazol-2-yl]pyrimidin-2-yl}-2,2-difluoro-N-[1-(4-fluorophenyl)-2-{3-[(methylsulfonyl)amino]phenyl}ethyl]acetamide [2243577-58-2],
N-[2-cyclopropyl-2-({5-[5-(difluoromethyl)-1,3,4-oxadiazol-2-yl]pyrimidin-2-yl}amino)ethyl]methane-sulfonamide [2243577-21-9],
N-[2-cyclopropyl-2-({5-[5-(difluoromethyl)-1,3,4-oxadiazol-2-yl]pyrimidin-2-yl}amino)ethyl]methane-sulfonamide [2243577-20-8],
N-[2-cyclopropyl-2-({5-[5-(difluoromethyl)-1,3,4-oxadiazol-2-yl]pyrimidin-2-yl}amino)ethyl]methane-sulfonamide [2243577-19-5],
N-[cyclopropyl(4-fluorophenyl)methyl]-N-{5-[5-(difluoromethyl)-1,3,4-oxadiazol-2-yl]pyrimidin-2-yl}-2,2-difluoroacetamide [2243576-38-5],
N-[cyclopropyl(4-fluorophenyl)methyl]-5-[5-(difluoromethyl)-1,3,4-oxadiazol-2-yl]pyrimidin-2-amine [2243576-35-2],
5-[5-(difluoromethyl)-1,3,4-oxadiazol-2-yl]-N-(1-isopropylcyclopropyl)pyrimidin-2-amine [2243576-06-7],
5-[5-(difluoromethyl)-1,3,4-oxadiazol-2-yl]-2-[(1-phenylcyclopropyl)oxy]pyrimidine [2243575-46-2] and
N-[(6-methylpyridin-2-yl)methyl]-5-[5-(trifluoromethyl)-1,3,4-oxadiazol-2-yl]pyrimidin-2-amine [2243575-26-8],
2-(difluoromethyl)-5-(4-iodophenyl)-1,3,4-oxadiazole [2244172-62-9],
2-isopropyl-5,6-dimethyl-3-{4-[5-(trifluoromethyl)-1,3,4-oxadiazol-2-yl]benzyl}pyridin-4-ol [2133324-02-2],
2-[4-(bromomethyl)-3-fluorophenyl]-5-(trifluoromethyl)-1,3,4-oxadiazole [2098919-34-5],
N-{4-[5-(difluoromethyl)-1,3,4-oxadiazol-2-yl]benzyl}butan-1-amine [2080363-69-3],
2-[4-(chloromethyl)phenyl]-5-(difluoromethyl)-1,3,4-oxadiazole [2071231-55-3],
2-[4-(bromomethyl)-3-fluorophenyl]-5-(difluoromethyl)-1,3,4-oxadiazole [2071227-85-3],
2-(difluoromethyl)-5-(3-fluoro-4-methylphenyl)-1,3,4-oxadiazole [2071227-84-2],
1-{4-[5-(trifluoromethyl)-1,3,4-oxadiazol-2-yl]phenyl}methanamine hydrochloride (1:1) [2071226-91-8],
N-{4-[5-(trifluoromethyl)-1,3,4-oxadiazol-2-yl]benzyl}methanesulfonamide [2071223-51-1],
methyl 4-[5-(trifluoromethyl)-1,3,4-oxadiazol-2-yl]benzoate [1352872-14-0],
2-(trifluoromethyl)-5-[4-(trifluoromethyl)phenyl]-1,3,4-oxadiazole [1352872-13-9],
2-(4-tert-butylphenyl)-5-(trifluoromethyl)-1,3,4-oxadiazole [1352872-12-8],
2-(4-methylphenyl)-5-(trifluoromethyl)-1,3,4-oxadiazole [1352872-11-7],
methyl {4-[5-(trifluoromethyl)-1,3,4-oxadiazol-2-yl]phenoxy}acetate [1227372-86-2],
ethyl 2-{4-[5-(trifluoromethyl)-1,3,4-oxadiazol-2-yl]phenoxy}propanoate [1227372-85-1],
2-(4-bromophenyl)-5-(trifluoromethyl)-1,3,4-oxadiazole [918476-23-0],
4-[5-(trifluoromethyl)-1,3,4-oxadiazol-2-yl]aniline [904643-35-2],
2-([biphenyl]-4-yl)-5-(trifluoromethyl)-1,3,4-oxadiazole [887267-97-2],
2-[4-(4,4,5,5-tetramethyl-1,3,2-dioxaborolan-2-yl)phenyl]-5-(trifluoromethyl)-1,3,4-oxadiazole [1056456-25-7],
2-(4-chlorophenyl)-5-(trifluoromethyl)-1,3,4-oxadiazole [627073-36-3],
2-(4-methoxyphenyl)-5-(trifluoromethyl)-1,3,4-oxadiazole [371950-64-0],
2-(dichloromethyl)-5-(4-methoxyphenyl)-1,3,4-oxadiazole [214195-06-9],
2-(4-tert-butylphenyl)-5-(dichloromethyl)-1,3,4-oxadiazole [160152-26-1],
2-(dichloromethyl)-5-(4-ethoxyphenyl)-1,3,4-oxadiazole [160152-21-6],
5-[5-(difluoromethyl)-1,3,4-oxadiazol-2-yl]-2-fluoropyridine [2137870-57-4],
4-[5-(difluoromethyl)-1,3,4-thiadiazol-2-yl]aniline [2275439-93-3],
2-({4-[5-(difluoromethyl)-1,3,4-oxadiazol-2-yl]benzoyl}oxy)-1H-isoindole-1,3(2H)-dione [2248417-20-9],
tert-butyl 4-[5-(difluoromethyl)-1,3,4-oxadiazol-2-yl]benzoate [2241139-66-0],
methyl 4-[5-(difluoromethyl)-1,3,4-oxadiazol-2-yl]benzoate [2230804-32-5],
4-[5-(trifluoromethyl)-1,3,4-thiadiazol-2-yl]aniline [2160335-34-0],
4-[5-(difluoromethyl)-1,3,4-oxadiazol-2-yl]benzaldehyde [2138236-86-7],
2-(difluoromethyl)-5-(4-fluorophenyl)-1,3,4-oxadiazole [2137866-38-5],
2-(4-bromophenyl)-5-(difluoromethyl)-1,3,4-oxadiazole [2137697-81-3],
4-[5-(pentafluoroethyl)-1,3,4-oxadiazol-2-yl]benzoic acid [1920768-68-8],
4-[5-(1,1,2,2-tetrafluoroethyl)-1,3,4-oxadiazol-2-yl]benzoic acid [1917442-65-9],
4-[5-(difluoromethyl)-1,3,4-oxadiazol-2-yl]benzoic acid [1282022-66-5] and
4-[5-(trifluoromethyl)-1,3,4-oxadiazol-2-yl]benzoic acid [1197226-72-4].

13. The compound of formula (I) according to claim 12, wherein
U is selected from CHF₂, CClF₂ and CF₃;
Q¹ is O or S;
W¹ and W² are independently N or CH;
A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
m is 0, 1 or 2; wherein, if m is 2, the two [CR¹R²] groups may be the same or different;
p is 0 or 1;
X is fluorine;
each R¹ and each R² are independently selected from the group consisting of hydrogen, halogen, cyano, C₁-C₈-alkyl, C₁-C₈-halogenoalkyl, C₂-C₈-alkynyl, C₃-C₇-cycloalkyl and aryl, wherein said C₁-C₈-alkyl may be substituted with one or more substituents selected from hydroxy and C₁-C₈-alkoxy, or
R¹ and R² may form, together with the carbon atom to which they are linked, a C₃-C₇-cycloalkyl or oxetanyl ring, or
two consecutive R¹, when m is 2, may form, together with the carbon atoms to which they are linked, a C₃-C₇-cycloalkyl ring;
R³ is selected from the group consisting of hydrogen, halogen, 1,3,2-dioxaborolan-2-yl, C₁-C₈-alkyl, C₁-C₈-haloalkyl, C₃-C₇-cycloalkyl, C₃-C₈-cycloalkenyl, aryl, heterocyclyl, heteroaryl, biphenyl, phenoxyphenyl and aryloxy,
wherein said 1,3,2-dioxaborolan-2-yl may be substituted with one to four C₁-C₃-alkyl substituents,
wherein said C₁-C₈-alkyl may be substituted with one C₁-C₈-alkoxy or C₁-C₈-haloalkoxy substituent, and
wherein said C₃-C₇-cycloalkyl, C₃-C₈-cycloalkenyl, aryl, heterocyclyl, heteroaryl, biphenyl, phenoxyphenyl and aryloxy may be substituted with one to three R^{3b} substituents independently selected from the group consisting of halogen, nitro, cyano, C₁-C₈-alkyl, C₃-C₇-cycloalkyl, C₁-C₈-haloalkyl having 1 to 5 halogen atoms, C₁-C₈-alkoxy, C₁-C₈-haloalkoxy having 1 to 5 halogen atoms and C₁-C₈-alkoxycarbonyl;
R⁴ and R⁵ are independently selected from the group consisting of hydrogen, hydroxy, C₁-C₈-alkyl, C₁-C₈-haloalkyl, C₁-C₈-alkoxy, C₁-C₈-haloalkoxy, C₃-C₈-alkynyl, C₁-C₈-alkylcarbonyl, C₁-C₈-halogenoalkyl-carbonyl and arylcarbonyl, wherein said arylcarbonyl may be substituted with one or two fluorine atoms,
provided that A is not NR⁴ when m is 1 or 2 and W¹ and W² are N.

14. The compound of formula (I) according to claim 12,
wherein
U is selected from CHF₂, CClF₂ and CF₃, optionally CHF₂ or CF₃;
Q¹ is O or S, optionally O;
W¹ and W² are independently N or CH;
A is a direct bond, O, S, NR⁴, —N(R⁴)—(C═O), —(C═O)—O—, —(C═O)—N(R⁵)—, —(C═O)—N(R⁴)—N(R⁵), —N(R⁴)—N(R⁵)—, —N(R⁴)—(C═O)—O— or —N(R⁴)—(C═O)—N(R⁵)—;
m is 0, 1 or 2; wherein, if m is 2, the two [CR¹R²] groups may be the same or different;
p is 0;
each R¹ is independently selected from the group consisting of hydrogen, fluorine, chlorine, cyano, methyl, ethyl, iso-propyl, n-propyl, n-butyl, iso-butyl, tert-butyl, hydroxymethyl, trifluoromethyl, difluoromethyl, ethenyl, ethynyl, phenyl, cyclopentyl, cyclobutyl and cyclopropyl,
or two consecutive R¹, when m is 2, may form, together with the carbon atoms to which they are linked, a cyclopropyl, cyclobutyl or cyclopentyl ring, and
each R² is independently selected from the group consisting of hydrogen, fluorine, chlorine, methyl, ethyl, trifluoromethyl and difluoromethyl,
or
R¹ and R² may form, together with the carbon atom to which they are linked, a cyclopropyl, cyclobutyl, cyclopentyl, cyclohexyl or oxetanyl ring;
R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and naphthyl; heterocyclyl selected from the group consisting of tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl and 1,3-dihydro-2H-isoindol-2-yl; heteroaryl selected from the group consisting of furyl (furanyl), thienyl, pyrrolyl, pyrazolyl, imidazolyl, triazolyl, isoxazolyl, oxazolyl, oxadiazolyl, isothiazolyl, thiazolyl, pyridinyl, pyridazinyl, pyrimidinyl, pyrazinyl, indolyl, isoindolyl, quinolinyl and isoquinolinyl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
optionally, R³ is selected from the group consisting of hydrogen, fluorine, bromine, chlorine, 1,3,2-dioxaborolan-2-yl, methyl, ethyl, trifluoromethyl, difluoromethyl, 2-methoxyethyl, 1-methoxyethyl, methoxymethyl; C₃-C₇-cycloalkyl selected from cyclopropyl, cyclobutyl, cyclopentyl and cyclohexyl; C₃-C₈-cycloalkenyl selected from cyclopentenyl and cyclohexenyl; aryl selected from phenyl and 2-naphthyl; heterocyclyl selected from the group consisting of piperidin-1-yl, piperazin-1-yl, tetrahydro-2H-pyran-4-yl, tetrahydrothiopyran-4-yl, morpholin-4-yl and 1,3-dihydro-2H-isoindol-2-yl; heteroaryl selected from the group consisting of 2-furyl (2-furanyl), 2-thienyl, 3-thienyl, 1H-pyrazol-5-yl, 1H-pyrazol-1-yl, 1H-imidazol-1-yl, 1H-1,2,3-triazol-1-yl, 1,2-oxazol-4-yl, 1,3,4-oxadiazol-2-yl, 1,2,4-oxadiazol-5-yl, 1,3-thiazol-4-yl, pyridin-2-yl, pyridin-3-yl, pyridin-4-yl, pyrimidin-2-yl and quinoline-2-yl; biphenyl, phenoxyphenyl and phenoxy;
and wherein said C₃-C₇-cycloalkyl, C₃-C₈-cycloalkenyl, aryl, heterocyclyl, heteroaryl, biphenyl, phenoxyphenyl and phenoxy may be substituted with one to three R^{3b} substituents independently selected from the group consisting of fluorine, chlorine, bromine, nitro, cyano, nitro, methyl, ethyl, iso-propyl, n-propyl, n-butyl, iso-butyl, tert-butyl, cyclopropyl, trifluoromethyl, difluoromethyl, methoxy, ethoxy, trifluoromethoxy, difluoromethoxy, methoxycarbonyl, ethoxycarbonyl and tert-butoxycarbonyl;
R⁴ and R⁵ are independently selected from the group consisting of hydrogen, hydroxy, C₁-C₄-alkyl, C₁-C₄-haloalkyl, C₁-C₄-alkoxy, C₁-C₄-haloalkoxy, C₃-C₄-alkynyl, C₁-C₄-alkylcarbonyl, C₁-C₄-halogenoalkyl-carbonyl and phenylcarbonyl, wherein said phenylcarbonyl may be substituted with one or two fluorine atoms, optionally R⁴ and R⁵ are independently selected from the group consisting of hydrogen, hydroxy, methyl, ethyl, trifluromethyl, difluoromethyl, 2,2-difluoroethyl, methoxy, ethoxy, prop-2-ynyl and phenylcarbonyl, wherein said phenylcarbonyl may be substituted with one or two fluorine atoms;
provided that A is not NR⁴ when m is 1 or 2 and W¹ and W² are N.

15. A composition comprising at least one compound of formula (I) according to claim 1 and at least one agriculturally acceptable auxiliary.

16. A method for controlling phytopathogenic fungi which comprises applying at least one compound according to claim 12 or a composition thereof to one or more plants, plant parts, seeds, fruits and/or to soil in which plants grow.

17. The method according to claim 16, wherein the phytopathogenic fungi are selected from the group consisting of the Puccinia species, the Uromyces species and the rust disease pathogens.
